(19) United States
(12) Patent Application Publication
(10) Pub. No.: US 2022/0380383 A1
(43) Pub. Date: Dec. 1, 2022

(54) NEUROPROTECTIVE PDI MODULATING SMALL MOLECULES AND METHODS OF USE THEREOF

(71) Applicant: THE TRUSTEES OF COLUMBIA UNIVERSITY IN THE CITY OF NEW YORK, New York, NY (US)

(72) Inventors: Brent R. Stockwell, New York, NY (US); Arie Zask, New York, NY (US); Anna Kaplan, New York, NY (US)

(21) Appl. No.: 17/332,058

(22) Filed: May 27, 2021

Related U.S. Application Data
(63) Continuation of application No. PCT/US2019/064024, filed on Dec. 2, 2019.
(60) Provisional application No. 62/842,038, filed on May 2, 2019.
(60) Provisional application No. 62/773,278, filed on Nov. 30, 2018.
(60) Provisional application No. 62/773,288, filed on Nov. 30, 2018.

(51) Int. Cl. C07D 513/04; A61K 45/06; A61P 43/00; A61P 25/28; A61K 31/496; A61K 31/437; A61K 31/5025; A61K 31/5377; A61K 31/4545

(52) U.S. Cl. CPC C07D 513/04; A61K 45/06; A61P 43/00; A61P 25/28; A61K 31/4545; A61K 31/437; A61K 31/5025; A61K 31/5377; A61K 31/496

(57) ABSTRACT

The present disclosure provides, inter alia, compounds and compositions having the formula (I):

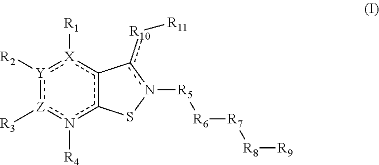

as defined herein. Methods of using and making such compounds and compositions are also provided. The present disclosure further provides screening methods, including detectable probes as well as diagnostic methods and methods for monitoring the progress of a disease, such as a neurodegenerative disease.

### CROSS REFERENCE TO RELATED APPLICATIONS

The present application is a continuation of PCT international application no. PCT/US2019/064024, filed on Dec. 2, 2019, which claims benefit of U.S. Provisional Patent Application Ser. No. 62/842,038, filed on May 2, 2019, U.S. Provisional Patent Application Ser. No. 62/773,266, filed on Nov. 30, 2018, U.S. Provisional Patent Application Ser. No. 62/773,278, filed on Nov. 30, 2018, and U.S. Provisional Patent Application Ser. No. 62/773,288, filed on Nov. 30, 2018, which applications are incorporated by reference herein in their entireties.

### FIELD OF THE DISCLOSURE

The present disclosure relates to modulators of protein disulfide isomerase (PDI). More particularly, the present disclosure provides small molecule inhibitors of PDI which are neuroprotective. The present disclosure also relates to methods for identifying a compound that targets a disease-related reactive oxygen species (ROS) regulator. The present disclosure further relates to methods for diagnosing a disease such as Alzheimer's disease in a subject in need thereof using a detectable probe. Also provided are methods for monitoring the progress of such disease.

### INCORPORATION BY REFERENCE OF SEQUENCE LISTING

This application contains references to amino acids and/or nucleic acid sequences that have been filed concurrently herewith as sequence listing text file “CU19227-seq.txt”, file size of 5 KB, created on Nov. 27, 2019. The aforementioned sequence listing is hereby incorporated by reference in its entirety pursuant to 37 C.F.R. § 1.52(e)(5).

### BACKGROUND OF THE DISCLOSURE

Neurodegenerative disorders constitute a class of diseases that express characteristic misfolded proteins that aggregate and induce neuronal toxicity and death. Huntington's disease (HD) is one such fatal protein misfolding disease that afflicts primarily medium spiny neurons in the striatum. HD is caused by expansion to more than 36 CAG trinucleotide repeats in the huntingtin gene. These CAG repeats translate into an expanded polyglutamine tract in the huntingtin protein, causing it to aggregate, and drive neuronal dysfunction and progressive neuronal loss. Currently there is no therapeutic avenue that can delay or stop the progression of the disease. In this context, there is a need to develop therapeutics and drug targets that can prevent or delay pathogenesis in neurodegenerative diseases, such as HD, involving protein misfolding.

Previously, it was reported that modulation of protein disulfide isomerase (PDI) by small molecules is beneficial in cell and brain slice models of HD (Hoffstrom et al., 2010). PDI is a thiol-oxidoreductase chaperone protein that is responsible for the isomerization, reduction, and oxidation of non-native disulfide bonds in unfolded proteins entering the endoplasmic reticulum (ER). Structurally, PDI consists of four domains with a thioredoxin fold: a, b, b′ and a′, an extended C-terminus with KDEL ER retention sequence, and an interdomain linker x between the b′ and a′ domains. The a and a′ domains are catalytically active, contain the WCGHC active site and independently can perform oxidation and reduction reactions (Darby & Creighton, 1995). However, all four domains are needed to achieve the isomerization and chaperone activity of PDI. Besides its catalytic role involving thiols and disulfides, PDI also serves an essential structural role as the beta subunit of prolyl-4-hydroxylase (Koivu et al., 1987) and as a microsomal triglyceride transfer protein (Wetterau et al., 1990).

PDI is upregulated in mouse models of, and in brains of patients with, neurological protein folding diseases (Yoo et al., 2002; Colla et al., 2012; Atkin et al., 2008). In addition, it has also been implicated in a number of cancers (Xu et al., 2012; Hashida et al., 2011; Lovat et al., 2008), HIV-1 pathogenesis (Barbouche et al., 2003), and blood clot formation (Cho et al., 2008), suggesting the growing importance of understanding this enzyme. One challenge has been the lack of available drug-like inhibitors, especially for in vivo evaluation in neurodegenerative disease models. Reported inhibitors of PDI are either (i) irreversible binders to the catalytic site cysteines (Hoffstrom et al., 2010; Xu et al., 2012; Ge et al., 2013), (ii) not cell permeable, because they were designed for the inhibition of extracellular PDI (Jasuja et al., 2012; Khan et al., 2011) or (iii) nonselective hormones and antibiotics, such as estrone and bacitracin, that act broadly on multiple target proteins (Khan et al., 2011; Karala & Ruddock, 2010). Irreversible inhibitors, although having promise in ovarian cancer, have mechanism-based toxicity that is not likely well tolerated in neurons. PDI is an essential protein, whose irreversible genetic silencing is cytotoxic to cells and probably in animal models as well, since no genetic PDI null has been generated. The related PDI A3 (ERp57) protein knockout resulted in embryonic lethality in mice (Garbi et al., 2006). Thus, irreversible inhibitors of PDI may exhibit the same level of cytotoxicity in vivo. It was hypothesized that reversible, non-covalent inhibitors of PDI might exhibit a therapeutic window upon PDI inhibition, and would have improved pharmaceutical properties. Some aspects of the present disclosure are directed towards these and other needs.

Reactive oxygen species (ROS) are oxygen-containing molecules which are primarily formed as a result of incomplete oxygen reduction. It has been shown that an imbalance in ROS production versus opposing antioxidant detoxification can lead to disruptions in cellular redox homeostasis. An increase in global ROS concentrations promotes the destructive interaction of these species with important cell functional components in a process leading to oxidative stress.

Accumulating data suggests that oxidative stress is involved in the pathogenesis of neurodegenerative diseases, and that antioxidant administration may be useful in the prevention and treatment of neurodegenerative diseases. To obtain efficacy in delaying disease progression, the candidate antioxidant must be given as early as possible, before irreversible neuronal loss. It is believed to be promising to target precise oxidative stress physiology by identifying the exact type of ROS involved in specific neurodegenerative diseases. Some aspects of the present disclosure are directed towards these and other needs.

PDIs also perform important physiological functions in protein quality control, cell death, and cell signaling. Protein misfolding is involved in the etiology of the most common neurodegenerative diseases, including Alzheimer's disease, Huntington's disease, Parkinson's disease, amyotrophic lateral sclerosis, and prion-related disorders, among others. Accumulating evidence indicate that altered expression of PDIs is a prominent and common feature of these neurodegenerative conditions. Some aspects of the present disclosure are directed to the therapeutic benefits of targeting PDIs in a disease context and their use as biomarkers.

### SUMMARY OF THE DISCLOSURE

In the present disclosure, there is provided a neuroprotective, reversible modulator of PDI that has nanomolar potency, high in vitro stability in liver microsomes and blood plasma, and is protective for medium spiny neurons in a brain slice model for HD. This scaffold represents a class of reversible modulators of PDI that can probe its potential as a drug target for neurological diseases with misfolded proteins.

The present disclosure provides a compound having the formula (I):

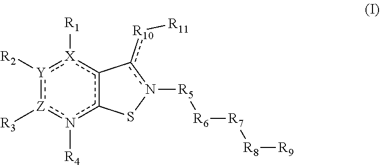

wherein a dashed line indicates the presence of an optional double bond, wherein X, Y and Z are independently selected from the group consisting of C, N, S and O, wherein R1, R2, R3, and R4 are independently selected from the group consisting of no atom, H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
with the proviso that the compound is not

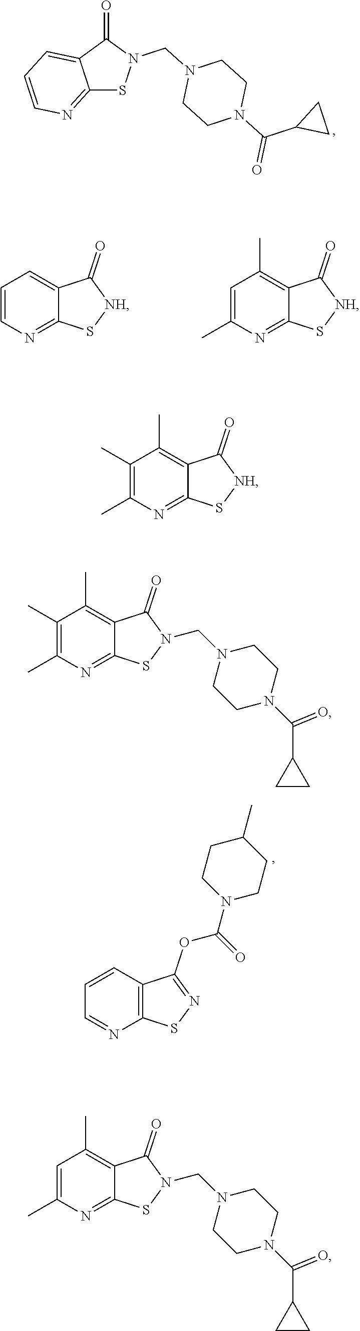

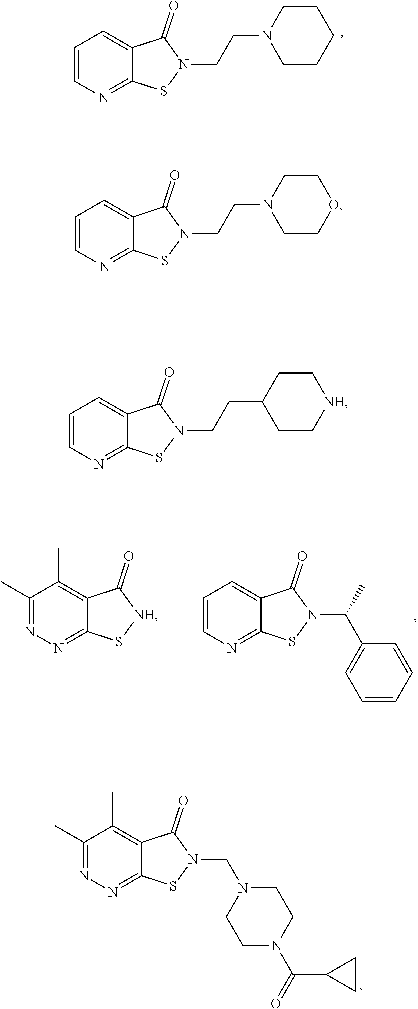 [SMILES: O=c1c2cccnc2sn1CCN1CCCCC1 ; O=c1c2cccnc2sn1CCN1CCOCC1 ; O=c1c2cccnc2sn1CCC1CCNCC1 ; Cc1nnc2s[nH]c(=O)c2c1C ; C[C@H](c1ccccc1)n1sc2ncccc2c1=O ; Cc1nnc2sn(CN3CCN(C(=O)C4CC4)CC3)c(=O)c2c1C]

or an N-oxide, crystalline form, hydrate thereof, or a pharmaceutically acceptable salt thereof.

The present disclosure also provides a compound having the formula (II):

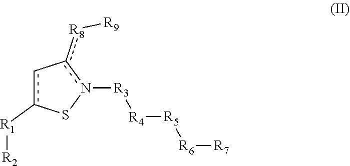

wherein a dashed line indicates the presence of an optional double bond, wherein R₁ is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, wherein R₃, R₄, R₅, R₆ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₇ and R₉ are independently selected from the group consisting of H, NR, N(R)₂, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof.

The present disclosure also provides a compound selected from the group consisting of:

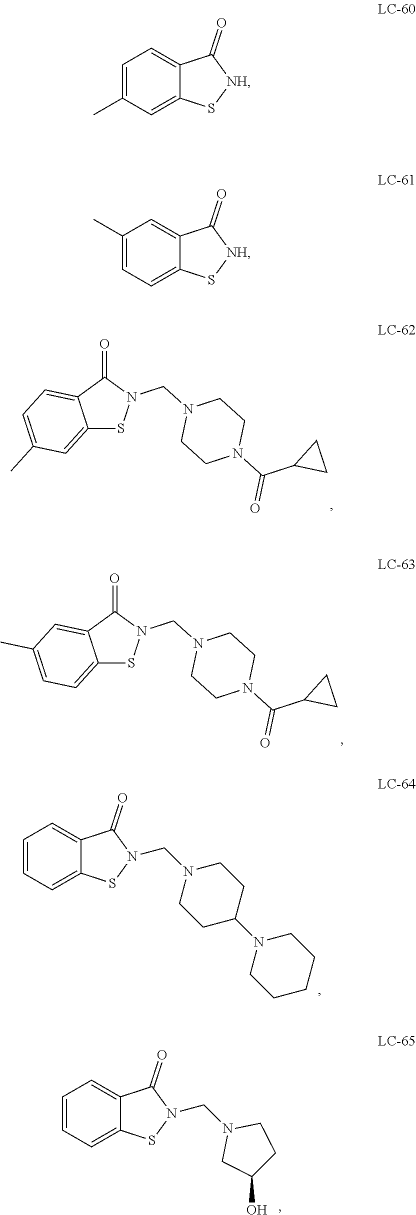 [SMILES: Cc1ccc2c(=O)[nH]sc2c1 ; Cc1ccc2s[nH]c(=O)c2c1 ; Cc1ccc2c(=O)n(CN3CCN(C(=O)C4CC4)CC3)sc2c1 ; Cc1ccc2sn(CN3CCN(C(=O)C4CC4)CC3)c(=O)c2c1 ; O=c1c2ccccc2sn1CN1CCC(N2CCCCC2)CC1 ; O=c1c2ccccc2sn1CN1CC[C@@H](O)C1]

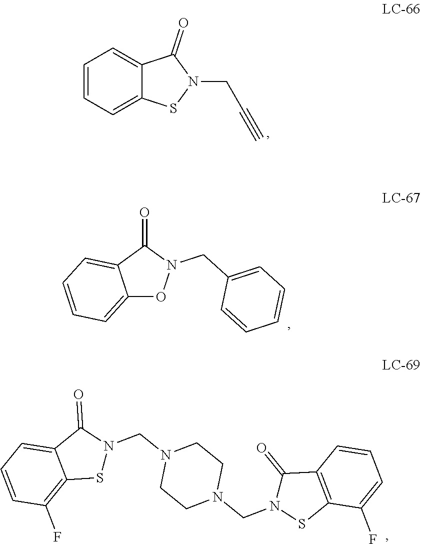 [SMILES: C#CCn1sc2ccccc2c1=O ; O=c1c2ccccc2on1Cc1ccccc1 ; O=c1c2cccc(F)c2sn1CN1CCN(Cn2sc3c(F)cccc3c2=O)CC1]

and combinations thereof,
or an N-oxide, crystalline form, hydrate thereof, or a pharmaceutically acceptable salt thereof.

The present disclosure also provides a composition comprising a compound of the present disclosure and a pharmaceutically acceptable carrier, adjuvant or vehicle.

The present disclosure also provides a pharmaceutically acceptable salt of a compound of the present disclosure.

The present disclosure also provides a composition comprising a pharmaceutically acceptable salt of the present disclosure and a pharmaceutically acceptable carrier, adjuvant or vehicle.

The present disclosure also provides a kit comprising a compound or composition of the present disclosure and instructions for use.

The present disclosure also provides a method for treating or ameliorating the effects of a neurodegenerative disease in a subject in need thereof comprising administering to the subject an effective amount of a compound selected from the group consisting of formula (I)

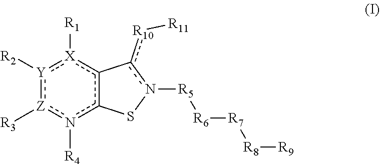

wherein a dashed line indicates the presence of an optional double bond,
wherein X, Y and Z are independently selected from the group consisting of C, N, S and O,
wherein R₁, R₂, R₃, and R₄ are independently selected from the group consisting of no atom, H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, with the proviso that the compound is not

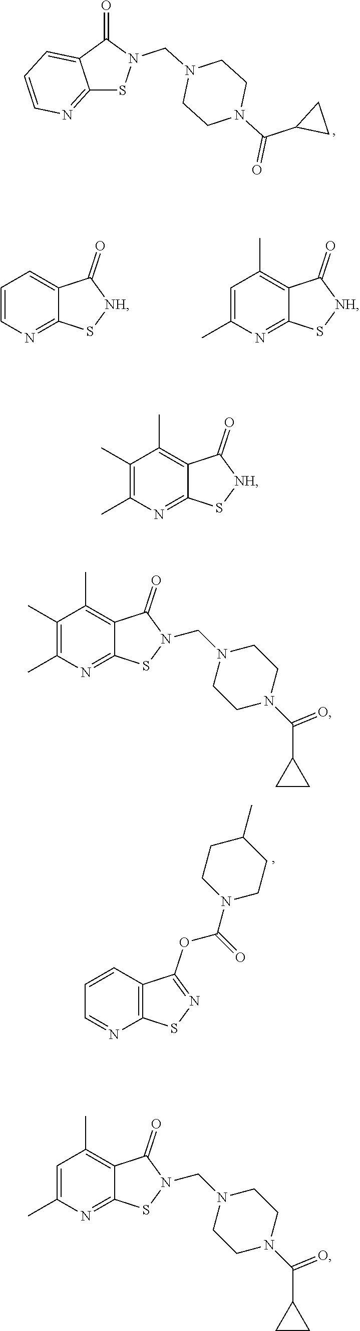

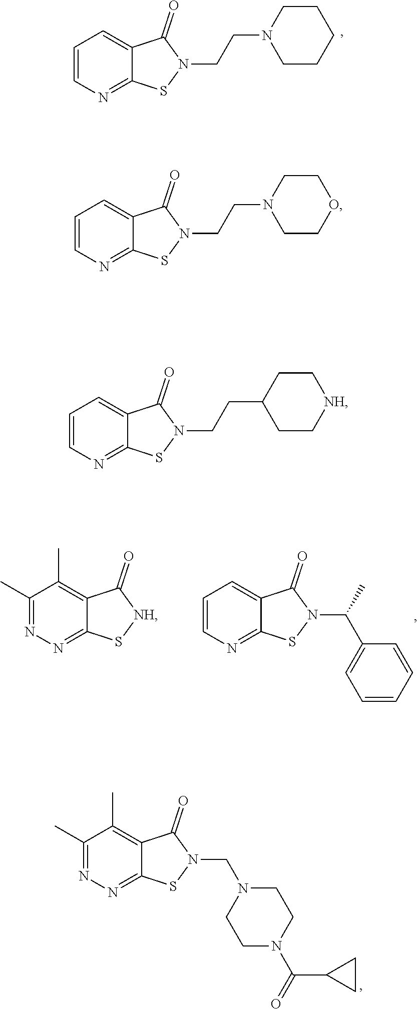 [SMILES: O=c1c2cccnc2sn1CCN1CCCCC1 ; O=c1c2cccnc2sn1CCN1CCOCC1 ; O=c1c2cccnc2sn1CCC1CCNCC1 ; Cc1nnc2s[nH]c(=O)c2c1C ; C[C@H](c1ccccc1)n1sc2ncccc2c1=O ; Cc1nnc2sn(CN3CCN(C(=O)C4CC4)CC3)c(=O)c2c1C]

or an N-oxide, crystalline form, hydrate thereof, or a pharmaceutically acceptable salt thereof; formula (II)

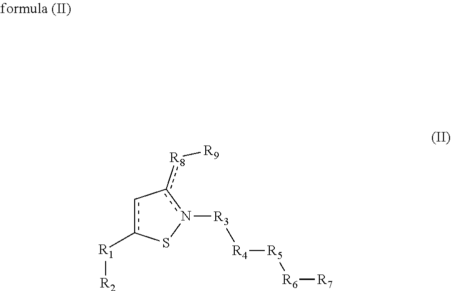

wherein a dashed line indicates the presence of an optional double bond,
wherein R₁ is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₃, R₄, R₅, R₆ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₇ and R₉ are independently selected from the group consisting of H, NR, N(R)₂, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof,
a compound selected from the group consisting of:

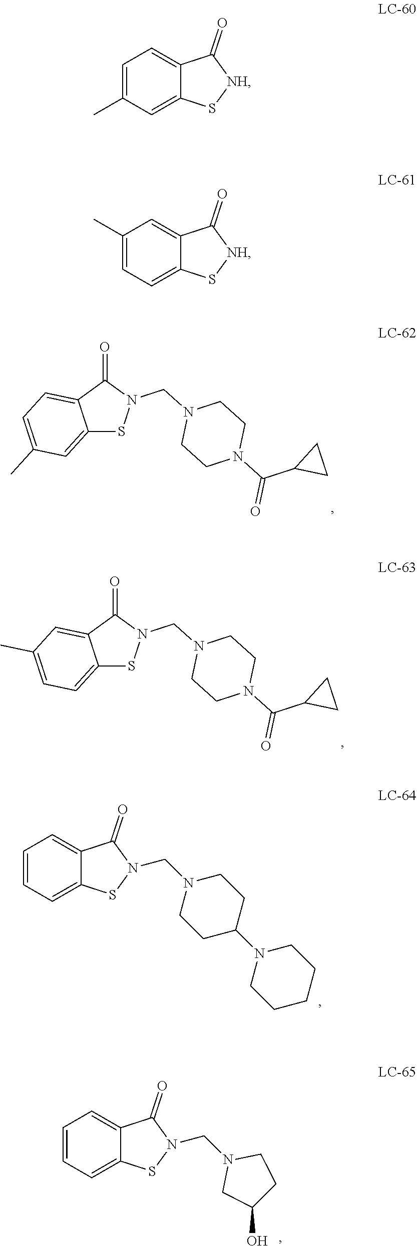 [SMILES: Cc1ccc2c(=O)[nH]sc2c1 ; Cc1ccc2s[nH]c(=O)c2c1 ; Cc1ccc2c(=O)n(CN3CCN(C(=O)C4CC4)CC3)sc2c1 ; Cc1ccc2sn(CN3CCN(C(=O)C4CC4)CC3)c(=O)c2c1 ; O=c1c2ccccc2sn1CN1CCC(N2CCCCC2)CC1 ; O=c1c2ccccc2sn1CN1CC[C@@H](O)C1]

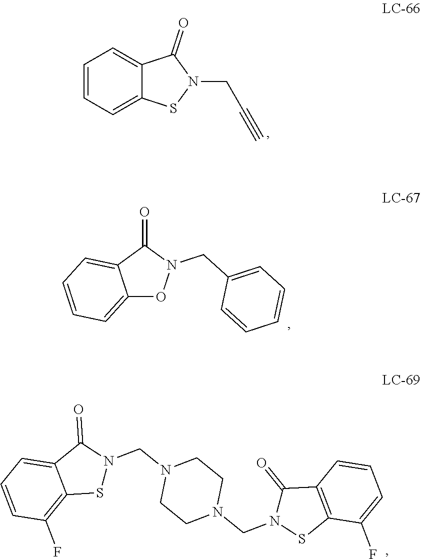 [SMILES: C#CCn1sc2ccccc2c1=O ; O=c1c2ccccc2on1Cc1ccccc1 ; O=c1c2cccc(F)c2sn1CN1CCN(Cn2sc3c(F)cccc3c2=O)CC1]

and combinations thereof,
or an N-oxide, crystalline form, hydrate thereof, or a pharmaceutically acceptable salt thereof.

The present disclosure also provides a method for treating or ameliorating the effects of a neurodegenerative disorder in a subject in need thereof comprising administering to the subject an effective amount of a composition of the disclosure.

The present disclosure also provides a method for treating or ameliorating the effects of a condition associated with increased protein disulfide isomerase (PDI) activity in a subject in need thereof comprising administering to the subject an effective amount of a compound selected from the group consisting of formula (I)

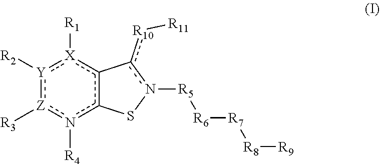

wherein a dashed line indicates the presence of an optional double bond,
wherein X, Y and Z are independently selected from the group consisting of C, N, S and O,
wherein R₁, R₂, R₃, and R₄ are independently selected from the group consisting of no atom, H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
with the proviso that the compound is not

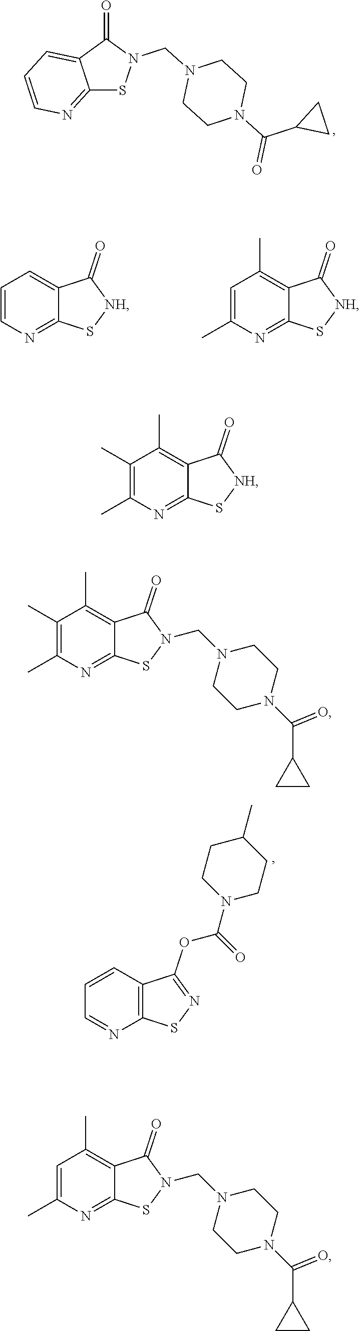

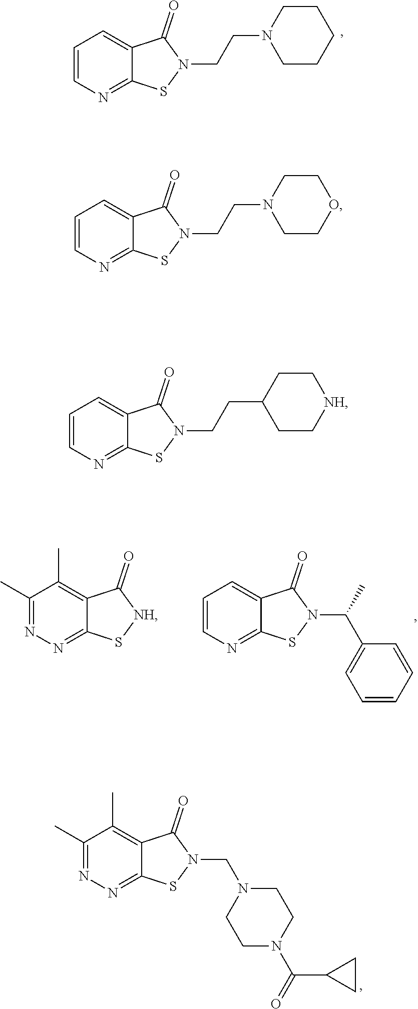 [SMILES: O=c1c2cccnc2sn1CCN1CCCCC1 ; O=c1c2cccnc2sn1CCN1CCOCC1 ; O=c1c2cccnc2sn1CCC1CCNCC1 ; Cc1nnc2s[nH]c(=O)c2c1C ; C[C@H](c1ccccc1)n1sc2ncccc2c1=O ; Cc1nnc2sn(CN3CCN(C(=O)C4CC4)CC3)c(=O)c2c1C]

or an N-oxide, crystalline form, hydrate thereof, or a pharmaceutically acceptable salt thereof,

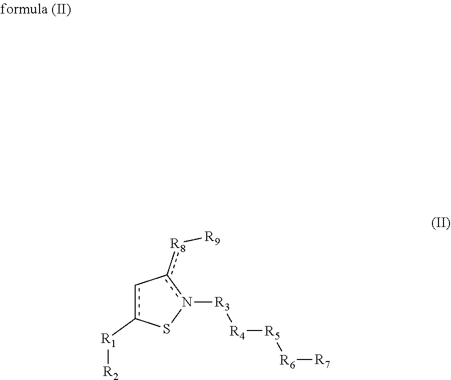

wherein a dashed line indicates the presence of an optional double bond,
wherein R₁ is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₃, R₄, R₅, R₆ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₇ and R₉ are independently selected from the group consisting of H, NR, N(R)₂, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, or an N-oxide, crystalline form, hydrate thereof, or a pharmaceutically acceptable salt thereof;
a compound selected from the group consisting of:

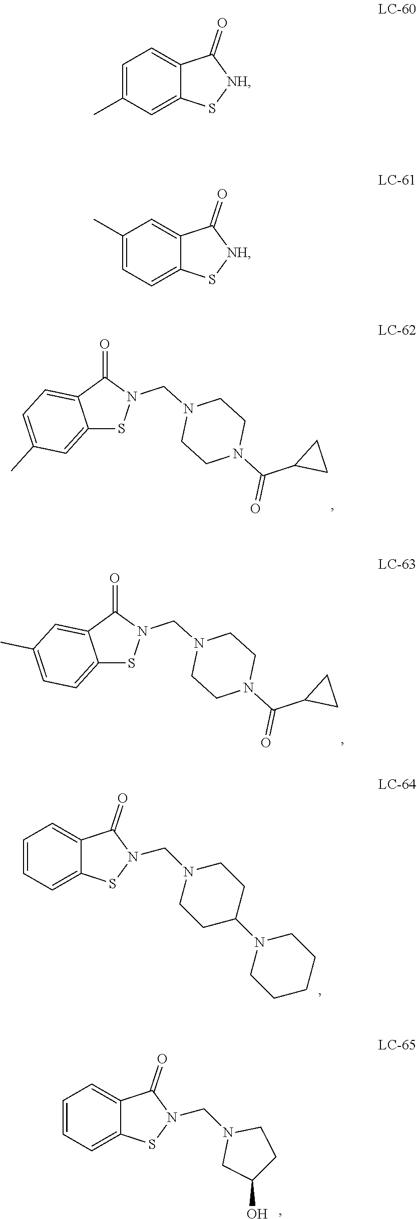 [SMILES: Cc1ccc2c(=O)[nH]sc2c1 ; Cc1ccc2s[nH]c(=O)c2c1 ; Cc1ccc2c(=O)n(CN3CCN(C(=O)C4CC4)CC3)sc2c1 ; Cc1ccc2sn(CN3CCN(C(=O)C4CC4)CC3)c(=O)c2c1 ; O=c1c2ccccc2sn1CN1CCC(N2CCCCC2)CC1 ; O=c1c2ccccc2sn1CN1CC[C@@H](O)C1]

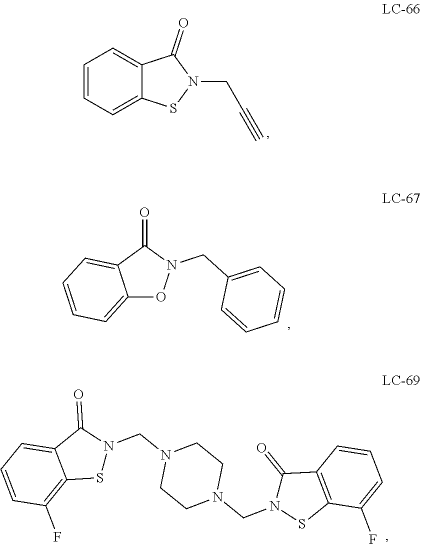 [SMILES: C#CCn1sc2ccccc2c1=O ; O=c1c2ccccc2on1Cc1ccccc1 ; O=c1c2cccc(F)c2sn1CN1CCN(Cn2sc3c(F)cccc3c2=O)CC1]

and combinations thereof,
or an N-oxide, crystalline form, hydrate thereof, or a pharmaceutically acceptable salt thereof.

The present disclosure also provides a method for treating or ameliorating the effects of a condition associated with increased protein disulfide isomerase (PDI) activity in a subject in need thereof comprising administering to the subject an effective amount of a composition of the present disclosure.

The present disclosure also provides a method of modulating PDI activity in a cell comprising administering to the subject an effective amount of a compound selected from the group consisting of formula (I)

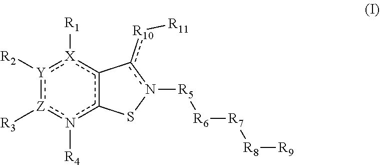

wherein a dashed line indicates the presence of an optional double bond,
wherein X, Y and Z are independently selected from the group consisting of C, N, S and O,
wherein R₁, R₂, R₃, and R₄ are independently selected from the group consisting of no atom, H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, with the proviso that the compound is not

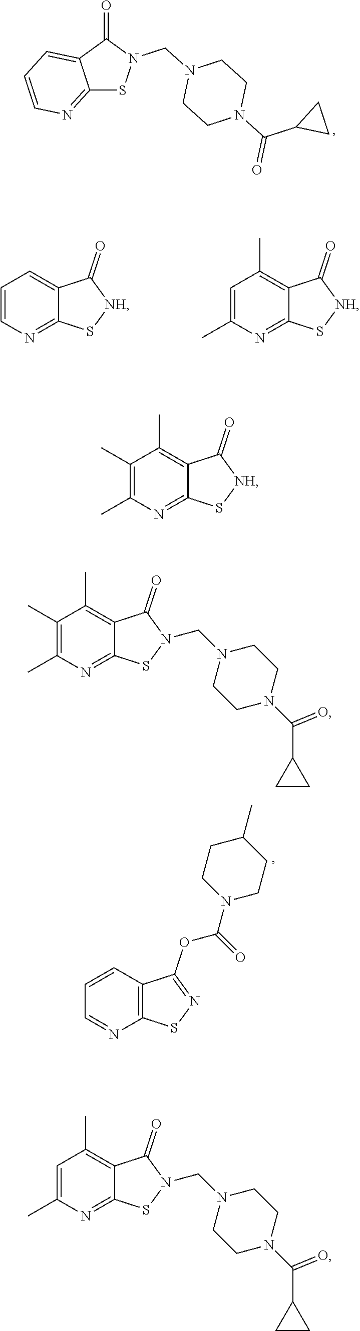

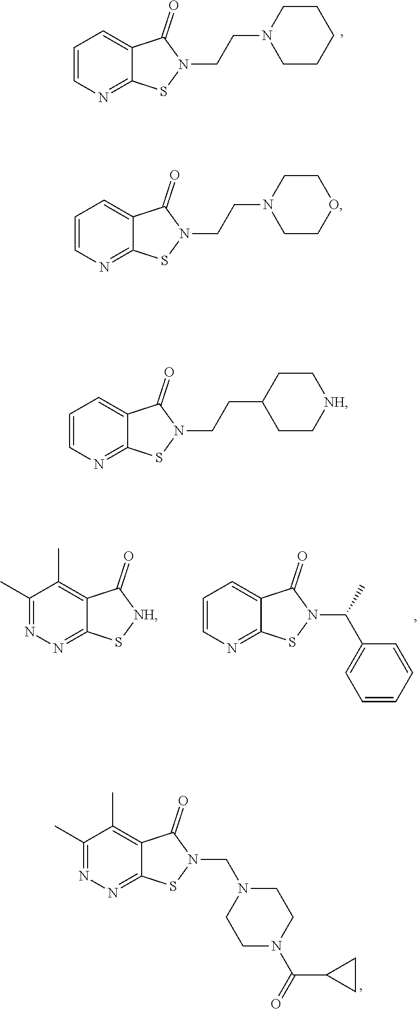 [SMILES: O=c1c2cccnc2sn1CCN1CCCCC1 ; O=c1c2cccnc2sn1CCN1CCOCC1 ; O=c1c2cccnc2sn1CCC1CCNCC1 ; Cc1nnc2s[nH]c(=O)c2c1C ; C[C@H](c1ccccc1)n1sc2ncccc2c1=O ; Cc1nnc2sn(CN3CCN(C(=O)C4CC4)CC3)c(=O)c2c1C]

or an N-oxide, crystalline form, hydrate thereof, or a pharmaceutically acceptable salt thereof;

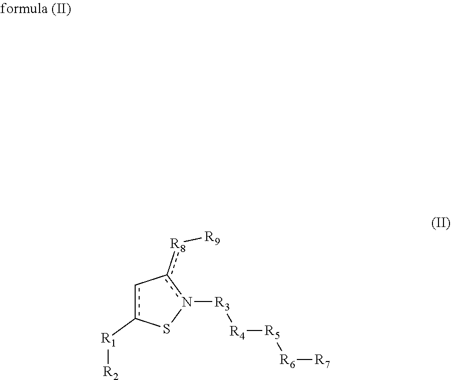

wherein a dashed line indicates the presence of an optional double bond,
wherein R₁ is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₃, R₄, R₅, R₆ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₇ and R₉ are independently selected from the group consisting of H, NR, N(R)₂, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, or an N-oxide, crystalline form, hydrate thereof, or a pharmaceutically acceptable salt thereof;
a compound selected from the group consisting of:

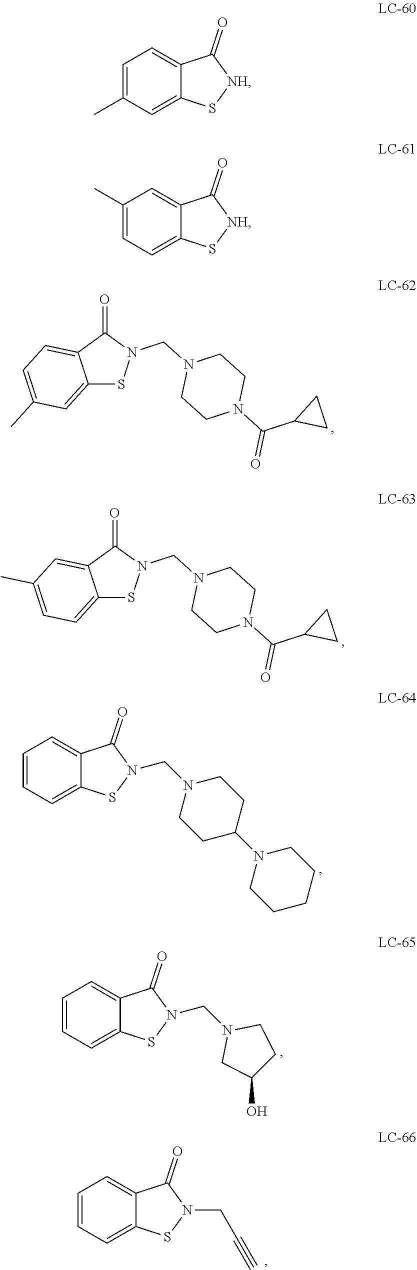

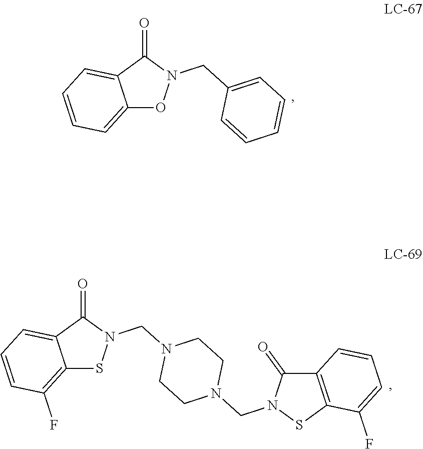 [SMILES: O=c1c2ccccc2on1Cc1ccccc1 ; O=c1c2cccc(F)c2sn1CN1CCN(Cn2sc3c(F)cccc3c2=O)CC1]

and combinations thereof,
or an N-oxide, crystalline form, hydrate thereof, or a pharmaceutically acceptable salt thereof.

The present disclosure also provides a method of modulating PDI activity in a cell comprising administering to the subject an effective amount of a composition of the present disclosure.

The present disclosure also provides a compound selected from the group consisting of:

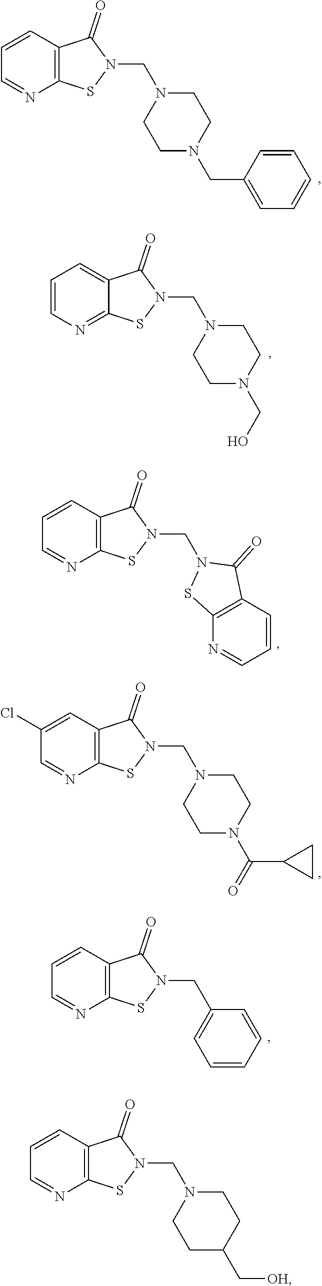 [SMILES: O=c1c2cccnc2sn1CN1CCN(Cc2ccccc2)CC1 ; O=c1c2cccnc2sn1CN1CCN(CO)CC1 ; O=c1c2cccnc2sn1Cn1sc2ncccc2c1=O ; O=C(C1CC1)N1CCN(Cn2sc3ncc(Cl)cc3c2=O)CC1 ; O=c1c2cccnc2sn1Cc1ccccc1 ; O=c1c2cccnc2sn1CN1CCC(CO)CC1]

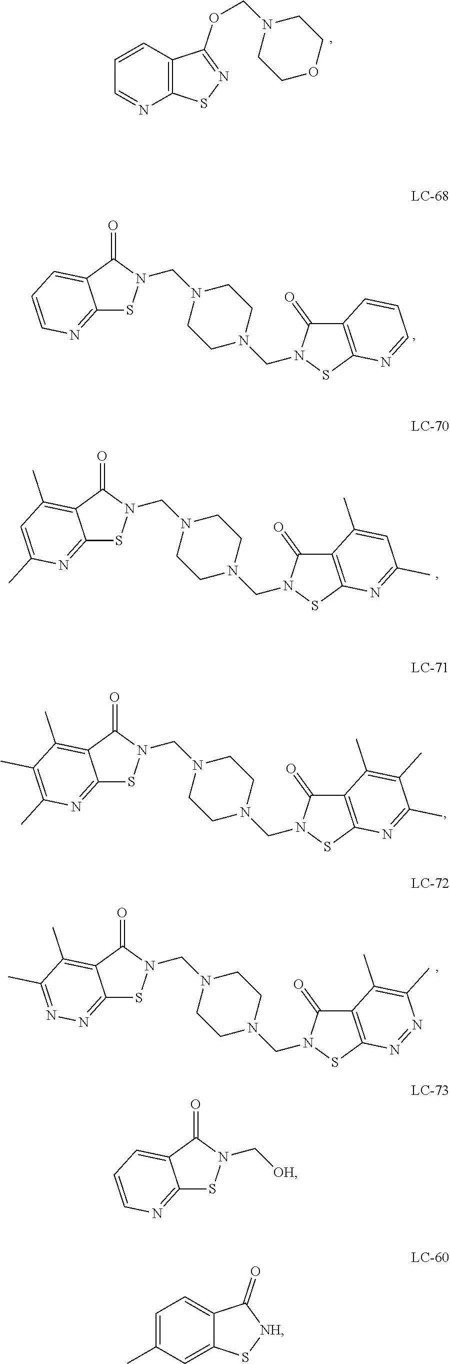

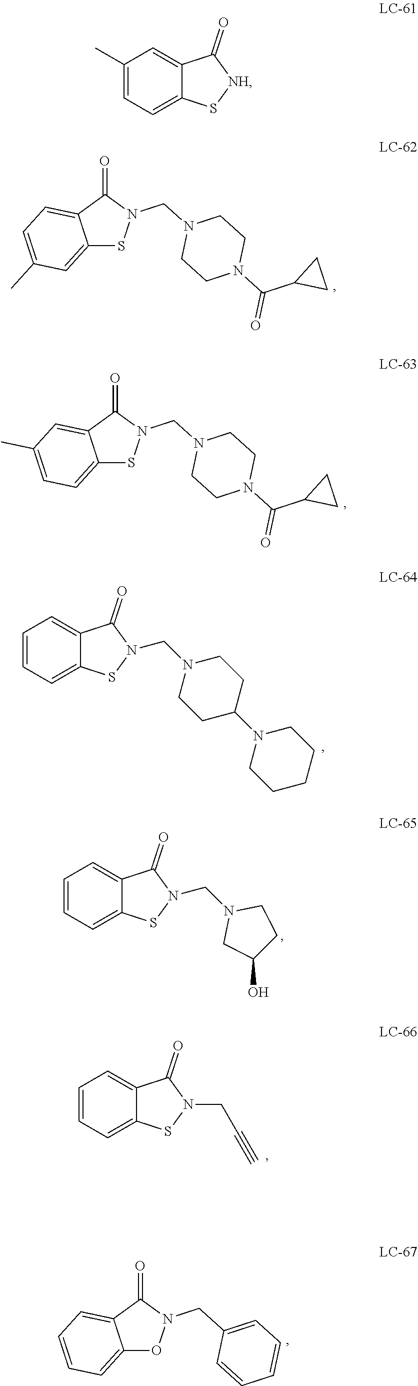

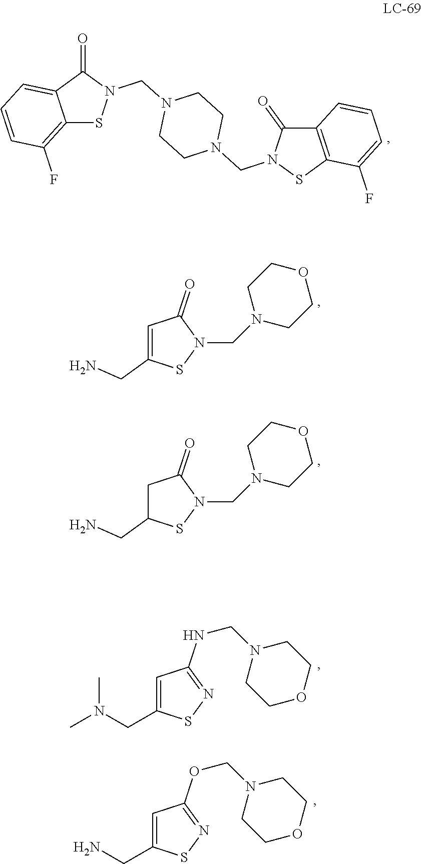 [SMILES: O=c1c2cccc(F)c2sn1CN1CCN(Cn2sc3c(F)cccc3c2=O)CC1 ; NCc1cc(=O)n(CN2CCOCC2)s1 ; NCC1CC(=O)N(CN2CCOCC2)S1 ; CN(C)Cc1cc(NCN2CCOCC2)ns1 ; NCc1cc(OCN2CCOCC2)ns1]

and combinations thereof,
or an N-oxide, crystalline form, hydrate thereof, or a pharmaceutically acceptable salt thereof.

The present disclosure also provides a compound having the structure of:

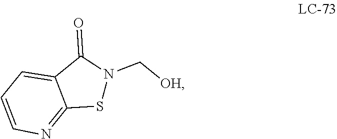 [SMILES: O=c1c2cccnc2sn1CO]

or an N-oxide, crystalline form, hydrate thereof, or a pharmaceutically acceptable salt thereof.

The present disclosure further provides compositions that include any of the foregoing compounds individually or in any combination, and a pharmaceutically acceptable carrier, adjuvant or vehicle.

Moreover, in the present disclosure, a chemistry platform is provided for identifying ROS regulators that are critical in specific diseases, and to further screen for compounds targeting these ROS regulators.

The present disclosure provides a method for identifying a compound that targets a disease-related reactive oxygen species (ROS) regulator. This method comprises:
(a) providing siRNA, gRNA, and/or cDNA libraries that target a plurality of ROS regulators;
(b) screening the libraries assembled in (a) for candidate ROS regulators that are associated with a disease;
(c) identifying desired ROS regulator(s) by validating candidate ROS regulators in cell assays specific for the disease;
(d) assembling a compound library based on a plurality of redox regulating scaffolds;
(e) screening the library assembled in (d) for candidate scaffolds that modulate the ROS regulator(s) identified in (c);
(f) identifying the desired scaffold(s) by evaluating safety and selectivity of the candidate scaffolds; and
(g) identifying a candidate compound that targets the disease-related ROS regulator based on the compound's ability to bind to the desired scaffold identified in (f).

The present disclosure also provides a method for treating or ameliorating the effects of a disease in a subject in need thereof. This method comprises administering to the subject an effective amount of a compound identified by the methods disclosed herein.

The present disclosure also provides compounds identified based on selective binding to the desired scaffold identified by the methods disclosed herein.

The present disclosure further provides compositions that include any of the foregoing compounds individually or in combination, and a pharmaceutically acceptable carrier, adjuvant or vehicle.

Furthermore, the present disclosure provides a detectable probe for measuring protein disulfide isomerase (PDI) abundance and target engagement in vitro and in vivo. The detectable probe comprises a probe portion that targets a protein disulfide isomerase (PDI), and a detectable label portion.

The present disclosure also provides a method of patient selection. This method comprises:
(a) administering to a subject with a neurodegenerative disease a detectable probe disclosed herein;
(b) determining the distribution of the protein disulfide isomerase (PDI) in the subject;
(c) comparing the distribution determined in step (b) with a reference distribution determined in a healthy subject; and
(d) selecting the subject for appropriate treatment based on the comparison result obtained in step (c).

The present disclosure also provides a method for measuring protein disulfide isomerase (PDI) target engagement of a compound in a subject. This method comprises:
(a) administering the compound to the subject;
(b) administering to the subject a detectable probe disclosed herein; and
(c) measuring the PDI target engagement of the compound.

The present disclosure also provides a method for determining efficacy of a compound in treating a neurodegenerative disease in a subject. This method comprises:
(a) obtaining a biological sample from the subject;
(b) contacting the sample with a detectable probe disclosed herein;
(c) determining the abundance of the PDI in the subject;
(d) administering the compound to the subject;
(e) after a certain period of time, repeating steps (a) to (c) to obtain an updated abundance of the PDI in the subject;
(f) if the abundance of the PDI determined in step (e) is significantly higher than the abundance of the PDI determined in step (c), the disease in the subject is progressing and the compound is not effective; and
(g) adjusting the treatment protocol for the subject diagnosed with progressing disease.

The present disclosure also provides a method for diagnosing a disease in a subject in need thereof. This method comprises:
(a) administering to the subject a detectable probe that targets a reactive oxygen species (ROS) regulator, which is associated with the disease;
(b) determining the distribution of the ROS regulator in the subject; and
(c) if the distribution determined in step (b) is significantly different from a reference distribution determined in a healthy subject, the subject is diagnosed with the disease.

The present disclosure also provides a method for monitoring the progress of a disease in a subject in need thereof. This method comprises:
(a) obtaining a biological sample from the subject;
(b) contacting the sample with a detectable probe that targets a reactive oxygen species (ROS) regulator, which is associated with the disease;
(c) determining the abundance of the ROS regulator in the subject;
(d) after a certain period of time, repeating steps (a) to (c) to obtain an updated abundance of the ROS regulator in the subject;
(e) if the abundance of the ROS regulator determined in step (d) is significantly higher than the abundance of the ROS regulator determined in step (c), the disease in the subject is progressing; and
(f) initiating a treatment protocol for the subject diagnosed with a progressing disease.

The present disclosure also provides a method for diagnosing Alzheimer's disease in a subject in need thereof. This method comprises:
(a) administering to the subject a detectable probe that targets a protein disulfide isomerase (PDI), which is associated with Alzheimer's disease;
(b) determining the distribution of the PDI in the subject by positron emission tomography (PET); and
(c) if the distribution determined in step (b) is significantly different from a reference distribution determined in a healthy subject, the subject is diagnosed with Alzheimer's disease.

The present disclosure also provides a method for monitoring the progress of Alzheimer's disease in a subject in need thereof. This method comprises:
(a) obtaining a biological sample from the subject;
(b) contacting the sample with a detectable probe that targets a protein disulfide isomerase (PDI), which is associated with Alzheimer's disease;
(c) determining the abundance of the PDI in the subject;
(d) after a certain period of time, repeating steps (a) to (c) to obtain an updated abundance of the PDI in the subject;
(e) if the abundance of the PDI determined in step (d) is significantly higher than the abundance of the PDI determined in step (c), Alzheimer's disease in the subject is progressing; and
(f) initiating a treatment protocol for the subject diagnosed with progressing Alzheimer's disease.

The present disclosure also provides a method for diagnosing Huntington's disease in a subject in need thereof. This method comprises:
(a) administering to the subject a detectable probe that targets a protein disulfide isomerase (PDI), which is associated with Huntington's disease;
(b) determining the distribution of the PDI in the subject by positron emission tomography (PET); and
(c) if the distribution determined in step (b) is significantly different from a reference distribution determined in a healthy subject, the subject is diagnosed with Huntington's disease.

The present disclosure also provides a method for monitoring the progress of Huntington's disease in a subject in need thereof. This method comprises:
(a) obtaining a biological sample from the subject;
(b) contacting the sample with a detectable probe that targets a protein disulfide isomerase (PDI), which is associated with Huntington's disease;
(c) determining the abundance of the PDI in the subject;
(d) after a certain period of time, repeating steps (a) to (c) to obtain an updated abundance of the PDI in the subject;
(e) if the abundance of the PDI determined in step (d) is significantly higher than the abundance of the PDI determined in step (c), Huntington's disease in the subject is progressing; and
(f) initiating a treatment protocol for the subject diagnosed with progressing Huntington's disease.

### BRIEF DESCRIPTION OF THE DRAWINGS

The application file contains at least one drawing executed in color. Copies of this patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1E show the results of LOC14 analogs binding to PDI A1a using isothermal titration calorimetry (ITC).

FIG. 1A shows abnormal ITC heat trace when PDI A1a was titrated into 0.8% NMP, as a control.

FIG. 1B shows no binding but normal ITC traces when PDI A1a was titrated into LC68 (in 0.8% NMP).

FIG. 1C shows abnormal ITC heat traces when PDI A1a was titrated into LC69 (in 0.8% NMP), indicating a Kd₁ of 700 nM.

FIG. 1D shows abnormal ITC heat traces when PDI A1a was titrated into LC70 (in 0.8% NMP), indicating a Kd₁ of 3.8 μM.

FIG. 1E shows abnormal ITC heat traces when PDI A1a was titrated into LC73 (in DMSO), indicating a Kd of 9.8 nM.

FIG. 2 shows the viability results of LOC14 and analogs LC68-LC73 in PC12 cells. Neuroprotection was assessed by each compound's ability to rescue cells from mutant-huntingtin-induced cell death (“Ind Q103” cells; blue). General cell toxicity of each compound was assessed in uninduced cells not expressing mutant-huntingtin protein (“Unind Q103” cells; red). NMP, DMSO, LOC14 in NMP and LOC14 in DMSO were used as controls.

FIG. 3 shows the results of a glutathione binding assay for several LOC14 analogs. LOC-14, selected analogs, and a positive control (NEM, Nethylmaleimide) were incubated with glutathione in a Tris-buffer (pH 8.5) and at selected time points, a solution of Ellman's reagent was added. Unreacted glutathione reacts with Ellman's reagent leading to the formation of 2-nitro-5-thiobenzoic acid (TNB), and its concentration can be measured spectrophotometrically.

FIG. 4 shows MALDI-TOF analysis indicating LOC14 covalently bound to PDI C39S mutant. Top, mass of PDI C39S mutant is shown as a peak at 15,405 Da. The second peak at 15,585 Da (+179 Da change) is the gluconoylation modification of PDI C39S at the His6× tag by E. coli. Middle, mass of PDI C39S treated with benzoisothiazolone. Bottom, mass of PDI C39S treated with LOC14. A shift of +152 Da to the C39S only peaks is observed in the treated samples corresponding to the bound benzoisothiazolone moiety. A peak for PDI C39S only is also observed in the compound treated samples, which corresponds to not fully saturated compound binding.

FIG. 5 shows ROS production in induced/uninduced PC12 cells to express mutant huntingtin protein with/without the rescue compound LOC14. Media section represents readouts without the cells to establish potential background; uninduced cells: cells not expressing mutant huntingtin and menadione was used as a positive control of ROS production; induced cells: cells expressing mutant huntingtin and subsequently treated with LOC14 at 3 μM and 5 μM.

FIG. 6 shows Cell RoX Deep Red confocal microscopy. Each row of panels show controls DMSO (vehicle), menadione (positive H₂O₂ production control), tebufenozide (eGFP expression) and tebufenozide with rescue compound LOC14 at 3 μM and 5 μM. Microscopy of cells was carried out using Zeiss LSM700 at room temperature. Fixed cells were imaged using excitation/emission: eGFP 375/540, CellRox Deep Red 644/665, DAPI 360/460. The final processing of images was performed using the ImageJ software.

FIG. 7 shows total ROS imaging using HyperRed, ER Blue Tracker and DAPI. Each row of panels show controls DMSO (vehicle), menadione (positive H₂O₂ production control), tebufenozide (eGFP expression) and tebufenozide with rescue compound LOC14 at 5 μM. Microscopy of cells was carried out using Zeiss LSM700 at room temperature. Fixed cells were imaged using excitation/emission: eGFP 375/540; Hyper Red BP 560/40, BP 645/75; DAPI 360/460; ER Blue Tracker: 374/430. The final processing of images was performed using the ImageJ software.

FIG. 8 shows total ROS imaging using HyperRed-Control pH, ER Blue Tracker and DAPI. Each row of panels show controls DMSO (vehicle), menadione (positive H₂O₂ production control), tebufenozide (eGFP expression) and tebufenozide with rescue compound LOC14 at 5 μM. Microscopy of cells was carried out using Zeiss LSM700 at room temperature. Fixed cells were imaged using excitation/emission: eGFP 375/540; Hyper Red BP 560/40, BP 645/75; DAPI 360/460; ER Blue Tracker: 374/430. The final processing of images was performed using the ImageJ software.

FIG. 9 shows PC12 gene expression after 24 h induction and treatment with rescue compound, where U—uninduced, I—induced with tebufenozided, I 3—induced and treated with 3 μM LOC14, I 5—induced and treated with 5 μM LOC14. Reference expression for PC12 cells: CyCa gene essential for the control of the cell cycle G1/M.

FIG. 10 shows Normalized MDA levels (μM) for cells treated with DMSO (uninduced), tebufenozide (induced) and tebufenozide+LOC14 5 μM.

FIGS. 11A-11C show that LOC14 binds to the catalytic cysteine 53 of PDI protein. FIG. 11A shows the chemical structures of LOC14 and active benzoisothiazolone (BIT) fragment. FIG. 11B shows the mechanism of trapping LOC14 on the mutant PDIa protein. FIG. 11C provides, Top, mass of PDIa C56S mutant is shown as a peak at 15,405 m/z. The second peak at 15,585 Da (+179 Da change) is the natural gluconoylation modification of PDIa C56S at the His6× tag by E. coli. Middle, mass of PDIa C56S treated with BIT (+152 Da change). Bottom, mass of PDIa C56S treated with LOC14. A shift of +152 is observed in the treated samples corresponding to the bound active fragment, BIT. A peak for PDIa C56S only is also observed in the compound treated samples, which corresponds to incomplete compound binding.

FIG. 11D shows that LOC14 binds covalently to PDIa C56S protein. PDIa C56S mutant was pre-treated with either LOC14, BIT, an irreversible modifier of cysteines, iodoacetamide (IAA), or DMSO vehicle for 30 min. Once the protein-inhibitor complexes were formed the samples were treated with 2.7 mM DTT and analyzed by MALDI-TOF mass spectrometry. Top mass of PDIa C56S mutant is shown as a peak at 15,403 m/z. The second peak at 15,582 (+179 change) is the natural gluconoylation modification of PDIa C56S at the His6× tag by E. coli. Top Middle, mass of PDIa C56S pre-treated with IAA shows a mass at 15,462 (+59 change) corresponding to bound ligand even after DTT treatment. Bottom Middle, mass of PDIa C56S pre-treated with BIT shows mass at 15,405 m/z corresponding to no ligand bound on the protein after DTT treatment. Bottom, mass of PDIa C56S treated with LOC14. No shift is observed in the pre-treated sample after DTT treatment indicating binding of LOC14 to PDIa C56S can be reversed with reducing agent.

FIG. 12 shows that LOC14 does not bind to glutathione. LOC14, benzoisothiazolone (BIT) and a positive control N-ethylmaleimide (NEM) were incubated with glutathione in a Tris-buffer (pH 8.5) and at selected timepoints, a solution of Ellman's reagent was added. Unreacted glutathione reacts with Ellman's reagent leading to the formation of 2-nitro-5-thiobenzoic acid (TNB), and its concentration can be measured spectrophotometrically.

FIGS. 13A-13C shows that LOC14 protects in multiple cell culture models of HD and in brain-slice model of AD.

FIG. 13A shows dose-response curves of LOC14's rescue of PC12 cells from mHTT^{Q103}-induced cell death, as measured by Presto blue fluorescence after 48-hour treatment. Data are plotted as mean percent of DMSO-treated uninduced cells±SD with n=3.

FIG. 13B shows that parental ST14A cells (blue) and ST14A expressing mHTT^{Q120} cells (red) were heat-shocked at 39° C., serum starved and co-treated with LOC14 for 48 hours after which time the viability was measured using CellTiter-Glo.

FIG. 13C shows that rat coronal brain slice explants co-transfected with YFP and Tau-4R were treated with LOC14, BIT, or DMSO-vehicle for 3 days. The cortical regions of each brain slice were then scored for numbers of healthy pyramidal neurons remaining. Data are plotted as means±SEM. Graph represents an average of 3 independent experimental runs. Significant difference is denoted with * with respect to the Tau-4R condition by ANOVA followed by Dunnett's post hoc comparison test at p<0.05.

FIGS. 14A-14D show that the neuroprotective effects of LOC14 is a result of reduction of ROS. FIG. 14A shows that the structure of the inactive isoxazolone analog of LOC14 differs by only a single S to O atom substitution. FIGS. 14B-14D show that PC12 mHTT^{Q103} cells were induced to express mHTT (I) or uninduced (U) and co-treated with vehicle (DMSO), 3 μM LOC14, or 3 μM OxyLOC14 for 24 hours. In FIG. 14B, cells were stained with 5 μM CeIIROX Orange dye to assay cellular ROS production. Representative data from one of three flow cytometry replicates are shown. In FIG. 14C, an aliquot from each sample was used for assessing viability, using Trypan blue dye on ViCell. FIG. 14D shows quantitation of population statistics from flow cytometry data. Data are plotted as mean±SD with n=3.

FIGS. 15A-15B show that addition of methyl groups to pyridine analogs reduces cellular toxicity.

FIG. 15A shows dose-response curves of LOC14 analogs that rescue PC12 cells from mHTT^{Q103}-induced cell death as measured by Presto blue fluorescence after 48-hour treatment. Data from cells induced to express mHTT^{Q103} (blue) and cells not expressing mHTT^{Q103} (red) are plotted as mean percent of DMSO-treated uninduced cells±SD with n=3.

FIG. 15B shows superimposed HSQC spectra of PDIa treated with either d6-DMSO vehicle (blue) or 2.5 mol. equiv. of LC-52 (red). Resonances with large chemical shifts are labeled in green.

FIGS. 16A-16E show that. LC-43 provides robust neuroprotection in AD and HD brain slices through modulation of PDI activity. FIG. 16A shows superimposed HSQC spectra of PDIa treated with either d6-DMSO vehicle (blue) or 5 mol. equiv. of LC-43 (red). Resonances with large chemical shifts are labeled in green. Rat coronal brain slice explants co-transfected with YFP and Tau-4R (FIG. 16B) or striatal brain slice explants co-transfected with YFP and mHTT (FIG. 16C) were treated with LC-43 or DMSO carrier-only for 3 days. The respective regions of each brain slice were then scored for numbers of healthy (FIG. 16B) pyramidal or (FIG. 16C) medium spiny neurons remaining. Data are normalized to the negative control condition set to 100% and plotted as means±SEM. In FIG. 16D, The confocal images shown are maximal z-stacks of 0.5 μm steps (˜20 images per stack). (Bottom) Reduction of neuronal redox state by LC-43. Left, Primary neurons were co-transfected with mCherry (to positively identify transfected neurons) and the genetically encoded ratiometric redox reporter roGFP2. Four days later, the culture medium was changed into an identical formulation except without conventional antioxidant additives. 24 hours after this medium change, LOC-43 or DMSO carrier-only were added to the neurons and images were taken after 30 minutes or 24 hours of treatment as indicated. Imaging was done using a Leica SP5 inverted confocal microscope through a 63× water-immersion objective. Images of 40 individual neurons for each condition were collected at 405 and 488 nm and analysis of the 405/488 ratio for each image pair was done using CellProfiler (Broad Institute). Right, in contrast, 30 minute treatment with the reducing agent DTT leads to an immediate and drastic reduction in the roGFP2 redox ratio. FIG. 16E shows that, at high concentration, above the concentration that is neuroprotective, LOC14 inhibits CYP3A4 and CYP1A2 enzymes. LC-43 does not show any inhibition against any of the five isoforms of CYP450.

### DETAILED DESCRIPTION OF THE DISCLOSURE

One embodiment of the present disclosure is a compound having the formula (I):

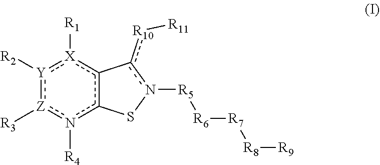

wherein a dashed line indicates the presence of an optional double bond, wherein X, Y and Z are independently selected from the group consisting of C, N, S and O, wherein R₁, R₂, R₃, and R₄ are independently selected from the group consisting of no atom, H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
with the proviso that the compound is not

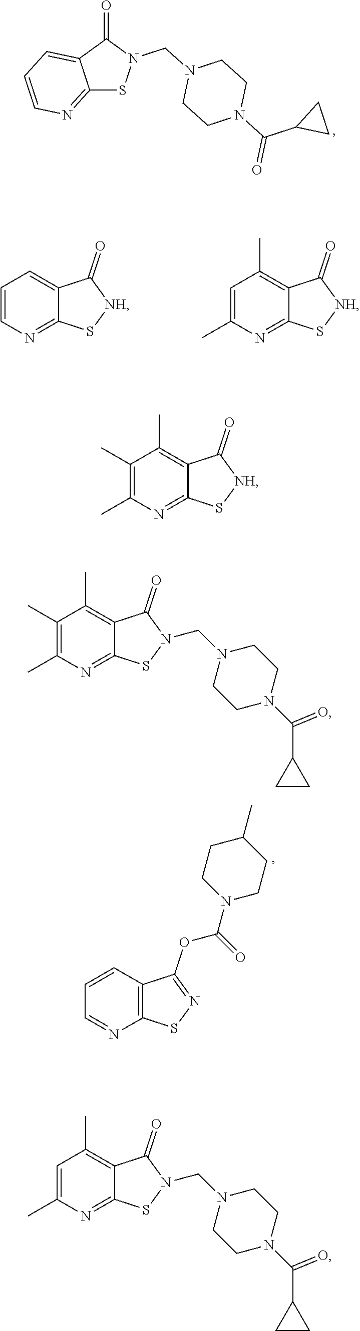

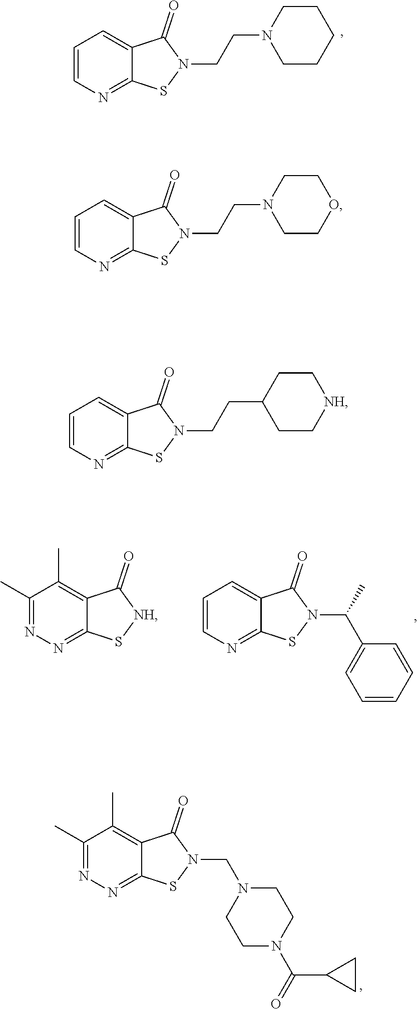 [SMILES: O=c1c2cccnc2sn1CCN1CCCCC1 ; O=c1c2cccnc2sn1CCN1CCOCC1 ; O=c1c2cccnc2sn1CCC1CCNCC1 ; Cc1nnc2s[nH]c(=O)c2c1C ; C[C@H](c1ccccc1)n1sc2ncccc2c1=O ; Cc1nnc2sn(CN3CCN(C(=O)C4CC4)CC3)c(=O)c2c1C]

or an N-oxide, crystalline form, hydrate thereof, or a pharmaceutically acceptable salt thereof.

As used herein, an “N-oxide” means a compound containing an N—O bond with three additional hydrogen and/or side chains attached to N, so that there is a positive charge on the nitrogen. The N-oxides of compounds of the present disclosure may be synthesized by simple oxidation procedures well known to those skilled in the art. For example, the oxidation procedure described by P. Brougham et al. (Synthesis, 1015-1017, 1987), allows the two nitrogen of a piperazine ring to be differentiated, enabling both the N-oxides and N,N′-dioxide to be obtained. Other oxidation procedures are disclosed in, e.g., U.S. Patent Publication No. 20070275977; S. L. Jain, J. K. Joseph, B. Sain, Synlett, 2006, 2661-2663; A. McKillop, D. Kemp, Tetrahedron, 1989, 45, 3299-3306; R. S. Varma, K. P. Naicker, Org. Lett., 1999, 1, 189-191; and N. K. Jana, J. G. Verkade, Org. Lett., 2003, 5, 3787-3790. Thus, the present disclosure includes these and other well-known procedures for making N-oxides, so long as the end product is sufficiently effective as set forth in more detail below.

The term “crystalline form”, as used herein, refers to the crystal structure of a compound. A compound may exist in one or more crystalline forms, which may have different structural, physical, pharmacological, or chemical characteristics. Different crystalline forms may be obtained using variations in nucleation, growth kinetics, agglomeration, and breakage. Nucleation results when the phase-transition energy barrier is overcome, thereby allowing a particle to form from a supersaturated solution. Crystal growth is the enlargement of crystal particles caused by deposition of the chemical compound on an existing surface of the crystal. The relative rate of nucleation and growth determine the size distribution of the crystals that are formed. The thermodynamic driving force for both nucleation and growth is supersaturation, which is defined as the deviation from thermodynamic equilibrium. Agglomeration is the formation of larger particles through two or more particles (e.g., crystals) sticking together and forming a larger crystalline structure.

As used herein, a “hydrate” means a compound that contains water molecules in a definite ratio and in which water forms an integral part of the crystalline structure of the compound. Methods of making hydrates are known in the art. For example, some substances spontaneously absorb water from the air to form hydrates. Others may form hydrates upon contact with water. In most cases, however, hydrates are made by changes in temperature or pressure. Additionally, the compounds of the present disclosure as well as their salts may contain, e.g., when isolated in crystalline form, varying amounts of solvents, such as water. Included within the scope of the disclosure are, therefore, all hydrates of the compounds and all hydrates of salts of the compounds of the present disclosure, so long as such hydrates are sufficiently effective as set forth in more detail below.

The term “aliphatic”, as used herein, refers to a group composed of carbon and hydrogen atoms that do not contain aromatic rings. Accordingly, aliphatic groups include alkyl, alkenyl, alkynyl, and carbocyclyl groups. Additionally, unless otherwise indicated, the term “aliphatic” is intended to include both “unsubstituted aliphatics” and “substituted aliphatics”, the latter of which refers to aliphatic moieties having substituents replacing a hydrogen on one or more carbons of the aliphatic group. Such substituents can include, for example, a halogen, a deuterium, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aromatic, or heteroaromatic moiety.

The term “alkyl” refers to the radical of saturated aliphatic groups that does not have a ring structure, including straight-chain alkyl groups, and branched-chain alkyl groups. In certain embodiments, a straight chain or branched chain alkyl has 6 or fewer carbon atoms in its backbone (e.g., C₁-C₆ for straight chains, C₃-C₆ for branched chains). Such substituents include all those contemplated for aliphatic groups, except where stability is prohibitive.

Moreover, unless otherwise indicated, the term “alkyl” as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Indeed, unless otherwise indicated, all groups recited herein are intended to include both substituted and unsubstituted options.

The term “alkenyl”, as used herein, refers to an aliphatic group containing at least one double bond and unless otherwise indicated, is intended to include both “unsubstituted alkenyls” and “substituted alkenyls”, the latter of which refers to alkenyl moieties having substituents replacing a hydrogen on one or more carbons of the alkenyl group. Such substituents include all those contemplated for aliphatic groups, as discussed below, except where stability is prohibitive. For example, substitution of alkenyl groups by one or more alkyl, carbocyclyl, aryl, heterocyclyl, or heteroaryl groups is contemplated.

The term “C_{x-y}” when used in conjunction with a chemical moiety, such as, alkyl and cycloalkyl, is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc.

The terms “amine” and “amino” are art-recognized and refer to both unsubstituted and substituted amines and salts thereof, e.g., a moiety that can be represented by

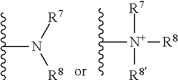

wherein R⁷, R⁸, and R^{8′} each independently represent a hydrogen or a hydrocarbyl group, or R⁷ and R⁸ taken together with the N atom to which they are attached complete a heterocycle having from 4 to 8 atoms in the ring structure. The term “primary” amine means only one of R⁷ and R⁸ or one of R⁷, R⁸, and R^{8′} is a hydrocarbyl group. Secondary amines have two hydrocarbyl groups bound to N. In tertiary amines, all three groups, R⁷, R⁸, and R^{8′}, are replaced by hydrocarbyl groups.

The term “aryl” as used herein includes substituted or unsubstituted single-ring aromatic groups in which each atom of the ring is carbon. Preferably the ring is a 3- to 8-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “alkyl-aryl” refers to an alkyl group substituted with at least one aryl group.

The terms “halo” and “halogen” are used interchangeably herein and mean halogen and include chloro, fluoro, bromo, and iodo.

The term “heterocycle” refers to substituted or unsubstituted non-aromatic ring structures, preferably 3- to 8-membered rings, whose ring structures include at least one heteroatom, preferably one to four heteroatoms, more preferably one or two heteroatoms. The term “heterocycle” also includes polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocycle groups include, for example, piperidine, piperazine, pyrrolidine, morpholine, lactones, lactams, and the like.

The term “heteroatom” as used herein means an atom of any element other than carbon or hydrogen. Preferred heteroatoms are nitrogen, oxygen, and sulfur; more preferably, nitrogen and oxygen.

The term “substituted” refers to moieties having substituents replacing a hydrogen on one or more carbons of the backbone. It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

As set forth previously, unless specifically stated as “unsubstituted,” references to chemical moieties herein are understood to include substituted variants. For example, reference to an “aryl” group or moiety implicitly includes both substituted and unsubstituted variants.

In one aspect of this embodiment, the compound is selected from the group consisting of:

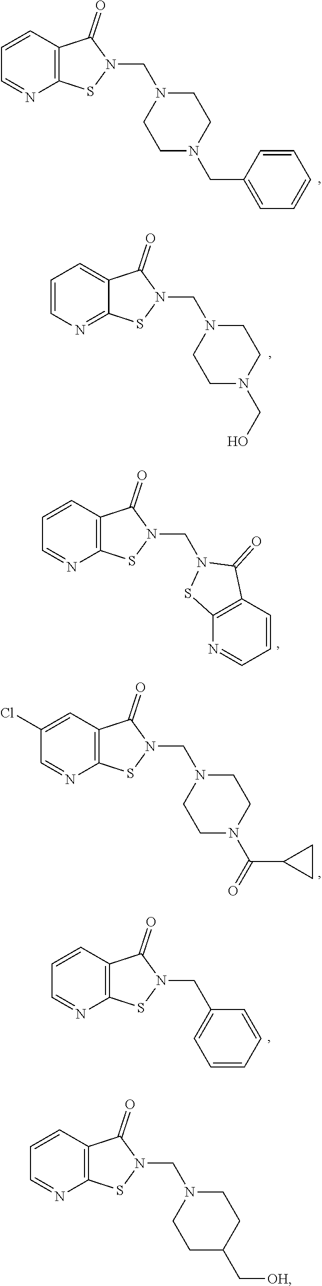 [SMILES: O=c1c2cccnc2sn1CN1CCN(Cc2ccccc2)CC1 ; O=c1c2cccnc2sn1CN1CCN(CO)CC1 ; O=c1c2cccnc2sn1Cn1sc2ncccc2c1=O ; O=C(C1CC1)N1CCN(Cn2sc3ncc(Cl)cc3c2=O)CC1 ; O=c1c2cccnc2sn1Cc1ccccc1 ; O=c1c2cccnc2sn1CN1CCC(CO)CC1]

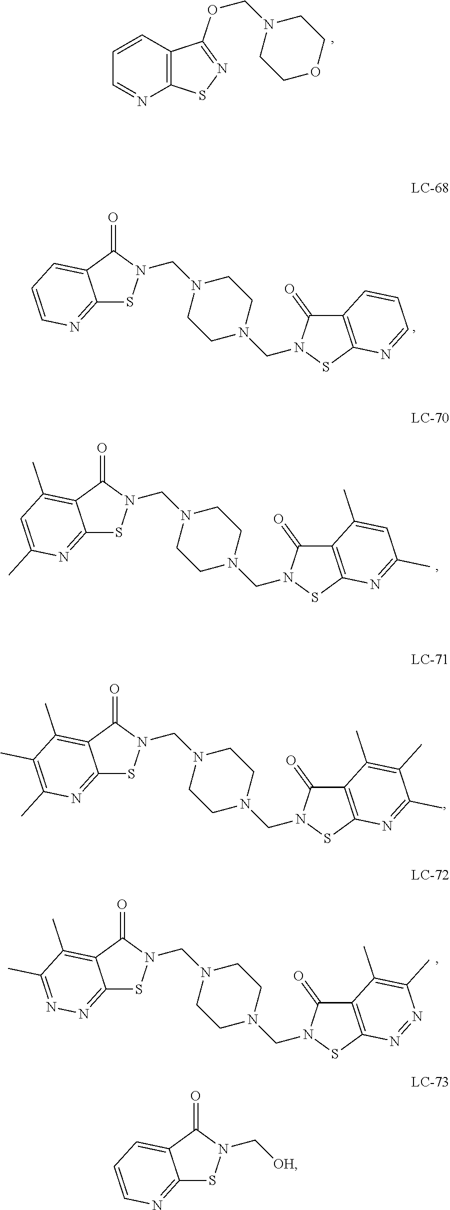

and combinations thereof,
or an N-oxide, crystalline form, hydrate thereof, or a pharmaceutically acceptable salt thereof.

Another embodiment of the present disclosure is a compound have the formula (II):

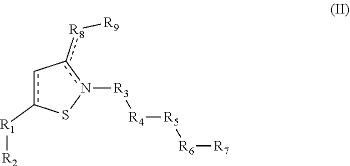

wherein a dashed line indicates the presence of an optional double bond,
wherein R₁ is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₃, R₄, R₅, R₆ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₇ and R₉ are independently selected from the group consisting of H, NR, N(R)₂, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, or an N-oxide, crystalline form, hydrate thereof, or a pharmaceutically acceptable salt thereof.

In one aspect of this embodiment, the compound is selected from the group consisting of:

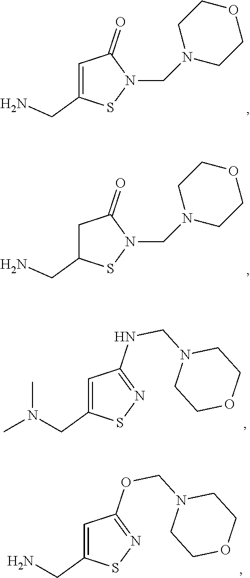 [SMILES: NCc1cc(=O)n(CN2CCOCC2)s1 ; NCC1CC(=O)N(CN2CCOCC2)S1 ; CN(C)Cc1cc(NCN2CCOCC2)ns1 ; NCc1cc(OCN2CCOCC2)ns1]

and combinations thereof,
or an N-oxide, crystalline form, hydrate thereof, or a pharmaceutically acceptable salt thereof.

Another embodiment of the present disclosure is a compound selected from the group consisting of:

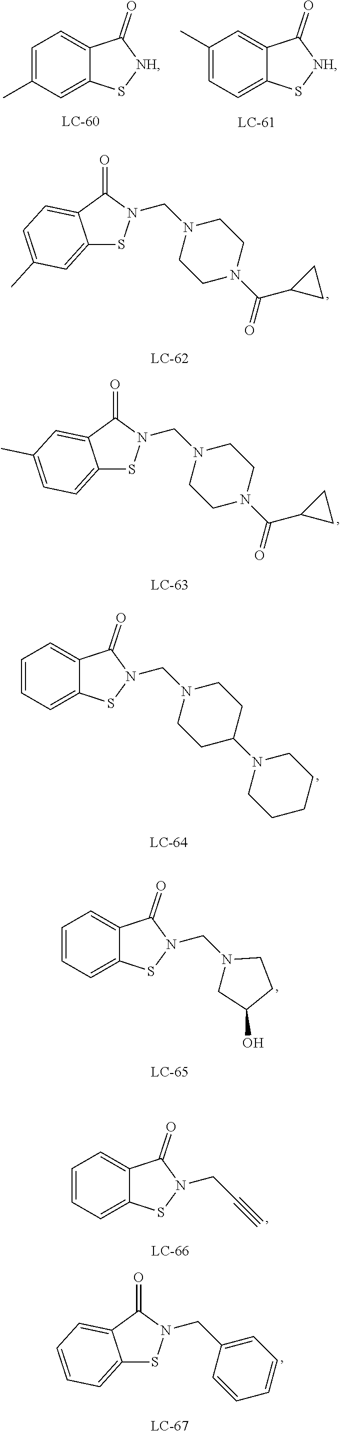

and combinations thereof,
or an N-oxide, crystalline form, hydrate thereof, or a pharmaceutically acceptable salt thereof.

Another embodiment of the present disclosure is a composition comprising a compound of the present disclosure and a pharmaceutically acceptable carrier, adjuvant or vehicle.

Another embodiment of the present disclosure is a pharmaceutically acceptable salt of a compound of the present disclosure.

Another embodiment of the present disclosure is a composition comprising a pharmaceutically acceptable salt of the present disclosure and a pharmaceutically acceptable carrier, adjuvant or vehicle.

Another embodiment of the disclosure is a kit comprising a compound or composition of the present disclosure and instructions for use.

In one aspect of this embodiment, the instructions for use are instructions for treating or ameliorating the effects of a neurodegenerative disorder in a subject. In another aspect of this embodiment, the instruction for use are instructions for treating or ameliorating the effects of a condition associated with increased protein disulfide isomerase (PDI) activity in a subject. In another aspect of this embodiment, the instructions for use are instructions for modulating PDI activity in a cell.

Another embodiment of the disclosure is a method for treating or ameliorating the effects of a neurodegenerative disease in a subject in need thereof comprising administering to the subject an effective amount of a compound selected from the group consisting of formula (I)

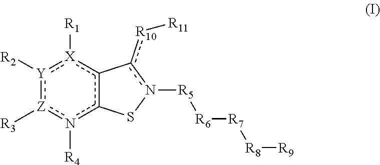

wherein a dashed line indicates the presence of an optional double bond,
wherein X, Y and Z are independently selected from the group consisting of C, N, S and O,
wherein R₁, R₂, R₃, and R₄ are independently selected from the group consisting of no atom, H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
with the proviso that the compound is not

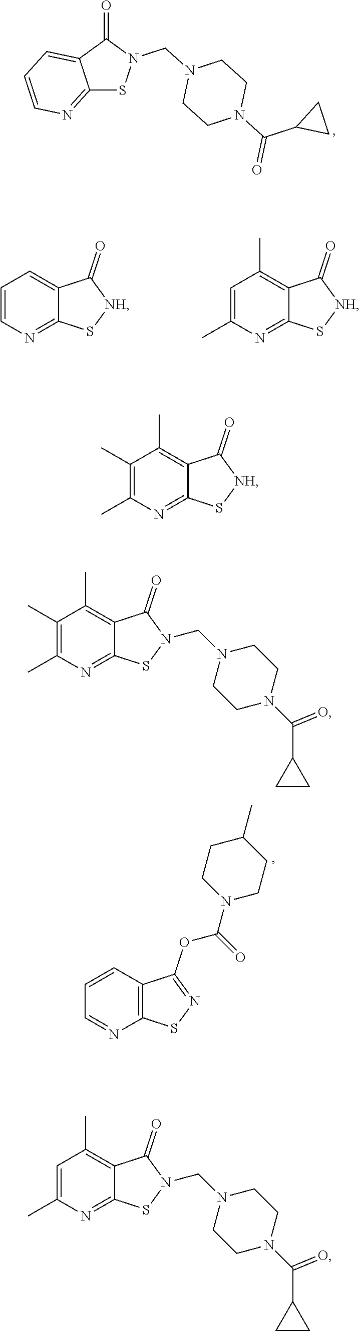

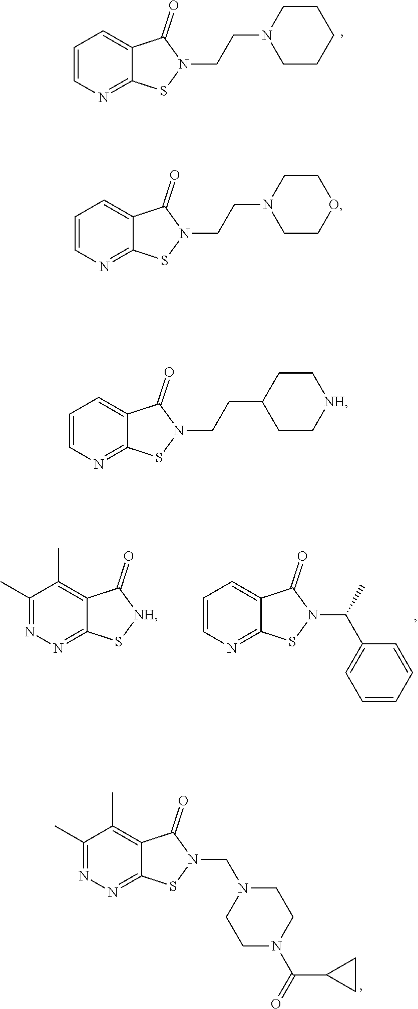 [SMILES: O=c1c2cccnc2sn1CCN1CCCCC1 ; O=c1c2cccnc2sn1CCN1CCOCC1 ; O=c1c2cccnc2sn1CCC1CCNCC1 ; Cc1nnc2s[nH]c(=O)c2c1C ; C[C@H](c1ccccc1)n1sc2ncccc2c1=O ; Cc1nnc2sn(CN3CCN(C(=O)C4CC4)CC3)c(=O)c2c1C]

or an N-oxide, crystalline form, hydrate thereof, or a pharmaceutically acceptable salt thereof;

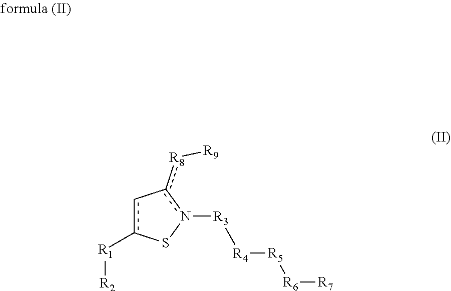

wherein a dashed line indicates the presence of an optional double bond,
wherein R₁ is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₃, R₄, R₅, R₆ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₇ and R₉ are independently selected from the group consisting of H, NR, N(R)₂, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof,
a compound selected from the group consisting of:

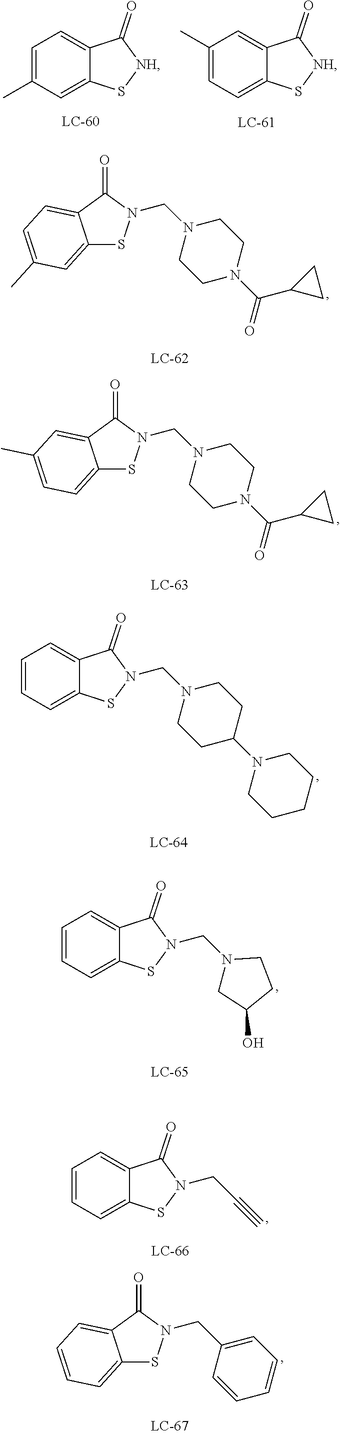

and combinations thereof,
or an N-oxide, crystalline form, hydrate thereof, or a pharmaceutically acceptable salt thereof.

In one aspect of this embodiment, the compound is selected from the group consisting of:

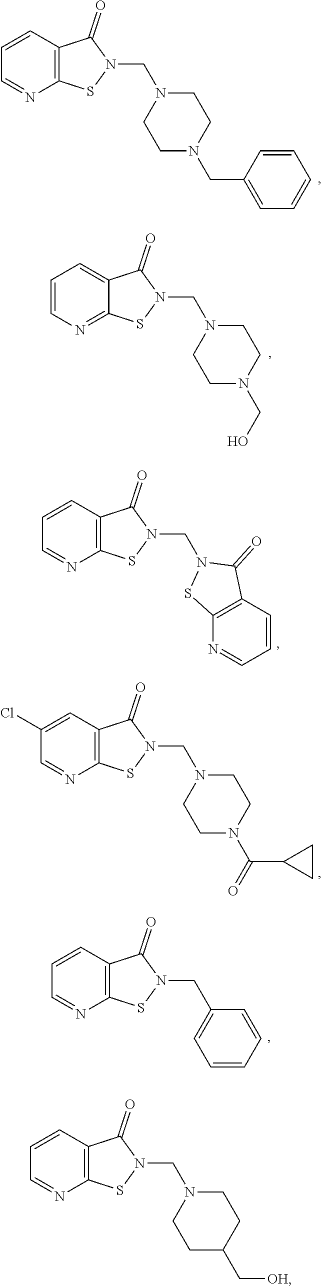 [SMILES: O=c1c2cccnc2sn1CN1CCN(Cc2ccccc2)CC1 ; O=c1c2cccnc2sn1CN1CCN(CO)CC1 ; O=c1c2cccnc2sn1Cn1sc2ncccc2c1=O ; O=C(C1CC1)N1CCN(Cn2sc3ncc(Cl)cc3c2=O)CC1 ; O=c1c2cccnc2sn1Cc1ccccc1 ; O=c1c2cccnc2sn1CN1CCC(CO)CC1]

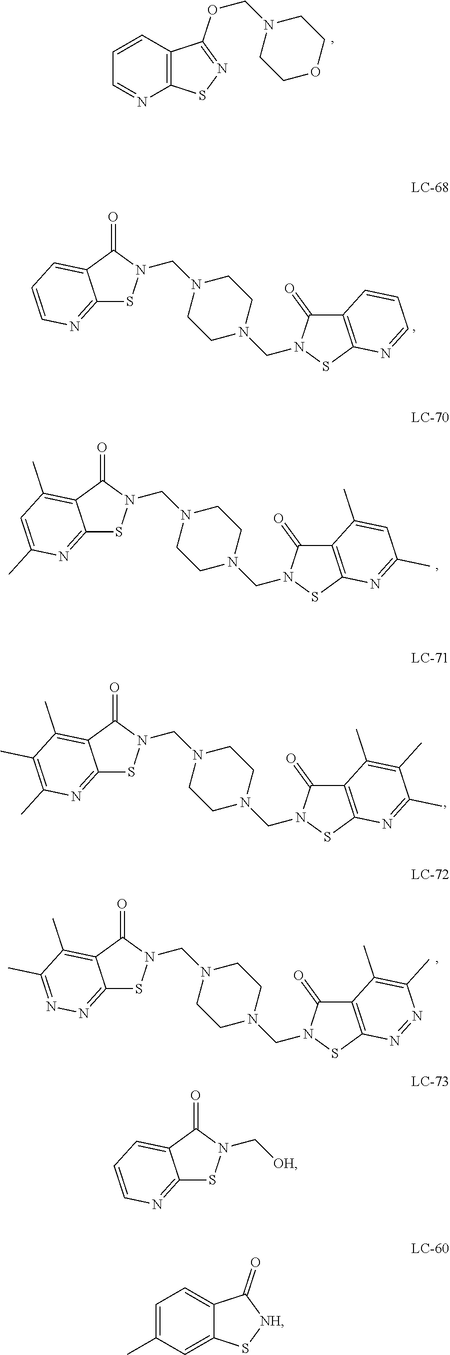

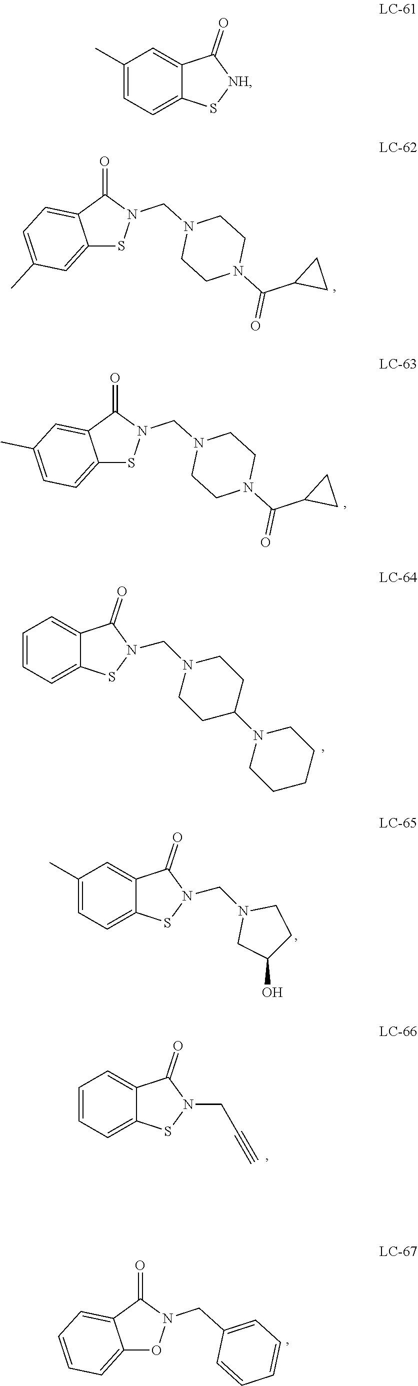

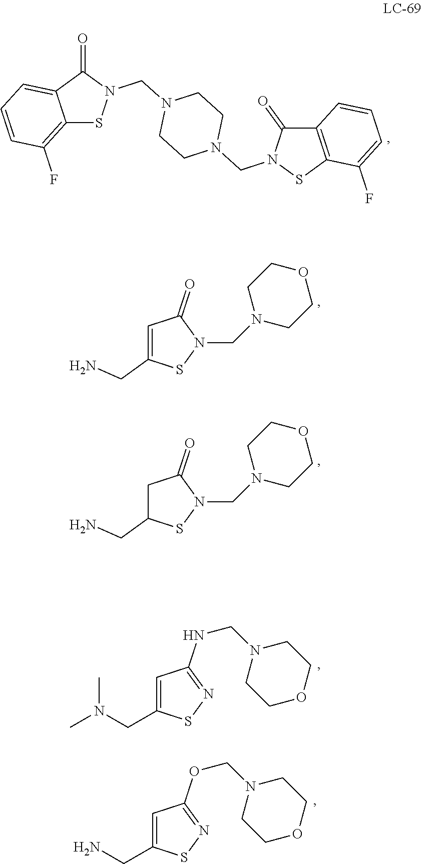 [SMILES: O=c1c2cccc(F)c2sn1CN1CCN(Cn2sc3c(F)cccc3c2=O)CC1 ; NCc1cc(=O)n(CN2CCOCC2)s1 ; NCC1CC(=O)N(CN2CCOCC2)S1 ; CN(C)Cc1cc(NCN2CCOCC2)ns1 ; NCc1cc(OCN2CCOCC2)ns1]

and combinations thereof,
or an N-oxide, crystalline form, hydrate thereof, or a pharmaceutically acceptable salt thereof.

Another embodiment of the present disclosure is a method for treating or ameliorating the effects of a neurodegenerative disorder in a subject in need thereof comprising administering to the subject an effective amount of a composition of the disclosure.

In one aspect of the above embodiments, the neurodegenerative disease is selected from the group consisting of Alzheimer's disease, Parkinson's disease, amyotrophic lateral sclerosis (ALS), Friedreich's ataxia, multiple sclerosis, Huntington's disease, transmissible spongiform encephalopathy, Charcot-Marie-Tooth disease, dementia with Lewy bodies, corticobasal degeneration, progressive supranuclear palsy, and hereditary spastic paraparesis. In some embodiments, the neurodegenerative disease is Huntington's disease. In some embodiments, the neurodegenerative disease is Alzheimer's disease.

In another aspect of the above embodiments, the subject is a mammal. In some aspects of this embodiment the mammal is selected from the group consisting of humans, veterinary animals, and agricultural animals. In another aspect of this embodiment the subject is a human.

In one aspect of the above embodiments, the method further comprises co-administering to the subject an effective amount of one or more additional therapeutic agents. Preferably, the one or more additional therapeutic agents are selected from the group consisting of 5-hydroxytryptophan, Activase, AFQ056 (Novartis), Aggrastat, Albendazole, alpha-lipoic acid/L-acetyl carnitine, Alteplase, Amantadine (Symmetrel), amlodipine, Ancrod, Apomorphine (Apokyn), Arimoclomol, Arixtra, Armodafinil, Ascorbic acid, Ascriptin, Aspirin, atenolol, Avonex, baclofen (Lioresal), Banzel, Benztropine (Cogentin), Betaseron, BGG492 (Novartis Corp.), Botulinum toxin, Bufferin, Carbatrol®, Carbidopa/levodopa immediate-release (Sinemet), Carbidopa/levodopa oral disintegrating (Parcopa), Carbidopa/levodopa/Entacapone (Stalevo), CERE-110: Adeno-Associated Virus Delivery of NGF (Ceregene), cerebrolysin, CinnoVex, citalopram, citicoline, Clobazam, Clonazepam, Clopidogrel, clozapine (Clozaril), Coenzyme Q, Creatine, dabigatran, dalteparin, Dapsone, Davunetide, Deferiprone, Depakene®, Depakote ER®, Depakote®, Desmoteplase, Diastat, Diazepam, Digoxin, Dilantin®, Dimebon, dipyridamole, divalproex (Depakote), Donepezil (Aricept), EGb 761, Eldepryl, ELND002 (Elan Pharmaceuticals), Enalapril, enoxaparin, Entacapone (Comtan), epoetin alfa, Eptifibatide, Erythropoietin, Escitalopram, Eslicarbazepine acetate, Esmolol, Ethosuximide, Ethyl-EPA (Miraxion™), Exenatide, Extavia, Ezogabine, Felbamate, Felbatol®, Fingolimod (Gilenya), fluoxetine (Prozac), fondaparinux, Fragmin, Frisium, Gabapentin, Gabitril®, Galantamine, Glatiramer (Copaxone), haloperidol (Haldol), Heparin, human chorionic gonadotropin (hCG), Idebenone, Inovelon®, insulin, Interferon beta 1a, Interferon beta 1b, ioflupane 123I (DATSCAN®), IPX066 (Impax Laboratories Inc.), JNJ-26489112 (Johnson and Johnson), Keppra®, Klonopin, Lacosamide, L-Alpha glycerylphosphorylcholine, Lamictal®, Lamotrigine, Levetiracetam, liraglutide, Lisinopril, Lithium carbonate, Lopressor, Lorazepam, losartan, Lovenox, Lu AA24493, Luminal, LY450139 (Eli Lilly), Lyrica, Masitinib, Mecobalamin, Memantine, methylprednisolone, metoprolol tartrate, Minitran, Minocycline, mirtazapine, Mitoxantrone (Novantrone), Mysoline®, Natalizumab (Tysabri), Neurontin®, Niacinamide, Nitro-Bid, Nitro-Dur, nitroglycerin, Nitrolingual, Nitromist, Nitrostat, Nitro-Time, Norepinephrine (NOR), Carbamazepine, octreotide, Onfi®, Oxcarbazepine, Oxybutinin chloride, PF-04360365 (Pfizer), Phenobarbital, Phenytek®, Phenytoin, piclozotan, Pioglitazone, Plavix, Potiga, Pramipexole (Mirapex), pramlintide, Prednisone, Primidone, Prinivil, probenecid, Propranolol, PRX-00023 (EPIX Pharmaceuticals Inc.), PXT3003, Quinacrine, Ramelteon, Rasagiline (Azilect), Rebif, ReciGen, remacemide, Resveratrol, Retavase, reteplase, riluzole (Rilutek), Rivastigmine (Exelon), Ropinirole (Requip), Rotigotine (Neupro), Rufinamide, Sabril, safinamide (EMD Serono), Salagen, Sarafem, Selegiline (1-deprenyl, Eldepryl), SEN0014196 (Siena Biotech), sertraline (Zoloft), Simvastatin, Sodium Nitroprussiate (NPS), sodium phenylbutyrate, Stanback Headache Powder, Tacrine (Cognex), Tamoxifen, tauroursodeoxycholic acid (TUDCA), Tegretol®, Tenecteplase, Tenormin, Tetrabenazine (Xenazine), THR-18 (Thrombotech Ltd.), Tiagabine, Tideglusib, tirofiban, tissue plasminogen activator (tPA), tizanidine (Zanaflex), TNKase, Tolcapone (Tasmar), Tolterodine, Topamax®, Topiramate, Trihexyphenidyl (formerly Artane), Trileptal®, ursodiol, Valproic Acid, valsartan, Varenicline (Pfizer), Vimpat, Vitamin E, Warfarin, Zarontin®, Zestril, Zonegran®, Zonisamide, Zydis selegiline HCL Oral disintegrating (Zelapar), and combinations thereof.

Another embodiment of the present disclosure is a method for treating or ameliorating the effects of a condition associated with increased protein disulfide isomerase (PDI) activity in a subject in need thereof comprising administering to the subject an effective amount of a compound selected from the group consisting of formula (I)

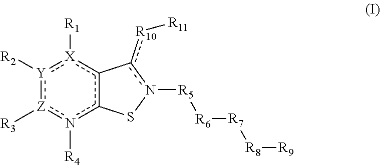

wherein a dashed line indicates the presence of an optional double bond,
wherein X, Y and Z are independently selected from the group consisting of C, N, S and O,
wherein R₁, R₂, R₃, and R₄ are independently selected from the group consisting of no atom, H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
with the proviso that the compound is not

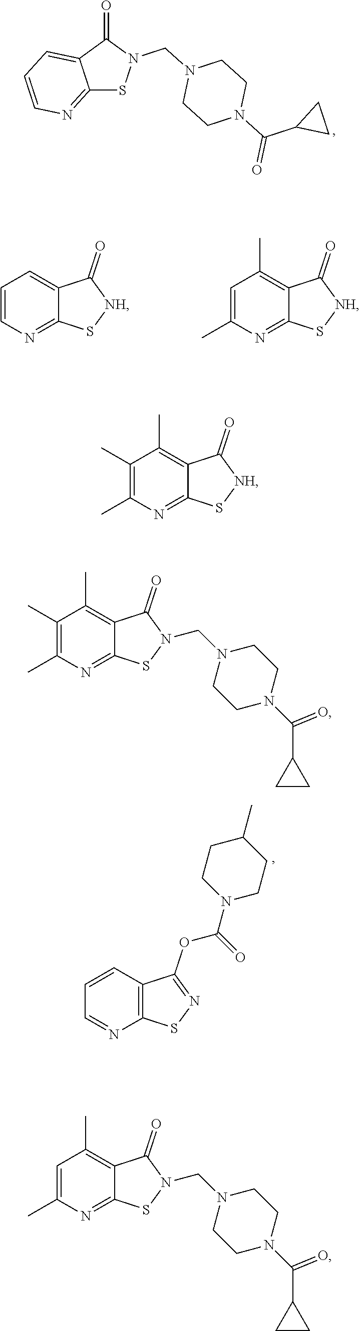

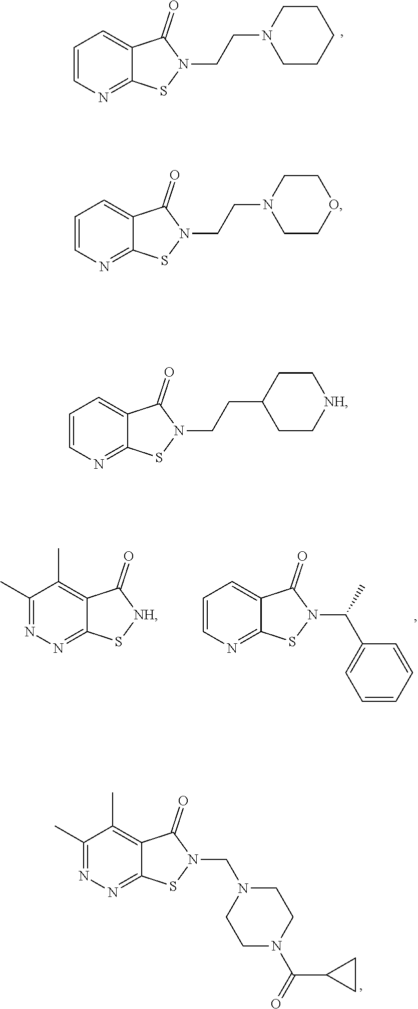 [SMILES: O=c1c2cccnc2sn1CCN1CCCCC1 ; O=c1c2cccnc2sn1CCN1CCOCC1 ; O=c1c2cccnc2sn1CCC1CCNCC1 ; Cc1nnc2s[nH]c(=O)c2c1C ; C[C@H](c1ccccc1)n1sc2ncccc2c1=O ; Cc1nnc2sn(CN3CCN(C(=O)C4CC4)CC3)c(=O)c2c1C]

or an N-oxide, crystalline form, hydrate thereof, or a pharmaceutically acceptable salt thereof,

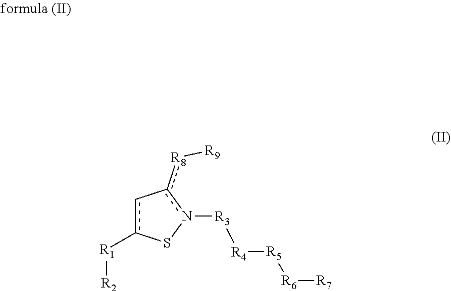

wherein a dashed line indicates the presence of an optional double bond,
wherein R₁ is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₃, R₄, R₅, R₆ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₇ and R₉ are independently selected from the group consisting of H, NR, N(R)₂, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
or an N-oxide, crystalline form, hydrate thereof, or a pharmaceutically acceptable salt thereof,
a compound selected from the group consisting of:

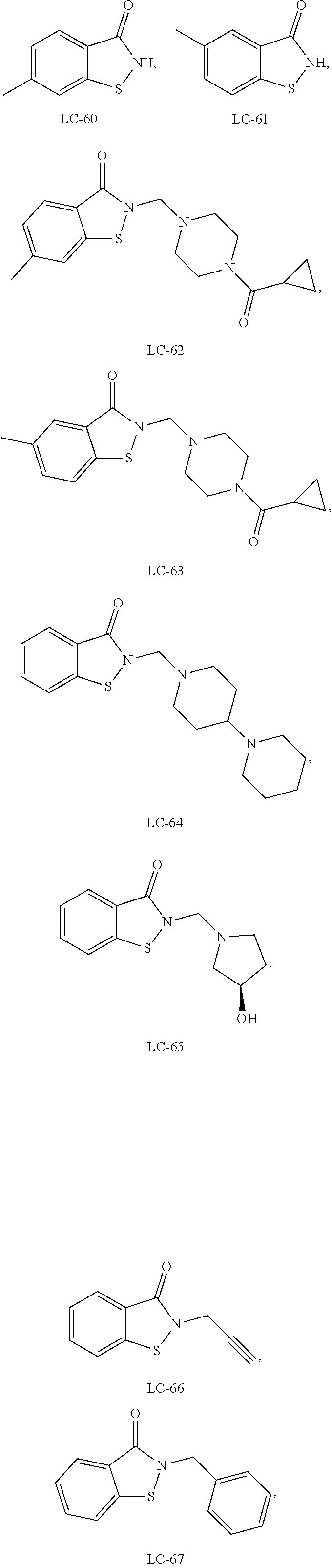

and combinations thereof,
or an N-oxide, crystalline form, hydrate thereof, or a pharmaceutically acceptable salt thereof.

In one aspect of this embodiment, the compound is selected from the group consisting of:

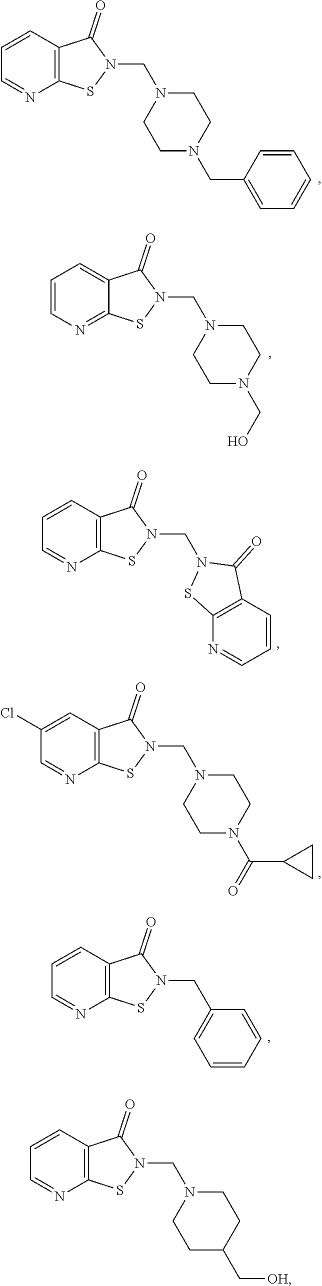 [SMILES: O=c1c2cccnc2sn1CN1CCN(Cc2ccccc2)CC1 ; O=c1c2cccnc2sn1CN1CCN(CO)CC1 ; O=c1c2cccnc2sn1Cn1sc2ncccc2c1=O ; O=C(C1CC1)N1CCN(Cn2sc3ncc(Cl)cc3c2=O)CC1 ; O=c1c2cccnc2sn1Cc1ccccc1 ; O=c1c2cccnc2sn1CN1CCC(CO)CC1]

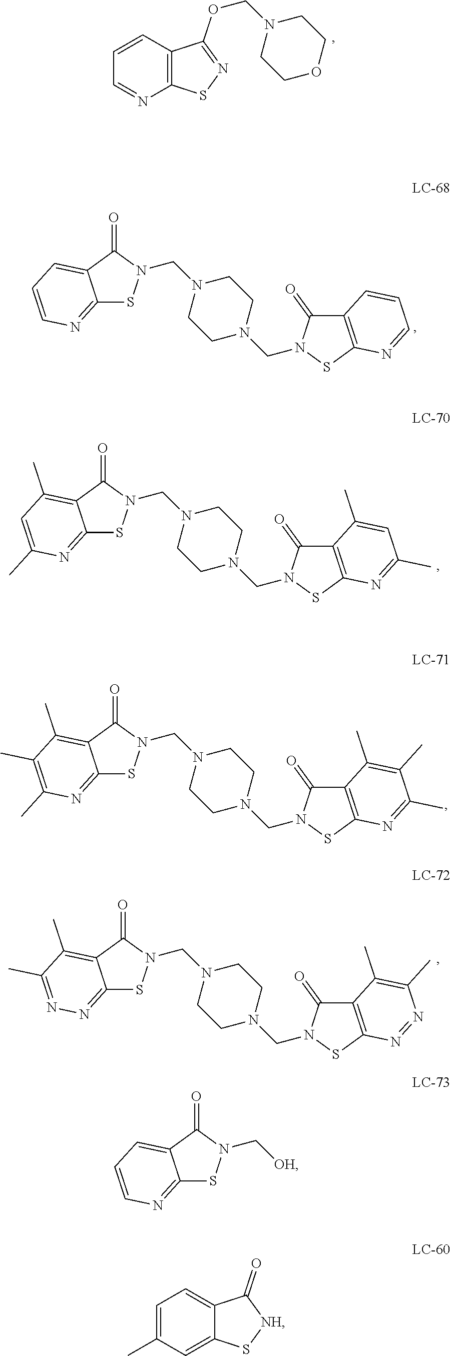

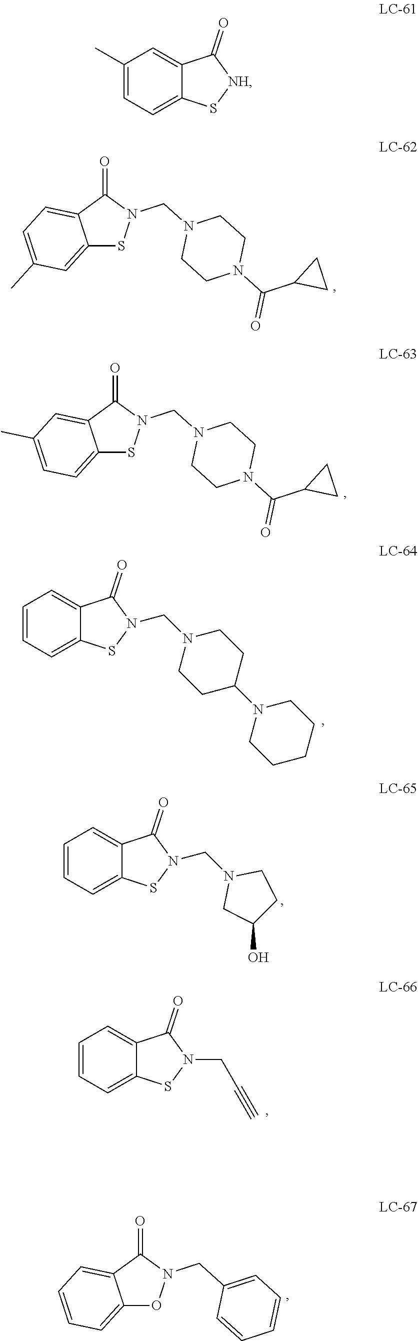

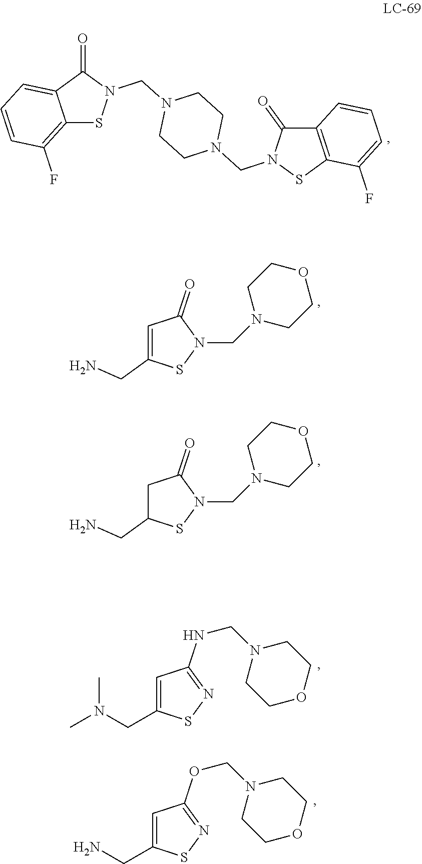 [SMILES: O=c1c2cccc(F)c2sn1CN1CCN(Cn2sc3c(F)cccc3c2=O)CC1 ; NCc1cc(=O)n(CN2CCOCC2)s1 ; NCC1CC(=O)N(CN2CCOCC2)S1 ; CN(C)Cc1cc(NCN2CCOCC2)ns1 ; NCc1cc(OCN2CCOCC2)ns1]

and combinations thereof, or an N-oxide, crystalline form, hydrate thereof, or a pharmaceutically acceptable salt thereof.

Another embodiment of the present disclosure is a method for treating or ameliorating the effects of a condition associated with increased protein disulfide isomerase (PDI) activity in a subject in need thereof comprising administering to the subject an effective amount of a composition of the present disclosure.

In another aspect of the above embodiments, the subject is a mammal. In some aspects of the above embodiments, the mammal is selected from the group consisting of humans, veterinary animals, and agricultural animals. In another aspect of the above embodiments, the subject is a human.

In one aspect of the above embodiments, the condition is a protein folding disorder. In another aspect of the above embodiments, the condition is cancer. In yet another aspect of the above embodiments, the condition is HIV. In yet another aspect of the above embodiments, the condition is a blood clot. In still another aspect of the above embodiments, the condition is an infection such as, e.g., influenza infection.

Another embodiment of the present disclosure is a method of modulating PDI activity in a cell comprising administering to the subject an effective amount of a compound selected from the group consisting of formula (I)

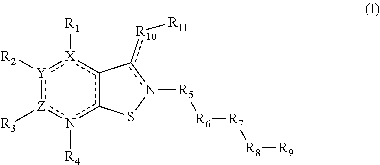

wherein a dashed line indicates the presence of an optional double bond,
wherein X, Y and Z are independently selected from the group consisting of C, N, S and O,
wherein R₁, R₂, R₃, and R₄ are independently selected from the group consisting of no atom, H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
with the proviso that the compound is not

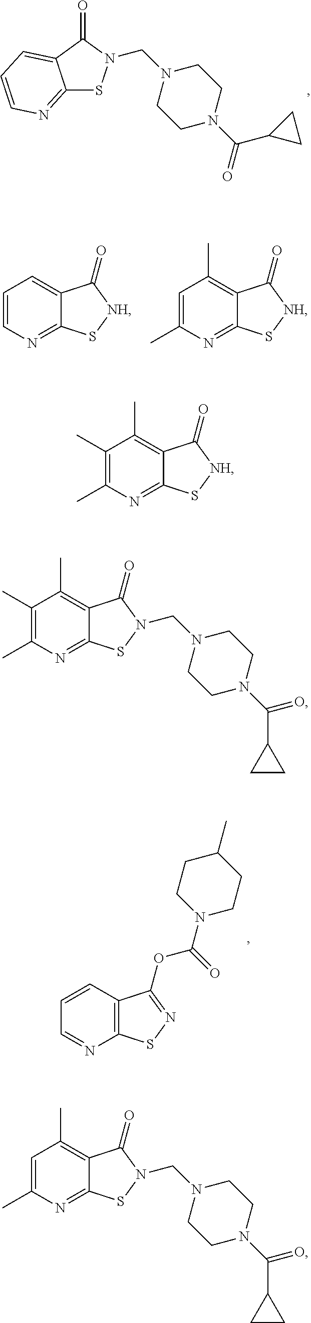

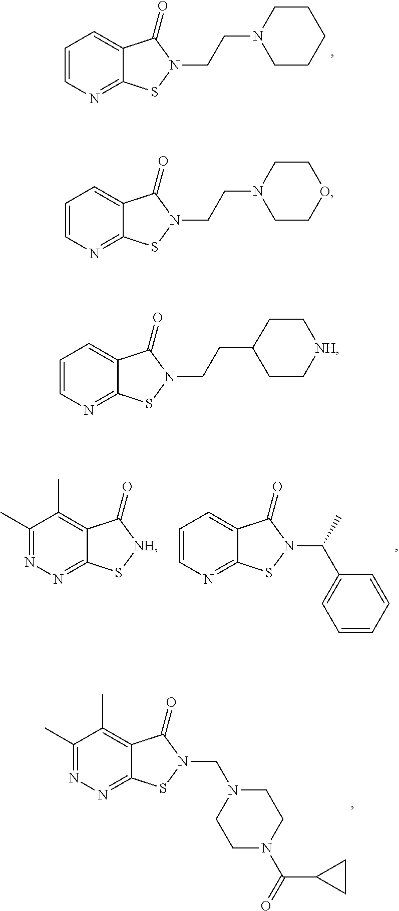

or an N-oxide, crystalline form, hydrate thereof, or a pharmaceutically acceptable salt thereof;

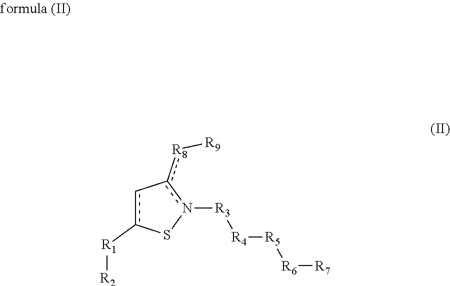

wherein a dashed line indicates the presence of an optional double bond,
wherein R₁ is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₃, R₄, R₅, R₆ and R₈ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₇ and R₉ are independently selected from the group consisting of H, NR, N(R)₂, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof, or an N-oxide, crystalline form, hydrate thereof, or a pharmaceutically acceptable salt thereof;
a compound selected from the group consisting of:

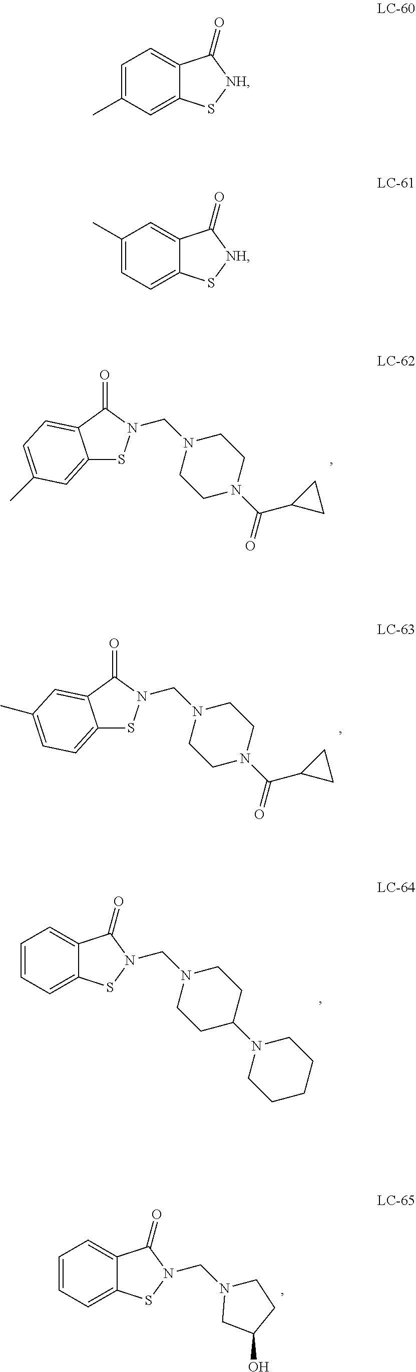 [SMILES: Cc1ccc2c(=O)[nH]sc2c1 ; Cc1ccc2s[nH]c(=O)c2c1 ; Cc1ccc2c(=O)n(CN3CCN(C(=O)C4CC4)CC3)sc2c1 ; Cc1ccc2sn(CN3CCN(C(=O)C4CC4)CC3)c(=O)c2c1 ; O=c1c2ccccc2sn1CN1CCC(N2CCCCC2)CC1 ; O=c1c2ccccc2sn1CN1CC[C@@H](O)C1]

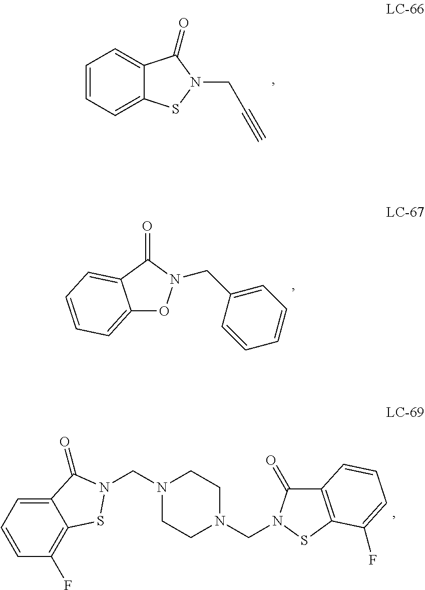 [SMILES: C#CCn1sc2ccccc2c1=O ; O=c1c2ccccc2on1Cc1ccccc1 ; O=c1c2cccc(F)c2sn1CN1CCN(Cn2sc3c(F)cccc3c2=O)CC1]

and combinations thereof,
or an N-oxide, crystalline form, hydrate thereof, or a pharmaceutically acceptable salt thereof.

In one aspect of this embodiment, the compound is selected from the group consisting of:

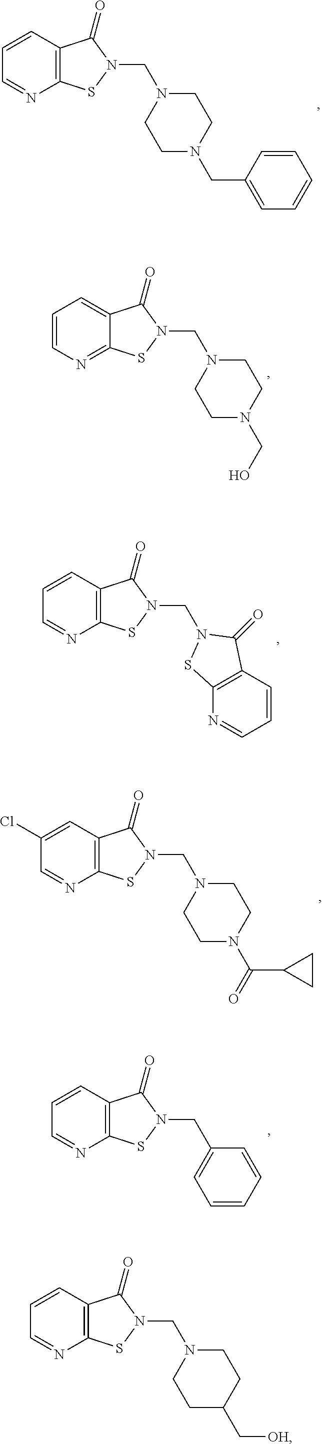 [SMILES: O=c1c2cccnc2sn1CN1CCN(Cc2ccccc2)CC1 ; O=c1c2cccnc2sn1CN1CCN(CO)CC1 ; O=c1c2cccnc2sn1Cn1sc2ncccc2c1=O ; O=C(C1CC1)N1CCN(Cn2sc3ncc(Cl)cc3c2=O)CC1 ; O=c1c2cccnc2sn1Cc1ccccc1 ; O=c1c2cccnc2sn1CN1CCC(CO)CC1]

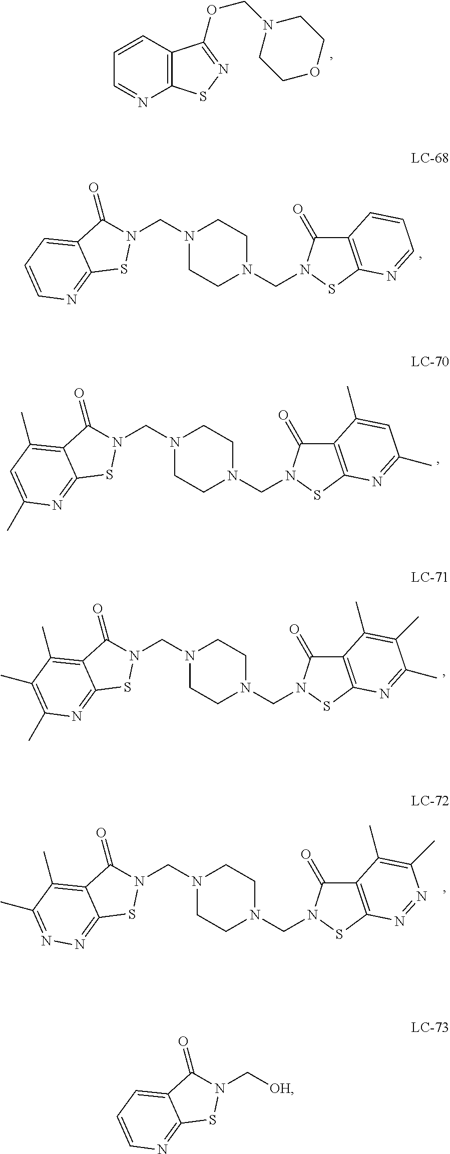

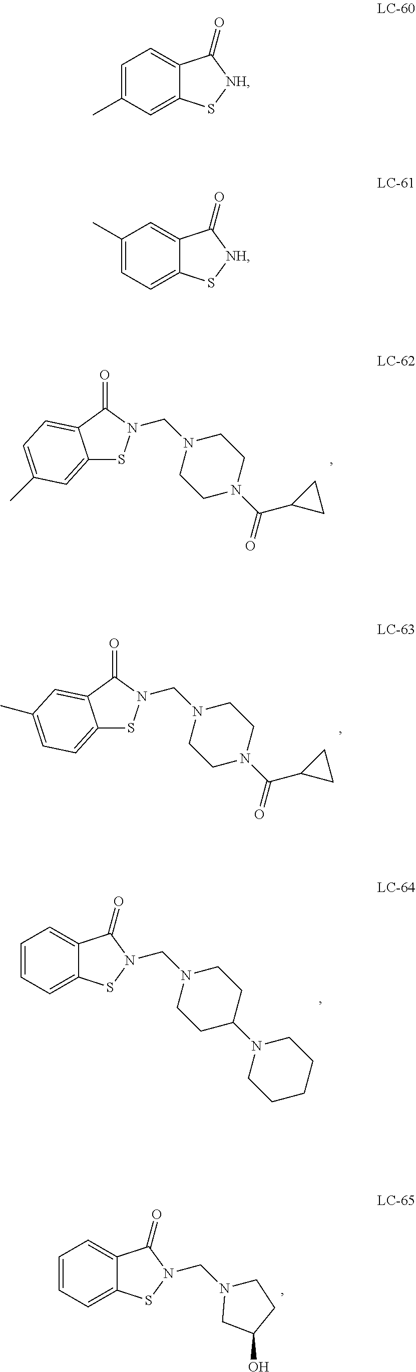 [SMILES: Cc1ccc2c(=O)[nH]sc2c1 ; Cc1ccc2s[nH]c(=O)c2c1 ; Cc1ccc2c(=O)n(CN3CCN(C(=O)C4CC4)CC3)sc2c1 ; Cc1ccc2sn(CN3CCN(C(=O)C4CC4)CC3)c(=O)c2c1 ; O=c1c2ccccc2sn1CN1CCC(N2CCCCC2)CC1 ; O=c1c2ccccc2sn1CN1CC[C@@H](O)C1]

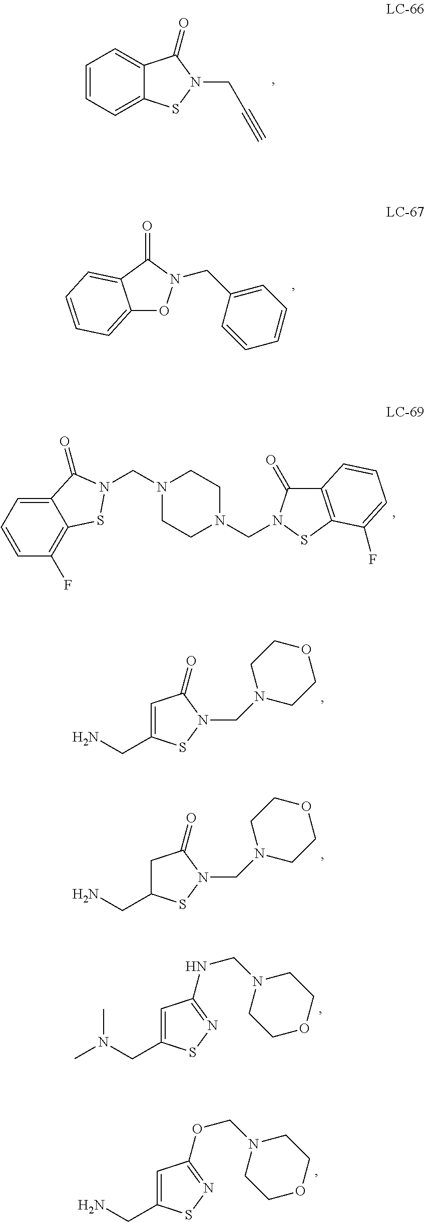

and combinations thereof, or an N-oxide, crystalline form, hydrate thereof, or a pharmaceutically acceptable salt thereof.

Another embodiment of the present disclosure is a method of modulating PDI activity in a cell comprising administering to the subject an effective amount of a composition of the present disclosure.

Another embodiment of the present disclosure is a compound selected from the group consisting of:

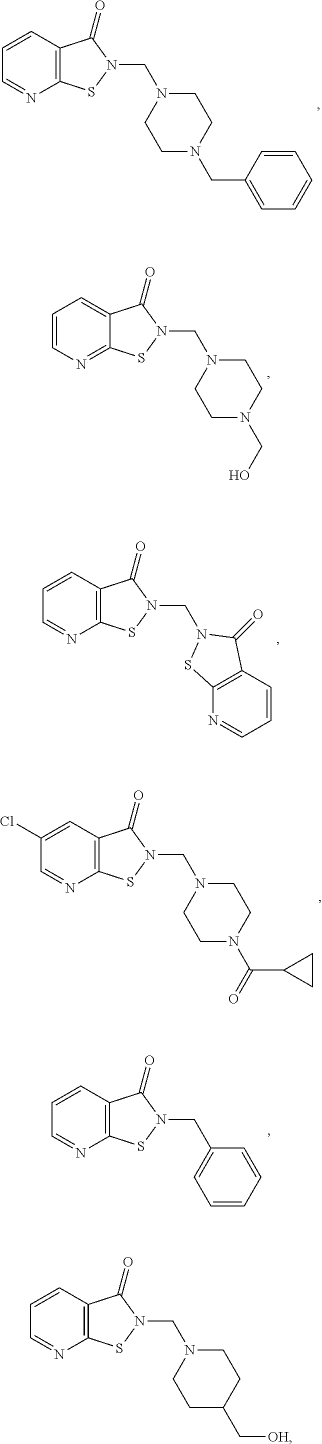 [SMILES: O=c1c2cccnc2sn1CN1CCN(Cc2ccccc2)CC1 ; O=c1c2cccnc2sn1CN1CCN(CO)CC1 ; O=c1c2cccnc2sn1Cn1sc2ncccc2c1=O ; O=C(C1CC1)N1CCN(Cn2sc3ncc(Cl)cc3c2=O)CC1 ; O=c1c2cccnc2sn1Cc1ccccc1 ; O=c1c2cccnc2sn1CN1CCC(CO)CC1]

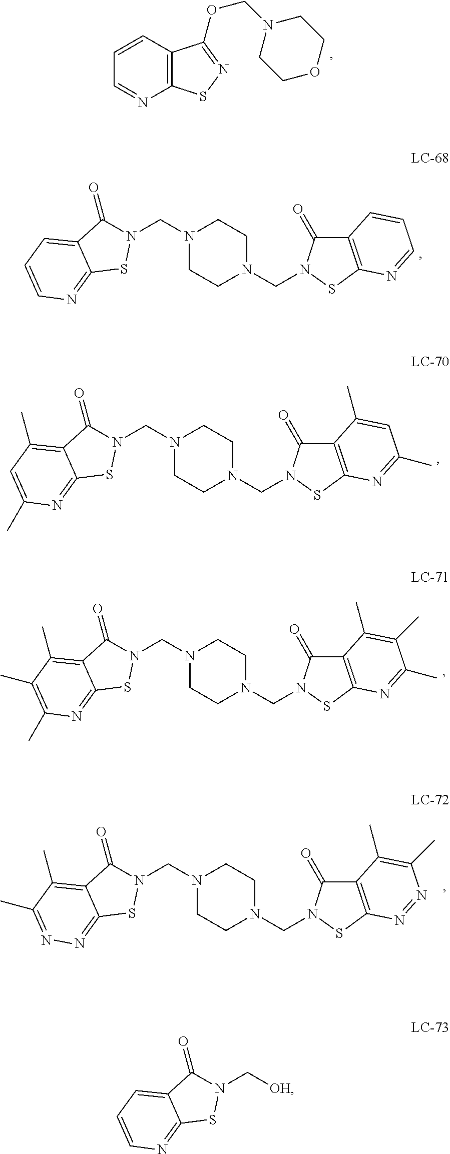

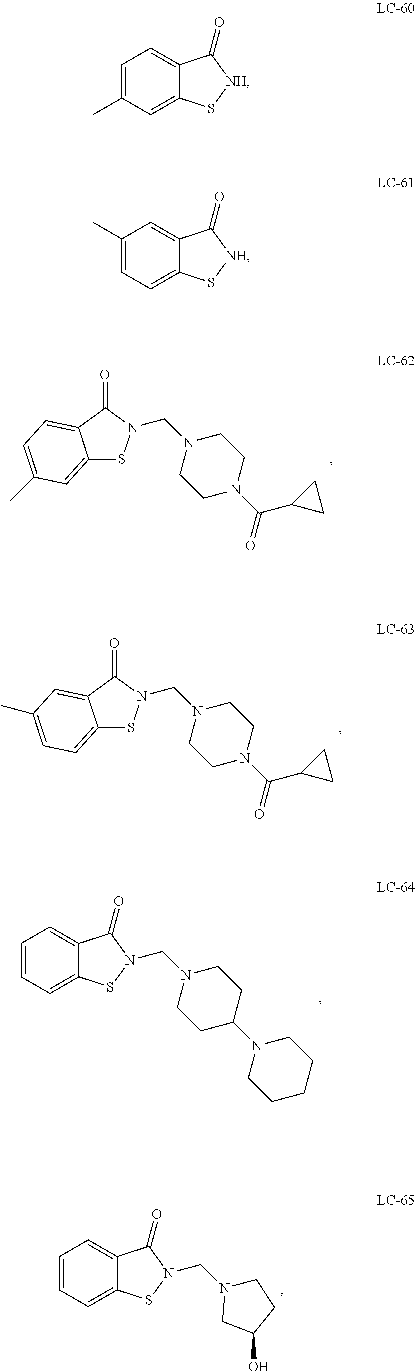 [SMILES: Cc1ccc2c(=O)[nH]sc2c1 ; Cc1ccc2s[nH]c(=O)c2c1 ; Cc1ccc2c(=O)n(CN3CCN(C(=O)C4CC4)CC3)sc2c1 ; Cc1ccc2sn(CN3CCN(C(=O)C4CC4)CC3)c(=O)c2c1 ; O=c1c2ccccc2sn1CN1CCC(N2CCCCC2)CC1 ; O=c1c2ccccc2sn1CN1CC[C@@H](O)C1]

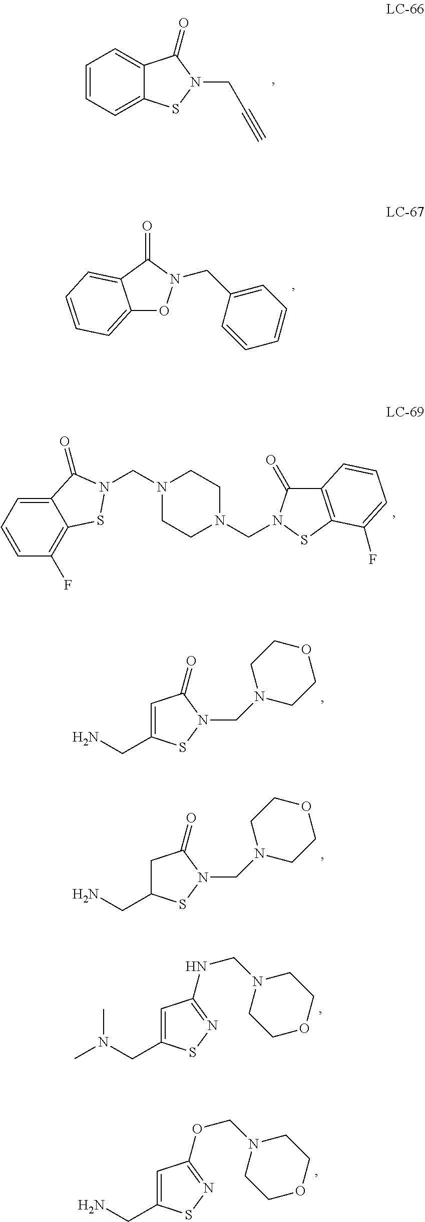

and combinations thereof,
or an N-oxide, crystalline form, hydrate thereof, or a pharmaceutically acceptable salt thereof.

Another embodiment of the present disclosure is a compound having the structure of:

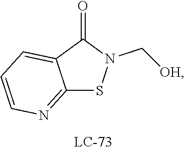 [SMILES: O=c1c2cccnc2sn1CO]

or an N-oxide, crystalline form, hydrate thereof, or a pharmaceutically acceptable salt thereof.

Compositions of the foregoing embodiments are also provided wherein one or more of the foregoing compounds are combined with a pharmaceutically acceptable carrier, adjuvant or vehicle.

In the various embodiments of the present disclosure, compounds may be recited as a group. It is to be understood that the present disclosure includes each individual compound alone or in any possible grouping of one, two, three or more compounds.

In the present disclosure, and as noted in the Examples, the residue numbering is based on the sequence of the mature PDI protein, dapeeedhvlvlrksnfaealaahkyllvefyapwcghckalapeyakaagklkaegseirlakvdateesdlaqqygvr gyptikffrngdtaspkeytagreaddivnwlkkrtgpaattlpdgaaaeslvessevavigffkdvesdsakqflqaaeaid dipfgitsnsdvfskyqldkdgvvlfkkfdegrnnfegevtkenlldfikhnqlplviefteqtapkifggeikthillflpksvsdyd gklsnfktaaesfkgkilfifidsdhtdnqrileffglkkeecpavrlitleeemtkykpeseeltaeritefchrflegkikphlmsq elpedwdkqpvkvlvgknfedvafdekknvfvefyapwcghckqlapiwdklgetykdheniviakmdstaneveavk vhsfptlkffpasadrtvidyngertldgfkkflesggqdgagddddledleeaeepdmeedddqkavkdel, (SEQ ID NO: 1) i.e., residue 1 of the mature PDI corresponds to residue 18 in the full length PDI. The first 17 amino acids in full length PDI are the signal sequence that is processed out to generate the mature PDI.

Another embodiment of the present disclosure is a method for identifying a compound that targets a disease-related reactive oxygen species (ROS) regulator. This method comprises:
(a) providing siRNA, gRNA, and/or cDNA libraries that target a plurality of ROS regulators;
(b) screening the libraries assembled in (a) for candidate ROS regulators that are associated with a disease;
(c) identifying desired ROS regulator(s) by validating candidate ROS regulators in cell assays specific for the disease;
(d) assembling a compound library based on a plurality of redox regulating scaffolds;
(e) screening the library assembled in (d) for candidate scaffolds that modulate the ROS regulator(s) identified in (c);
(f) identifying the desired scaffold(s) by evaluating safety and selectivity of the candidate scaffolds; and
(g) identifying a candidate compound that targets the disease-related ROS regulator based on the compound's ability to bind to the desired scaffold identified in (f).

As used herein, “reactive oxygen species” or “ROS” refers to radical and non-radical oxygen species formed by the partial reduction of oxygen. Examples of ROS include peroxides, superoxide, hydroxyl radical, and singlet oxygen. Non-limiting examples of a ROS regulator include glutathione peroxidases (GPX), glutathione (GSH), NF-E2-related factor 2 (Nrf2), superoxide dismutases (SOD), peroxiredoxins, catalases, glutaredoxins, thioredoxins, protein disulfide isomerases (PDI). In some embodiments of the present disclosure, the ROS regulator is a thiol-containing protein. In some embodiments of the present invention, the ROS regulator is a protein from the thioredoxin superfamily. In preferred embodiments, the ROS regulator is a protein disulfide isomerase (PDI).

As used herein, an “siRNA” is a duplex RNA oligonucleotide, that is a short, double-stranded RNA molecule, that interferes with the expression of a gene in a cell that produces RNA, after the molecule is introduced into the cell. For example, it targets and binds to a complementary nucleotide sequence in a single stranded (ss) target RNA molecule, such as an mRNA or a micro RNA (miRNA). The target RNA is then degraded by the cell.

As used herein, the term “guide RNA” or “gRNA” refers to the RNAs that guide the insertion or deletion of uridine residues into mitochondrial mRNAs in kinetoplastid protists in a process known as RNA editing. In the case of Cas9 in DNA editing, “guide RNA” or “gRNA” generally refers to an RNA molecule (or a group of RNA molecules collectively) that can bind to a Cas protein and aid in targeting the Cas protein to a specific location within a target polynucleotide (e.g., a DNA).

As used herein, “complementary DNA” or “cDNA” refers to DNA synthesized from a single stranded RNA (e.g., messenger RNA (mRNA) or microRNA) template in a reaction catalyzed by the enzyme reverse transcriptase. cDNA is often used to clone eukaryotic genes in prokaryotes. When expressing a specific protein in a cell that does not normally express that protein (i.e., heterologous expression), one will transfer the cDNA that codes for the protein to the recipient cell. cDNA is also produced naturally by retroviruses (such as HIV-1, HIV-2, simian immunodeficiency virus, etc.) and then integrated into the host's genome, where it creates a provirus. The term cDNA is also used, typically in a bioinformatics context, to refer to an mRNA transcript's sequence, expressed as DNA bases (GCAT) rather than RNA bases (GCAU). cDNA is derived from mRNA, it contains only exons, with no introns.

In one aspect of this embodiment, the disease is a neurodegenerative disease. Non-limiting examples of a neurodegenerative disease include Alzheimer's disease, Parkinson's disease, amyotrophic lateral sclerosis (ALS), Friedreich's ataxia, multiple sclerosis, Huntington's disease, transmissible spongiform encephalopathy, Charcot-Marie-Tooth disease, dementia with Lewy bodies, corticobasal degeneration, progressive supranuclear palsy, and hereditary spastic paraparesis. In some embodiments, the neurodegenerative disease is Huntington's disease. In some embodiments, the neurodegenerative disease is Alzheimer's disease.

Another embodiment of the present disclosure is a method for treating or ameliorating the effects of a disease in a subject in need thereof. This method comprises administering to the subject an effective amount of a compound identified by the method disclosed herein.

In one aspect of this embodiment, the disease is a neurodegenerative disease. Non-limiting examples of a neurodegenerative disease include Alzheimer's disease, Parkinson's disease, amyotrophic lateral sclerosis (ALS), Friedreich's ataxia, multiple sclerosis, Huntington's disease, transmissible spongiform encephalopathy, Charcot-Marie-Tooth disease, dementia with Lewy bodies, corticobasal degeneration, progressive supranuclear palsy, and hereditary spastic paraparesis. In some embodiments, the neurodegenerative disease is Huntington's disease. In some embodiments, the neurodegenerative disease is Alzheimer's disease.

In another aspect of this embodiment, the subject is a mammal. In some aspects of this embodiment the mammal is selected from the group consisting of humans, veterinary animals, and agricultural animals. In another aspect of this embodiment the subject is a human.

In yet another aspect of this embodiment, the method further comprises co-administering to the subject an effective amount of one or more additional therapeutic agents. Preferably, the one or more additional therapeutic agents are selected from the group consisting of 5-hydroxytryptophan, Activase, AFQ056 (Novartis), Aggrastat, Albendazole, alpha-lipoic acid/L-acetyl carnitine, Alteplase, Amantadine (Symmetrel), amlodipine, Ancrod, Apomorphine (Apokyn), Arimoclomol, Arixtra, Armodafinil, Ascorbic acid, Ascriptin, Aspirin, atenolol, Avonex, baclofen (Lioresal), Banzel, Benztropine (Cogentin), Betaseron, BGG492 (Novartis Corp.), Botulinum toxin, Bufferin, Carbatrol®, Carbidopa/levodopa immediate-release (Sinemet), Carbidopa/levodopa oral disintegrating (Parcopa), Carbidopa/levodopa/Entacapone (Stalevo), CERE-110: Adeno-Associated Virus Delivery of NGF (Ceregene), cerebrolysin, CinnoVex, citalopram, citicoline, Clobazam, Clonazepam, Clopidogrel, clozapine (Clozaril), Coenzyme Q, Creatine, dabigatran, dalteparin, Dapsone, Davunetide, Deferiprone, Depakene®, Depakote ER®, Depakote®, Desmoteplase, Diastat, Diazepam, Digoxin, Dilantin®, Dimebon, dipyridamole, divalproex (Depakote), Donepezil (Aricept), EGb 761, Eldepryl, ELND002 (Elan Pharmaceuticals), Enalapril, enoxaparin, Entacapone (Comtan), epoetin alfa, Eptifibatide, Erythropoietin, Escitalopram, Eslicarbazepine acetate, Esmolol, Ethosuximide, Ethyl-EPA (Miraxion™), Exenatide, Extavia, Ezogabine, Felbamate, Felbatol®, Fingolimod (Gilenya), fluoxetine (Prozac), fondaparinux, Fragmin, Frisium, Gabapentin, Gabitril®, Galantamine, Glatiramer (Copaxone), haloperidol (Haldol), Heparin, human chorionic gonadotropin (hCG), Idebenone, Inovelon®, insulin, Interferon beta 1a, Interferon beta 1b, ioflupane 1231 (DATSCAN®), IPX066 (Impax Laboratories Inc.), JNJ-26489112 (Johnson and Johnson), Keppra®, Klonopin, Lacosamide, L-Alpha glycerylphosphorylcholine, Lamictal®, Lamotrigine, Levetiracetam, liraglutide, Lisinopril, Lithium carbonate, Lopressor, Lorazepam, losartan, Lovenox, Lu AA24493, Luminal, LY450139 (Eli Lilly), Lyrica, Masitinib, Mecobalamin, Memantine, methylprednisolone, metoprolol tartrate, Minitran, Minocycline, mirtazapine, Mitoxantrone (Novantrone), Mysoline®, Natalizumab (Tysabri), Neurontin®, Niacinamide, Nitro-Bid, Nitro-Dur, nitroglycerin, Nitrolingual, Nitromist, Nitrostat, Nitro-Time, Norepinephrine (NOR), Carbamazepine, octreotide, Onfi®, Oxcarbazepine, Oxybutinin chloride, PF-04360365 (Pfizer), Phenobarbital, Phenytek®, Phenytoin, piclozotan, Pioglitazone, Plavix, Potiga, Pramipexole (Mirapex), pramlintide, Prednisone, Primidone, Prinivil, probenecid, Propranolol, PRX-00023 (EPIX Pharmaceuticals Inc.), PXT3003, Quinacrine, Ramelteon, Rasagiline (Azilect), Rebif, ReciGen, remacemide, Resveratrol, Retavase, reteplase, riluzole (Rilutek), Rivastigmine (Exelon), Ropinirole (Requip), Rotigotine (Neupro), Rufinamide, Sabril, safinamide (EMD Serono), Salagen, Sarafem, Selegiline (1-deprenyl, Eldepryl), SEN0014196 (Siena Biotech), sertraline (Zoloft), Simvastatin, Sodium Nitroprussiate (NPS), sodium phenylbutyrate, Stanback Headache Powder, Tacrine (Cognex), Tamoxifen, tauroursodeoxycholic acid (TUDCA), Tegretol®, Tenecteplase, Tenormin, Tetrabenazine (Xenazine), THR-18 (Thrombotech Ltd.), Tiagabine, Tideglusib, tirofiban, tissue plasminogen activator (tPA), tizanidine (Zanaflex), TNKase, Tolcapone (Tasmar), Tolterodine, Topamax®, Topiramate, Trihexyphenidyl (formerly Artane), Trileptal®, ursodiol, Valproic Acid, valsartan, Varenicline (Pfizer), Vimpat, Vitamin E, Warfarin, Zarontin®, Zestril, Zonegran®, Zonisamide, Zydis selegiline HCL Oral disintegrating (Zelapar), and combinations thereof.

Another embodiment of the present disclosure is a compound identified by the method disclosed herein. In preferred embodiments, the desired scaffold comprises one of the following pharmacophores:

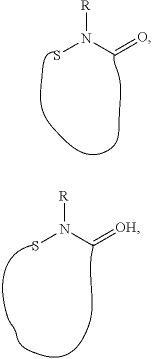

wherein R indicates a functional group.

Another embodiment of the present disclosure is a composition comprising a compound of the present disclosure, individually or in combination, and a pharmaceutically acceptable carrier, adjuvant or vehicle.

As used herein, the term “pharmacophore” is an abstract description of molecular features that are necessary for molecular recognition of a ligand by a biological macromolecule. According to IUPAC, a pharmacophore is “an ensemble of steric and electronic features that is necessary to ensure the optimal supramolecular interactions with a specific biological target and to trigger (or block) its biological response”. A pharmacophore model explains how structurally diverse ligands can bind to a common receptorsite. Furthermore, pharmacophore models can be used to identify through de novo design or virtual screening novel ligands that will bind to the same receptor.

Another embodiment of the present disclosure is a detectable probe for measuring protein disulfide isomerase (PDI) abundance and target engagement in vitro and in vivo. The detectable probe comprises a probe portion that targets a protein disulfide isomerase (PDI), and a detectable label portion.

In one aspect of this embodiment, the probe portion includes

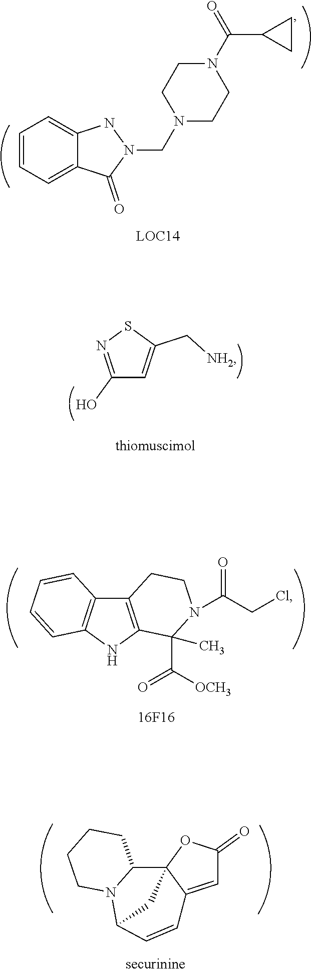

and other structurally related compounds. Preferably, the probe portion includes LOC14. Preferably, the detectable label portion is selected from the group consisting of radiolabels, fluorescent labels, an enzyme, a hapten, a phosphorescent molecule, a chemiluminescent molecule, a chromophore, a luminescent molecule, a photoaffinity molecule, a color particle or a ligand. In some embodiments, the detectable probe is radiolabeled. Non-limiting examples of radiolabels used include Oxygen-15 water, Nitrogen-13 ammonia, [⁸²Rb] Rubidium-82 chloride, [¹¹C] Acetate, [¹¹C] 25B-NBOMe, [¹⁸F] Altanserin, [¹¹C] Carfentanil, [¹¹C] DASB, [¹¹C] DTBZ or [¹⁸F] Fluoropropyl-DTBZ, [¹¹C] ME@HAPTHI, [¹⁸F] Fallypride, [¹⁸F] Florbetaben, [¹⁸F] Flubatine, [¹⁸F] Fluspidine, [¹⁸F] Florbetapir, [¹⁸F] or [¹¹C] Flumazenil, [¹⁸F] Flutemetamol, [¹⁸F] Fluorodopa, [¹⁸F] Desmethoxyfallypride, [¹⁸F] Mefway, [¹⁸F] MPPF, [¹⁸F] Nifene, Pittsburgh compound B, [¹¹C] Raclopride, [¹⁸F] Setoperone, [¹⁸F] or [¹¹C] N-Methylspiperone, [¹¹C] Verapamil, [¹¹C] Martinostat, Fludeoxyglucose (¹⁸F)(FDG)-glucose analogue, [¹¹C] Acetate, [¹¹C] Methionine, [¹¹C] choline, [¹⁸F] Fluciclovine, [¹⁸F] Fluorocholine, [¹⁸F] FET, [¹⁸F] FMISO, [¹⁸F] Fluorothymidine F-18, [⁶⁸Ga] DOTA-pseudopeptides, [⁶⁸Ga] PSMA, and [⁶⁸Ga] CXCR4.

Another embodiment of the present disclosure is a method of patient selection. This method comprises:
(a) administering to a subject with a neurodegenerative disease a detectable probe disclosed herein;
(b) determining the distribution of the protein disulfide isomerase (PDI) in the subject;
(c) comparing the distribution determined in step (b) with a reference distribution determined in a healthy subject; and
(d) selecting the subject for appropriate treatment based on the comparison result obtained in step (c).

Another embodiment of the present disclosure is a method for measuring protein disulfide isomerase (PDI) target engagement of a compound in a subject. This method comprises:
(a) administering the compound to the subject;
(b) administering to the subject a detectable probe disclosed herein; and
(c) measuring the PDI target engagement of the compound.

Another embodiment of the present disclosure is a method for determining efficacy of a compound in treating a neurodegenerative disease in a subject. This method comprises:
(a) obtaining a biological sample from the subject;
(b) contacting the sample with a detectable probe disclosed herein;
(c) determining the abundance of the PDI in the subject;
(d) administering the compound to the subject;
(e) after a certain period of time, repeating steps (a) to (c) to obtain an updated abundance of the PDI in the subject;
(f) if the abundance of the PDI determined in step (e) is significantly higher than the abundance of the PDI determined in step (c), the disease in the subject is progressing and the compound is not effective; and
(g) adjusting the treatment protocol for the subject diagnosed with progressing disease.

Preferably, in the above disclosed methods, the neurodegenerative disease is Alzheimer's disease or Huntington's disease.

Another embodiment of the present disclosure is a method for diagnosing a disease in a subject in need thereof. This method comprises:
(a) administering to the subject a detectable probe that targets a reactive oxygen species (ROS) regulator, which is associated with the disease;
(b) determining the distribution of the ROS regulator in the subject; and
(c) if the distribution determined in step (b) is significantly different from a reference distribution determined in a healthy subject, the subject is diagnosed with the disease.

As used herein, “reactive oxygen species” or “ROS” refers to radical and non-radical oxygen species formed by the partial reduction of oxygen. Examples of ROS include peroxides, superoxide, hydroxyl radical, and singlet oxygen. Non-limiting examples of a ROS regulator include glutathione peroxidases (GPX), glutathione (GSH), NF-E2-related factor 2 (Nrf2), superoxide dismutases (SOD), peroxiredoxins, catalases, glutaredoxins, thioredoxins, protein disulfide isomerases (PDI), and combinations thereof. In some embodiments of the present disclosure, the ROS regulator is a thiol-containing protein. In some embodiments of the present disclosure, the ROS regulator is a protein from the thioredoxin superfamily. In preferred embodiments, the ROS regulator is a protein disulfide isomerase (PDI).

In one aspect of this embodiment, the disease is a neurodegenerative disease. Non-limiting examples of a neurodegenerative disease include Alzheimer's disease, Parkinson's disease, amyotrophic lateral sclerosis (ALS), Friedreich's ataxia, multiple sclerosis, Huntington's disease, transmissible spongiform encephalopathy, Charcot-Marie-Tooth disease, dementia with Lewy bodies, corticobasal degeneration, progressive supranuclear palsy, and hereditary spastic paraparesis. In a preferred embodiment, the neurodegenerative disease is Alzheimer's disease. In another preferred embodiment, the neurodegenerative disease is Huntington's disease.

In another aspect of this embodiment, the subject is a mammal. In some aspects of this embodiment the mammal is selected from the group consisting of humans, veterinary animals, and agricultural animals. In another aspect of this embodiment the subject is a human.

In one aspect of this embodiment, the detectable probe comprises a probe portion and a detectable label portion. In this embodiment, the probe portion includes compounds that target PDI, thioredoxin scaffolds and other related molecules. Non-limiting examples of such probe portions include

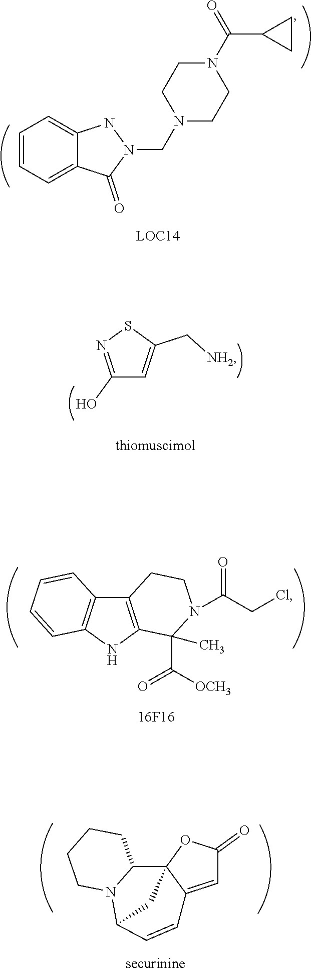

and other structurally related compounds. Preferably, the probe portion includes LOC14. Preferably, the detectable label portion is selected from the group consisting of radiolabels, fluorescent labels, an enzyme, a hapten, a phosphorescent molecule, a chemiluminescent molecule, a chromophore, a luminescent molecule, a photoaffinity molecule, a color particle or a ligand. In some embodiments, the detectable probe is radiolabeled. Non-limiting examples of radiolabels used include Oxygen-15 water, Nitrogen-13 ammonia, [⁸²Rb] Rubidium-82 chloride, [¹¹C] Acetate, [¹¹C] 25B-NBOMe, [¹⁸F] Altanserin, [¹¹C] Carfentanil, [¹¹C] DASB, [¹¹C] DTBZ or [¹⁸F] Fluoropropyl-DTBZ, [¹¹C] ME@HAPTHI, [¹⁸F] Fallypride, [¹⁸F] Florbetaben, [¹⁸F] Flubatine, [¹⁸F] Fluspidine, [¹⁸F] Florbetapir, [¹⁸F] or [¹¹C] Flumazenil, [¹⁸F] Flutemetamol, [¹⁸F] Fluorodopa, [¹⁸F] Desmethoxyfallypride, [¹⁸F] Mefway, [¹⁸F] MPPF, [¹⁸F] Nifene, Pittsburgh compound B, [¹¹C] Raclopride, [¹⁸F] Setoperone, [¹⁸F] or [¹¹C] N-Methylspiperone, [¹¹C] Verapamil, [¹¹C] Martinostat, Fludeoxyglucose (¹⁸F)(FDG)-glucose analogue, [¹¹C] Acetate, [¹¹C] Methionine, [¹¹C] choline, [¹⁸F] Fluciclovine, [¹⁸F] Fluorocholine, [¹⁸F] FET, [¹⁸F] FMISO, [¹⁸F] Fluorothymidine F-18, [⁶⁸Ga] DOTA-pseudopeptides, [⁶⁸Ga] PSMA, and [⁶⁸Ga] CXCR4.

In another aspect of this embodiment, the determination in step (b) is carried out by positron emission tomography (PET).

Another embodiment of the present disclosure is a method for monitoring the progress of a disease in a subject in need thereof. This method comprises:
(a) obtaining a biological sample from the subject;
(b) contacting the sample with a detectable probe that targets a reactive oxygen species (ROS) regulator, which is associated with the disease;
(c) determining the abundance of the ROS regulator in the subject;
(d) after a certain period of time, repeating steps (a) to (c) to obtain an updated abundance of the ROS regulator in the subject;
(e) if the abundance of the ROS regulator determined in step (d) is significantly higher than the abundance of the ROS regulator determined in step (c), the disease in the subject is progressing; and
(f) initiating a treatment protocol for the subject diagnosed with a progressing disease.

In one aspect of this embodiment, the ROS regulator is selected from the group consisting of glutathione peroxidases (GPX), glutathione (GSH), NF-E2-related factor 2 (Nrf2), superoxide dismutases (SOD), peroxiredoxins, catalases, glutaredoxins, thioredoxins, protein disulfide isomerases (PDI), and combinations thereof. In some embodiments of the present disclosure, the ROS regulator is a thiol-containing protein. In some embodiments of the present disclosure, the ROS regulator is a protein from the thioredoxin superfamily. In preferred embodiments, the ROS regulator is a protein disulfide isomerase (PDI).

In one aspect of this embodiment, the disease is a neurodegenerative disease. Non-limiting examples of a neurodegenerative disease include Alzheimer's disease, Parkinson's disease, amyotrophic lateral sclerosis (ALS), Friedreich's ataxia, multiple sclerosis, Huntington's disease, transmissible spongiform encephalopathy, Charcot-Marie-Tooth disease, dementia with Lewy bodies, corticobasal degeneration, progressive supranuclear palsy, and hereditary spastic paraparesis. In a preferred embodiment, the neurodegenerative disease is Alzheimer's disease. In another preferred embodiment, the neurodegenerative disease is Huntington's disease.

In another aspect of this embodiment the subject is a mammal. In some aspects of this embodiment the mammal is selected from the group consisting of humans, veterinary animals, and agricultural animals. In another aspect of this embodiment the subject is a human.

In one aspect of this embodiment, the detectable probe comprises a probe portion and a detectable label portion. In this embodiment, the probe portion includes compounds that target PDI, thioredoxin scaffolds and other related molecules. Non-limiting examples of such probe portions include

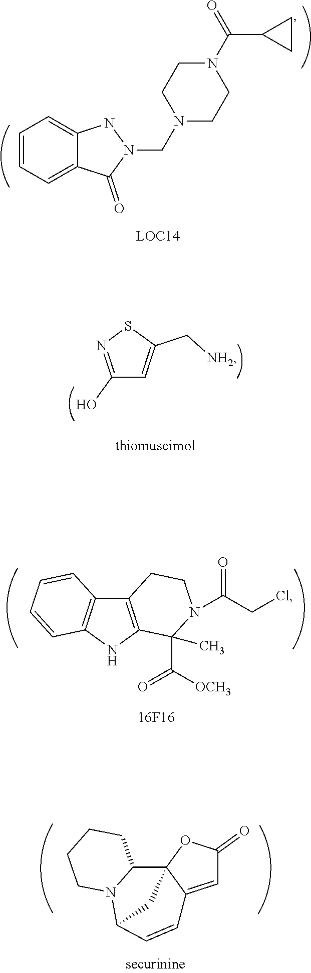

and other structurally related compounds. Preferably, the probe portion includes LOC14. Preferably, the detectable label portion is selected from the group consisting of radiolabels, fluorescent labels, an enzyme, a hapten, a phosphorescent molecule, a chemiluminescent molecule, a chromophore, a luminescent molecule, a photoaffinity molecule, a color particle or a ligand. In some embodiments, the detectable probe is radiolabeled. Non-limiting examples of radiolabels used include Oxygen-15 water, Nitrogen-13 ammonia, [⁸²Rb] Rubidium-82 chloride, [¹¹C] Acetate, [¹¹C] 25B-NBOMe, [¹⁸F] Altanserin, [¹¹C] Carfentanil, [¹¹C] DASB, [¹¹C] DTBZ or [¹⁸F] Fluoropropyl-DTBZ, [¹¹C] ME@HAPTHI, [¹⁸F] Fallypride, [¹⁸F] Florbetaben, [¹⁸F] Flubatine, [¹⁸F] Fluspidine, [¹⁸F] Florbetapir, [¹⁸F] or [¹¹C] Flumazenil, [¹⁸F] Flutemetamol, [¹⁸F] Fluorodopa, [¹⁸F] Desmethoxyfallypride, [¹⁸F] Mefway, [¹⁸F] MPPF, [¹⁸F] Nifene, Pittsburgh compound B, [¹¹C] Raclopride, [¹⁸F] Setoperone, [¹⁸F] or [¹¹C] N-Methylspiperone, [¹¹C] Verapamil, [¹¹C] Martinostat, Fludeoxyglucose (¹⁸F)(FDG)-glucose analogue, [¹¹C] Acetate, [¹¹C] Methionine, [¹¹C] choline, [¹⁸F] Fluciclovine, [¹⁸F] Fluorocholine, [¹⁸F] FET, [¹⁸F] FMISO, [¹⁸F] Fluorothymidine F-18, [⁶⁸Ga] DOTA-pseudopeptides, [⁶⁸Ga] PSMA, and [⁶⁸Ga] CXCR4.

Another embodiment of the present disclosure is a method for diagnosing Alzheimer's disease in a subject in need thereof. This method comprises:
(a) administering to the subject a detectable probe that targets a protein disulfide isomerase (PDI), which is associated with Alzheimer's disease;
(b) determining the distribution of the PDI in the subject by positron emission tomography (PET); and
(c) if the distribution determined in step (b) is significantly different from a reference distribution determined in a healthy subject, the subject is diagnosed with Alzheimer's disease.

Yet another embodiment of the present disclosure is a method for monitoring the progress of Alzheimer's disease in a subject in need thereof. This method comprises:
(a) obtaining a biological sample from the subject;
(b) contacting the sample with a detectable probe that targets a protein disulfide isomerase (PDI), which is associated with Alzheimer's disease;
(c) determining the abundance of the PDI in the subject;
(d) after a certain period of time, repeating steps (a) to (c) to obtain an updated abundance of the PDI in the subject;
(e) if the abundance of the PDI determined in step (d) is significantly higher than the abundance of the PDI determined in step (c), Alzheimer's disease in the subject is progressing; and
(f) initiating a treatment protocol for the subject diagnosed with progressing Alzheimer's disease.

Another embodiment of the present disclosure is a method for diagnosing Huntington's disease in a subject in need thereof. This method comprises:
(a) administering to the subject a detectable probe that targets a protein disulfide isomerase (PDI), which is associated with Huntington's disease;
(b) determining the distribution of the PDI in the subject by positron emission tomography (PET); and
(c) if the distribution determined in step (b) is significantly different from a reference distribution determined in a healthy subject, the subject is diagnosed with Huntington's disease.

Yet another embodiment of the present disclosure is a method for monitoring the progress of Huntington's disease in a subject in need thereof. This method comprises:
(a) obtaining a biological sample from the subject;
(b) contacting the sample with a detectable probe that targets a protein disulfide isomerase (PDI), which is associated with Huntington's disease;
(c) determining the abundance of the PDI in the subject;
(d) after a certain period of time, repeating steps (a) to (c) to obtain an updated abundance of the PDI in the subject;
(e) if the abundance of the PDI determined in step (d) is significantly higher than the abundance of the PDI determined in step (c), Huntington's disease in the subject is progressing; and
(f) initiating a treatment protocol for the subject diagnosed with progressing Huntington's disease.

The following examples are provided to further illustrate certain aspects of the present disclosure. These examples are illustrative only and are not intended to limit the scope of the disclosure in any way.

### EXAMPLES

### Example 1

### Materials and Methods

### Cell Culture

PC12 mHTT^{Q103} cells were a gift from Erik S. Schweitzer (UCLA School of Medicine, Los Angeles, Calif.). These cells are stably transfected with the first exon of human HTT gene containing the pathogenic 103 CAG/CAA repeat expansion, under the control of the ecdysteroid promoter (Aiken et al., 2004). The plasmid also contains a Bombyx mori ecdysone receptor gene fused at N-terminal with VP16 transactivation domain (Suhr et al., 1998; Vilaboa et al., 2011). Addition of the ecdysone analog, tebufenozide, to the cell culture medium is used to initiate the transcription of mutant HTT (Aiken et al., 2004).

PC12 mHTT^{Q103} cells were cultured in DMEM containing 4.5 g/I glucose, 25 mM HEPES, sodium pyruvate, and no L-glutamine (Mediatech, cat. no. 15-018-CV), supplemented with 10% (v/v) Cosmic Calf serum, 2 mM L-glutamine, 100 units/mL of penicillin-streptomycin, and 0.5 mg/ml active geneticin. Cells were grown at 37° C., 9.5% CO₂, and the medium was replaced with fresh medium every 2-3 days. To induce mHTT^{Q103} expression for experiments, tebufenozide, a gift from Lynne Moore and Fred H. Gage (The Salk Institute for Biological Studies, La Jolla, Calif.), was added to the medium at 200 nM final concentration from 1 mM stock in 85% ethanol.

### High-Throughput Screen of LOC Library

LOC mother plates with compounds at 4 mg/ml were thawed and spun down (1000 rpm, 20° C., 1 min) prior to use. Biomek FX (Beckman Coulter) robotic liquid dispenser was used to handle all liquid transferring and mixing. Replica daughter plates (D1) were prepared by transferring 2 μl of compound from the mother plate into 384-deep-well clear, round bottom, polypropylene plates (Grenier cat. no. 781270) containing 98 μl of PC12 medium without selective agent geneticin to obtain compound concentrations at 80 μl/ml in 2% DMSO. Two fold serial dilution was performed across five daughter plates by transferring 50 μl of compounds (at 80 μl/ml) from the D1 plate into 50 μl of PC12 medium in daughter plate D2, mixing, and then repeating the process for the remaining three plates. Daughter plate D1 with compounds at 80 μl/ml, daughter plate D3 with compounds at 20 μl/ml and daughter plate D5 with compounds at 5 μl/ml were then used for the screen. Assay plates were set up by seeding tebufenozide-induced PC12 mHTT^{Q103} cells into 384-well black, clear-bottom plates (Corning Inc. cat. no. 3712) at a density of 7,500 cells per well in 57 μl PC12 medium without geneticin. Three microliters of compound from the daughter plates (D1, D3, and D5) were added to the assay plates for a final compound concentration of 4 μl/ml, 1 μl/ml, and 0.25 μl/ml. Four wells containing uninduced PC12 mHTT^{Q103} cells and four wells containing medium only, were also included on each plate as controls. The assay plates were incubated at 37° C., 9.5% CO₂ for 48 hours. Twenty microliters of 40% Alamar blue (Life Technologies cat. no. DAL1100) solution in PC12 medium was added to each well (1:10 final dilution) and the plates were incubated for an additional 12-24 hours at 37° C., 9.5% CO₂. Alamar blue fluorescence was read on a fluorescence plate reader (PerkinElmer Victor3) with 530 nm excitation filter and 590 nm emission filter. Each compound concentration was tested in triplicate.

The follow-up testing of primary screen hits was performed in a similar way. Fresh powder stocks of primary hits were re-ordered from vendors, dissolved in DMSO and tested in a two-fold serial dilution across 10 wells in both tebufenozide-induced and uninduced PC12 mHTT^{Q103} cells. Dose-response curves were plotted as mean±SD and fit to either four-parameter sigmoidal (for uninduced cells) or bell-shaped (induced) function using Prism (GraphPad Software).

### High-Throughput Glutathione Binding Assay

High-throughput glutathione binding assay was performed to assess chemical and biological reactivity of cysteine-reactive compounds. Detailed procedure was described in Leach et al. Toxicol. Res., 2013, 2, 235.

Reagents:
Stock solution of compounds in DMSO
400 mM Tris, pH 8.5
Reduced glutathione (GSH) 400 μM in 400 mM Tris, pH 8.5 (prepared fresh)
5-5′-dithiobis-2-nitrobenzoic acid DTNB, Ellman's reagent (20 mM in Methanol)
EDTA
N-ethylmaleimide (NEM), positive control

Ellman's Reagent Assay:

Stock solutions of the test compounds, prepared in DMSO, were diluted to 600 μM in 400 mM Tris, pH 8.5. Reduced glutathione (400 μM) solution in 400 mM Tris, pH 8.5 was prepared fresh.

5-5′-dithiobis-2-nitrobenzoic acid DTNB, Ellman's reagent (20 mM in Methanol) was prepared fresh and kept away from light. Test compounds (50 μL) were incubated with 400 μM glutathione (50 μL) in 96-well plates at room temperature. DMSO, at a concentration of 2% (v/v), 400 mM Tris, pH 8.5 was used as the vehicle control. Reactions were carried out at room temperature and DTNB was used to determine the thiol concentration present in the glutathione assay at T₀, T₆₀, T₁₈₀ minutes.

Thiols react with DTNB to form a mixed disulfide and 2-nitro-5-thiobenzoic acid (TNB), and the concentration of TNB can be measured spectroscopically. DNTB was diluted to a final concentration of 0.5 mM in 400 mM Tris, 1 mM EDTA, pH 10, and 100 μL added to each well corresponding to the time point of interest and incubated for another 15 min. The absorbance of TNB was read at 412 nm in a plate reader.

Protocol:
50 μL Stock solution of compounds diluted to 600 μM in 400 mM Tris, pH 8.5
add 50 μL 400 μM glutathione
one well per time point.
To measure the glutathione concentration:
   add 100 μL DTNB solution (0.5 mM in 400 mM Tris, 1 mM EDTA, pH 10) to the well
   incubate 10 min
   measure absorbance at 412 nm

### Molecular Cloning, Protein Expression and Purification of PDIa

Sequence for the human catalytic PDI A1 a domain (PDIa) (SEQ ID NO: 1) was generated by PCR from Ultimate ORF Clone IOH9865 (Life Technologies) as an Nde I-BamH/fragment. The amplified a domain (amino acids 18-134 in the full length PDI sequence) was then subcloned into Nde I-BamH/sites of pET-15b vector (Novagen) containing the N-terminal His6 tag and confirmed by DNA sequencing (GeneWiz, Inc.).

The PDIa construct was transformed into Escherichia coli BL21-Gold (DE3) competent cells (Agilent Technologies) and grown at 37° C. in LB medium with 100 μg/ml ampicillin until OD₆₀₀ₙₘ reached 0.5. Expression was induced with 0.5 mM IPTG at 37° C. for overnight (usually 12-15 hr). Cells were pelleted (4,000×g, 20 minutes at 4° C.) and lysed by sonication in buffer containing 50 mM Tris-HCl, pH 8.0, 150 mM NaCl, 1 mM TCEP and 5 mM MgCl₂. Cell lysate was then centrifuged at 12,000× rpm for 30 minutes at 4° C. The supernatant was loaded onto a chromatography column containing Ni Sepharose 6 Fast Flow beads (GE Life Sciences) equilibrated with PDI Suspension Buffer (50 mM Tris-HCl, pH 8.0, 150 mM NaCl and 1 mM TCEP). The bound PDIa was eluted with 250 mM imidazole in the same buffer. Recombinant PDIa was further purified using gel filtration Superdex 100 column (GE Life Sciences) in a buffer containing 20 mM Tris-HCl, pH 8.0, 150 mM NaCl, 1 mM TCEP. The fractions containing PDIa were concentrated, flash frozen, and stored at −80° C. Protein concentration was determined using absorbance at 280 nm with molar extinction coefficient (ε) 19940 M⁻¹ cm⁻¹ (for reduced PDIa with N-terminal His6 tag as calculated from amino acid sequence by ExPASy ProtParam). PDIa purity was verified by SDS-PAGE as more than 98% pure.

For NMR studies, uniformly ¹⁵N-labeled PDIa protein with an N-terminal His6 tag was prepared. The PDIa construct was transformed into Escherichia coli BL21-Gold (DE3) competent cells (Agilent Technologies). Cells were grown at 37° C. in 1 L of M9 minimal medium supplemented with 2 mM MgSO₄, 0.1 mM CaCl₂, 100 μg/ml ampicillin, 22.2 mM glucose, metals 44 solution, 30 mg nicotinic acid, 3 mg p-aminobenzoic acid, 0.3 mg biotin, 0.5 mg thiamine hydrochloride, and 0.6 g ¹⁵NH₄Cl as the sole nitrogen source. When OD₆₀₀ₙₘ reached 0.9, the temperature was reduced to 20° C. and protein expression was induced with 0.5 mM IPTG for overnight. Protein was purified as described above except the histidine tag was removed after size exclusion chromatography. Thrombin was added at 5 U/mg protein to cleave the N-terminal His6 tag. The reaction was allowed to proceed overnight at 4° C. The next day, protein solution was passed over Ni Sepharose 6 Fast Flow beads (GE Life Sciences) equilibrated with PDI Suspension Buffer and flow-through containing the ¹⁵N-labeled PDIa protein without histidine tag was concentrated, and incubated with 5 mM TCEP overnight with gentle shaking at 4° C. The next day, the protein was dialyzed into MilliQ water, flash frozen and stored at −80° C. ¹⁵N-labeled PDIa purity was verified by SDS-PAGE as more than 98% pure.

### Isothermal Titration Calorimetry (ITC)

All ITC experiments were carried out at 25° C. on MicoCal Auto-ITC200 system (GE Healthcare). Reduced PDIa was dialyzed into ITC buffer (20 mM sodium phosphate buffer pH 7.8) and loaded into a sample cell at 40 μM concentration. The compound solution was loaded into a syringe at 400 μM in the same ITC buffer with a final DMSO concentration at 0.4% (v/v). ITC titration experiments were carried out at 25° C. with 19 injections, 2 μl per injection, and 180 seconds between each injection. The reference cell power was set to 5 μcal/sec. A control experiment was performed for each compound, where each compound was titrated into buffer to account for heat released due to dilution. This background was subtracted from test data before a final dissociation constant was obtained. Data were analyzed using a one-site binding model in Origin 7.1 software. The dissociation constant, K_{d}, was calculated according to equation K_{d}=1/Kₐ. Gibbs free energy, ΔG, was calculated from equation ΔG=ΔH-TΔS. All other parameters, Kₐ, n, ΔH, ΔS, were determined directly from the titration data.

For ITC experiments with 16F16 pre-treatment, 40 μM PDIa (after dialysis into ITC buffer) was treated with 200 μM 16F16 for 12-15 hours at 4° C. Next day the whole solution was loaded into the sample cell. LOC14 at 400 μM in ITC buffer was loaded into syringe and titrated into PDIa+16F16 loaded cell. For control experiments, ITC buffer with 200 μM 16F16 was used in the cell, while 400 μM LOC14 in ITC buffer was used in the syringe.

### Size-Exclusion Buffer Dialysis Experiments for Reversibility of Binding

Prior to dialysis, fluorescence emission spectra were recorded from 315 nm-550 nm wavelength with excitation at 280 nm on a Tecan Infinite 200 microplate reader. Fluorescence readings were carried out in a 384-well low volume, black bottom plate. Each well contained 40 μl of either LOC14 (300 μM), PDIa (20 μM), or PDIa (20 μM) treated with LOC14 (300 μM) overnight. All samples were dissolved in buffer B (20 mM sodium phosphate buffer pH 7.8). After recording the initial fluorescence spectra, samples were then transferred to Amicon Ultra 10 kDa size exclusion filter spin columns for dialysis. 400 μl of buffer B was added to the spin column, the samples were centrifuged for 8 minutes on a table top microcentrifuge (12,000 rpm, 4° C.), and afterwards the flow-through was transferred to a new tube. This step was repeated three more times with fresh buffer B. 40 μl of the collected samples from the flow-through and the spin-column chamber were transferred to a new 384-well plate and the emission spectra recorded as described above.

### NMR Spectroscopy

The residue numbering in all HSQC spectra are based on the sequence of the mature PDI protein (SEQ ID NO: 1) i.e., residue 1 of the mature PDI corresponds to residue 18 in the full length PDI. The first 17 amino acids in full length PDI are the signal sequence that is processed out to generate the mature PDI.

The ¹H-¹⁵N HSQC spectra were performed on Bruker Avance III 500 Ascend (500 MHz) spectrometers at 300 K. The uniformly ¹⁵N-labeled PDIa was dissolved at 50 μM or 100 μM in 90% H₂O/10% D20 (v/v), pH 5.1. The ¹H carrier frequency was positioned at the water resonance. The ¹⁵N carrier frequency was positioned at 115 ppm. The spectral width in the ¹H dimension was 7500 Hz and the width in ω₁ (¹⁵N) dimension was 1824.6 Hz. Suppression of water signal was accomplished using the WATERGATE sequence. Heteronuclear decoupling was accomplished using GARP decoupling scheme.

The 3D NMR experiments were performed on a Bruker Avance 500 MHz spectrometer equipped with a 5 mm TXI cryogenic probe. The ¹⁵N-NOESY-HSQC and ¹⁵N-TOCSY-HSQC spectra were recorded at 300 K on the uniformly ¹⁵N-labeled PDIa that was dissolved at 500 μM in 90% H₂O/10% D20 (v/v), pH 5.1. The proton carrier frequency was positioned at the water resonance. The ¹⁵N carrier frequency was positioned at 118 ppm. The spectral width in the ¹H dimension was 7501.9 Hz and the width in ω₂ (¹⁵N) dimension was 2027.3 Hz. Suppression of water signal was accomplished using the WATERGATE sequence. The ¹⁵N-NOESY-HSQC was recorded using the mixing time of 150 msec. The ¹⁵N-TOCSY-HSQC was recorded using the mixing time of 60 msec (Kemmink et al., 1995). All the data was processed and analyzed using TopSpin 3.1 (Bruker). The assignments were performed using Sparky (T. D. Goddard and D. G. Kneller, UCSF). The mean chemical shift difference for ¹H and ¹⁵N (Δ δ_{NH}) was calculated using formula (Williamson, 2013):

$$Δ {δ}_{NH} = \frac{\sqrt{{( Δ δ {H}_{N} )}^{2} + {( ( \frac{Δ δ N}{10} ) )}^{2}}}{2}$$

### Mouse Microsome Stability Assay

Test compound (0.5 μM) was incubated at 37° C. for up to 45 minutes in 100 mM of potassium phosphate buffer (pH 7.4) containing microsomal protein (0.5 mg/mL) and an NADPH generating system (0.34 mg/mL P-nicotinamide adenine dinucleotide phosphate (NADP), 1.56 mg/mL glucose-6-phosphate, and 1.2 units/mL glucose-6-phosphate dehydrogenase). At 0, 5, 15, 30 and 45 minute intervals, an aliquot was taken and quenched with acetonitrile (ACN) containing internal standard. No-cofactor controls at 45 minutes were prepared. Following completion of the experimentation, the samples were analyzed by LC-MS/MS. The half-life (t_{1/2}) was calculated using the following equation: t_{1/2}=0.693/k, where k is the elimination rate constant of test compounds obtained by fitting the data to the equation: C=initial×exp (−k×t). Intrinsic clearance (CLᵢₙₜ) was calculated as liver clearance from the half-life using the following equation: CLᵢₙₜ=k×(ml incubation/0.5 mg protein)×(52.5 mg protein/g liver). Results were reported as peak area ratios of analyte to internal standard. The intrinsic clearance (CLᵢₙₜ) was determined from the first order elimination constant by non-linear regression.

### In Vitro Drug Metabolism Studies: Mouse Plasma Stability Assay

Test compound (1 μM) was incubated at 37° C. for up to 120 minutes in mouse plasma. At 0, 15, 30, 60, and 120 minute intervals an 100 μL aliquot was taken and quenched with 200 μL acetonitrile (ACN) containing internal standard. Following completion of the experimentation, the samples were analyzed by LC-MS/MS. The half-life (t_{1/2}) was calculated using the following equation: t_{1/2}=0.693/k, where, k is the elimination rate constant of test compounds obtained by fitting the data to the equation: C=initial×exp (−k×t). Results were reported as peak area ratios of analyte to internal standard.

### In Vitro Drug Metabolism Studies: Mouse Plasma Protein Binding Assay

A test compound at the concentration of 2000 ng/mL in plasma was added into the sample chamber, and a dialysis buffer phosphate-buffered saline (PBS) was added into the buffer chamber, covering the unit with sealing tape and incubating for 4 hours at 37° C. at approximately 100 rpm on an orbital shaker. The incubation samples were taken from both plasma and buffer chamber at the end of the incubation, and then samples were analyzed by LC-MS/MS. Protein binding and free fraction percentage were determined using peak area ratio of analyte to internal standard. Fraction bound percent was calculated as: % Bound=100*(Cₚₗₐₛₘₐ−C_{PBS})/Cₚₗₐₛₘₐ. The fraction recovered percent was calculated as: % Recovery=(V_{PBS}*C_{PBS}+Vₚₗₐₛₘₐ*Cₚₗₐₛₘₐ)/(Vₚₗₐₛₘₐ*Cₛₚᵢₖₑ) where V_{PBS} is Volume of PBS, Vₚₗₐₛₘₐ is Volume of Plasma, C_{PBS} is Drug concentration in PBS (Analyte/IS peak area ratio), Cₚₗₐₛₘₐ is Drug concentration in plasma (Analyte/IS peak area ratio), Cₛₚᵢₖₑ is Drug concentration in spiked plasma (Analyte/IS peak area ratio).

### In Vitro Drug Metabolism Studies

The microsome stability assay, plasma stability assay, and plasma protein binding assay were each performed by Alliance Pharma, Inc. (Malvern, Pa.).

### Liquid Chromatography-Mass Spectrometry

Proteins were separated by one-dimensional SDS-PAGE electrophoresis and digested with trypsin as described previously (Cardinale et al., 2008). Peptides were separated with a NanoAcquity UPLC as described previously (Yang et al., 2014) except that Solvent B was increased in a 30 minute linear gradient between 5 and 40% and post-gradient cycled to 95% B for 7 min, followed by post-run equilibration at 5% B.

Spectra were recorded in sensitivity positive ion mode with a Synapt G2 quadrupole-time-of-flight HDMS mass spectrometer (Waters Corp). Spectra were acquired for the first 59 minutes of the chromatographic run. Source settings were capillary voltage (3.2 kV), extraction cone (4 V), sampling cone (30 V), and source temperature of 80° C. The cone gas N2 flow was 30 L/hour. Analyzer settings included quadrupole profile set at manual with mass 1 as 400 (dwell time 25%, ramp time 25%), mass 2 as 500 (dwell time 25%, ramp time 25%) and mass 3 as 600. A reference sprayer was operated at 500 nL/minute to produce a lockmass spectrum with Glu-1-Fibrinopeptide B (EGVNDNEEGFFSAR) (m/z 785.8426) leucine enkephalin (YGGFL) at m/z 556.2771 every 30 s.

Data were collected by data-dependent acquisition with a scan time of 0.25 seconds. A survey scan was conducted over the range of 300 to 2000 Da. Acquisition was performed in sensitivity mode and switched when individual ion counts exceeded 1000 count/second. MS/MS spectra were acquired over the range of 50 to 2000 m/z. A maximum of the five most intense ions were selected from a single MS survey scan. Return to MS survey scan was triggered when the intensity exceeded 60,000 counts/second or 3 seconds had elapsed. Charge state peak detection was enabled for +2, +3, and +4. Collision energy in the trap was ramped from 12 to 20 volts for low mass (300 Da) and 40 to 60 V for high mass (2000 Da).

Raw spectrum processing was performed with the PLGS software (Vers. 2.5, RC9). The electrospray survey was calibrated to a lock mass of Glu-1-Fibrinopeptide B at 785.8426 m/z, averaging three scans with a tolerance of 0.1 Da, adaptive background subtraction with slow algorithm deisotoping function with 30 iterations and a 3% threshold. MS/MS spectra were calibrated to singly-charged leucine enkephalin at m/z 556.2771 with the same settings as for the survey scan. Spectra were processed and exported as .pkl files that were then imported into the Mascot database search program (Vers. 2.3.02) (Matrix Science, London, UK.). Observed masses were searched with Mascot against the full NCBI nr protein database of Jan. 4, 2012 (16,826,875 sequences; 5,780,204,515 residues). In addition, a custom database was used with sequence of the protein disulfide isomerase construct used for the reaction. Decoy search was enabled and no taxonomic restriction and up to one missed trypsin cleavage with no fixed modifications. Charge states of +1, +2, or +3 were considered. Monoisotopic mass tolerance for precursor peptides was 10 ppm, and for products, 0.02 Da. Variable modifications included carbamidomethyl (C), oxidation (M) and a custom modification of C representing inhibitor 16F16 with a monoisotopic mass shift of 284.1160 Da and an elemental composition C(16) H(16) N(2) 0(3).

All raw data files and DDA .pkl files were deposited in a public repository at www.chorusproject.org.

### HD Brain Slice Assay

Brain slice explants were prepared and transfected as previously described (Reinhart et al., 2011). Briefly, brains were taken from postnatal day 10 CD Sprague-Dawley rat pups and cut into 250 μm coronal slices on a vibratome (Vibratome Co., St. Louis, Mo.). Brain slices containing striatum and cortex were then placed in individual wells of 12-well plates atop culture medium set in 0.5% agarose and maintained at 32° C. under 5% CO₂. Co-transfection with YFP and Htt exon-1 containing a 73 polyglutamine repeat was done using a biolistic device (Bio-Rad Helios Gene Gun, Hercules, Calif.). Positive controls were transfected with YFP only or treated with a combination of 50 μM KW-6002 (istradefylline) and 50 μM SP600125. Negative control brain slices were treated with 0.1% DMSO carrier only. Striatal medium spiny neurons (MSNs) expressing YFP were visualized under fluorescence microscopy and identified based on their location within brain slices and their characteristic morphology. MSNs exhibiting normal-sized cell bodies, and even and continuous expression of YFP in at least 2 discernible primary dendrites at least 2 cell body diameters long were scored as healthy.

### Example 2

### Compound Synthesis

Starting materials were purchased form Sigma-Aldrich, Fisher Scientific, Ark Pharm, Oakwood Chemical, Cambridge Isotope Laboratory, or AK Scientific and were used as received unless stated otherwise. All solvents were reagent grade. Column chromatography was performed on a Teledyne ISCO CombiFlash® Rf+ using RediSep® Normal-phase silica flash columns. Thin layer chromatography (TLC) was performed on Silicycle SiliaPlate™ Glass TLC Plates (250 μm, 20×20 cm). ¹H NMR spectra were recorded at ambient temperature using 400 MHz, or 500 MHz spectrometers as indicated. Chemical shifts are reported in ppm relative to the residual solvent peaks (¹H NMR: DMSO-d6, δ 2.50; chloroform-d, δ 7.26; methanol-d4, δ 3.31). The following abbreviations are used to indicate multiplicity: s (singlet), d (doublet), t (triplet), q (quartet), hept (heptuplet), m (multiplet), br (broad). High resolution mass spectra (HRMS) were acquired on a time-of-flight spectrometer with atmospheric pressure chemical ionization (APCI) or electrospray ionization (ESI), as indicated, and were obtained by peak matching. All reactions were run under an atmosphere of nitrogen or argon in glassware that was flame-dried under argon unless otherwise stated. Aqueous solutions were prepared from nanopure water with a resistivity over 18 MΩ·cm. Unless otherwise noted, all reagents were commercially available.

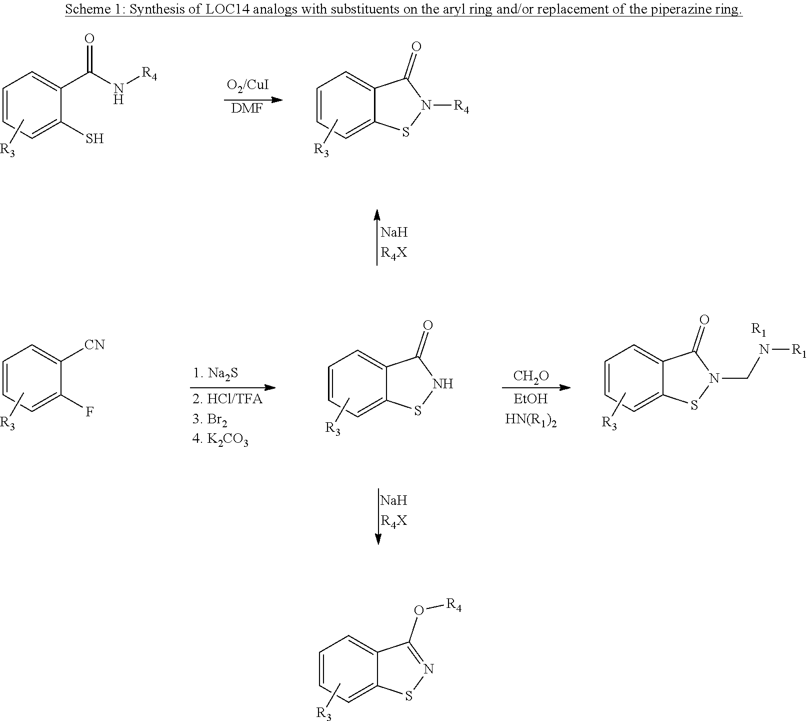

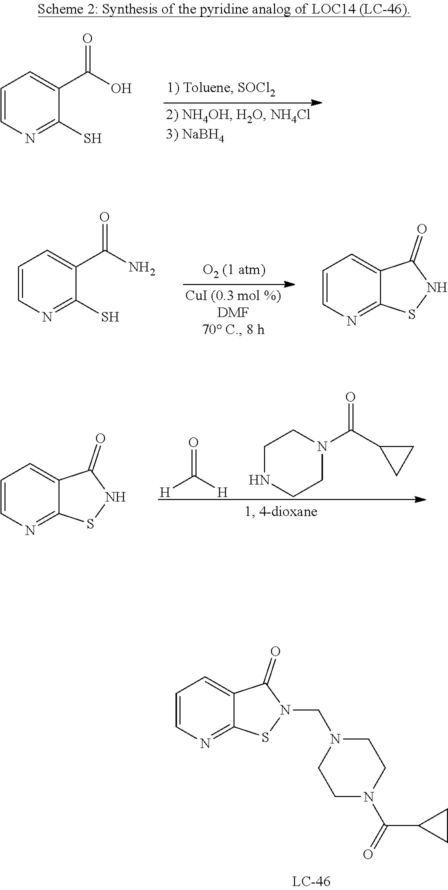

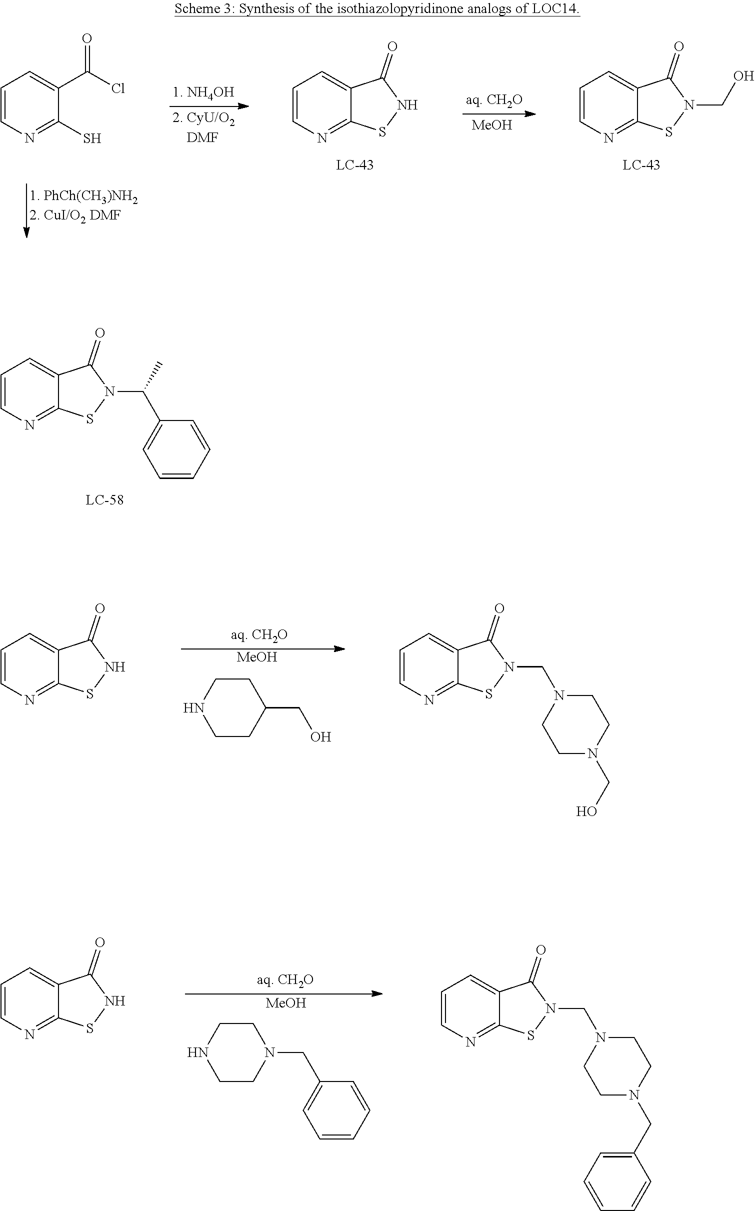

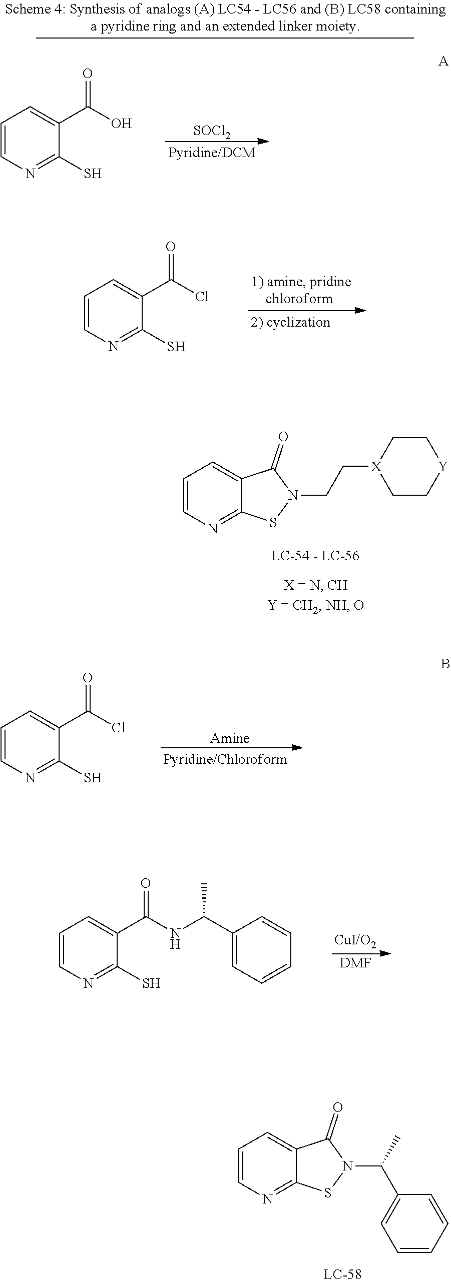

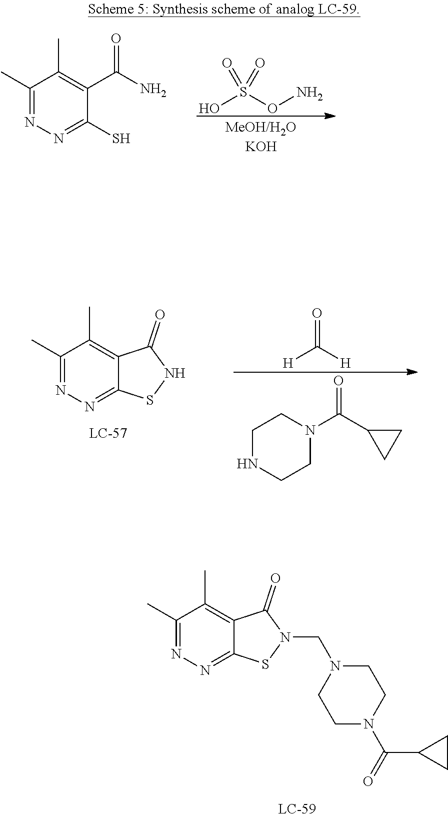

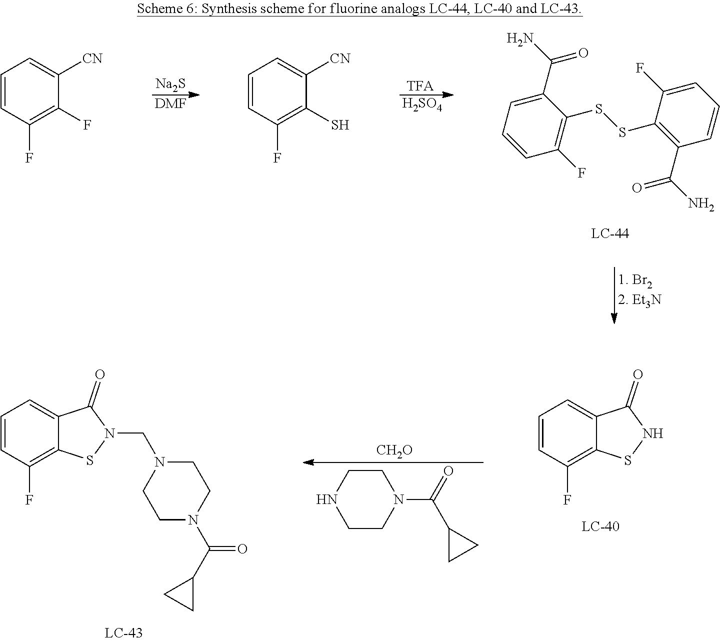

### Synthesis of 4,5-dimethylisothiazolo[5,4-c]pyridazin-3(2H)-one (LC-57)

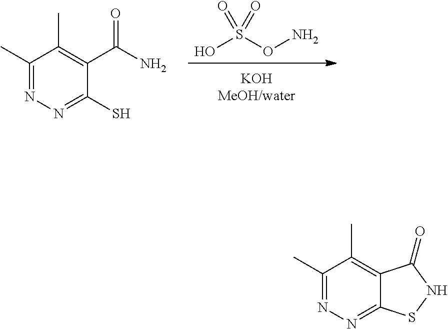

5,6-dimethyl-3-sulfanylpyridazine-4-carboxamide (183 mg, 1.0 mmol, 1.0 eq) was dissolved in a mixture of methanol (10 mL) and water (10 mL). Hydroxylamine-O-sulfonic acid (170 mg, 1.5 mmol, 1.5 eq) was dissolved in a solution of KOH (112 mg, 2.0 mmol, 2.0 eq) in water (10 mL) and added dropwise to the starting material solution at 0° C. under N2. After stirring for 3h, the reaction mixture was extracted with dichloromethane (3×) and the combined organic layers were dried with MgSO₄, filtered, and the solvent evaporated. The crude material was purified by column chromatography on silica (0-15% MeOH in DCM) to give 4,5-dimethylisothiazolo[5,4-c]pyridazin-3(2H)-one (LC-57) as a pale yellow solid (86.4 mg, 48% yield). ¹H NMR (400 MHz, Chloroform-d) δ 2.83 (s, 3H), 2.81 (s, 3H) ppm. HRMS (ESI⁺, m/z): calcd. for C₇H₈N₃OS [M+H]⁺: 182.0388, found: 182.0388.

### Synthesis of isothiazolo[5,4-b]pyridin-3(2H)-one (LC-47)

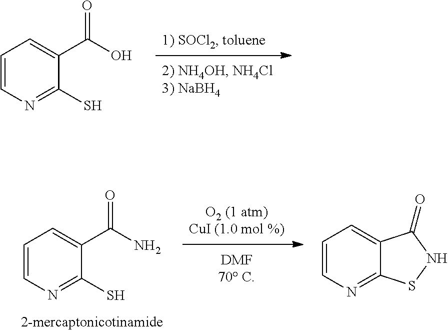 [SMILES: O=C(O)c1cccnc1S ; NC(=O)c1cccnc1S ; O=c1[nH]sc2ncccc12]

2-Mercaptonicotinic acid (10 g, 64.4 mmol, 1.0 eq) was suspended in toluene (100 mL), thionyl chloride (30 mL) was added and the reaction mixture was heated to reflux until homogenous (˜3 hours). After cooling to room temperature, the acid chloride precipitated. The reaction mixture was concentrated and the residue was resuspended in toluene and concentrated again to strip off excess thionyl chloride. The crude acid chloride was carefully added to a mixture of concentrated NH₄OH (83 mL), water (28 mL) and NH₄Cl (11.7 g). The resulting mixture was stirred for 18 h at room temperature followed by treatment with NaBH₄ (1.26 g, 33.3 mmol) and stirred for an additional 2 h. The reaction mixture was carefully acidified with aqueous HCl (3.0 M) and the product (2-mercaptonicotinamide) was collected by filtration as a light-brown solid (5.98 g, 60% yield). 2-Mercaptonicotinamide: ¹H NMR (400 MHz, Methanol-d₄) δ 8.61 (dd, J=7.6, 1.9 Hz, 1H), 7.85 (dd, J=6.1, 1.9 Hz, 1H), 6.99 (dd, J=7.6, 6.1 Hz, 1H).

The 2-mercaptonicotinamide (771 mg, 5.0 mmol, 1.0 eq) was dissolved in DMF (20.0 mL) and CuI (9.5 mg, 0.05 mmol, 1.0 mol %) was added and the reaction mixture was stirred under an 02 atmosphere at 70° C. for 8 h. After cooling to room temperature, water was added and the reaction mixture was extracted with ethyl acetate (3×). The combined organic layers were dried with Na₂SO4, filtered, and the solvent evaporated. The crude product was purified by column chromatography on silica (0-10% MeOH in DCM) to give the product as an off-white solid (548 mg, 72% yield). ¹H NMR (400 MHz, Chloroform-d) δ 8.81 (dd, J=4.7, 1.7 Hz, 1H), 8.33 (dd, J=7.9, 1.7 Hz, 1H), 7.39 (dd, J=7.9, 4.7 Hz, 1H) ppm. HRMS (ESI⁺, m/z): calcd. for C₆H₅N₂OS [M+H]⁺: 153.0123, found: 153.0123.

### Synthesis of LC-58

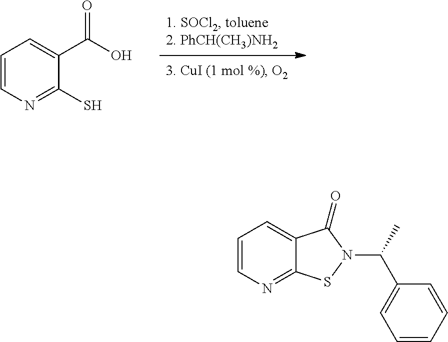 [SMILES: O=C(O)c1cccnc1S ; C[C@H](c1ccccc1)n1sc2ncccc2c1=O]

To an ice-cold suspension of 2-mercaptonicotinic acid (1.0 g, 6.44 mmol) in a mixture of pyridine (3.0 mL) and dichloromethane (8.0 mL) was added a solution of SOCl₂ (2.0 mL) in dichloromethane (8.0 mL) and stirred at 0° C. for 1h. The reaction mixture was concentrated and the residue was resuspended in toluene and concentrated again to strip off excess thionyl chloride (2×). The resulting 2-mercaptonicotinoyl chloride was used in the next step without further purification. The acid chloride (173.6 mg, 1.0 mmol, 1.0 eq) was dissolved in chloroform (2.0 mL), and pyridine (350 μL) was added followed by addition of a solution of (R)-1-phenylethan-1-amine (1.0 mmol, 1.0 eq) in chloroform (2.0 mL) at 0° C. The reaction was stirred overnight while slowly warming to room temperature. The solvent was evaporated and the crude product was used in the next step without additional purification. For the final cyclization step, the starting material (1.0 mmol, 1.0 eq) was dissolved in DMF (10.0 mL) and CuI (1.9 mg, 0.01 mmol, 1.0 mol %) was added and the reaction mixture was stirred under an 02 atmosphere at 70° C. for 8 h. After cooling to room temperature, water was added and the reaction mixture was extracted with ethyl acetate (3×). The combined organic layers were dried with Na₂SO₄, filtered, and the solvent evaporated. The crude product was purified by column chromatography on silica (0-10% MeOH in DCM) to give (R)-2-(1-phenylethyl)isothiazolo[5,4-b]pyridin-3(2H)-one (LC-58) as a yellow oil that solidified upon standing. ¹H NMR (400 MHz, Chloroform-d) δ 8.71 (dd, J=4.7, 1.7 Hz, 1H), 8.28 (dd, J=7.9, 1.7 Hz, 1H), 7.48-7.41 (m, 2H), 7.41-7.29 (m, 4H), 6.10 (q, J=7.0 Hz, 1H), 1.82 (d, J=7.0 Hz, 3H) ppm. HRMS (ESI⁺, m/z): calcd. for C₁₄H₁₃N₂OS [M+H]⁺: 257.0749, found: 257.0738.

### Synthesis of LC-60, LC-61, LC-62 and LC-63

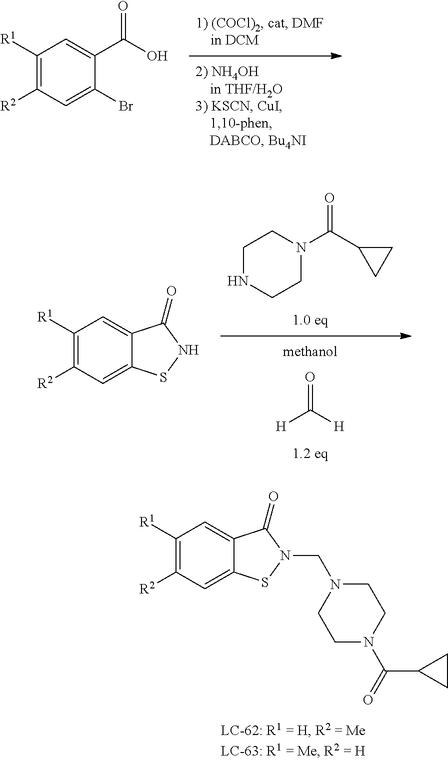

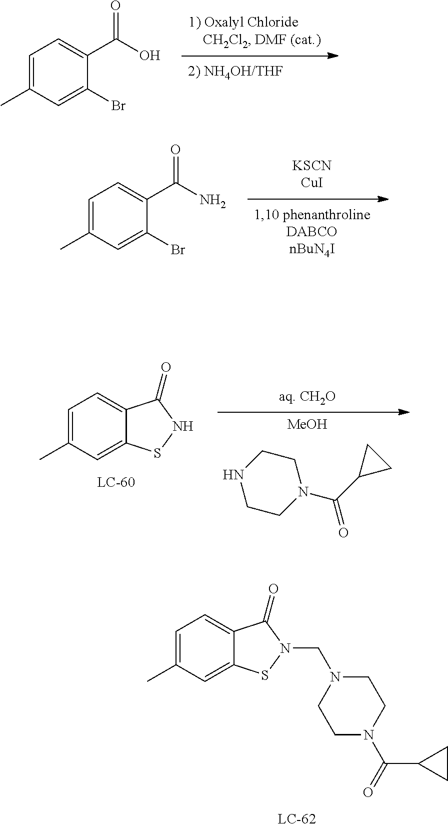

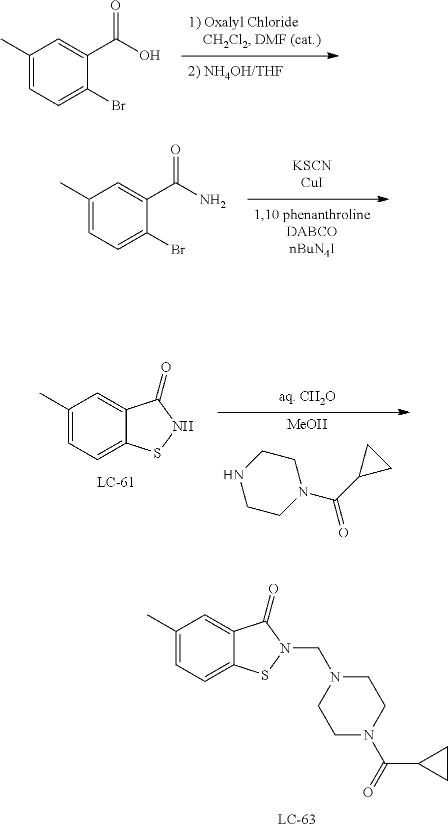

2Bromo-4-methylbenzoic acid (2.15 g, 10 mmol, 1.0 eq) or 2-Bromo-5-methylbenzoic acid (2.15 g, 10 mmol, 1.0 eq) was suspended in dichloromethane (20 mL) and treated with oxalyl chloride (2.54 g, 1.7 mL, 20 mmol, 2.0 eq) at roam temperature, followed by addition of a drop of DMF. The resulting mixture was stirred for 4 h at roam temperature. The solvent and excess reagents were evaporated in vacuo. The crude acid chloride was dissolved in tetrahydrofuran (20 mL) and added to a mixture of tetrahydrofuran (40 mL), water (20 mL), and concentrated ammonium hydroxide (2.0 mL). The reaction mixture was stirred at room temperature overnight, diluted with ethyl acetate, washed with brine, dried over sodium sulfate, filtered, and the solvent evaporated to yield the corresponding 2-bromomethylbenzamide in quantitative yield.

The benzamide (214 mg, 1.0 mmol, 1.0 eq) was dissolved in water (2.0 mL) in a high-pressure tube, followed by addition of: KSCN (194 mg, 2.0 mmol, 2.0 eq), CuI (19 mg, 0.1 mmol, 0.1 eq), 1,10-phenanthroline (36 mg, 0.2 mmol, 0.2 eq), DABCO (224 mg, 2.0 mmol, 2.0 eq), and n-Bu₄NI (74 mg, 0.2 mmol, 0.2 eq). The reaction mixture was stirred at rt for 30 min under argon and turned a purple color. The high-pressure tube was sealed and heated in an oil bath at 140° C. for 48 h. After cooling to room temperature, water was added, and the mixture was extracted with ethyl acetate (3×). The organic layer was dried with sodium sulfate, filtered, and the solvent evaporated. The crude material was purified by column chromatography on silica (0-70% EtOAc in hexanes) to give the methyl-substituted benzisothiazolone. 6-Methylbenzo[d]isothiazol-3(2H)-one (LC-60): ¹H NMR (400 MHz, Chloroform-d) δ 7.94 (d, J=8.1 Hz, 1H), 7.41 (dt, J=1.4, 0.7 Hz, 1H), 7.24 (ddd, J=8.1, 1.4, 0.7 Hz, 1H), 2.51 (s, 3H) ppm. 5-Methylbenzo[d]isothiazol-3(2H)-one (LC-61): ¹H NMR (400 MHz, Chloroform-d) δ 7.87 (dt, J=1.7, 0.7 Hz, 1H), 7.52 (dd, J=8.3, 0.7 Hz, 1H), 7.47 (ddd, J=8.3, 1.7, 0.7 Hz, 1H), 2.49 (d, J=0.7 Hz, 3H) ppm.

The methyl-substituted benzisothiazolone (44 mg, 0.266 mmol, 1.0 eq) was dissolved in methanol (5.0 mL), followed by addition of 1-(cyclopropylcarbonyl)piperazine (41 mg, 38 μL, 0.266 mmol, 1.0 eq) and formaldehyde (37% wt. % in water, 24 μL, 0.32 mmol, 1.2 eq). The reaction mixture was stirred at room temperature overnight. The solvent was evaporated and the crude product triturated from hexanes/EtOAc to give LC-62 or LC-63 as a colorless solid (77 mg, 87% yield).

### 2-((4-(cyclopropanecarbonyl)piperazin-1-yl)methyl)-6-methylbenzo[d]isothiazol-3(2H)-one (LC-62)

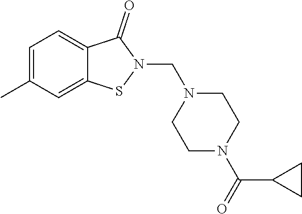 [SMILES: Cc1ccc2c(=O)n(CN3CCN(C(=O)C4CC4)CC3)sc2c1]

Colorless solid. ¹H NMR (400 MHz, Chloroform-d) δ 7.91 (d, J=8.1 Hz, 1H), 7.33 (dt, J=1.4, 0.7 Hz, 1H), 7.21 (ddd, J=8.1, 1.4, 0.7 Hz, 1H), 4.72 (s, 2H), 3.68 (bs, 4H), 2.73 (bs, 4H), 2.49 (s, 3H), 1.68 (tt, J=8.0, 4.7 Hz, 1H), 1.01-0.92 (m, 2H), 0.78-0.68 (m, 2H) ppm. HRMS (ESI⁺, m/z): calcd. for C₁₇H₂₂N₃O₂S [M+H]⁺: 332.1433, found: 332.1425.

### 2-((4-(cyclopropanecarbonyl)piperazin-1-yl)methyl)-5-methylbenzo[d]isothiazol-3(2H)-one (LC-63)

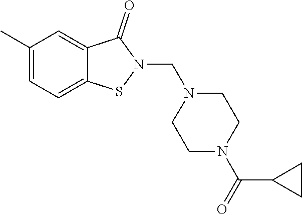 [SMILES: Cc1ccc2sn(CN3CCN(C(=O)C4CC4)CC3)c(=O)c2c1]

Colorless solid. ¹H NMR (500 MHz, Chloroform-d) δ 7.83 (dt, J=1.7, 0.7 Hz, 1H), 7.45 (dd, J=8.3, 1.7 Hz, 1H), 7.43 (dd, J=8.3, 0.7 Hz, 1H), 4.73 (s, 2H), 3.67 (bs, 4H), 2.73 (bs, 4H), 2.46 (s, 3H), 1.68 (tt, J=8.0, 4.7 Hz, 1H), 0.97-0.92 (m, 2H), 0.76-0.69 (m, 2H) ppm. HRMS (ESI⁺, m/z): calcd. for C₁₇H₂₂N₃O₂S [M+H]⁺: 332.1433, found: 332.1425.

### Synthesis of LC-64

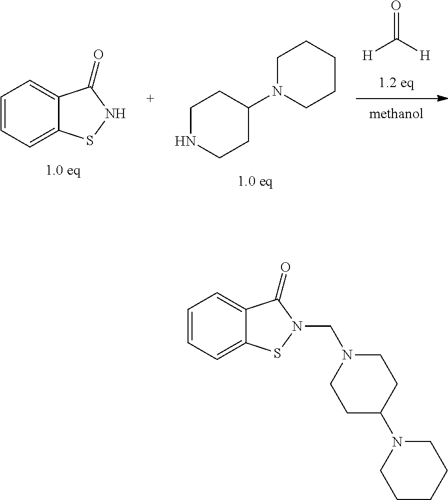

Benzisothiazolone (302 mg, 2.0 mmol, 1.0 eq), 4-piperidinopiperidine (337 mg, 2.0 mmol, 1.0 eq) were dissolved in methanol (3.0 mL), and formaldehyde (37% wt. % in water, 178 μL, 2.4 mmol, 1.2 eq) was added. The reaction mixture was stirred overnight at room temperature. The solvent was evaporated and the crude product purified by column chromatography on silica to give 2-([1,4′-bipiperidin]-1′-ylmethyl)benzo[d]isothiazol-3(2H)-one (214 mg, 32% yield) as a colorless solid. ¹H NMR (400 MHz, Chloroform-d) δ 8.03 (dt, J=7.9, 1.0 Hz, 1H), 7.60 (ddd, J=8.2, 7.0, 1.3 Hz, 1H), 7.53 (dt, J=8.1, 1.0 Hz, 1H), 7.38 (ddd, J=8.0, 7.0, 1.1 Hz, 1H), 4.68 (s, 2H), 3.16-3.02 (m, 2H), 2.50 (t, J=5.3 Hz, 4H), 2.39 (td, J=11.9, 2.4 Hz, 2H), 2.23 (tt, J=11.6, 3.7 Hz, 1H), 1.87-1.76 (m, 2H), 1.64-1.52 (m, 6H), 1.47-1.37 (m, 2H) ppm.

### Synthesis of LC-65

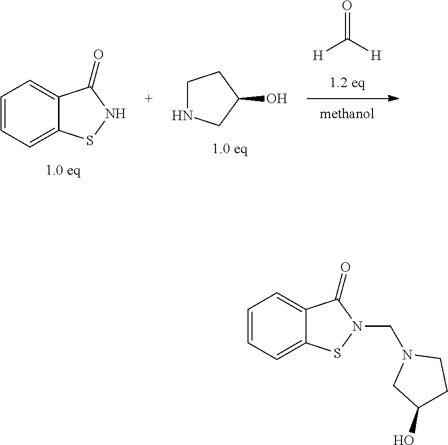 [SMILES: O=c1[nH]sc2ccccc12 ; O[C@@H]1CCNC1 ; C=O ; O=c1c2ccccc2sn1CN1CC[C@@H](O)C1]

Benzisothiazolone (302 mg, 2.0 mmol, 1.0 eq), (R)-3-pyrrolidinol (174 mg, 162 μL, 2.0 mmol, 1.0 eq) were dissolved in methanol (3.0 mL), and formaldehyde (37% wt. % in water, 178 μL, 2.4 mmol, 1.2 eq) was added. The reaction mixture was stirred overnight at room temperature. The solvent was evaporated and the crude product purified by column chromatography on silica to give (R)-2-((3-hydroxypyrrolidin-1-yl)methyl)benzo[d]isothiazol-3(2H)-one (435 mg, 87% yield) as a colorless solid. ¹H NMR (400 MHz, Chloroform-d) δ 8.04 (dt, J=7.9, 1.0 Hz, 1H), 7.61 (ddd, J=8.2, 7.0, 1.3 Hz, 1H), 7.54 (dt, J=8.1, 0.9 Hz, 1H), 7.39 (ddd, J=8.0, 7.0, 1.1 Hz, 1H), 4.95-4.78 (m, 2H), 4.37 (ddt, J=6.9, 4.8, 2.4 Hz, 1H), 3.10 (ddd, J=9.1, 8.1, 6.0 Hz, 1H), 2.97-2.85 (m, 2H), 2.74 (td, J=9.0, 5.7 Hz, 1H), 2.14 (ddt, J=13.7, 8.9, 6.3 Hz, 1H), 2.04-1.62 (m, 1H) ppm.

### Synthesis of LC-66

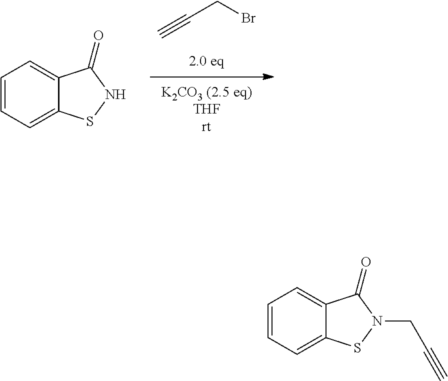

Benzisothiazolone (756 mg, 5.0 mmol, 1.0 eq) was dissolved in THF (15 mL). K₂CO₃ (1.73 g, 12.5 mmol, 2.5 eq) and propargyl bromide (1.49 g, 1.11 mL, 10.0 mmol, 2.0 eq) were added and the reaction mixture was stirred at room temperature overnight. The solvent was evaporated and ethyl acetate was added. The reaction mixture was partitioned between dichloromethane and water and the layers were separated. The aqueous layer was extracted with dichloromethane (2×), the combined organic layers dried with sodium sulfate, filtered and the solvent evaporated in vacuo. The crude material was purified by column chromatography on silica gel (0-60% EtOAc in hexanes) to give 2-(prop-2-yn-1-yl)benzo[d]isothiazol-3(2H)-one as a colorless solid (402 mg, 42% yield). ¹H NMR (500 MHz, Chloroform-d) δ 8.03 (dt, J=7.9, 1.0 Hz, 1H), 7.62 (ddd, J=8.2, 7.0, 1.3 Hz, 1H), 7.56 (dt, J=8.1, 1.0 Hz, 1H), 7.40 (ddd, J=8.0, 7.1, 1.0 Hz, 1H), 4.69 (d, J=2.6 Hz, 2H), 2.44 (t, J=2.6 Hz, 1H) ppm. ¹³C NMR (125 MHz, CDCl₃) b 165.1, 140.6, 132.3, 126.9, 125.8, 124.1, 120.6, 77.3, 74.5, 33.3 ppm. HRMS (APCI⁺, m/z): calcd. for C₁₀H₈NOS [M+H]⁺: 190.0327, found: 190.0329.

### Synthesis of LC-67

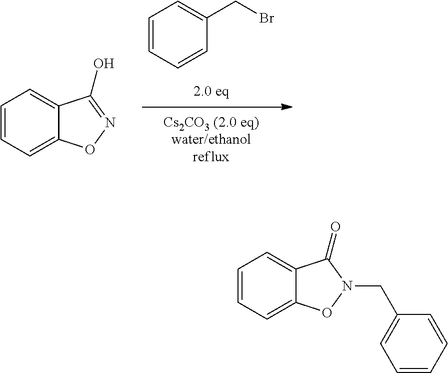 [SMILES: Oc1noc2ccccc12 ; BrCc1ccccc1 ; O=c1c2ccccc2on1Cc1ccccc1]

3-Hydroxybenzisoxazole (338 mg, 2.5 mmol, 1.0 eq) was dissolved in a mixture of water (18 mL) and ethanol (18 mL). Cs₂CO₃ (1.63 g, 5.0 mmol, 2.0 eq) and benzyl bromide (855 mg, 595 μL, 5.0 mmol, 2.0 eq) were added and the reaction mixture was stirred at reflux overnight. The ethanol was evaporated and water was added. The reaction mixture was extracted with diethyl ether (3×), the combined organic layers dried with sodium sulfate, filtered and the solvent evaporated in vacuo. The crude material was purified by column chromatography on silica gel (0-50% EtOAc in hexanes) to give 2-benzylbenzo[d]isoxazol-3(2H)-one as a colorless solid (139 mg, 25% yield). ¹H NMR (500 MHz, Chloroform-d) δ 7.70 (dt, J=7.8, 1.0 Hz, 1H), 7.41 (ddd, J=8.5, 7.2, 1.3 Hz, 1H), 7.26-7.13 (m, 5H), 7.12-7.07 (m, 1H), 7.01 (d, J=8.5 Hz, 1H), 5.04 (s, 2H). ¹³C NMR (125 MHz, CDCl₃) δ 162.6, 160.3, 134.9, 133.5, 128.9, 128.3, 128.3, 124.5, 123.6, 116.6, 110.1, 49.7 ppm. HRMS (APCI⁺, m/z): calcd. for C₁₄H₁₂NO₂ [M+H]⁺: 226.0868, found: 226.0872.

### Synthesis of LC-68

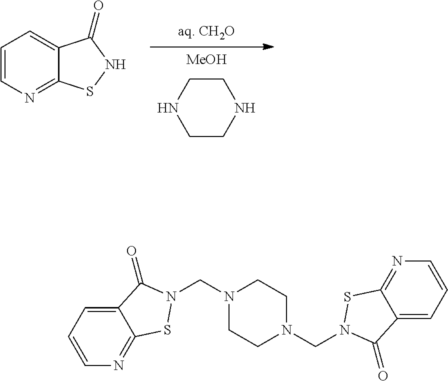 [SMILES: "MeOH" ; O=c1[nH]sc2ncccc12 ; C1CNCCN1 ; O=c1c2cccnc2sn1CN1CCN(Cn2sc3ncccc3c2=O)CC1]

A mixture of isothiazolo[5,4-b]pyridin-3(2H)-one (LC-47) (25 mg, 0.16 mmol, 1.0 eq), piperazine (14 mg, 0.16 mmol, 1.0 eq), and formaldehyde (37% wt. % in water, 0.25 mL) in absolute ethanol (5.0 mL) was heated at 80C for 12 h. The solvent was evaporated in vacuo and the crude product triturated with ethanol. The solid was filtered and washed with ethanol to give 2,2′-(piperazine-1,4-diylbis(methylene))bis(isothiazolo[5,4-b]pyridin-3(2H)-one) (LC-68) as an orange powder (16 mg). ¹H NMR (400 MHz, Chloroform-d) δ 8.76 (dd, J=4.7, 1.7 Hz, 2H), 8.27 (dd, J=7.9, 1.7 Hz, 2H), 7.34 (dd, J=7.9, 4.7 Hz, 2H) 4.71 (s, 4H), 2.79 (s, 8H).

### Synthesis of 7-fluorobenzo[d]isothiazol-3(2H)-one (LC-40)

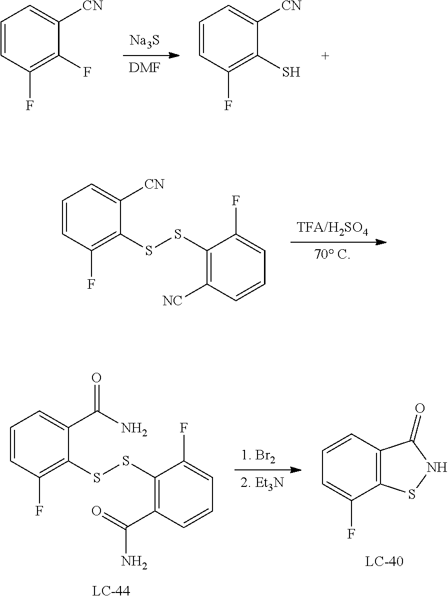

To 2,3-difluorobenzonitrile (1.0 g, 7.19 mmol) in DMF (5 ML) was added Na₂S (617 mg). After 18 h, 1 N aq. NaOH and EtOAc were added and the aqueous phase extracted with EtOAc. The aqueous phase was acidified with 1 N HCl and extracted with EtOAc. Drying of the organic layers with Na₂SO₄ and concentration in vacuo gave a mixture of yellow oil and crystalline solid (1.04 g) that was used in the next step without purification. Of this material, 256 mg was treated with 4:1 TFA:H₂SO₄ (2 mL) and heated at 70° C. overnight. After cooling to room temperature water was added. The resulting mixture was extracted with EtOAc and dried over MgSO₄. The amber oil obtained (354 mg) was purified by silica gel chromatography (CH₂Cl₂/MeOH) to give 2,2′-disulfanediylbis(3-fluorobenzamide) (LC-44) as a gray solid. To 2,2′-disulfanediylbis(3-fluorobenzamide) (60 mg, 0.176 mmol) in CH₂Cl₂ (2.5 mL) was added bromine (9.1 uL). After 22 h, Et₃N was added. Aqueous workup with EtOAc gave a brown crystalline solid (49 mg). Silica gel chromatography (EtOAC/0-10% MeOH) gave 7-fluorobenzo[d]isothiazol-3(2H)-one (LC-40) as an off-white solid (37 mg).

### Synthesis of LC-69

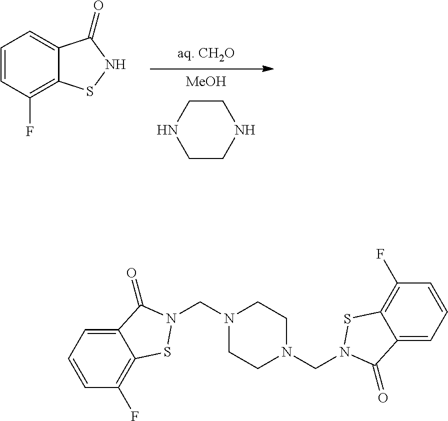 [SMILES: "MeOH" ; O=c1[nH]sc2c(F)cccc12 ; C1CNCCN1 ; O=c1c2cccc(F)c2sn1CN1CCN(Cn2sc3c(F)cccc3c2=O)CC1]

A mixture of 7-fluorobenzo[d]isothiazol-3(2H)-one (LC-40) (27 mg, 0.16 mmol, 1.0 eq), piperazine (14 mg, 0.16 mmol, 1.0 eq), and formaldehyde (37% wt. % in water, 0.25 mL) in absolute ethanol (5.0 mL) was heated at 80C for 12 h. The solvent was evaporated in vacuo and the crude product triturated with ethanol. The solid was filtered and washed with ethanol to give 2,2′-(piperazine-1,4-diylbis(methylene))bis(7-fluorobenzo[d]isothiazol-3(2H)-one) (LC-69) as a tan powder. ¹H NMR (400 MHz, Chloroform-d) δ 7.84 (dd, J=7.7, 1.0 Hz, 2H), 7.39 (ddd, J=7.7, 4.7, 8.0 Hz, 2H), 7.30 (ddd, J=9.1, 8.0, 1.0 Hz, 2H) 4.69 (s, 4H), 2.78 (s, 8H).

### Synthesis of LC-70

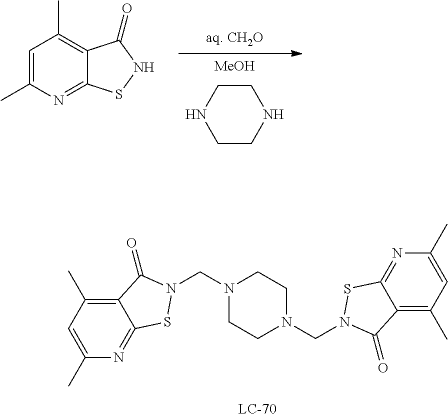 [SMILES: "MeOH" ; Cc1cc(C)c2c(=O)[nH]sc2n1 ; C1CNCCN1 ; Cc1cc(C)c2c(=O)n(CN3CCN(Cn4sc5nc(C)cc(C)c5c4=O)CC3)sc2n1]

A mixture of 4,6-dimethylisothiazolo[5,4-b]pyridin-3(2H)-one (50 mg, 0.28 mmol, 1.0 eq), piperazine (24 mg, 0.28 mmol, 1.0 eq), and formaldehyde (37% wt. % in water, 0.35 mL) in absolute ethanol (5.0 mL) was heated at 80C for 12 h. The solvent was evaporated in vacuo and the crude product triturated with ethanol. The solid was filtered and washed with ethanol to give 2,2′-(piperazine-1,4-diylbis(methylene))bis(4,6-dimethylisothiazolo[5,4-b]pyridin-3(2H)-one) (LC-70). H NMR (400 MHz, Chloroform-d) δ 6.94 (s, 2H), 4.67 (s, 2H) 2.78 (s, 8H), 2.74 (s, 6H), 2.61 (s, 6H).

### Synthesis of LC-71

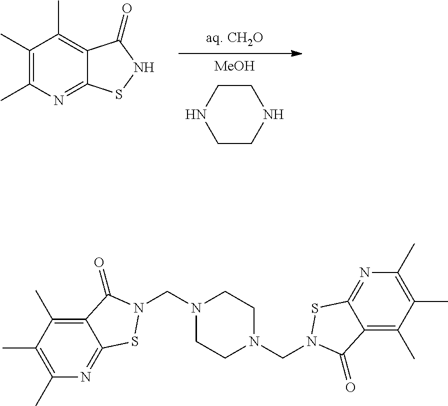

A mixture of 4,5,6-trimethylisothiazolo[5,4-b]pyridin-3(2H)-one (50 mg, 0.28 mmol, 1.0 eq), piperazine (24 mg, 0.28 mmol, 1.0 eq), and formaldehyde (37% wt. % in water, 0.35 mL) in absolute ethanol (5.0 mL) was heated at 80C for 12 h. The solvent was evaporated in vacuo and the crude product triturated with ethanol. The solid was filtered and washed with ethanol to give 2,2′-(piperazine-1,4-diylbis(methylene))bis(4,5,6-trimethylisothiazolo[5,4-b]pyridin-3(2H)-one) (LC-71). ¹H NMR (400 MHz, Chloroform-d) δ 4.67 (s, 4H) 2.78 (s, 8H), 2.78 (s, 6H), 2.62 (s, 6H), 2.29 (s, 6H).

### Synthesis of LC-72

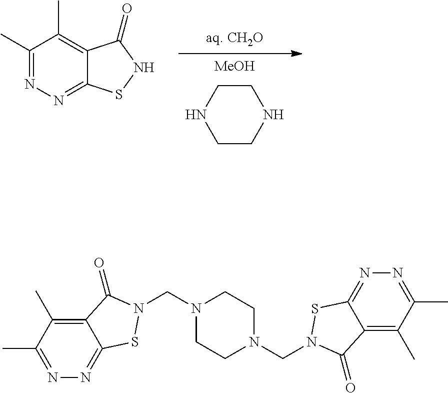

A mixture of 4,5-dimethylisothiazolo[5,4-c]pyridazin-3(2H)-one (LC-57) (29 mg, 0.16 mmol, 1.0 eq), piperazine (14 mg, 0.16 mmol, 1.0 eq), and formaldehyde (37% wt. % in water, 0.20 mL) in absolute ethanol (3.0 mL) was heated at 80C for 12 h. The solvent was evaporated in vacuo and the crude product triturated with ethanol. The solid was filtered and washed with ethanol to give 2,2′-(piperazine-1,4-diylbis(methylene))bis(4,5-dimethylisothiazolo[5,4-c]pyridazin-3(2H)-one) (LC-72).

### Synthesis of LC-73

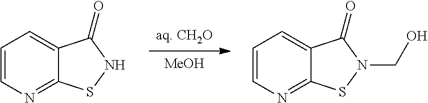 [SMILES: "MeOH" ; O=c1[nH]sc2ncccc12 ; O=c1c2cccnc2sn1CO]

A mixture of isothiazolo[5,4-b]pyridin-3(2H)-one (LC-47) (26 mg, 0.17 mmol, 1.0 eq) and formaldehyde (37% wt. % in water, 24 uL) in methanol (1.0 mL) was stirred overnight. The solvent was evaporated in vacuo and the crude product triturated with methanol. The solid was filtered and washed with methanol to give 2-(hydroxymethyl)isothiazolo[5,4-b]pyridin-3(2H)-one (LC-73) as a white powder (16 mg).

### Example 3

### Design and Evaluation of LOC14 Analogs

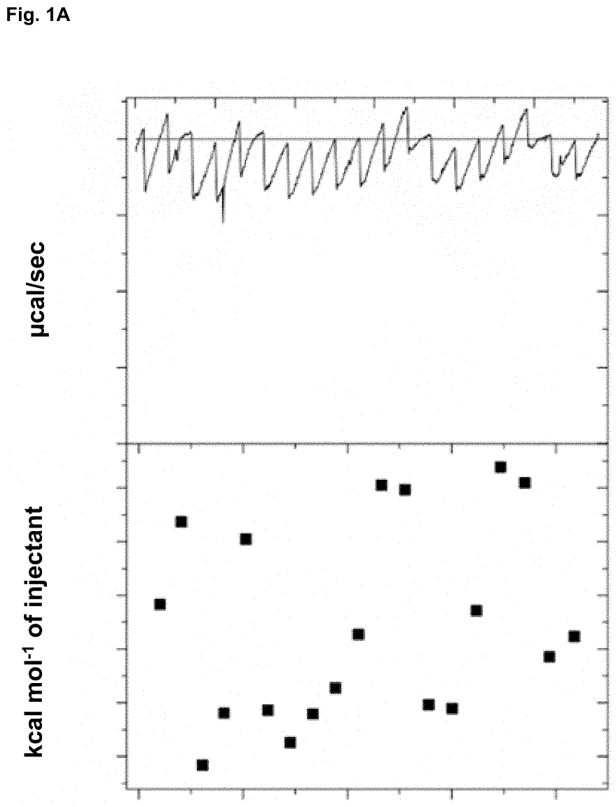

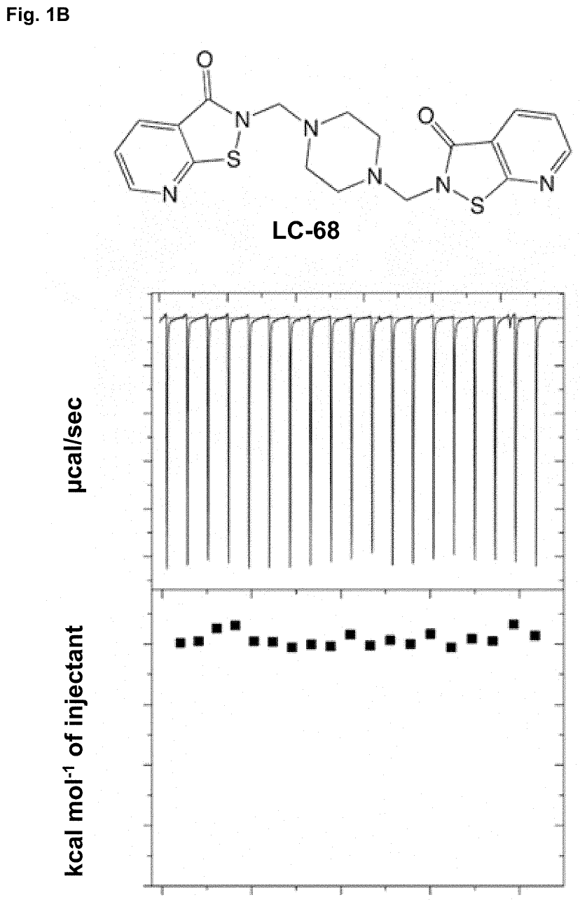

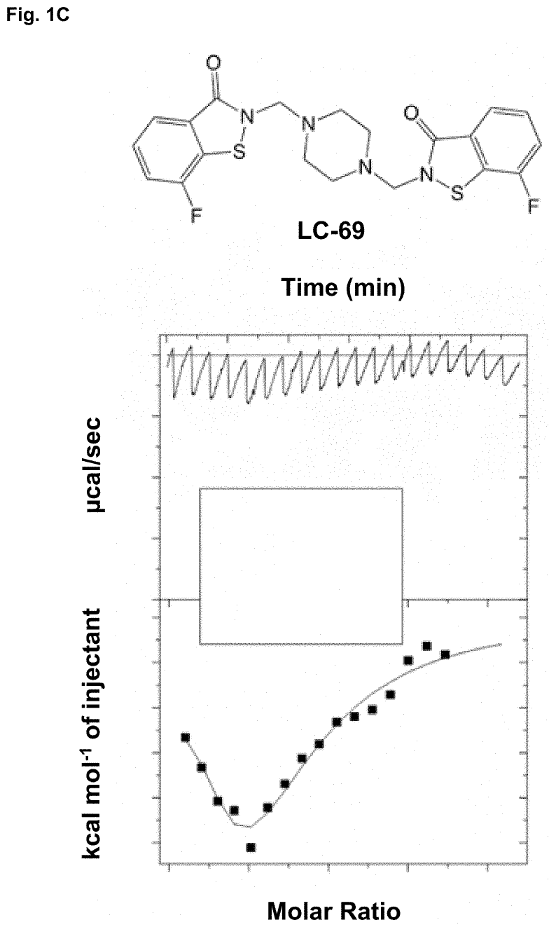

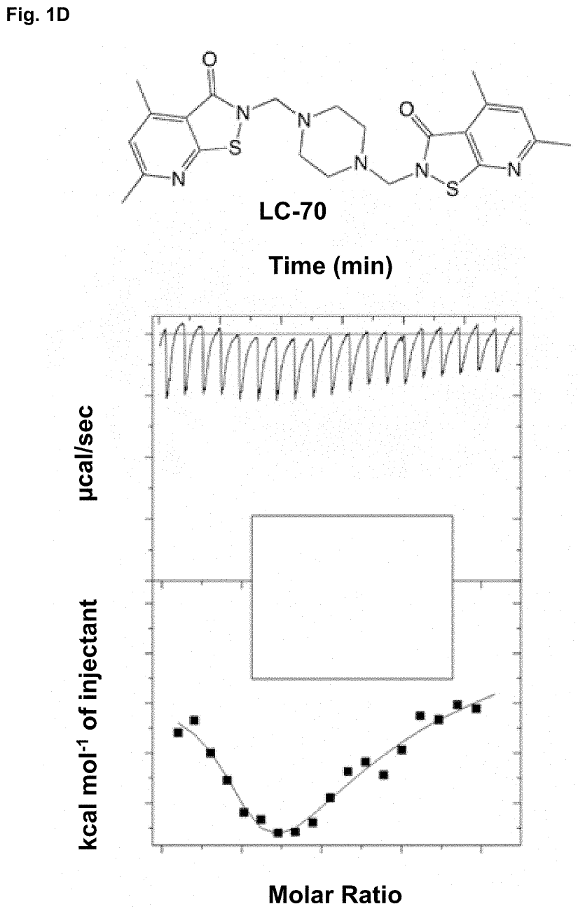

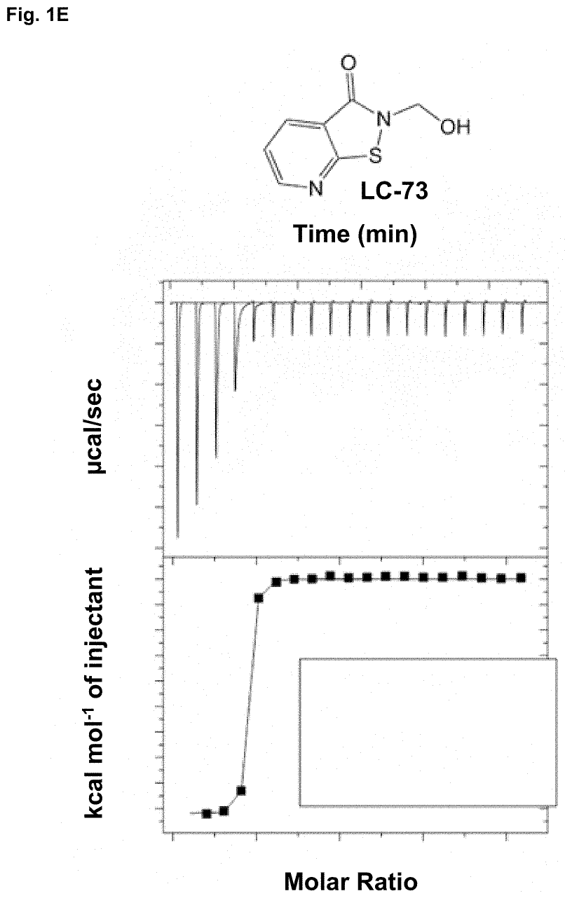

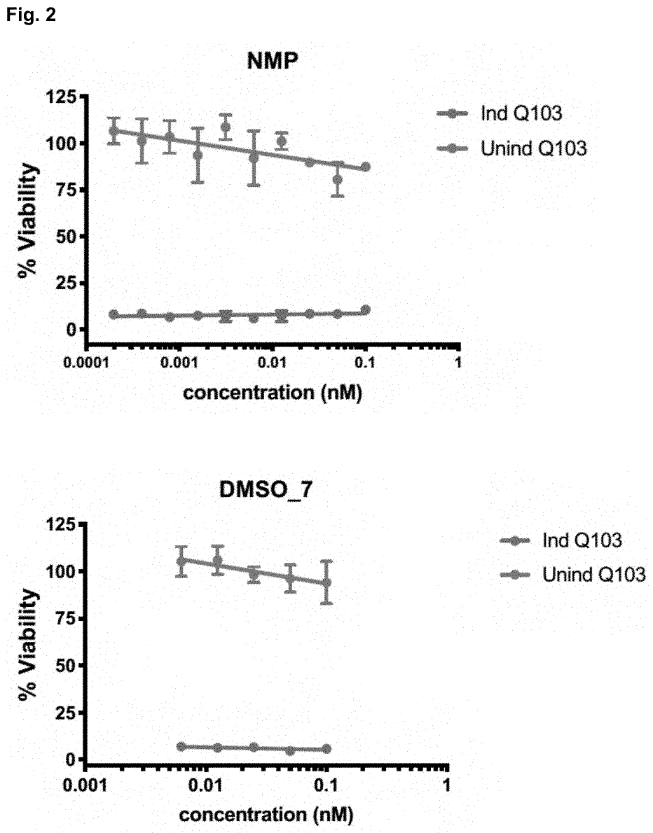

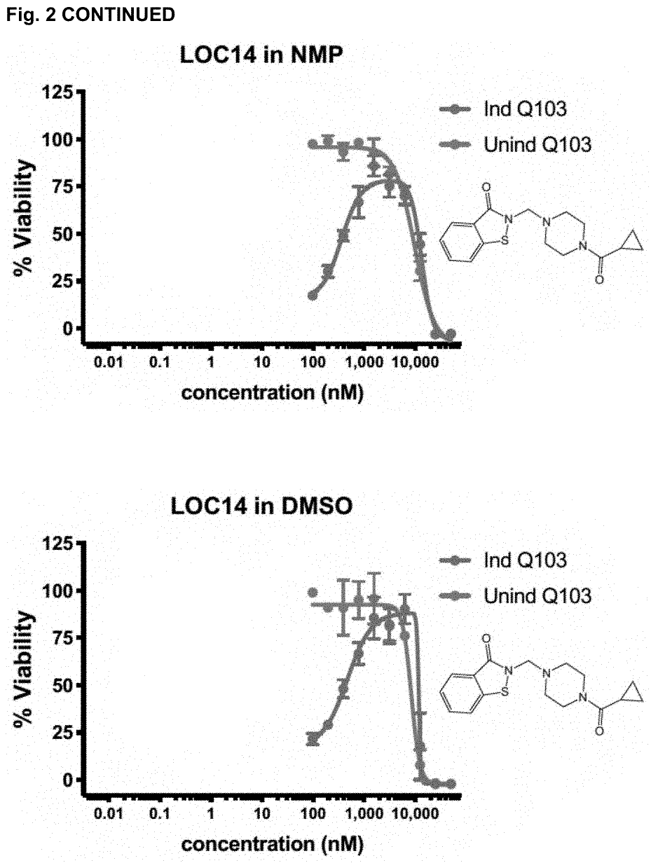

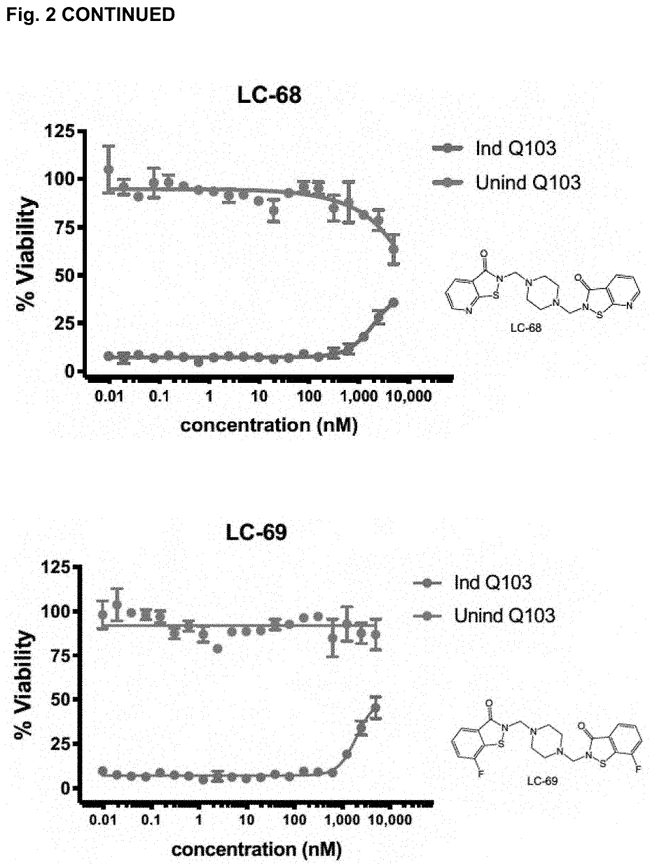

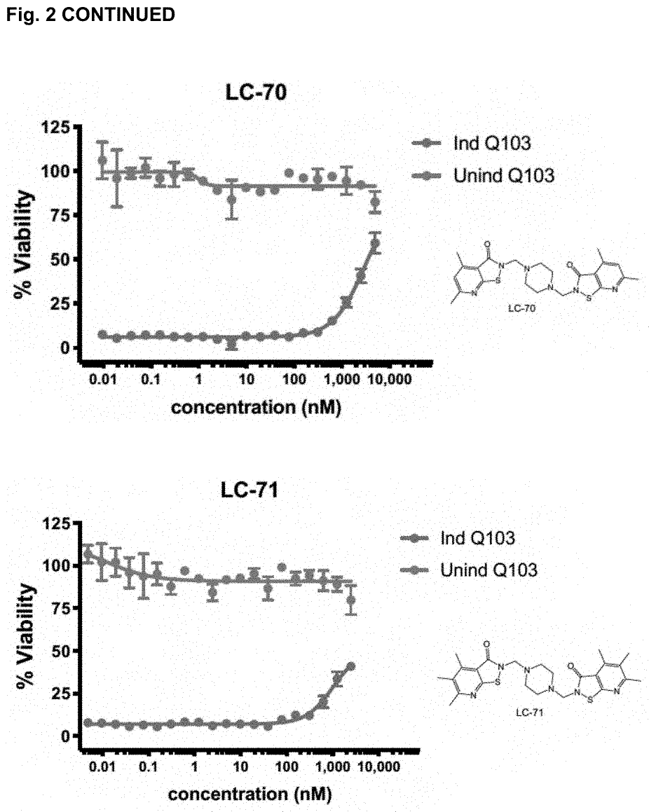

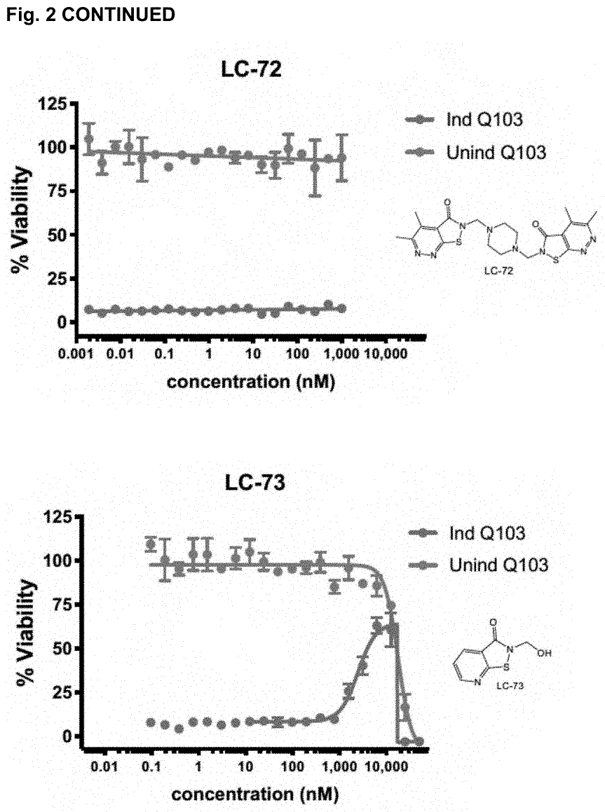

LOC14 analogs were designed and synthesized as described in the synthetic schemes. The analogs were evaluated for their binding to PDIa using isothermal titration calorimetry (ITC) (results of certain compounds are shown in FIGS. 1A-1E). The Kd values are given in the Table 1 below. Next, the analogs were tested for their ability to rescue PC12 cells from mHTT^{Q103} induced cell death in a dose-dependent manner (results of certain compounds are shown in FIG. 2).

TABLE 1
Analysis of LOC14 analogs.

| | | | | | | | PC12 |
|---|---|---|---|---|---|---|---|
| | | | | | | PC12 | cells |
| | | | | | | cells | Max % |
| Our ID | Name | Structure | MW | Solubility | ITC | EC50 | Viability |
| LOC14 | MMG509 MMG524 MMG821 MMG828 | 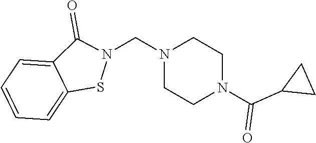 [SMILES: O=C(C1CC1)N1CCN(Cn2sc3ccccc3c2=O)CC1] | 317.41 | 100 mM in DMSO | KD = 113.4 ± 43.4 nM | EC50 = 352 nM | 66.25% |
| LC-1 | MMG 832A | 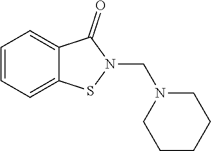 [SMILES: O=c1c2ccccc2sn1CN1CCCCC1] | 248.1 | 100 mM in DMSO | KD = 107.3 ± 14.4 nM | EC50 = 459 nM | 66.57% |
| LC-2 | MMG 832B | 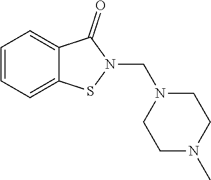 [SMILES: CN1CCN(Cn2sc3ccccc3c2=O)CC1] | 263.36 | 100 mM in DMSO | KD = 133.9 ± 20.4 nM | EC50 = 414.9 nM | 63.29% |
| LC-3 | MMG 832C | 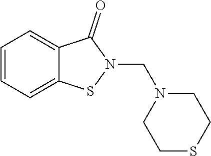 [SMILES: O=c1c2ccccc2sn1CN1CCSCC1] | 266.05 | 100 nM in DMSO | KD = 97.1 ± 15.9 nM | EC50 = 462.7 nM | 63.05% |
| LC-4 | MMG 832D | 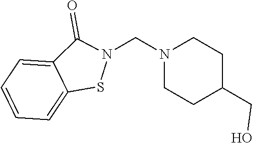 [SMILES: O=c1c2ccccc2sn1CN1CCC(CO)CC1] | 278.37 | 100 mM in DMSO | KD = 265.3 ± 36.0 nM | EC50 = 342.5 nM | 60.18% |
| LC-5 | PHB-5011 | 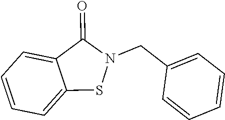 [SMILES: O=c1c2ccccc2sn1Cc1ccccc1] | 241.31 | 100 mM in DMSO | KD1 = 42.7 ± 36.5 nM; KD2 = 2267.6 ± 1511.7 mM | EC50 = 768.3 nM | 46.14% |
| LC-6 | PHB-5012 | 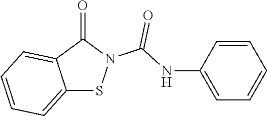 [SMILES: O=C(Nc1ccccc1)n1sc2ccccc2c1=O] | 270.31 | 30 mM in DMSO | KD = N/A irreversible or not active | EC50 = 476.3 nM | 66.47% |
| LC-7 | MMG 836C | 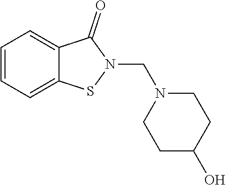 [SMILES: O=c1c2ccccc2sn1CN1CCC(O)CC1] | 264.3 | 100 mM in DMSO | KD = 216.0 ± 30.5 nM | EC50 = 441.1 nM | 71.98% |
| LC-8 | MMG 836D | 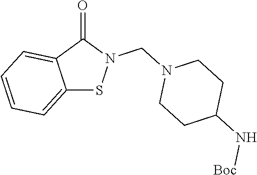 [SMILES: CC(C)(C)OC(=O)NC1CCN(Cn2sc3ccccc3c2=O)CC1] | 363.48 | 50 mM in DMSO | KD = 94.3 ± 20.4 nM | EC50 = 383.5 nM | 68.37% |
| LC-9 | AZII67A | 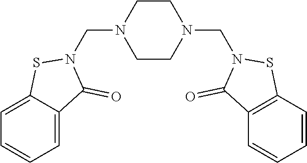 [SMILES: O=c1c2ccccc2sn1CN1CCN(Cn2sc3ccccc3c2=O)CC1] | 412.5 | 5 mM in DMSO | poor solubility KD = 12.7 nM | EC50 = 173.0 nM | 66.07% |
| LC-10 | AZII65 | 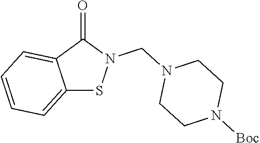 [SMILES: CC(C)(C)OC(=O)N1CCN(Cn2sc3ccccc3c2=O)CC1] | 287.14 | 100 mM in DMSO | KD = 123.3 ± 33.6 nM | EC50 = 560.7 nM | 71.82% |
| LC-11 | PHB-5007 | 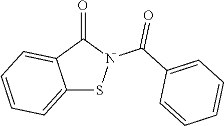 [SMILES: O=C(c1ccccc1)n1sc2ccccc2c1=O] | 255.29 | 100 mM in DMSO | KD = N/A irreversible or not active | EC50 >50,000 nM | 27.72% |
| LC-12 | MMG838A | 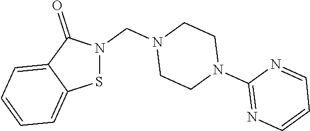 [SMILES: O=c1c2ccccc2sn1CN1CCN(c2ncccn2)CC1] | 327.41 | 100 mM in DMSO | KD = 140.3 ± 28.5 nM | EC50 = 415.7 nM | 68.35% |
| LC-13 | MMG838B | 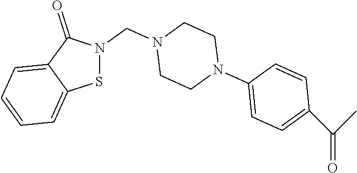 [SMILES: CC(=O)c1ccc(N2CCN(Cn3sc4ccccc4c3=O)CC2)cc1] | 367.14 | 100 mM in DMSO | KD = 189.8 ± 26.9 nM | EC50 = 513.6 nM | 63.14% |
| LC-14 | PHB-5013 (old) PHB5028 (new) | 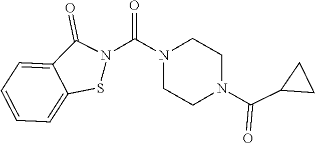 [SMILES: O=C(C1CC1)N1CCN(C(=O)n2sc3ccccc3c2=O)CC1] | 331.39 | 30 mM in DMSO | KD = N/A irreversible or not active | EC50 = >50,000 nM | 33.58% |
| LC-15 | AZII74 | 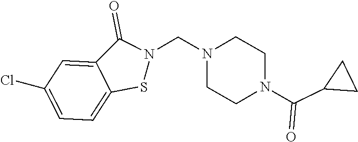 [SMILES: O=C(C1CC1)N1CCN(Cn2sc3ccc(Cl)cc3c2=O)CC1] | 351.8 | 15 mM in DMSO | KD = 113.6 ± 17.3 nM | EC50 = 487.3 nM | 56.92% |
| LC-16 | AZII84T | 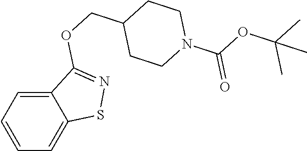 [SMILES: CC(C)(C)OC(=O)N1CCC(COc2nsc3ccccc23)CC1] | 348.5 | 50 mM in DMSO | KD = N/A irreversible or not active | EC50 >50,000 nM | 30.24% |
| LC-17 | AZII83 | 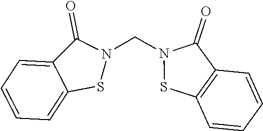 [SMILES: O=c1c2ccccc2sn1Cn1sc2ccccc2c1=O] | 314.4 | 100 mM in DMSO | KD = 160.0 ± 43.0 nM | EC50 = 229.4 nM | 67.75% |
| LC-18 | MMG849A | 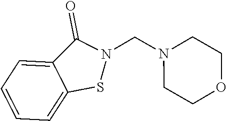 [SMILES: O=c1c2ccccc2sn1CN1CCOCC1] | 250.3 | 100 mM in DMSO | KD = 253.2 ± 42.6 nM | EC50 = 546.9 nM | 69.00% |
| LC-19 | PHB-5019 | 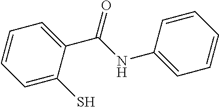 [SMILES: O=C(Nc1ccccc1)c1ccccc1S] | 229.3 | 50 mM in DMSO | KD = N/A irreversible or not active | EC50 = 1,635 nM | 48.03% |
| LC-20 | PHB-5020 | 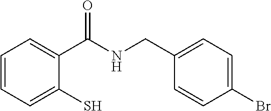 [SMILES: O=C(NCc1ccc(Br)cc1)c1ccccc1S] | 322.22 | 100 mM in DMSO | KD = N/A irreversible or not active | EC50 >50,000 nM | 42.11% |
| LC-21 | PHB-5021 | 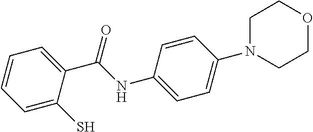 [SMILES: O=C(Nc1ccc(N2CCOCC2)cc1)c1ccccc1S] | 314.4 | 100 mM in DMSO | KD = N/A irreversible or not active | EC50 = 695.7 nM | 31.98% |
| LC-22 | PHB-5022 | 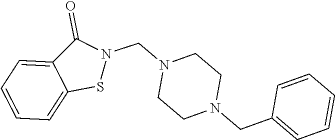 [SMILES: O=c1c2ccccc2sn1CN1CCN(Cc2ccccc2)CC1] | 339.46 | 100 mM in DMSO | KD = 173.3 ± 32.4 nM | EC50 = 309.8 nM | 61.51% |
| LC-23 | PHB-5024 | 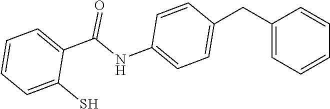 [SMILES: O=C(Nc1ccc(Cc2ccccc2)cc1)c1ccccc1S] | 319.42 | 100 mM in DMSO | KD = N/A irreversible or not active | EC50 = 3,883 nM | 56.53% |
| LC-24 | PHB-5039 | 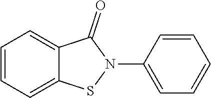 [SMILES: O=c1c2ccccc2sn1-c1ccccc1] | 227.28 | 50 mM in DMSO | KD1 = 8.1 ± 6.6 nM; KD2 = 1137.7 ± 529.4 nM | EC50 = 1.134 nM | 30.83% |
| LC-25 | PHB-5040 | 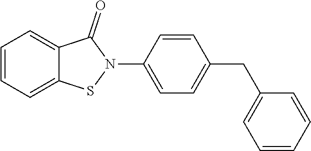 [SMILES: O=c1c2ccccc2sn1-c1ccc(Cc2ccccc2)cc1] | 317.41 | 100 mM in DMSO | KD = N/A irreversible or not active | EC50 = 1,591 nM | 36.83% |
| LC-26 | PHB-5041 | 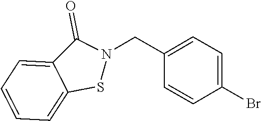 [SMILES: O=c1c2ccccc2sn1Cc1ccc(Br)cc1] | 320.2 | 100 mM in DMSO | KD = N/A irreversible or not active | EC50 = 778.1 nM | 37.88% |
| LC-27 | PHB-5042 | 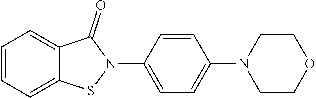 [SMILES: O=c1c2ccccc2sn1-c1ccc(N2CCOCC2)cc1] | 312.39 | 30 mM in DMSO | KD = N/A irreversible or not active | EC50 = 1,828 nM | 28.37% |
| LC-28 | PHB-5047 | 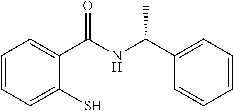 [SMILES: C[C@@H](NC(=O)c1ccccc1S)c1ccccc1] | 257.35 | 100 mM in DMSO | KD = N/A irreversible or not active | EC50 = 808 nM | 55.52% |
| LC-29 | PHB-5052 | 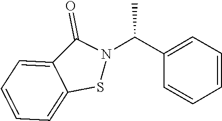 [SMILES: C[C@H](c1ccccc1)n1sc2ccccc2c1=O] | 255.34 | 100 mM in DMSO | KD = 657.9 ± 223.8 nM | EC50 = 669.1 nM | 42.02% |
| LC-30 | PHB-5048 | 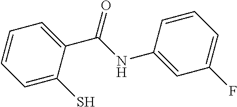 [SMILES: O=C(Nc1cccc(F)c1)c1ccccc1S] | 247.29 | 100 mM in DMSO | KD = N/A irreversible or not active | EC50 = 2,992 nM | 62.38% |
| LC-31 | PHB-5053 | 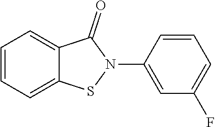 [SMILES: O=c1c2ccccc2sn1-c1cccc(F)c1] | 245.27 | 100 mM in DMSO | KD = N/A irreversible or not active | EC50 = 1.765 nM | 36.24% |
| LC-32 | PHB-5045 | 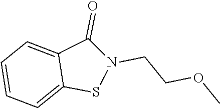 | 209.26 | 100 mM in DMSO | KD = 158.2 ± 60.8 nM | EC50 = 700.1 nM | 41.96% |
| LC-33 | PHB-5049 | 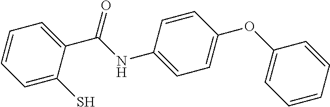 [SMILES: O=C(Nc1ccc(Oc2ccccc2)cc1)c1ccccc1S] | 321.39 | 100 mM in DMSO | KD = N/A irreversible or not active | EC50 = 2,937 nM | 54.43% |
| LC-34 | PHB-5051 | 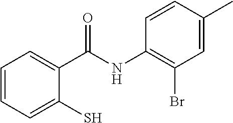 [SMILES: Cc1ccc(NC(=O)c2ccccc2S)c(Br)c1] | 322.22 | 100 mM in DMSO | KD = N/A irreversible or not active | EC50 = 3767 nM | 41.95% |
| LC-35 | MI | 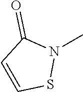 | 115.15 | 100 mM in water | KD = N/A irreversible or not acitve | Not Active | 14.63% |
| L-36 | PHB-5055 | 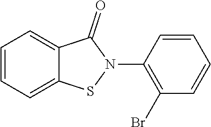 [SMILES: O=c1c2ccccc2sn1-c1ccccc1Br] | 320.2 | 100 mM in DMSO | KD = 25.3 ± 10.7 nM | EC50 = 1594 nM | 15.08% |
| LC-37 | PHB-5056 | 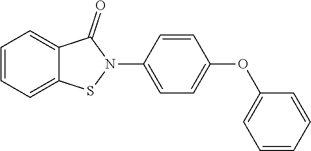 [SMILES: O=c1c2ccccc2sn1-c1ccc(Oc2ccccc2)cc1] | 319.38 | 100 mM in DMSO | KD = N/A irreversible or not active | EC50 = 1679 nM | 21.77% |
| LC-38 | AZIII24 | 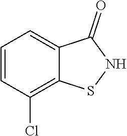 [SMILES: O=c1[nH]sc2c(Cl)cccc12] | 185.6 | 100 mM in DMSO | KD = N/A irreversible or not active | Not Active | 7.63% |
| LC-39 | AZIII26C | 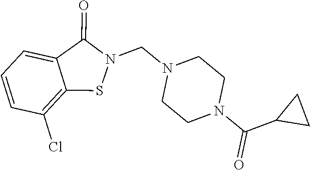 [SMILES: O=C(C1CC1)N1CCN(Cn2sc3c(Cl)cccc3c2=O)CC1] | 351.8 | 100 mM in DMSO | KD = N/A irreversible or not active | Not Active | 7.02% |
| LC-40 | AZIII33 | 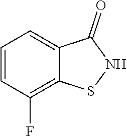 [SMILES: O=c1[nH]sc2c(F)cccc12] | 169.2 | 100 mM in DMSO | KD = N/A irreversible or not active | EC50 = 9325 nM | 72.31% |
| LC-41 | AZIII32 | 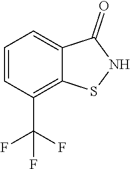 [SMILES: O=c1[nH]sc2c(C(F)(F)F)cccc12] | 219.2 | 100 mM in DMSO | KD = N/A irreversible or not active | Not Active | 9.16% |
| LC-42 | AZIII34 | 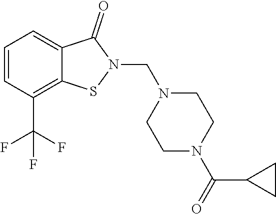 [SMILES: O=C(C1CC1)N1CCN(Cn2sc3c(C(F)(F)F)cccc3c2=O)CC1] | 385.4 | 100 mM in DMSO | KD = N/A irreversible or not active | Not Active | 8.17% |
| LC-43 | AZIII35 | 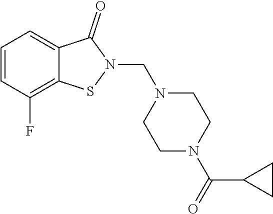 [SMILES: O=C(C1CC1)N1CCN(Cn2sc3c(F)cccc3c2=O)CC1] | 335.4 | 100 mM in DMSO | KD = N/A irreversible or not active | EC50 = 7409 nM | 56.48% |
| LC-44 | AZIII29 | 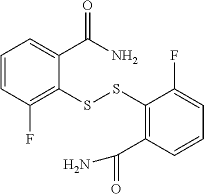 [SMILES: NC(=O)c1cccc(F)c1SSc1c(F)cccc1C(N)=O] | 340.4 | 100 mM in DMSO | KD = N/A irreversible or not active | EC50 = 7066 nM | 87.19% |
| LC-45 | AZIII28 | 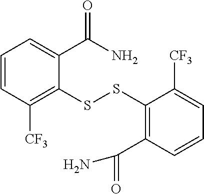 [SMILES: NC(=O)c1cccc(C(F)(F)F)c1SSc1c(C(N)=O)cccc1C(F)(F)F] | 440.4 | 100 mM in DMSO | KD = N/A irreversible or not active | Not Active | 16.98% |
| LC-46 | PHB-5062 | 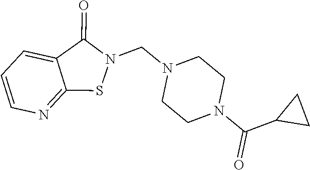 [SMILES: O=C(C1CC1)N1CCN(Cn2sc3ncccc3c2=O)CC1] | 318.4 | 50 mM in DMSO | KD = 9.6 +/− 3.2 nM | EC50 = 2831 nM | 39.23% |
| LC-47 | PHB-5063 | 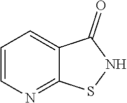 | 152.17 | 50 mM in DMSO | KD = N/A irreversible or not active | EC50 >50,000 nM | 36.31% |
| LC-48 | AZIII60A | 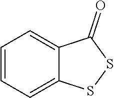 | 168.2 | 100 mM in DMSO | KD = N/A irreversible or not active | Not Active | 8.14% |
| LC-49 | AZIII60B | 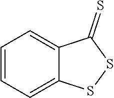 | 184.3 | 100 mM in DMSO | KD = N/A irreversible or not active | Not Active | 8.75% |
| LC-50 | AZIII60D | 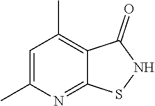 | 180.2 | 100 mM in DMSO | KD = 3831 +/− 1327 nM | EC50 = 5,180 nM | 65.19% |
| LC-51 | AZIII60C | 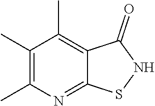 | 194.3 | 10 mM in DMSO | KD = N/A irreversible or not active | EC50 = 2,200 nM | 63.14% |
| LC-52 | AZIII92A | 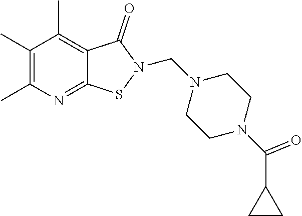 [SMILES: Cc1nc2sn(CN3CCN(C(=O)C4CC4)CC3)c(=O)c2c(C)c1C] | 360.5 | 10 nM in DMSO | KD = N/A irreversible or not active | EC50 = 3,570 nM | 68.30% |
| LC-53 | AZIII93 | 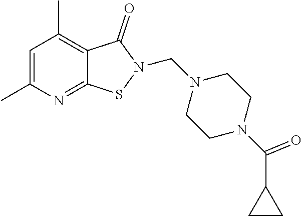 [SMILES: Cc1cc(C)c2c(=O)n(CN3CCN(C(=O)C4CC4)CC3)sc2n1] | 346.4 | 25 mM in DMSO | KD = N/A irreversible or not active | EC50 = 4.249 nM | 67.25% |
| LC-54 | PHB5077 | 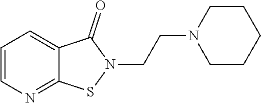 [SMILES: O=c1c2cccnc2sn1CCN1CCCCC1] | 263.36 | 100 mM in DMSO | KD <20 nM | Not Active | 10.67% |
| LC-55 | PHB5078 | 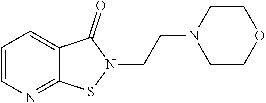 [SMILES: O=c1c2cccnc2sn1CCN1CCOCC1] | 265.33 | 100 mM in DMSO | KD = 20.1 +/− 7.8 nM | Not Active | 13.20% |
| LC-56 | PB5079 | 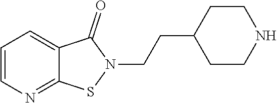 [SMILES: O=c1c2cccnc2sn1CCC1CCNCC1] | 263.36 | 100 mM in DMSO | KD = N/A irreversible or not active | Not Active | 14.03% |
| LC-57 | PHB5088 | 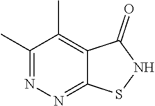 | 181.21 | 100 mM in DMSO | KD = 17.5 +/− 1.8 nM | EC50 = 12,150 nM | 54.15% |
| LC-58 | PHB5084 | 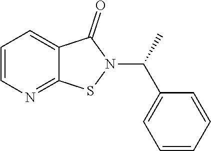 [SMILES: C[C@H](c1ccccc1)n1sc2ncccc2c1=O] | 256.32 | 100 mM in DMSO | KD = 0.3 +/− 3.5 nM | Not Active | 13.62% |
| LC-59 | PHB5089 | 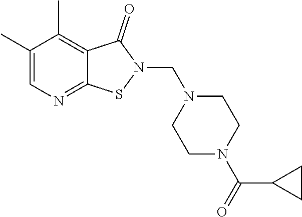 [SMILES: Cc1cnc2sn(CN3CCN(C(=O)C4CC4)CC3)c(=O)c2c1C] | 347.44 | 100 mM in DMSO | KD = 8.2 +/− 3.2 nM | EC50 = 10,573 nM | 59.20% |
| LC-60 | PHB5093 | 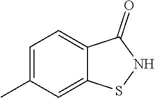 | 165.21 | 100 mM in DMSO | KD = 347.2 nM | EC50 = 561.5 nM | 59.49% |
| LC-61 | PHB5094 | 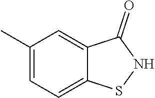 | 165.21 | 100 mM in DMSO | KD = 266.7 nM | EC50 = 590.8 nM | 64.68% |
| LC-62 | PBH5098 | 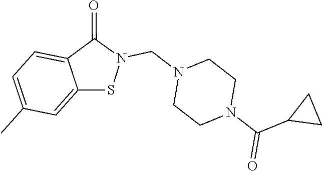 [SMILES: Cc1ccc2c(=O)n(CN3CCN(C(=O)C4CC4)CC3)sc2c1] | 331.43 | 50 mM in DMSO | KD = 662.3 nM | EC50 = 1576 nM | 54.31% |
| LC-63 | PHB5100 | 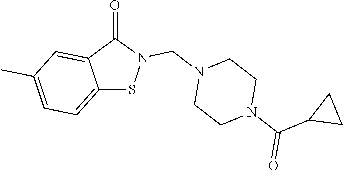 [SMILES: Cc1ccc2sn(CN3CCN(C(=O)C4CC4)CC3)c(=O)c2c1] | 331.43 | 50 mM in DMSO | KD = 239.8 nM | EC50 = 658.8 nM | 6 [symbol] .33% |
| LC-64 | PHB-5104 | 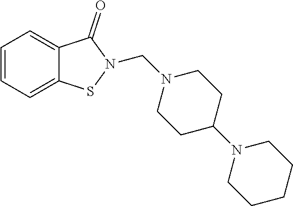 [SMILES: O=c1c2ccccc2sn1CN1CCC(N2CCCCC2)CC1] | 331.48 | 50 mM in DMSO | KD = 1.21 uM | EC50 = 393 nM | 81.43% |
| LC-65 | PHB-5105 | 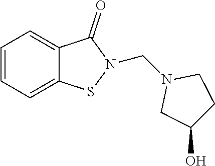 [SMILES: O=c1c2ccccc2sn1CN1CC[C@@H](O)C1] | 250.32 | 100 mM in DMSO | Kd = 162 nM | EC50 = 456 nM | 77.01% |
| LC-66 | PHB-5113 | 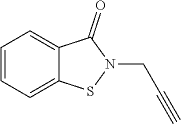 | 189.23 | 100 mM in DMSO | Kd1 = 30.7 nM; KD2 = 14.4 uM | EC50 = 763 nM | 40.34% |
| LC-67 | PHB-5114 | 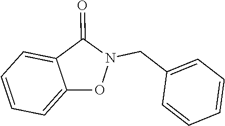 [SMILES: O=c1c2ccccc2on1Cc1ccccc1] | 225.25 | 100 mM in DMSO | Not active or irreversible | not active | 8.05% |
| LC-68 | AZVI-63 | 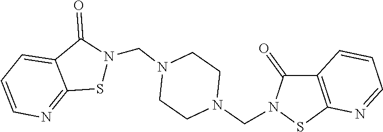 [SMILES: O=c1c2cccnc2sn1CN1CCN(Cn2sc3ncccc3c2=O)CC1] | 414.5 | 5 mM in NMP | Not active or irreversible | EC50 = 2061 nM | 35.78% |
| LC-69 | AZVI-67 | 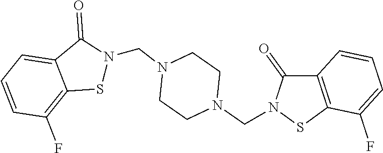 [SMILES: O=c1c2cccc(F)c2sn1CN1CCN(Cn2sc3c(F)cccc3c2=O)CC1] | 448.51 | 5 mM in NMP | Kd = 700 nM (prelim) | EC50 = 1974 nM | 45.50% |
| LC-70 | AZVII-7 | 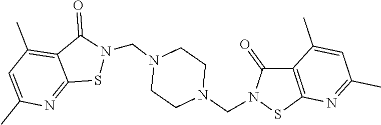 | 470.61 | 5 mM in NMP | KD = 3.8 uM (prelim) | EC50 = 3088 uM | 59.18% |
| LC-71 | AZVII-8 | 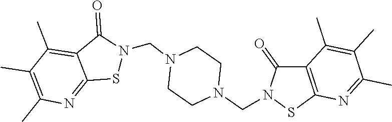 | 498.66 | 2.5 mM in NMP | poor solubility | EC50 = 1019 uM | 40.98% |
| LC-72 | AZVII-10 | 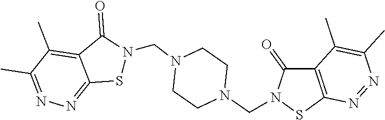 | 472.59 | 1 mM in NMP | poor solubility | Not Active | 10.26% |
| LC-73 | | 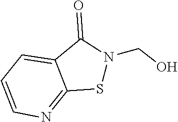 [SMILES: O=c1c2cccnc2sn1CO] | 182.2 | 100 mM in DMSO | Kd = 9.8 nM | EC50 = 2475 nM | 63.16% |
| Oxy- LOC14 | | 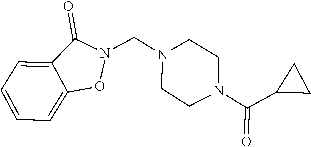 [SMILES: O=C(C1CC1)N1CCN(Cn2oc3ccccc3c2=O)CC1] | 301.35 | 100 mM in DMSO | KD = N/A | Not Active | 8.16% |

*N/A - Not active [symbol]
indicates data missing or illegible when filed

### Example 4

### Evaluation of LOC14 and its Analogs for Potential Off-Target Effects

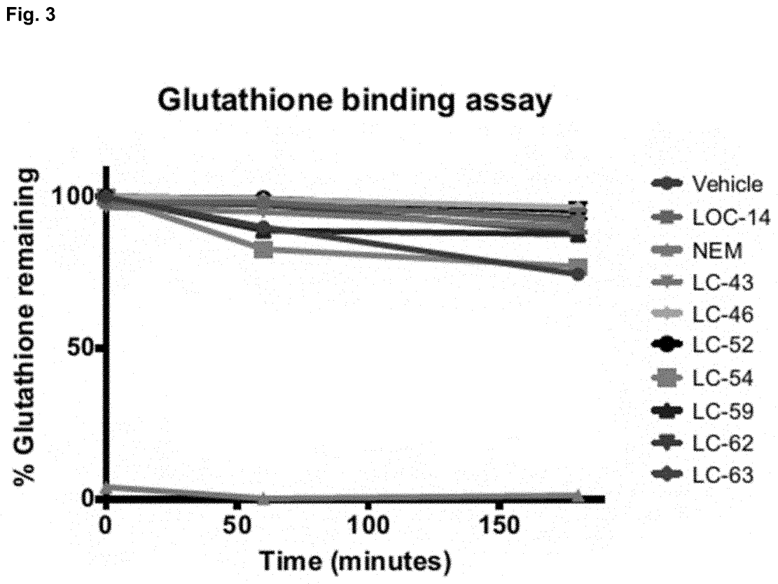

To assess potential off-target reactivity, we evaluated LOC14 and some of its promising analogs in their ability to interact with glutathione (GSH). Glutathione binding of a compound results in compound excretion into the bile, and serves as an important pathway of elimination. To asses the ability of LOC-14 and its analogs to bind to GSH we used a high throughput assay in which the reduction in glutathione levels upon compound binding was quantitated using Ellman's reagent (FIG. 3). The results from this assay show that LOC-14 and the analogs do not bind to glutathione at pH 8.5 in Tris-buffer. The vehicle control shows that the glutathione concentration slowly diminishes over time, presumably due to the oxidation of GSH to GSSG in buffer. Incubation of the positive control (NEM) with glutathione led to an immediate depletion of glutathione.

In addition to testing LOC14 analogs, we also focused our attention on further characterizing LOC14's properties. We tested LOC14 in the GSH trapping assay, inhibition of CYP enzymes (study conducted by CRO Cyprotex), and against a broad panel of enzymes to evaluate its selectivity to PDI. LOC14 did not react with glutathione and it did not bind to any of the 30 protein targets in the broad panel, indicating its binding to PDI is selective (Table 2). Furthermore, we observed that LOC14 does not bind to the hERG receptor, indicating it is not predicted to possess potentially fatal cardiotoxic effects in vivo. We have also evaluated the effects of LOC14 and fluorine analog LC-43 in their ability to inhibit CYP450 enzymes. At high concentration of 30 μM, above the concentration that is neuroprotective, LOC14 inhibits CYP3A4 and CYP1A2 enzymes.

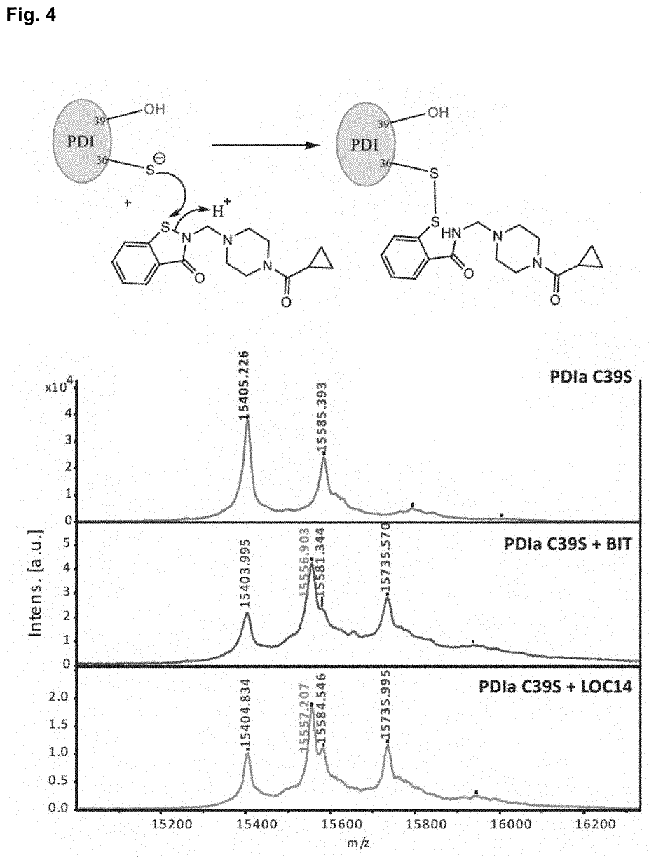

We also explored the feasibility of generating a crystal structure of PDI bound to one of these inhibitors for structure-based analog design. LOC14 is a reversible inhibitor of PDI and therefore may not remain bound to the protein in crystallization trays. To trap the LOC14-PDI complex, we generated a PDI C39S mutant that should not release the compound from PDI. To confirm this mechanism of binding, we performed MALDI-TOF mass-spectrometry experiments with PDI C39S in the presence and absence of LOC14 (FIG. 4). The C39S mutant showed two mass peaks: a main mass peak at 15,405 Da and a second peak at 15,585 (+179 Da change) corresponding to the normal gluconoylation modification to C39S protein at the His6× tag by E. coli. PDI C39S treated with LOC14 gave 4 peaks: main mass peak at 15,557 Da (change of +152 which corresponds to the bound benzoisothiazolone moiety to PDI C39S), a peak at 15,735 Da (corresponding to the benzoisothiazolone moiety bound to C39S-gluconoylated protein), a peak at 15,404 (the mass of unbound C39S protein), and a peak at 15,584 Da (the mass of unbound C39S-gluconoylated protein). These data indicate that LOC14 does bind to the catalytic cysteine of PDI. The incomplete binding is most likely due to the presence of reducing agent in the buffer. We can use PDI C39S in complex with LOC14 in a crystallization screen to obtain a detailed binding pose of the inhibitor to the protein.

TABLE 2
Evaluation of LOC14 for potential off-target effects.

| Assay Name | Species | Rep. | Conc. | % Inh. |
|---|---|---|---|---|
| Adenosine A1 | human | 2 | 3 μM | 8 |
| Adenosine A2A | human | 2 | 3 μM | −4 |
| Adrenergic α1A | rat | 2 | 3 μM | −14 |
| Adrenergic α1B | rat | 2 | 3 μM | 2 |
| Adrenergic α2A | human | 2 | 3 μM | 7 |
| Adrenergic β1 | human | 2 | 3 μM | 7 |
| Adrenergic β2 | human | 2 | 3 μM | 8 |
| Calcium Channel L-Type, | rat | 2 | 3 μM | 5 |
| Dihydropyridine | | | | |
| Cannabinoid CB1 | human | 2 | 3 μM | −22 |
| Dopamine D1 | human | 2 | 3 μM | 34 |
| Dopamine D2S | human | 2 | 3 μM | 0 |
| GABAA, Flunitrazepam, Central | rat | 2 | 3 μM | 6 |
| GABAA, Muscimol, Central | rat | 2 | 3 μM | −3 |
| Glutamate, NMDA, Phencyclidine | rat | 2 | 3 μM | 7 |
| Histamine H1 | human | 2 | 3 μM | 27 |
| Imidazoline I2, Central | rat | 2 | 3 μM | −20 |
| Muscarinic M2 | human | 2 | 3 μM | −3 |
| Muscarinic M3 | human | 2 | 3 μM | −4 |
| Nicotinic Acetylcholine | human | 2 | 3 μM | 1 |
| Nicotinic Acetylcholine α1, | human | 2 | 3 μM | 11 |
| Bungarotoxin | | | | |
| Opiate (OP3, MOP) | human | 2 | 3 μM | −1 |
| Phorbol Ester | mouse | 2 | 3 μM | −3 |
| Potassium Channel [KATP] | hamster | 2 | 3 μM | 14 |
| Potassium Channel hERG | human | 2 | 3 μM | 10 |
| Prostanoid EP4 | human | 2 | 3 μM | 32 |
| Rolipram | rat | 2 | 3 μM | 16 |
| Serotonin (5-Hydroxytryptamine) | human | 2 | 3 μM | 36 |
| 5-HT2B | | | | |
| Sigma σ1 | human | 2 | 3 μM | −8 |
| Sodium Channel, Site 2 | rat | 2 | 3 μM | 1 |
| Transporter, Norepinephrine (NET) | human | 2 | 3 μM | 24 |

### Example 5

### Pharmacodynamic Screens for LOC14

LOC14 was also submitted for testing in CEREP's Safety47 panel. CEREP is a CRO specializing in preclinical research, discovery and development of new pharmaceutical drugs. The purpose of Safety 47 panel was to do in vitro pharmacological profiling on LOC14, to determine any off-target liabilities that might exist against 47 human targets. The targets were selected based on safety recommendations by major pharmaceutical companies (Bowes et al. 2012) and involved a broad range of 47 targets including receptors, ion channels, transporters, enzymes and second messengers. The results of the Safety4l panel are shown in Tables 3 and 4. This screen helped to confirm that there are no significant off-target effects for LO14. While muscarinic acetylcholine receptor M2 showed higher response to LOC14, this interaction did not reach the response threshold of 95% to be considered significant. The current data showed that LOC14 is a good lead compound with favourable characteristics for further rounds of optimization.

TABLE 3
LOC14's affinity for safety-relevant
G protein-coupled receptors (GPCRs).

| | | |
|---|---|---|
| Gene Symbol | Assay Mode | LOC14 3 μM |

| | | |
|---|---|---|
| GPCRs | Average % Response | SD |

| | | | |
|---|---|---|---|
| ADORA2A | Agonist | 2.6 | 1.9 |
| ADORA2A | Antagonist | 2.4 | 3.3 |
| ADRA1A | Agonist | 1.9 | 0.8 |
| ADRA1A | Antagonist | 1.7 | 2.4 |
| ADRA2A | Agonist | 58.3 | 1.0 |
| ADRA2A | Antagonist | 0.0 | 0.0 |
| ADRB1 | Agonist | 0.0 | 0.0 |
| ADRB1 | Antagonist | 50.8 | 9.4 |
| ADRB2 | Agonist | 0.0 | 0.0 |
| ADRB2 | Antagonist | 41.6 | 3.7 |
| AVPR1A | Agonist | 3.7 | 1.9 |
| AVPR1A | Antagonist | 0.0 | 0.0 |
| CCKAR | Agonist | 3.0 | 0.5 |
| CCKAR | Antagonist | 0.0 | 0.0 |
| CHRM1 | Agonist | 0.3 | 0.4 |
| CHRM1 | Antagonist | 7.6 | 5.8 |
| CHRM2 | Agonist | 71.5 | 2.0 |
| CHRM2 | Antagonist | 0.0 | 0.0 |
| CHRM3 | Agonist | 2.5 | 1.5 |
| CHRM3 | Antagonist | 0.6 | 0.9 |
| CNR1 | Agonist | 50.1 | 1.2 |
| CNR1 | Antagonist | 0.0 | 0.0 |
| CNR2 | Agonist | 58.7 | 2.0 |
| CNR2 | Antagonist | 0.0 | 0.0 |
| DRD1 | Agonist | 0.0 | 0.0 |
| DRD1 | Antagonist | 49.2 | 2.3 |
| DRD2S | Agonist | 48.3 | 5.3 |
| DRD2S | Antagonist | 0.0 | 0.0 |
| EDNRA | Agonist | 3.2 | 0.5 |
| EDNRA | Antagonist | 0.0 | 0.0 |
| HRH1 | Agonist | 1.2 | 0.0 |
| HRH1 | Antagonist | 0.0 | 0.0 |
| HRH2 | Agonist | 0.0 | 0.0 |
| HRH2 | Antagonist | 62.1 | 0.2 |
| HTR1A | Agonist | 44.4 | 1.0 |
| HTR1A | Antagonist | 0.0 | 0.0 |
| HTR1B | Agonist | 53.0 | 1.8 |
| HTR1B | Antagonist | 0.0 | 0.0 |
| HTR2A | Agonist | 7.3 | 0.3 |
| HTR2A | Antagonist | 4.1 | 3.1 |
| HTR2B | Agonist | 0.4 | 0.6 |
| HTR2B | Antagonist | 2.7 | 1.8 |
| OPRD1 | Agonist | 52.2 | 1.9 |
| OPRD1 | Antagonist | 0.0 | 0.0 |
| OPRK1 | Agonist | 39.0 | 0.7 |
| OPRK1 | Antagonist | 0.0 | 0.0 |
| OPRM1 | Agonist | 43.5 | 0.1 |
| OPRM1 | Antagonist | 0.0 | 0.0 |

TABLE 4
LOC14's affinity for safety-relevant targets.

LOC14 3 μM

| | Gene Symbol | Assay Mode | Average | SD |
|---|---|---|---|---|
| Nuclear Hormone Receptors | | | | |
| | AR | Agonist | 0.0 | 0.0 |
| | AR | Antagonist | 5.4 | 3.4 |
| | GR | Agonist | 0.0 | 0.0 |
| | GR | Antagonist | 0.1 | 0.1 |
| Transporters | | | | |
| | DAT | Blocker | 0.0 | 0.0 |
| | NET | Blocker | 0.0 | 0.0 |
| | SERT | Blocker | 5.0 | 7.0 |
| Ion Channels | | | | |
| | CAV1.2 | Blocker | 1.2 | 0.5 |
| | GABAA | Opener | 0.0 | 0.0 |
| | GABAA | Blocker | 5.1 | 7.2 |
| | hERG | Blocker | 5.9 | 2.0 |
| | HTR3A | Opener | 2.5 | 1.1 |
| | HTR3A | Blocker | 0.0 | 0.0 |
| | KvLQT1/minK | Opener | 9.8 | 1.7 |
| | KvLQT1/minK | Blocker | 0.0 | 0.0 |
| | nAChR(a4/b2) | Opener | 0.0 | 0.0 |
| | nAChR(a4/b2) | Blocker | 10.1 | 7.3 |
| | NAV1.5 | Blocker | 7.0 | 9.9 |
| | NMDAR (1A/2B) | Opener | 1.7 | 2.4 |
| | NMDAR (1A/2B) | Blocker | 0.0 | 0.0 |
| Non-Kinase Enzymes | | | | |
| | AChE | Inhibitor | 2.8 | 3.9 |
| | COX1 | Inhibitor | 1.4 | 1.4 |
| | COX2 | Inhibitor | 2.8 | 1.8 |
| | MAOA | Inhibitor | 9.9 | 2.9 |
| | PDE3A | Inhibitor | 0.0 | 0.0 |
| | PDE4D2 | Inhibitor | 0.0 | 0.0 |
| Kinases | | | | |
| | INSR | Inhibitor | 22.7 | 5.4 |
| | LCK | Inhibitor | 16.1 | 7.6 |
| | ROCK1 | Inhibitor | 13.5 | 0.6 |
| | VEGFR2 | Inhibitor | 17.2 | 13.2 |

### Example 6

### ROS Assay Development and Optimization

In order to define the role of PDIA1-generated H₂O₂ in ER linked oxidative stress and neurodegeneration, it was necessary to develop a framework for the investigation of abnormal ROS production. Previous efforts to establish ROS levels were not conclusive and this time a number of optimization steps were explored to find the optimal conditions for assays. First, ROS-Glo assay was used to determine the generation and changes in ROS levels in the ER in the in vitro model of HD under normal and ER stress conditions. ROS-Glo is a bioluminescent assay measuring the level of H₂O₂ and can be performed directly in cell culture. This assay is based on a derivatized luciferin substrate which reacts directly with H₂O₂ generating a luciferin precursor which later in the subsequent assay reactions is converted to luciferin. Luciferin is the substrate for Ultra-Glo™ Recombinant Luciferase and this reaction produce light signal that is proportional to the level of H₂O₂ present in the sample.

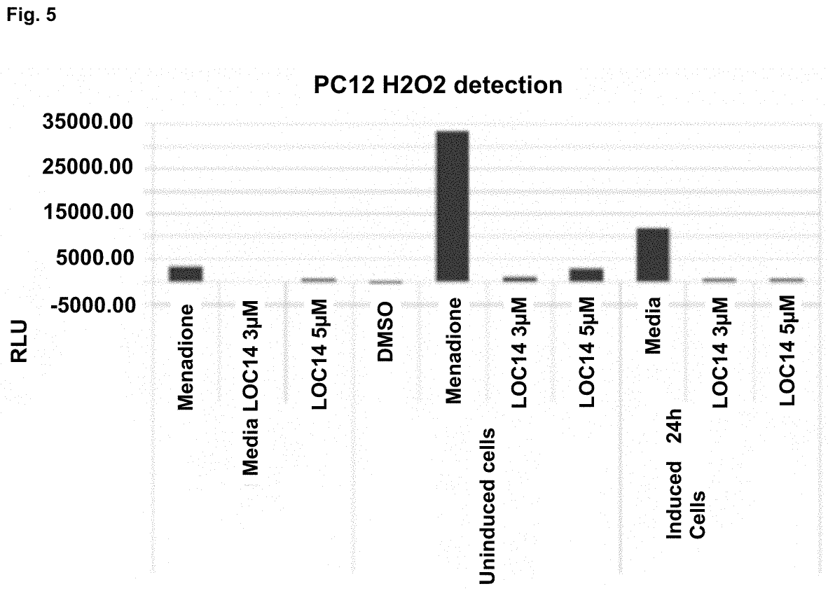

Improved ROS-Glo protocol produced less variable results (although some outliers exist, presumably because of variable GFP expression). As can be seen, LOC14 at both concentrations significantly reduces H₂O₂ production, while cells not treated with the compound had a marked upregulation of ROS (FIG. 5). These results support our hypothesis that LOC14 functions as a suppressor of H₂O₂ production by bypassing PDI/Ero1 catalytic cycling and subsequent hydrogen peroxide production.

CellRox Deep Red confocal microscopy analysis is a different type of analysis selected for the purpose not only to employ different detection methods but also to increase the reliability of readouts. Briefly, CellRox Deep Red Reagent is a novel fluorogenic probe used to measure cellular oxidative stress in both live and fixed cell imaging. The reagent is cell-permeant dye and does not fluoresce when in a reduced state; after oxidation, deep red reagent exhibits bright fluorescence absorption/emission maxima at ˜644/665 nm).

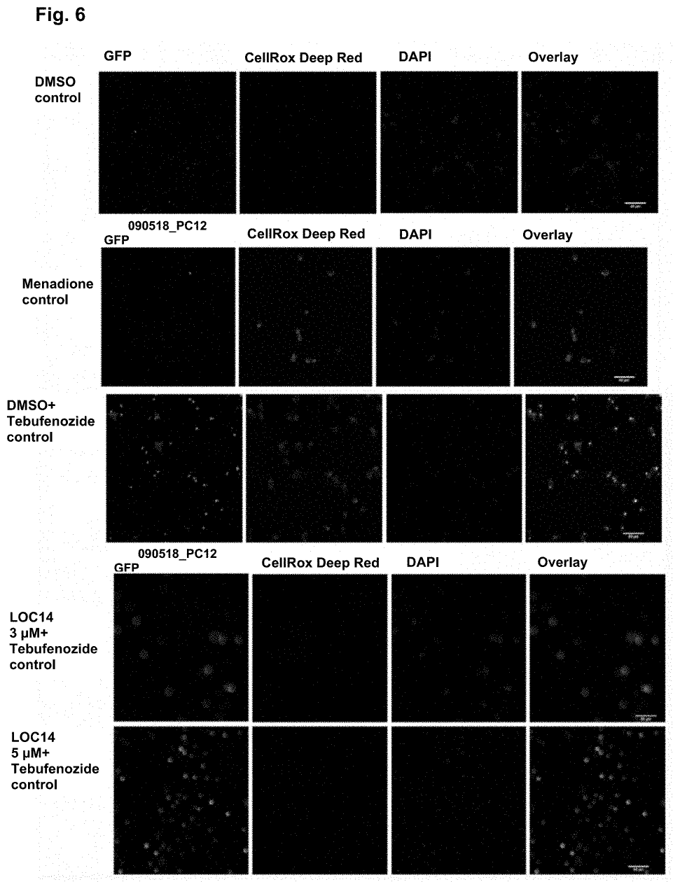

Detecting ROS is complicated because of time dependence and cell metabolic sensitivity. However, after rounds of optimization imaging of tebufenozide induced cells show reduced background and cells treated with LOC14 3 μM and 5 μM have almost completely reduced ROS production while induced cell controls (menadione for H₂O₂ production and tebufenozide for GFP-mtHtt expression) show ROS production concentrated about nucleus and presumably the ER (FIG. 6).

CellRox Dye Deep Red is very sensitive to oxidation and extraneous factors, also it might be possible to further optimize imaging times to reduce background and increase sensitivity of ROS detection. In addition, confocal imaging will bleach dye and it has a very short (2h) window of optimal imaging. Moreover, there is always some leaky expression of GFP-mtHtt which can create a background and optimization is also necessary to produce better quality images. All imaging was done using compensation algorithms included in Zeiss confocal microscopy package to improve the image quality, reduce colour leakage to other channels and improve reliability of data.

While further rounds of experiments will be necessary to explore other optimization possibilities, the results are promising and in agreement with data established using other methodologies.

### Example 7

### Hyper H₂O₂ Biosensor Analysis

Since a quarter of the ROS generated in the cell comes from oxidative disulfide bond formation in the ER with a subsequent formation of H₂O₂ as a byproduct (Tu et al., 2004; Xu et al., 2012), it is necessary to establish how mitochondria and other organelles, such as peroxisomes, contribute to ROS via oxidative phosphorylation (Malinouski et al., 2011; Murphy, 2009; Sandalio et al., 2013). Moreover, there is evidence that the ER redox state can be further perturbed by the transport of ROS from other cellular compartments (Yoboue et al., 2018).

HyPer is the optogenetic sensor capable of detecting intracellular H₂O₂. Developed on the basis of yellow fluorescent protein inserted into the regulatory domain of E. coli protein OxyR (OxyR-RD). This sensor shows sub-micromolar affinity to H₂O₂ and is insensitive to other oxidants tested, such as superoxide, oxidized glutathione, nitric oxide, and peroxinitrite. HyPer does not cause artifactual ROS generation and can detect fast changes of H₂O₂ concentration in different cell compartments under various physiological and pathological conditions.

A detailed analysis was started to optimize ROS imaging using Hyper Red H₂O₂ biosensor and this should allow us to address the following questions: how ROS transduction between organelles causes neurotoxicity; which organelle ROS species predominate in this state and what kinetics best describes oxidative stress in the cell. The rationale for this analysis is based on the hypothesis that changes in ER ROS levels can be transduced to other organelles in a cross-talk manner. That is, misfolded protein accumulation in the ER and subsequent induction of ER stress and increased activity of ER chaperones drives H₂O₂ production which is the highest in the ER and spills over to other cellular compartments.

To analyze the intracellular sources of H₂O₂ during ER stress, differentially targeted constructs of the H₂O₂ biosensors, HyPer2, HyPer Red and TRiPer will be used for cell transfection upon different ER stress challenge. We started our analysis with HyPer Red as it allows the best analysis of our currently used PC12 HD model. For ER stress experiments, cells were transfected with the specific HyperRed constructs and after 24 hours cells were subjected to the mutant huntingtin induced ER stress. Confocal live imaging microscopy was used to image H₂O₂ production at different time points in fixed cells stopping the metabolic activity of the cells. It is important to note that previously there were no reports of fixed cell analysis using these biosensors and in this experiment we explored cell fixation with formaldehyde since we speculated that HyperRed function will be preserved even after fixation and it will allow to capture ROS production at selected time points.

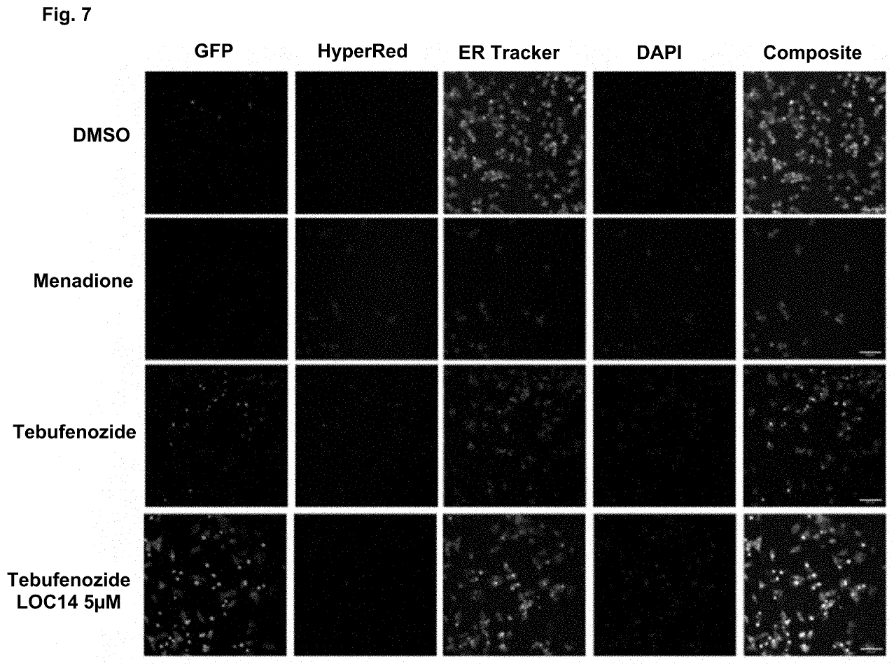

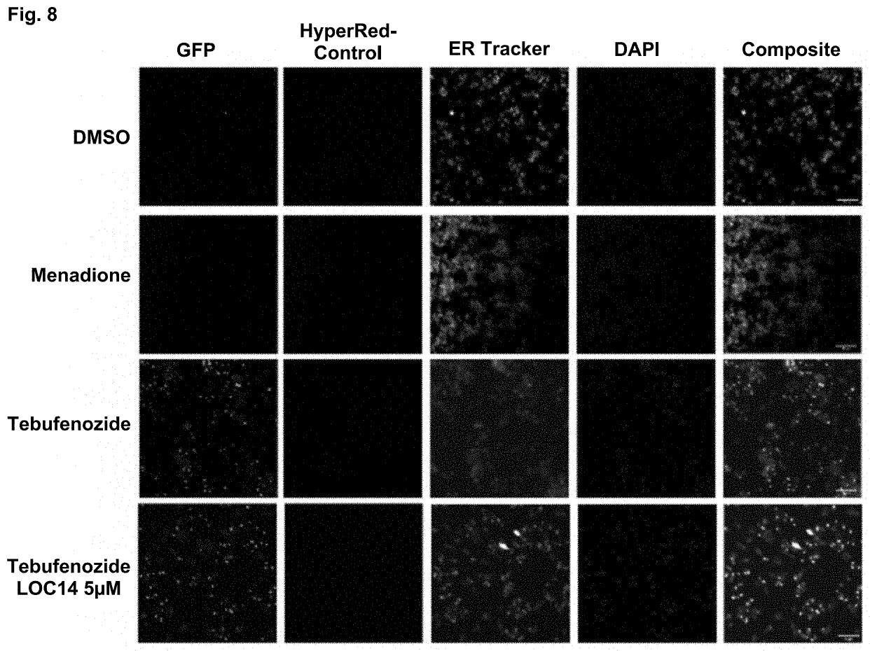

Normalizing all channels and compensating for auto-fluorescence as well as GFP, DAPI, ER Blue Tracker and HyPer Red, it is possible to produce images that reflect changes in cellular ROS (FIG. 7 and FIG. 8). Further optimization of Hyper Red might be necessary but initial results look promising. DMSO control groups (both HyPerRed and HyPerRed-control) show some leaky GFP expression and low levels of ROS which can be attributed to normal cellular metabolism. HyPerRed detection of leaky expression corresponds to the relative expression of intensity and location of GFP tagged mutant huntingtin protein. ER Blue Tracker and DAPI stains the ER and chromatin, respectively. ER Blue Tracker outlines the nucleus and there is some overlay with DAPI. Since the ER consists of membrane system the imaging is a bit more complex and has lower resolution. However, this ER dye helps to co-localize both mt-Htt and HyPerRed. Menadione positive control for hydrogen peroxide production induction shows significant response of HyPerRed to H₂O₂ production in the ER and close proximity to the ER.

Tebufenozide positive control inducing ROS through GFP-tagged mutant huntingtin expression correlates with increased ROS production detectable with HyPer Red. The location seems to be concentrated in the ER and structures around nucleus and while it is not as intense as menadione induced H₂O₂ production mt-Htt drives hydrogen peroxide production which was also confirmed by CellRox Deep Red imaging (FIG. 5). Tebufenozide and LOC14 5 μM conditions show significant reduction in observable ROS production, which is very similar to control. These results were consistent over several biological replicates as well as in earlier experiments with CellRox Deep Red (FIG. 5). This data indicates that HyperRed is a good system to image H₂O₂ at selected time points and is sensitive to hydrogen peroxide and not pH changes as was confirmed by control (FIG. 7) where non-functional HyperRed pH sensor was used to transfect cells. Moreover, LOC14 seems to show a consistent reduction in ROS levels.

In summary, treatment of induced cells with a rescue compound, LOC14, showed that pharmacological intervention of PDI/Ero1a cycling can alter hydrogen peroxide production in cellular compartments. This analysis will help further refine the role of H₂O₂ production in causing neurotoxicity, ER stress induction and kinetics of ROS transfer between different cellular compartments. In addition, it might be possible to establish whether H₂O₂ is involved in intra-cellular signalling to regulate redox kinetics in the cell and once the balance is disturbed the system deteriorates to pathological levels of H₂O₂.

### Example 8

### Gene Expression Analysis

To address potential biomarkers of oxidative ER stress in neurodegenerative diseases we selected to investigate several genes of interest based on meta-analysis and supplementary pathway analysis using bioinformatics methods.

We first selected gene SLC7A11 that encodes for a sodium-independent cystineglutamate antiporter for analysis. The protein couples the uptake of one molecule of cystine with the release of one molecule of glutamate. This gene can potentially show good biomarker characteristics for huntingtin protein overexpression and ER function perturbation based on the importance of glutamate metabolism in neural cells and cysteine role in cellular ROS kinetics. Similarly, PDI expression was found to be important in the initial computational analysis. Moreover, it was necessary to establish potential variability of PDI expression when ER function is perturbed and look into potential correlations with other genes involved in ROS overproduction response. Finally, another candidate was selected -NFE2L2 or Nrf2. This gene is a basic leucine zipper (bZIP) transcription factor that regulates the expression of antioxidant proteins and is ubiquitously expressed. Under normal conditions Nrf2 has a half-life of only 20 minutes (Kobayashi et al., 2004). We speculated that it might prove to be interesting to see how the levels of the expression of this master regulator change in response to ROS. Moreover, it is possible to infer that upstream regulator is the first to respond to any significant ROS changes.

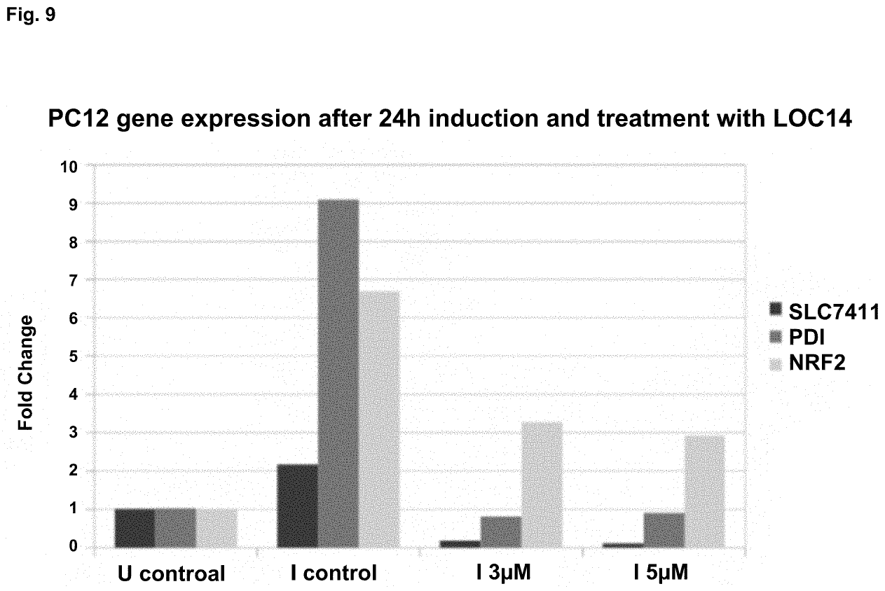

For initial rounds of analysis 24 h treatment was selected to indicate cumulative metabolic changes. While SLC7A11 does not show significant changes in expression with or without ER stress induction (just a slight increase when treated with tebufenozide), there is a significant drop when PC12 cells are treated with LOC14, which might indicate that LOC14 has substantial ROS buffering capacity reducing the need for the glutathione. PDI showed the most striking result in the upregulation when cells were treated with tebufenozide inducing ER stress, while there was significant decrease in PDI expression when cells were treated with LOC14 compounds. This result might indicate that LOC14 successfully reduce the need for cells to produce more PDI to cope with ER stress. This was followed by NRF2 expression, which was less dramatic than PDI but followed similar pattern. This might point to fact that both PDI and NRF2 have varying modes of expression and belong to a different hierarchy in terms of response to ER stress and ROS production (FIG. 9).

### Example 9

### Lipidomics Analysis

Accumulating evidence suggests that the UPR is important in maintaining lipid homeostasis and metabolism (Kammoun et al., 2009; Han et al., 2016). During ER stress, this organelle is actively reshaped to accommodate protein refolding and the expanding membrane network is accompanied by the increase in lipid synthesis (Mandl et al., 2013). Moreover, a number of proteins depend on proper lipidation and perturbed ER function as well as changes in lipogenation profile may potentially modulate protein function and aggregation.

In preparation for extensive lipidome analysis, lipid Peroxidation (MDA) assay (Colorimetric/Fluorometric) (ab118970) was performed to establish peroxidation product formation in PC12 cells and evaluate major effects when treating with the rescue compound. This assay provides a convenient tool for sensitive detection of the malondialdehyde (MDA) present in a variety of samples. MDA, together with 4-hydroxynonenal (4-HNE), is a natural bi-product of lipid peroxidation and its quantification is generally used as marker for lipid peroxidation. The MDA in the sample reacts with thiobarbituric acid (TBA) to generate a MDA-TBA adduct. The MDA-TBA adduct can be easily quantified calorimetrically (OD=532 nm) orfluorometrically (Ex/Em=532/553 nm). This assay detects MDA levels as low as 1 nmol/well colorimetrically and 0.1 nmol/well fluorometrically. The MDA assay is also more specific than the TBARS assay. MDA lipid peroxidation assay for PC12 cells with induced ER stress allowed to determine baseline for lipid peroxide products after ER stress and ROS production induction, determine if LOC14 has any detectable effect on lipid peroxidation after 24h treatment, and compare normal PC12 cells and cells with induced ER stress in terms lipid peroxide products.

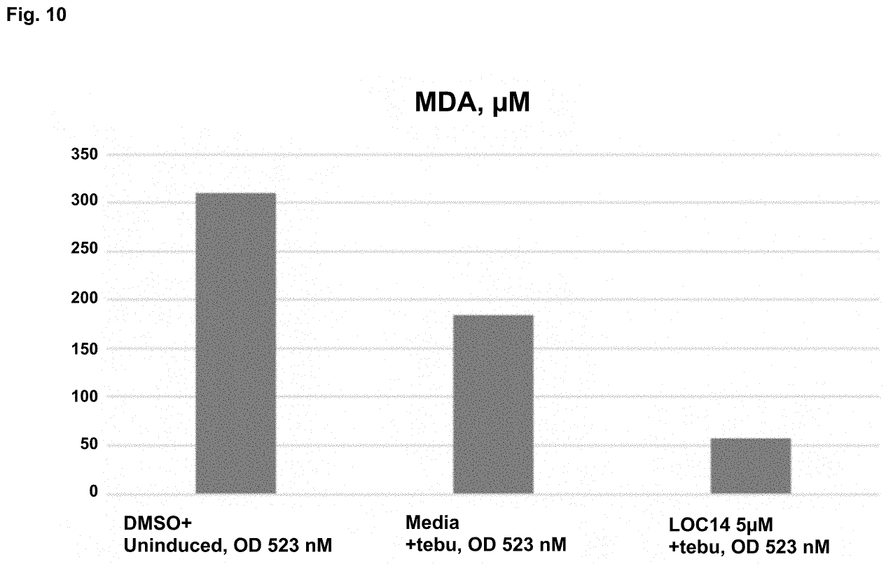

MDA assay (FIG. 10) revealed that treatment with LOC14 significantly reduces MDA levels in treated cells, which could be attributed to reduction of ROS mediated via LOC14 action. However, control without any treatment (DMSO) shows almost two times higher concentration of MDA than the positive control (Tebufenozide). There might be several explanations for this, first of all, tebufenozide subjects cells to increased ER stress and subsequently antioxidation pathways are induced to control cell's ROS levels, which might account for the observed reduction (24 h hours). Also, it's possible that a large portion of cells that were the most affected by lipid peroxidation were not analyzed as they died and were removed with the media exchange before cell harvesting. Finally, it is necessary to consider other approaches for further optimization of the assay, such as different normalization method, different treatment times or different controls (e.g. menadione). Despite these shortcomings, the initial assay looks promising with significant rescue MDA suppression by LOC14 treatment which is encouraging results to proceed with more in depth analysis of PC12 HD model lipidome.

### Example 10

### Reduction of ER-Generated-ROS is Neuroprotective in Alzheimer's Disease

Overproduction of reactive oxygen species (ROS) is at the heart of ageing, cancer, stroke and neurodegenerative diseases. Chemical agents that are not just antioxidant scavengers but can reduce the formation of ROS species in the cell, provide a highly desirable therapeutic strategy to combat these diseases. Here, it is shown that one such molecule, LOC14, protects in Huntington's and Alzheimer's disease models by reducing the formation of ROS in the endoplasmic reticulum (ER). It is also shown that the mechanism of neuroprotection of LOC14 involves it binding reversibly to the catalytic cysteine 53 of reduced protein disulfide isomerase (PDI), a chaperone protein in the ER that is up-regulated in the presence of misfolded proteins, resulting in an oxidized PDI conformation. The oxidation of PDI by LOC14 eliminates the need for activation of PDI's binding partner, ER oxidoreductin 1 (ERO1) which is a source of a significant amount of ROS in the ER. Furthermore, using a medicinal chemistry approach 73 analogs of LOC14 were synthesized to develop a structure-activity-relationship and obtain optimized lead compounds with improved drug-like properties. These studies suggested that LOC14 and its analogs represent useful tools for studying ER ROS in the context of neurodegenerative diseases and opens up a new therapeutic avenue for treatment of diseases such as Huntington's and Alzheimer's disease.

Neurodegenerative disorders constitute a class of diseases that express misfolded proteins that aggregate and induce neuronal toxicity and death. Alzheimer's disease (AD) is one such fatal protein misfolding disease that afflicts neurons and synapses in the cerebral cortex and subcortical regions of the brain (Behl 1999). A promising new drug target for AD is protein disulfide isomerase (PDI) (Hoffstrom et al. 2010), a chaperone protein that is responsible for the isomerization, reduction, and oxidation of non-native disulfide bonds in unfolded proteins entering the endoplasmic reticulum (ER). Previously, we discovered a neuroprotective, reversible, small molecule modulator of PDI, compound LOC14, that has nanomolar potency in cells, high in vitro stability in liver microsomes and blood plasma, and that protects in brain slice and mouse models of Huntington's disease (HD) (Kaplan et al. 2015; Zhou et al. 2018). Since HD is another neurodegenerative protein misfolding disease, we hypothesized that perhaps AD and HD share a common cell death pathway for which PDI modulation with LOC14 might be neuroprotective. In this report we investigate the neuroprotective effects of LOC14 in HD and AD models and investigate the molecular mechanism of how LOC14 interacts with PDI and why that leads to neuroprotection.

Furthermore, despite the fact that LOC14 is orally bioavailable, blood-brain-barrier penetrant, and is tolerated at a high dose of 20 mg/kg in mice, it has sub-optimal pharmacokinetic properties (half-life of 2.2 hours in the brain via oral gavage) and a narrow window of efficacy due to toxicity at higher concentrations (Kaplan et al. 2015; Zhou et al. 2018) that make it not an ideal candidate for the next steps in developing it as a drug. Therefore, further optimization of the LOC14 scaffold was performed through thorough analog design to obtain optimal lead compounds that can properly be used to evaluate the therapeutic potential of modulating PDI in AD and other neurodegenerative diseases driven by and/or involving protein misfolding.

### LOC14 is a Selective, Reversible, Oxidant of PDI

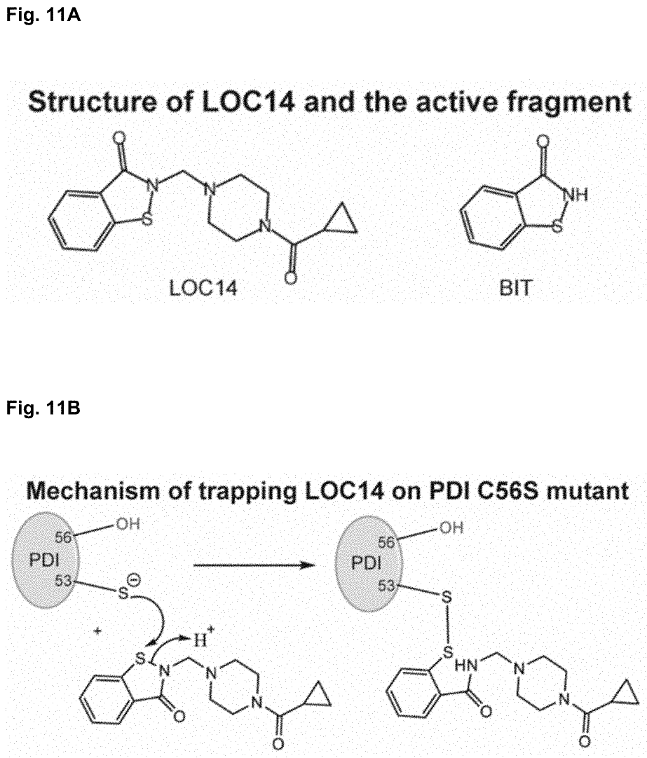

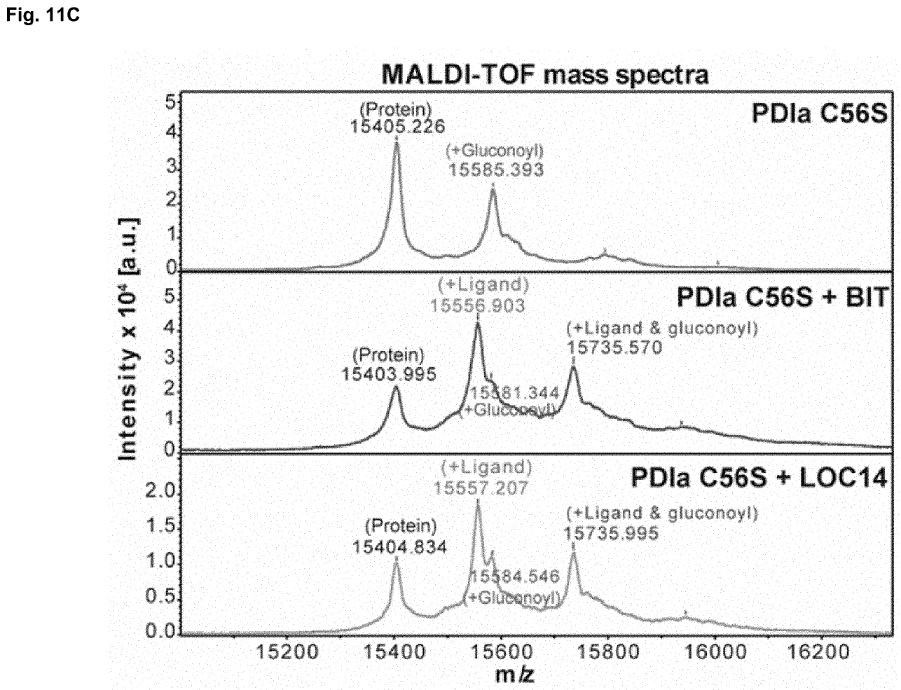

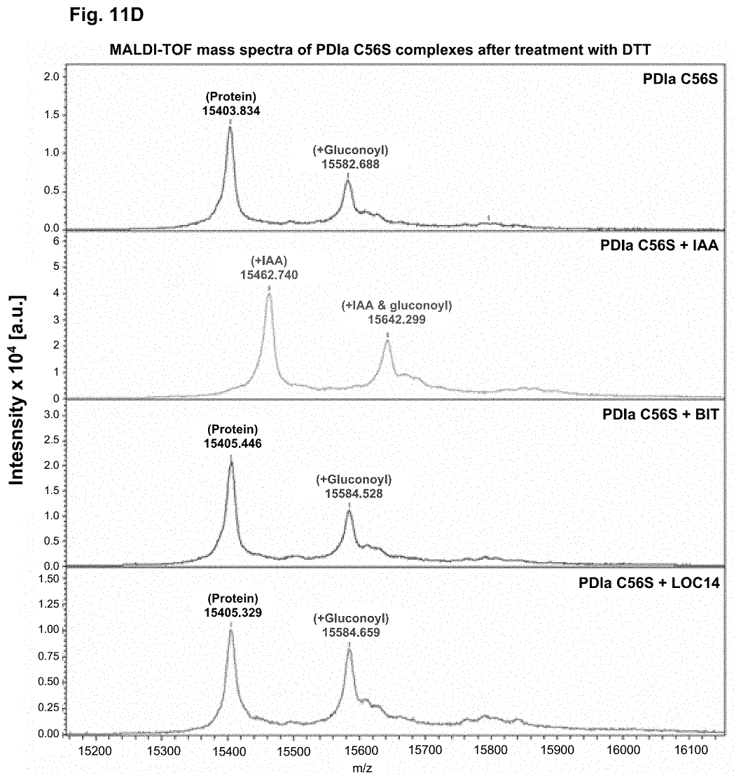

LOC14 is a neuroprotective compound that targets PDI. The structure of LOC14 is shown in FIG. 11A. It was previously showed through NMR studies, that LOC14 induced the largest chemical shift changes in the active site region of the recombinant catalytic a domain of human PDI A1 (referred hereto as PDIa) causing it to adopt a conformation similar to its oxidized state (Kaplan et al. 2015). Here, we sought to validate the mechanism of binding, and to identify specific moieties on LOC14 that bind to on PDI.

It was hypothesized that LOC14 would form a transient interaction with the catalytic Cys53, mimicking the dithiol-disulfide chemistry of a native PDI substrate. This N-terminal cysteine, C53, in the CGHC active site of PDI, has an extremely low pKa of 4.5-5.6 (Kortemme et al. 1996; Ruddock et al. 1996) making it the primary nucleophile for attacking substrates. Once C53 forms an intermolecular disulfide bond with the substrate, the thiolate of the second cysteine, C56, forms an intramolecular disulfide bond with C53 to release PDI and the substrate.

To test this mechanism and trap the LOC14-PDI complex, we generated a PDIa C56S mutant that would not be capable of releasing the compound from PDI according to the proposed mechanism (FIG. 11B). We performed MALDI-TOF mass-spectrometry experiments with PDIa C56S in the presence and absence of either LOC14 or the benzoisothiazolone (BIT) moiety of LOC14 (FIG. 11C). BIT is the active fragment of LOC14 that is formed under ionizing conditions of mass spectrometry. Such mass fragmentation is common and is consistent with properties reported for Mannich bases (Holla et al. 1998; Fuchslueger et al. 1999). The predicted mass for the PDIa C56S mutant was 15,404 m/z (without H⁺). Experimentally, the PDIa C56S mutant showed two mass peaks: a main protein mass peak at 15,405 [M+] and a second peak at 15,585 (+179 change) corresponding to the common gluconoylation modification to C56S protein on the His6× tag by E. coli (Geoghegan et al. 1999). PDIa C56S treated with LOC14 yielded four peaks: the main mass peak at 15,557 m/z (change of +152 which corresponds to bound BIT on PDIa C56S), a peak at 15,735 Da (corresponding to the benzoisothiazolone moiety bound to C56S-gluconoylated protein), a peak at 15,404 (the mass of unbound C56S protein), and a peak at 15,584 Da (the mass of unbound C56S-gluconoylated protein). These data indicate that LOC14 interacts and binds to PDI, most likely on the catalytic C₅₃ nucleophile. The incomplete binding is most likely due to the presence of reducing agent in the buffer.

To confirm that binding of LOC14 to PDI is reversible, we pre-treated a C56S mutant, as before, with LOC14, BIT, or iodoacetamide, an irreversible modifier of cysteines. Once the PDI-inhibitor complexes were formed, we treated the samples with 2.7 mM DTT, a reducing agent, and analyzed the complex by MALDI-TOF. PDIa C56S sample treated with iodoacetamide and DTT showed two peaks with carboxyamidomethylcysteine modification (one for main PDIa C56S peak and one for gluconoylated PDIa C56S peak), indicating that iodoacetamide binding to cysteine is irreversible and cannot be removed with DTT. The samples of PDIa C56S treated with BIT or LOC14, however, showed only the mass for unmodified cysteine after DTT treatment (FIG. 11D). These data indicate that LOC14 and its active fragment, BIT, bind reversibly to C53, and this transient bond can be removed with reducing agent.

Having validated the mechanism of binding of LOC14 to PDI, we tested whether LOC14 bound selectively to PDI. To assess this, we tested LOC14 against a panel of 30 diverse targets and saw no binding or inhibition to any other targets at 3 μM (Table 2). Additionally, we tested LOC14 in CEREP's Safety47 panel, an in vitro pharmacological profiling to determine off-target liabilities that might exist against 47 human targets associated with safety issues (Table 3 and Table 4). The targets were selected based on safety recommendations by major pharmaceutical companies of frequently hit targets (Bowes et al. 2012) and involved a broad range of proteins, including receptors, ion channels, transporters, enzymes and second messengers. In this panel, LOC14 showed a significant effect of only moderate 70% activity against muscarinic acetylcholine receptor M2 (Table 3). This is a low 2.1% promiscuity value (percent targets hit compared to total targets tested) (Bowes et al. 2012), indicating that LOC14 predominantly targets PDI; however, future analogs could be sought that reduce binding to muscarinic acetylcholine receptor M2.

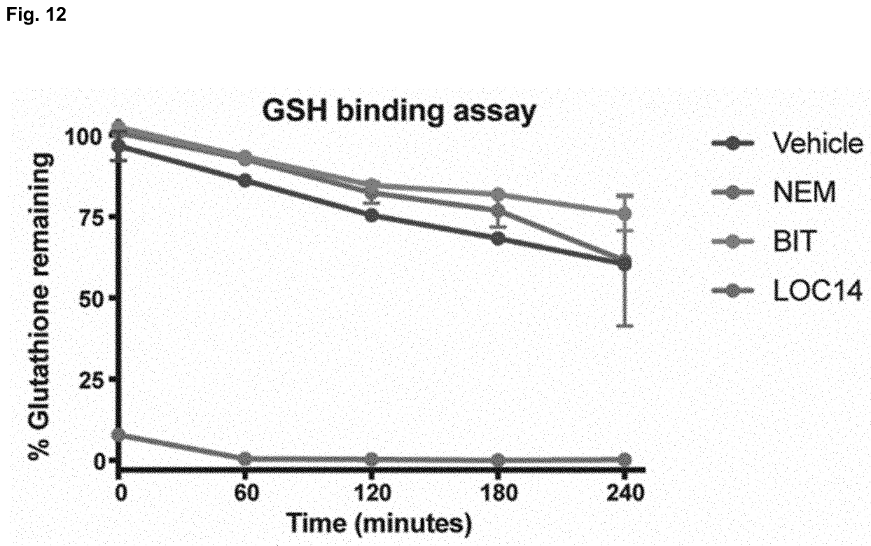

Additionally, we evaluated LOC14 for its ability to interact with glutathione (GSH), the most abundant thiol in cells (Forman et al. 2009). Glutathione binding to a compound results in the compound's excretion into bile, and serves as an important pathway of elimination. To assess the ability of LOC14 to bind to GSH, we used a fluorometric assay in which the reduction in glutathione levels upon compound binding was quantified using Ellman's reagent. The results from this assay showed that LOC14 does not bind to glutathione (FIG. 12). The vehicle control shows that the glutathione concentration slowly diminishes over time, presumably due to the oxidation of GSH to GSSG in buffer. Incubation of the positive control, N-ethylmaleimide (NEM), with glutathione led to an immediate depletion of glutathione (FIG. 12). This supports the conclusion that LOC14 is selective for binding to PDI.

### LOC14 is Neuroprotective in Multiple Neurodegeneration Models

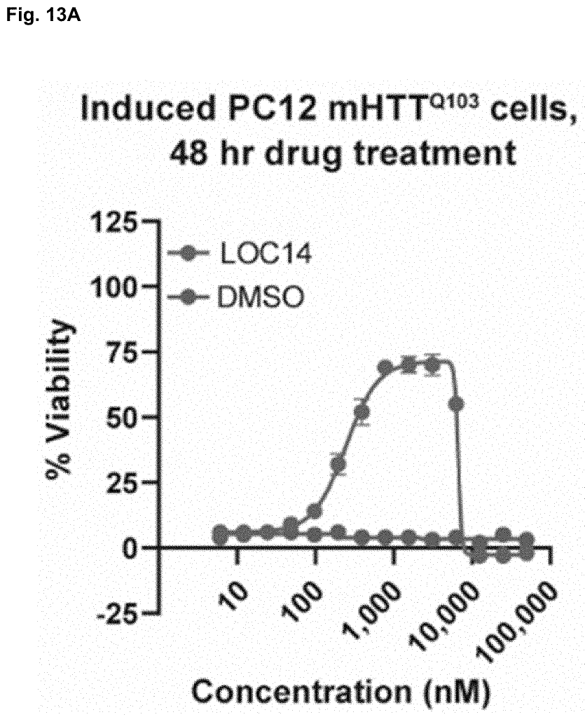

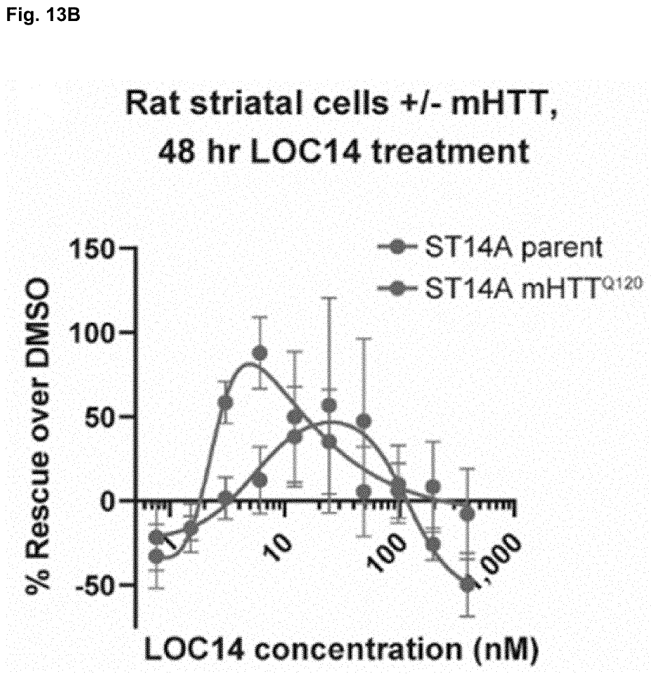

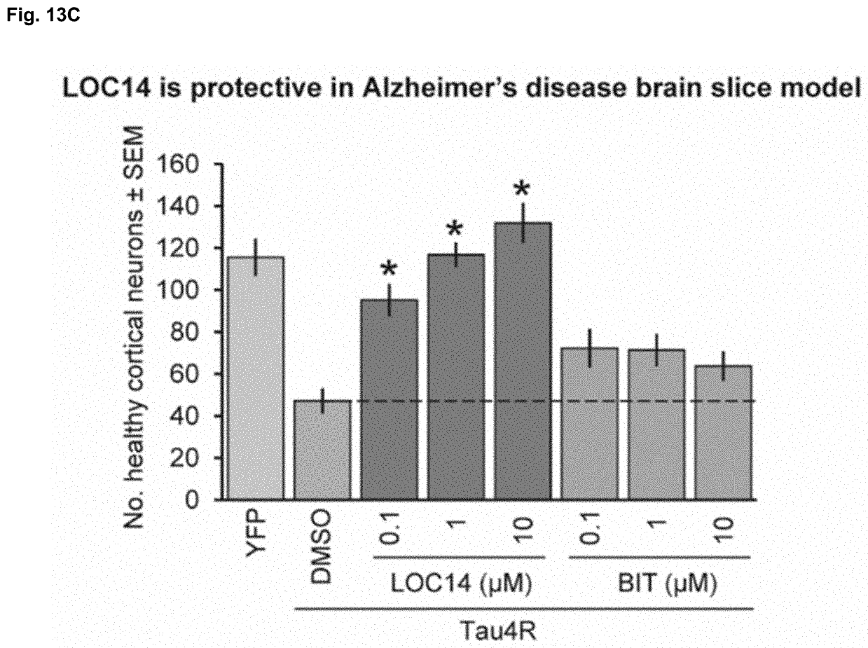

Having elucidated the molecular mechanism of LOC14's binding to PDI, we next tested whether PDI modulation with LOC14 would be effective in neurodegenerative disease models. First, we evaluated LOC14's neuroprotection in rat neuron-like PC12 cells stably transfected with an inducible plasmid for truncated mutant huntingtin protein (mHTT^{Q103}). In this model of Huntington's disease (HD), LOC14 protected in a dose-dependent manner from mHTT toxicity with EC₁₀₀ at 3.1 μM (FIG. 13A). We next evaluated the activity of LOC14 in cells that more closely represent the medium-size spiny neurons affected in Huntington Disease patients. We used rat embryonic day 14 striatal neuronal cells (ST14A) immortalized by transduction of temperature-sensitive SV40 large T antigen (Cattaneo and Conti, 1998; Ehrlich et al. 2001; Rigamonti et al. 2000). Under serum deprivation and at 39° C., where large T antigen is degraded, ST14A cells undergo cell death within 48-72 hours. This death was rescued by treatment with LOC14 (FIG. 13B). Furthermore, LOC14 was more potent at rescuing ST14A cells transfected with 120 polyQ in an N-terminal HTT (mHTT^{Q120}) fragment compared with parental ST14A cells (without mutant huntingtin). In ST14A mHTT^{Q120} cells, LOC14 had a potent EC₁₀₀ of 6 nM, four-fold lower than in parental ST14A cells (EC₁₀₀ at 24 nM) and much more potent than in PC12 mHTT^{Q103} cells (FIG. 13A). This suggests that medium-size spiny neurons affected by mutant huntingtin toxicity are much more sensitive to LOC14's mechanism of protection than other cell types or ones not afflicted with the disease protein.

To see if the mechanism of LOC14 protection can ameliorate other neurodegenerative disease phenotypes, we used a brain slice model of Alzheimer's disease (AD) in which the degeneration of cortical pyramidal neurons is induced via transfected tau protein containing four repeats of the microtubulin-binding domain (Tau-4R). Rat coronal brain slice explants were co-transfected with YFP and Tau-4R and treated with LOC14, benzoisothiazolone, or DMSO vehicle-only for 3 days. The cortical region of each brain slice was then scored for number of healthy pyramidal neurons remaining. In this model, LOC14 protected pyramidal neurons from Tau-4R-induced toxicity in a dose-dependent manner (FIG. 13C). These results together support the notion that protein misfolding neurodegenerative diseases, such as HD and AD, share common pathways for which PDI modulation with LOC14 is a promising therapeutic approach.

### Mechanism of Neuronal LOC14 Protection Causes Reduction of ROS

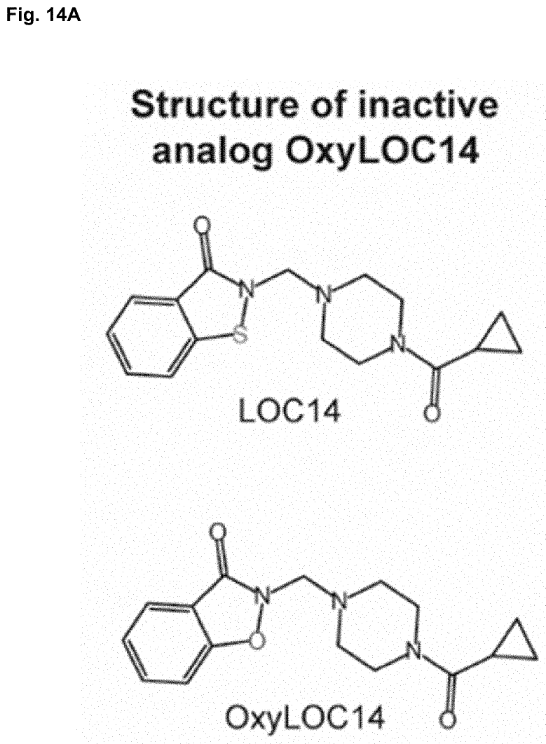

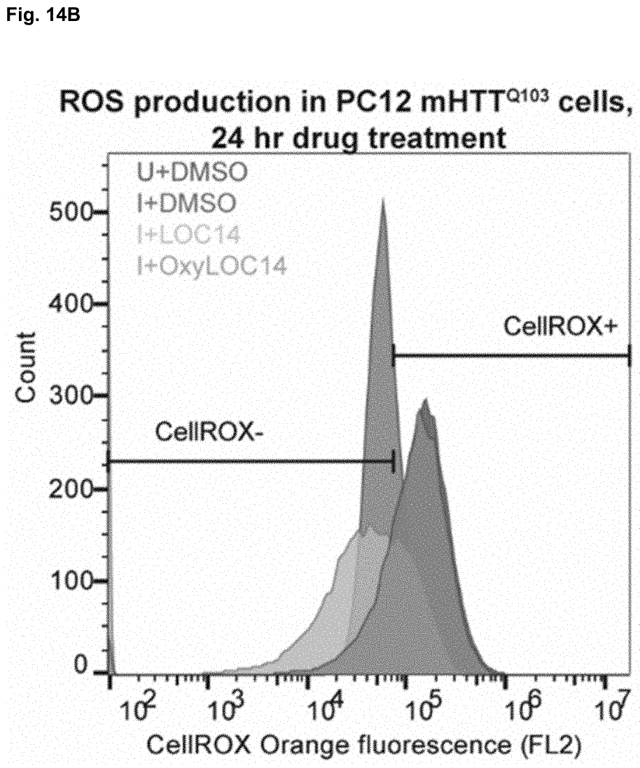

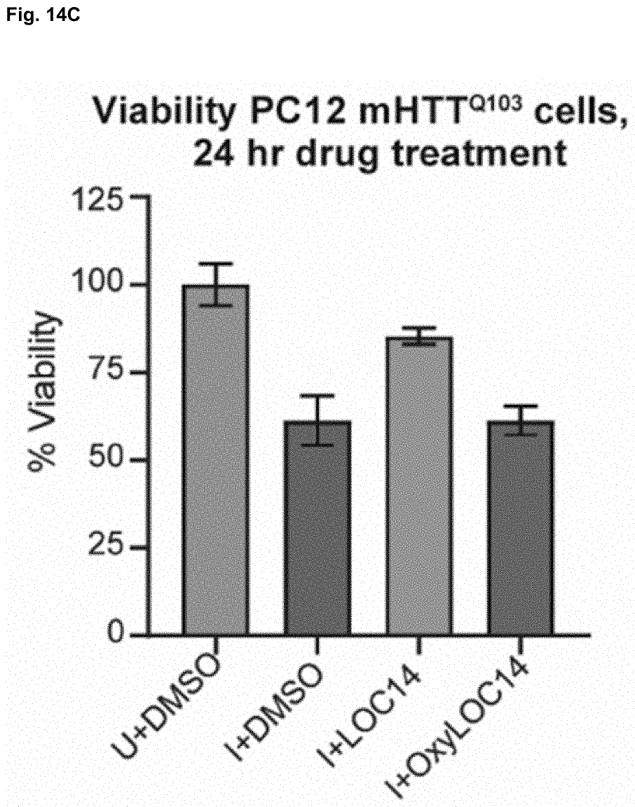

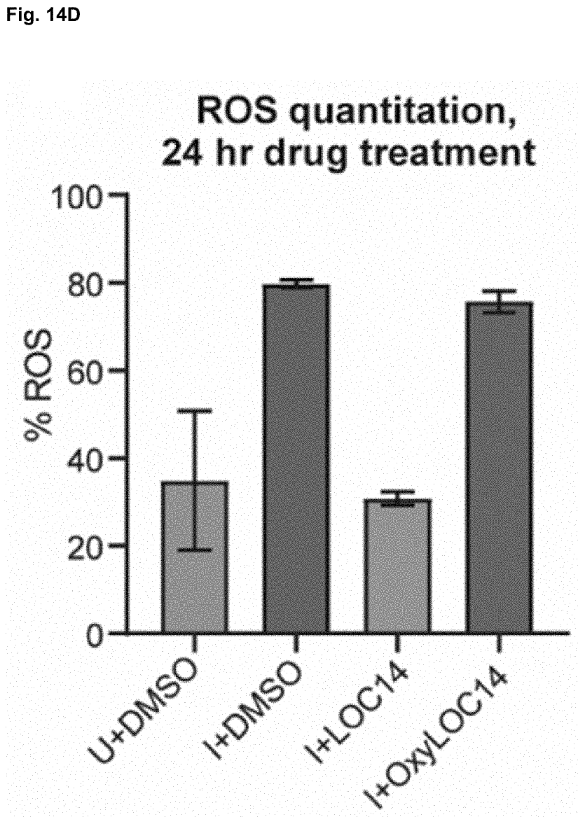

One goal was to further understand why modulating PDI activity can be neuroprotective in AD and HD models. Previously, we saw that LOC14 alters PDI's redox state from the reduced to the oxidized conformation (Kaplan et al. 2015). In vivo, there are oxidoreductase enzymes, such as ERO1, that regulate PDI's redox states and, as a byproduct of the redox reaction, pass electrons to oxygen as a final electron acceptor, generating H₂O₂. We hypothesized that LOC14 might function in place of oxidoreductases such as ERO1, oxidizing PDI without generating H₂O₂ and that this decrease in ROS production is what is causing the neuroprotection in cells and neurons. To test this model, we induced mHTT expression in PC12 cells and treated with DMSO vehicle, LOC14, or an inactive isoxazolone analog of LOC14, termed OxyLOC14 for 24 hours. Cells were then stained with the fluorescent ROS-sensitive dye, CeIIROX Orange, and ROS assayed by flow cytometry. Induced mHTT-expressing cells had elevated levels of ROS compared to cells not expressing mHTT (uninduced cells) and these ROS were decreased in cells treated with LOC14 (FIG. 14B). Quantitation of flow cytometry data showed that LOC14-treated cells had the same level of ROS as mHTT non-expressing cells (FIG. 14D). Treating the cells with OxyLOC14, which differs from LOC14 by a single atom substitution from sulfur to oxygen, did not decrease ROS (FIGS. 14A-14D). Unlike LOC14, the inactive analog also did not show any protection from mHTT in the trypan blue viability assay (FIG. 14C). This supports the hypothesis that oxidizing PDI with LOC14 leads to a reduction of ROS production in the cell and results in neuroprotection.

LOC14 analog design

Having shown that LOC14 protects cells and neurons in models of HD and AD by selectively and reversibly binding to PDI, oxidizing it so it is available to continue forming native disulfide bonds in the ER, and thus reducing the cellular ROS levels, we next asked if the compound can be further optimized through a through analog design.

Analogs of the cyclopropylcarbonyloiperazine group

Our initial synthesis efforts focused on derivatization of the 1-cyclopropylcarbonylpiperazine group. By altering the cyclopropylcarbonyl group on the piperazine ring, as well as the piperazine ring itself, we aimed to test whether modifications of this group could lead to better properties of analogs. However, these modifications did not improve the potency of the compounds. The biochemical and cell activity data of analogs that retained the piperazine ring and had a modification of the cyclopropylcarbonyl group (analogs: LC-2, LC-10, LC-12, LC-13, and LC-22) showed no change in activity compared to LOC14 (Table 5).

TABLE 5
Modifying cyclopropylcarbonyl group has no improvement over LOC14.

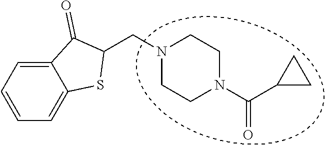 [SMILES: O=C1c2ccccc2SC1CN1CCN(C(=O)C2CC2)CC1]

| Compound | R | Solubility (mM) | Kd (nM) | EC50 (nM) | Max Viability (%) |
|---|---|---|---|---|---|
| LOC14 | 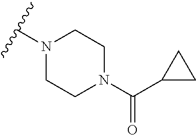 [SMILES: *N1CCN(C(=O)C2CC2)CC1] | 100 | 113 ± 43 | 352 | 66 |
| LC-2 | 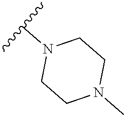 [SMILES: *N1CCN(C)CC1] | 100 | 134 ± 20 | 415 | 63 |
| LC-10 | 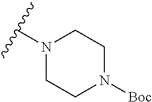 [SMILES: *N1CCN(C(=O)OC(C)(C)C)CC1] | 100 | 123 ± 34 | 581 | 72 |
| LC-12 | 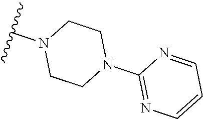 [SMILES: *N1CCN(c2ncccn2)CC1] | 100 | 140 ± 29 | 416 | 68 |
| LC-13 | 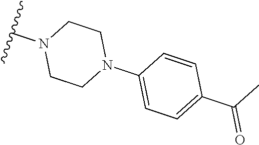 [SMILES: *N1CCN(c2ccc(C(C)=O)cc2)CC1] | 100 | 190 ± 27 | 514 | 63 |
| LC-22 | 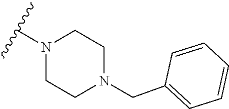 [SMILES: *N1CCN(Cc2ccccc2)CC1] | 100 | 173 ± 32 | 310 | 62 |

Altering the piperazine ring to piperidine (LC-1) also had no effect on activity (Table 6). Interestingly, adding a hydroxyl group to the piperidine moiety (LC-4 and LC-7) or changing from piperazine to a morpholine ring (LC-18), led to a two-fold decrease in binding to PDI, without a significant change in EC₅₀ values, while addition of a secondary amine to the piperidine ring (LC-8) had the same activity as LOC14. Analog LC-64 with a tertiary amine had poor binding to PDI, most likely due to the bulky bis-piperidine moiety. Thiomorpholine (LC-3) or pyrrolidine (LC-65) ring-modifications were tolerated without a change in biochemical or cell activity. However, changing the piperazine ring to a benzyl group (analogs: LC-5 and LC-26) led to a weaker and non-specific binding to PDI (as seen by two-site binding of LC-5) and a decrease in protection from mHTT-induced toxicity in cells. Together, these data indicate that the cyclopropylcarbonylpiperazine moiety can tolerate many modifications without a significant effect on K_{D} or potency in cells (Table 6), which could be valuable in optimizing ADME properties. However, complete elimination of cyclopropylcarbonylpiperazine was not tolerated as it led to a loss of protection from expanded Tau4R-toxicity in a brain slice model of AD (FIG. 13C).

TABLE 6
LOC14 analogs with modified piperazine ring.

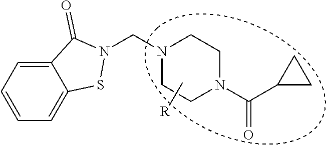

| Compound | R | Solubility (mM) | Kd (nM) | EC50 (nM) | Max Viability (%) |
|---|---|---|---|---|---|
| LOC14 | 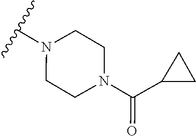 [SMILES: *N1CCN(C(=O)C2CC2)CC1] | 100 | 113 | 352 | 66 |
| LC-4 | 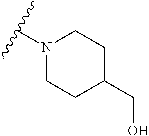 [SMILES: *N1CCC(CO)CC1] | 100 | 265 | 343 | 60 |
| LC-7 | 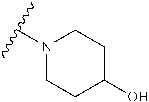 [SMILES: *N1CCC(O)CC1] | 100 | 216 | 441 | 72 |
| LC-8 | 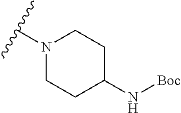 [SMILES: *N1CCC(NC(=O)OC(C)(C)C)CC1] | 50 | 94 | 384 | 68 |
| LC-64 | 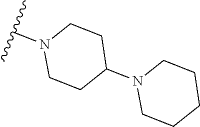 [SMILES: *N1CCC(N2CCCCC2)CC1] | 50 | 1210 | 393 | 81 |
| LC-1 | 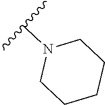 [SMILES: *N1CCCCC1] | 100 | 107 | 459 | 67 |
| LC-18 | 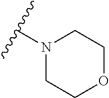 [SMILES: *N1CCOCC1] | 100 | 253 | 547 | 69 |
| LC-3 | 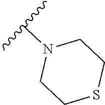 [SMILES: *N1CCSCC1] | 100 | 92 | 463 | 63 |
| LC-65 | 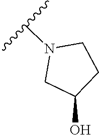 [SMILES: *N1CC[C@@H](O)C1] | 100 | 162 | 456 | 77 |
| LC-5 | 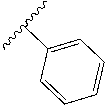 [SMILES: *c1ccccc1] | 100 | 43; 2268 | 768 | 46 |
| LC-26 | 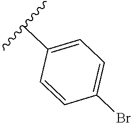 [SMILES: *c1ccc(Br)cc1] | 100 | N.A. | 778 | 38 |

Derivitalizing benzoisothiazolone moiety

Next, we investigated altering the benzoisothiazolone moiety of LOC14. Since the mechanism of binding involves the interaction of the sulfur group in the benzoisothiazolone moiety with Cys53 of PDI, we wanted to test if such an interaction would still occur with the ring-opened analogs (analogs: LC-19, LC-20, LC-21, LC23, LC28, LC-30, LC-33, and LC-34). In each case, the ring-opened analogs had complete loss of binding and inhibition of PDI compared to their ring-closed counterparts (Table 7). Interestingly, LC-23, LC-28, LC-30, and LC-33, were 3-10 fold less potent in cells than LOC14 but still maintained a protective effects on mHTT-toxicity. This result suggests that either these analogs are protecting cells through a different mechanism not relevant to PDI inhibition or there is an oxidizing cofactor/enzyme present in cells that can oxidatively cyclize the ring-opened analogs. To test whether PDI can catalyze this interaction, we performed ITC binding studies with ring-opened analogs and oxidized PDI, but no binding was observed, indicating PDI does not catalyze this reaction.

TABLE 7
Ring-opened analogs have loss of PDI binding activity and decreased potency in cells.

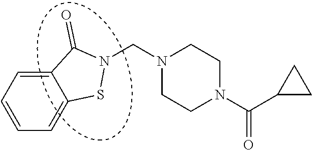 [SMILES: O=C(C1CC1)N1CCN(Cn2sc3ccccc3c2=O)CC1]

| | | Solubility | Kd | EC50 | Max |
|---|---|---|---|---|---|
| Compound | | (mM)* | (nM) | (nM) | Viability (%) |
| LC-19 | 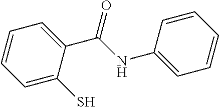 [SMILES: O=C(Nc1ccccc1)c1ccccc1S] | 50 | NA | 1,635 | 48 |
| LC-20 | 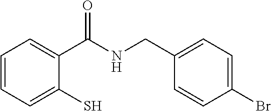 [SMILES: O=C(NCc1ccc(Br)cc1)c1ccccc1S] | 100 | NA | >50,000 | 42 |
| LC-21 | 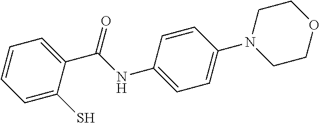 [SMILES: O=C(Nc1ccc(N2CCOCC2)cc1)c1ccccc1S] | 100 | NA | 696 | 32 |
| LC-23 | 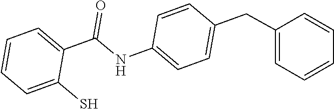 [SMILES: O=C(Nc1ccc(Cc2ccccc2)cc1)c1ccccc1S] | 100 | NA | 3,883 | 57 |
| LC-28 | 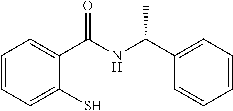 [SMILES: C[C@@H](NC(=O)c1ccccc1S)c1ccccc1] | 100 | NA | 808 | 56 |
| LC-30 | 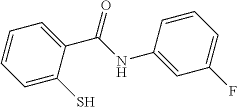 [SMILES: O=C(Nc1cccc(F)c1)c1ccccc1S] | 100 | NA | 2,992 | 62 |
| LC-33 | 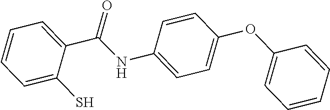 [SMILES: O=C(Nc1ccc(Oc2ccccc2)cc1)c1ccccc1S] | 100 | NA | 2,937 | 54 |
| LC-34 | 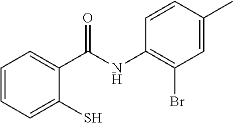 [SMILES: Cc1ccc(NC(=O)c2ccccc2S)c(Br)c1] | 100 | NA | 3,767 | 42 |

We tried modifying the benzoisothiazolone ring to isothiazolone (LC-35), benzodithiolone (LC-48), or benzodithiolethione (LC-49) and saw loss of binding and inhibition of PDI and no activity in cells (Table 8).

TABLE 8
Modification of benzoisothiazolone moiety leads to a loss of activity.

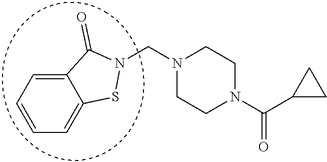 [SMILES: O=C(C1CC1)N1CCN(Cn2sc3ccccc3c2=O)CC1]

| Compound | | Solubility (mM) | Kd (nM) | EC50 (nM) | Max Viability (%) |
|---|---|---|---|---|---|
| BIT | 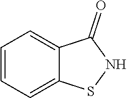 | 100 | 143 | 362 | 66 |
| LC-35 | 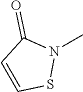 | 100 | NA | NA | 15 |
| LC-48 | 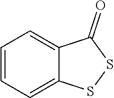 | 100 | NA | NA | 8 |
| LC-49 | 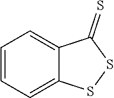 | 100 | NA | NA | 9 |

We also derivatized the benzene ring of the benzoisothiazolone moiety in order to explore the effect of electronics and sterics on the activity of LOC14 (Table 9). We hypothesized that electron withdrawing and donating groups can affect both the binding to PDI and the reactivity of the isothiazolone ring to the cysteine in PDI, while lipophilic substituents may make hydrophobic interactions with the enzyme. We found that analogs that contained any substituent at the ortho position (analogs: LC-38, LC-39, LC-40, LC-41, LC-42 and LC-43) showed a complete loss of activity in cells and in targeting PDI in vitro (except the fluorine group, described later). Analog LC-15, containing a chloro group para to the sulfur of the isothiazolone ring, however, had the same activity as LOC14 in cells and at inhibiting PDI in the biochemical assays. Its counterpart, analog LC-39, with the chloro ortho to the sulfur of the isothiazolone ring, was completely inactive (Table 9). This suggests that substitution at the ortho position adjacent to sulfur sterically decreases the accessibility of the isothiazolone sulfur to cysteine, leading to a decrease in reactivity and binding. Methyl substitution groups para to the sulfur of the isothiazolone ring (LC-61 and LC-63) were two-fold less active than LOC14, while methyl substitutions meta to the sulfur (LC-60 and LC-62) were three-to-six fold less active in cells and at binding to PDI in vitro (Table 9).

TABLE 9
Ortho substitutions on the ring decrease cysteine accessibility.

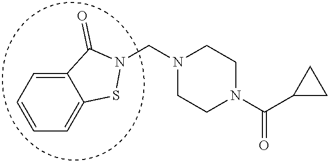 [SMILES: O=C(C1CC1)N1CCN(Cn2sc3ccccc3c2=O)CC1]

| | | Solubility | Kd | EC50 | Max |
|---|---|---|---|---|---|
| Compound | | (mM) | (nM) | (nM) | Viability (%) |
| LC-38 | 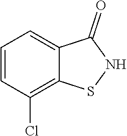 [SMILES: O=c1[nH]sc2c(Cl)cccc12] | 100 | NA | NA | 8 |
| LC-39 | 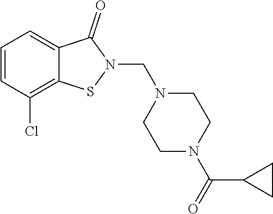 [SMILES: O=C(C1CC1)N1CCN(Cn2sc3c(Cl)cccc3c2=O)CC1] | 100 | NA | NA | 7 |
| LC-15 | 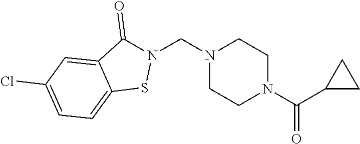 [SMILES: O=C(C1CC1)N1CCN(Cn2sc3ccc(Cl)cc3c2=O)CC1] | 15 | 114 | 487 | 57 |
| LC-41 | 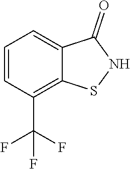 [SMILES: O=c1[nH]sc2c(C(F)(F)F)cccc12] | 100 | NA | NA | 9 |
| LC-42 | 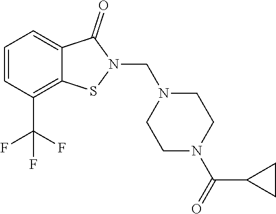 [SMILES: O=C(C1CC1)N1CCN(Cn2sc3c(C(F)(F)F)cccc3c2=O)CC1] | 100 | NA | NA | 8 |
| LC-40 | 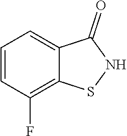 [SMILES: O=c1[nH]sc2c(F)cccc12] | 100 | NA** | 9,325 | 72 |
| LC-43 | 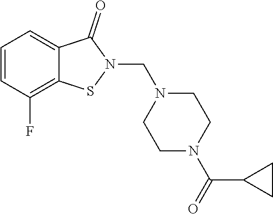 [SMILES: O=C(C1CC1)N1CCN(Cn2sc3c(F)cccc3c2=O)CC1] | 100 | NA** | 7,409 | 56 |
| LC-60 | 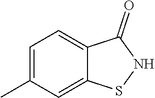 | 100 | 347 | 562 | 60 |
| LC-62 | 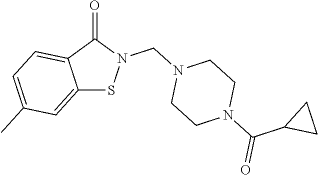 [SMILES: Cc1ccc2c(=O)n(CN3CCN(C(=O)C4CC4)CC3)sc2c1] | 50 | 662 | 1,576 | 54 |
| LC-61 | 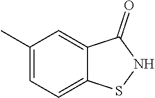 | 100 | 267 | 591 | 65 |
| LC-63 | 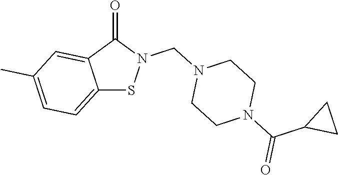 [SMILES: Cc1ccc2sn(CN3CCN(C(=O)C4CC4)CC3)c(=O)c2c1] | 50 | 240 | 659 | 66 |

NA** compounds active in enzymatic assay for inhibition of PDI using insulin as substrate

### Pyridine and Pyridazine Analogs

Next, we tested whether adding a single nitrogen to the benzoisothiazolone ring would have an effect on activity, and found that pyridine analogs, LC-46 and LC-73, had substantially improved biochemical properties over LOC14, with a K_{D} of 10 nM in the ITC assay (Table 10). We found, however, that despite tight binding of LC-46 and LC-73 to PDI, there was a loss of potency in the cell viability assay compared to their benzoisothiazolone counterparts (LOC14 and LC-17) (Table 10). We investigated whether addition of a second nitrogen to the pyridine ring would have an improvement in neuroprotective activity in cells. The pyridazine analogs, LC-57 and LC-59, were five-to-six times less potent in PC12 cells, but also had a decrease in cellular toxicity in the PC12 model (Table 10).

TABLE 10
Pyridine and pyridazine analogs have stronger affinity for PDI.

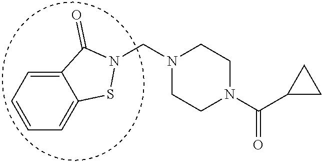 [SMILES: O=C(C1CC1)N1CCN(Cn2sc3ccccc3c2=O)CC1]

| | | Solubility | Kd | EC50 | Max |
|---|---|---|---|---|---|
| Compound | | (mM) | (nM) | (nM) | Viability (%) |
| LC-46 | 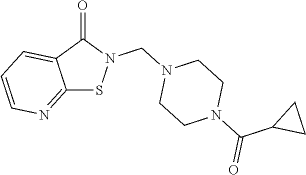 [SMILES: O=C(C1CC1)N1CCN(Cn2sc3ncccc3c2=O)CC1] | 50 | 10 | 2,831 | 39 |
| LC-47 | 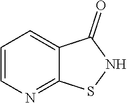 | 50 | NA | >50,000 | 36 |
| LC-73 | 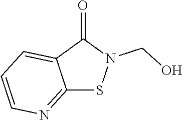 [SMILES: O=c1c2cccnc2sn1CO] | 100 | 10 | 2,475 | 63 |
| LC-17 | 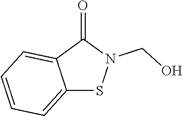 [SMILES: O=c1c2ccccc2sn1CO] | 100 | 160 | 229 | 68 |
| LC-50 | 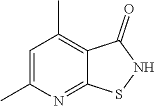 | 100 | 3,831 | 5,180 | 65 |
| LC-53 | 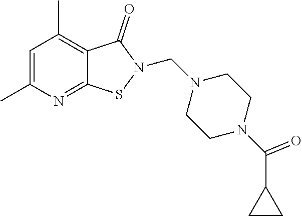 [SMILES: Cc1cc(C)c2c(=O)n(CN3CCN(C(=O)C4CC4)CC3)sc2n1] | 25 | NA** | 4,249 | 67 |
| LC-51 | 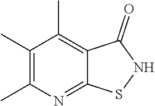 | 10 | NA** | 2,200 | 63 |
| LC-52 | 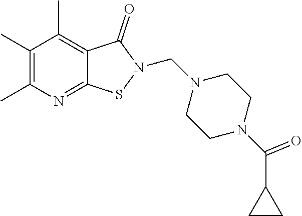 [SMILES: Cc1nc2sn(CN3CCN(C(=O)C4CC4)CC3)c(=O)c2c(C)c1C] | 10 | NA** | 3,570 | 68 |
| LC-57 | 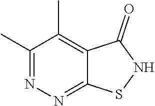 | 100 | 18 | 12,150 | 54 |
| LC-59 | 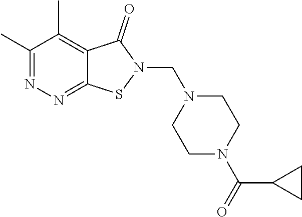 [SMILES: Cc1nnc2sn(CN3CCN(C(=O)C4CC4)CC3)c(=O)c2c1C] | 100 | 8 | 10,573 | 59 |

NA** compounds active in enzymatic assay for inhibition of PDI using insulin as substrate

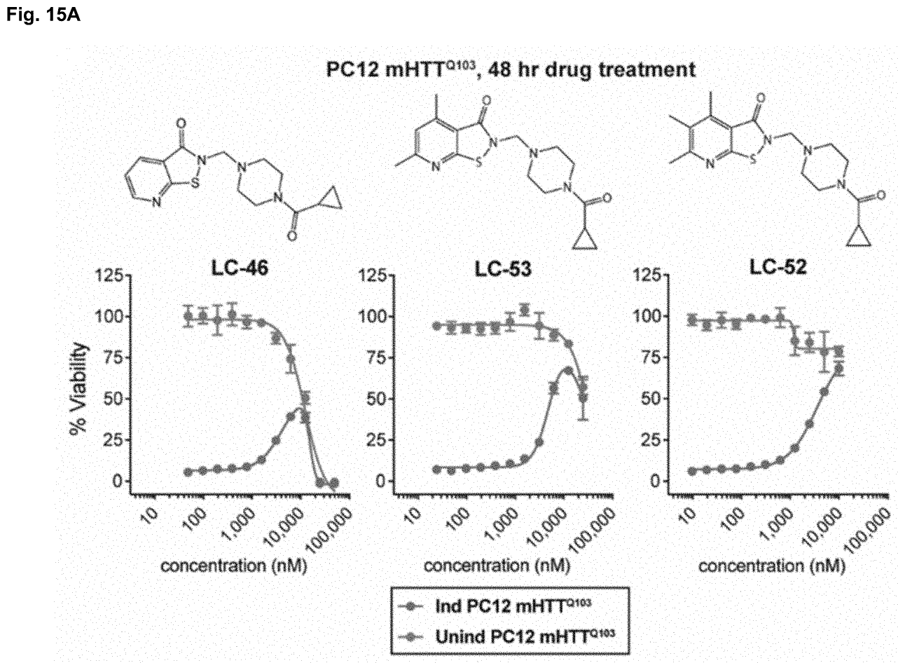

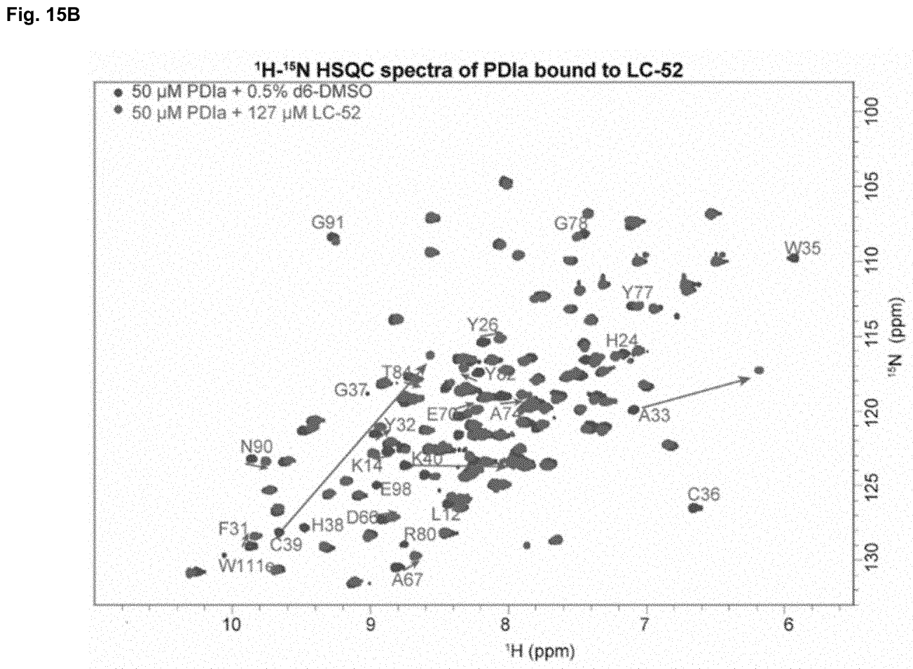

To further analyze this improvement in viability of LC-59 over the LC-46 analog, we synthesized pyridine analogs with two methyl groups (LC-50 and LC-53) and three methyl groups (LC-51 and LC-52) on the ring. Addition of the methyl groups led to complete loss of toxicity in the PC12 cell culture assay (FIG. 15A). Interestingly, binding of LC-51, LC-52 and LC-53, could not be detected by ITC, but did inhibit the enzymatic activity of PDI and induced the same chemical shifts in PDI as LOC14, as observed in the ¹H-¹⁵N HSQC spectra (FIG. 15B). This may be because the binding of these compounds is entropy-driven, with small enthalpic changes, or because the compounds bind with a very slow-on rate. Further detailed biochemical studies will be needed to examine this question.

### Modifications to Methylene Group

Next, we investigated the impact of derivatizing the methylene group. We hypothesized that modification of this moiety would increase the leaving group ability of the amide during ring opening by Cys53 of PDI (for example by converting the methylene group to a carbonyl) leading to a stronger interaction with Cys53 and tighter binding to PDI. However, based on our cell and biochemical data, analogs that contained a carbonyl at this position (analogs: LC-14, LC-11, and LC-6) had complete loss of binding to PDI and were not neuroprotective in cells (Table 11). LC-6, which did show activity in PC12 cells without binding to PDI, was the exception, likely because it was previously reported as a caspase-inhibitor (Liu et al. 2013).

TABLE 11
Analogs with a carbonyl or shorter methylene group lose selectivity for PDI.

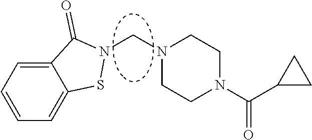 [SMILES: O=C(C1CC1)N1CCN(Cn2sc3ccccc3c2=O)CC1]

| | | Solubility | Kd | EC50 | Max |
|---|---|---|---|---|---|
| Compound | | (mM) | (nM) | (nM) | Viability (%) |
| LC-14 | 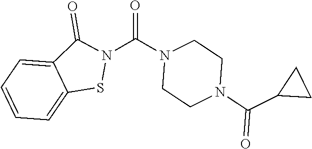 [SMILES: O=C(C1CC1)N1CCN(C(=O)n2sc3ccccc3c2=O)CC1] | 30 | NA | >50,000 | 34 |
| LC-6 | 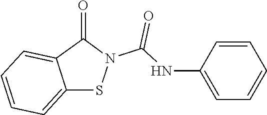 [SMILES: O=C(Nc1ccccc1)n1sc2ccccc2c1=O] | 30 | NA | 476 | 66 |
| LC-11 | 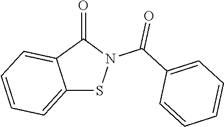 [SMILES: O=C(c1ccccc1)n1sc2ccccc2c1=O] | 100 | NA | >50,000 | 28 |
| LC-24 | 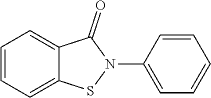 [SMILES: O=c1c2ccccc2sn1-c1ccccc1] | 50 | 8; 1,138** | 1,134 | 31 |
| LC-31 | 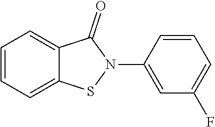 [SMILES: O=c1c2ccccc2sn1-c1cccc(F)c1] | 100 | NA | 1,765 | 36 |
| LC-36 | 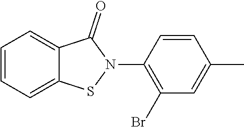 [SMILES: Cc1ccc(-n2sc3ccccc3c2=O)c(Br)c1] | 100 | 25** | 1,594 | 15 |
| LC-25 | 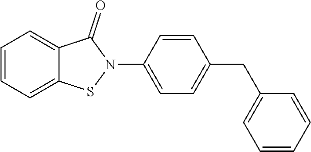 [SMILES: O=c1c2ccccc2sn1-c1ccc(Cc2ccccc2)cc1] | 100 | NA | 1,591 | 37 |
| LC-37 | 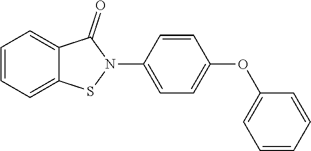 [SMILES: O=c1c2ccccc2sn1-c1ccc(Oc2ccccc2)cc1] | 100 | NA | 1,679 | 22 |
| LC-27 | 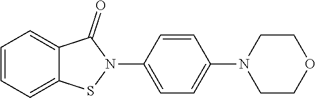 [SMILES: O=c1c2ccccc2sn1-c1ccc(N2CCOCC2)cc1] | 30 | NA | 1,828 | 28 |

**Compounds not active at inhibiting PDI's reductase activity in enzymatic assay

Removing the methylene group altogether (analogs: LC-24, LC-31, LC-36, LC-25, LC-37, and LC-27) and replacing it with a substituted phenyl ring directly on the benzisothiazolinone resulted in a three-to-five-fold loss in potency in the cell viability assay and in most cases loss of binding to PDI (Table 11). The two exceptions were analog LC-24, which bound to PDI at two binging sites with a K_{D1}=8.1 nM and K_{D2}=1137.7 nM and analog LC-36, with K_{D2}=25 nM. Both of these analogs, however, did not inhibit PDI's reductase activity, indicating that they bind to an alternate site of the protein and not to its active site. Substituting the methylene group with a methyl group (analog LC-29 and LC-58) had a small effect on the compounds performance in cell and biochemical assays, but was not an improvement over LOC14 (Table 12).

Next, we evaluated LOC14 pyridine analogs with a longer linker between the isothiazolopyridinone and the substituted piperazine (analogs LC-54, LC-55 and LC-56). These analogs were suspected of being more stable compared to the aminal moiety found in LOC14. Analysis of these analogs in PC12 cells, however, showed that modifying the linker region led to complete loss of cell neuroprotection (Table 12). Non-pyridine analogs without the aminal moiety (analogs LC-32 and LC-17) had similar activity as LOC14 (Table 12). We also looked at an analog alkylated on the oxygen instead of the nitrogen (analog LC-16) and found that this modification resulted in loss of cell viability and loss of binding to PDI (Table 12). Together, these structure activity relationship (SAR) data suggest that shortening the methylene region, or adding a carbonyl group will lead to loss of cell activity and possibly selectivity for PDI, while addition of a methyl group or longer linker can be tolerated, but not with pyridine analogs or benzyl groups.

TABLE 12
Analogs with extended linker region are tolerated, but not on pyridine ring.

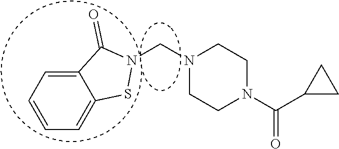

| | | Solubility | Kd | EC50 | Max |
|---|---|---|---|---|---|
| Compound | | (mM) | (nM) | (nM) | Viability (%) |
| LC-5 | 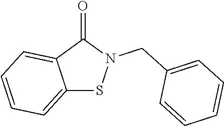 [SMILES: O=c1c2ccccc2sn1Cc1ccccc1] | 100 | 43; 2,268 | 768 | 46 |
| LC-29 | 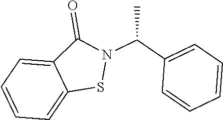 [SMILES: C[C@H](c1ccccc1)n1sc2ccccc2c1=O] | 100 | 658 | 669 | 42 |
| LC-58 | 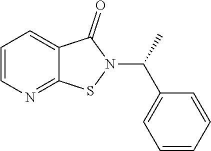 [SMILES: C[C@H](c1ccccc1)n1sc2ncccc2c1=O] | 100 | 0.3 | NA | 14 |
| LC-54 | 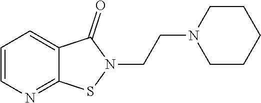 [SMILES: O=c1c2cccnc2sn1CCN1CCCCC1] | 100 | 20 | NA | 11 |
| LC-55 | 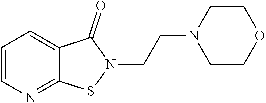 [SMILES: O=c1c2cccnc2sn1CCN1CCOCC1] | 100 | 20 | NA | 13 |
| LC-56 | 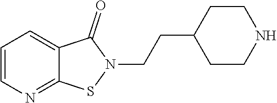 [SMILES: O=c1c2cccnc2sn1CCC1CCNCC1] | 100 | NA | NA | 14 |
| LC-32 | 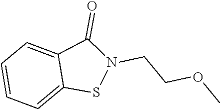 | 100 | 158 | 700 | 42 |
| LC-17 | 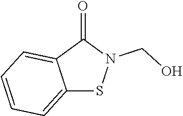 [SMILES: O=c1c2ccccc2sn1CO] | 100 | 160 | 229 | 68 |
| LC-16 | 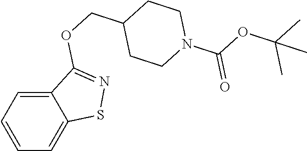 [SMILES: CC(C)(C)OC(=O)N1CCC(COc2nsc3ccccc23)CC1] | 50 | NA | >50,000 | 30 |

Fluorine analogs of LOC14

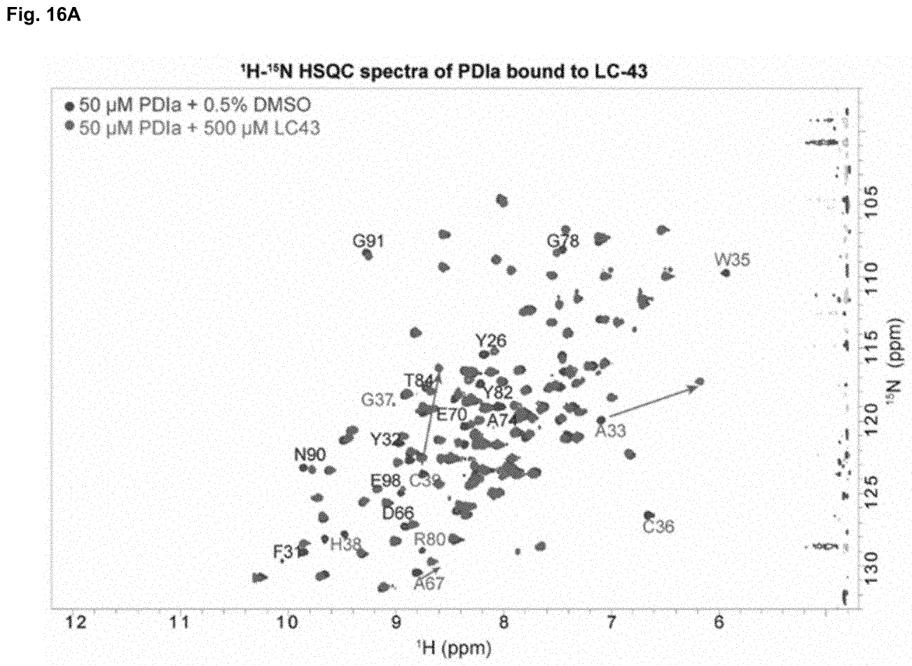

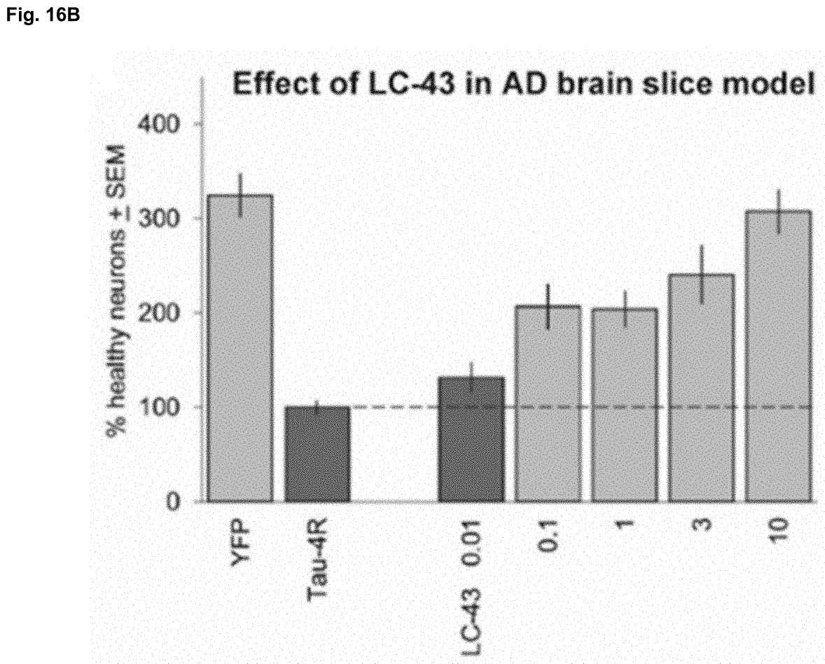

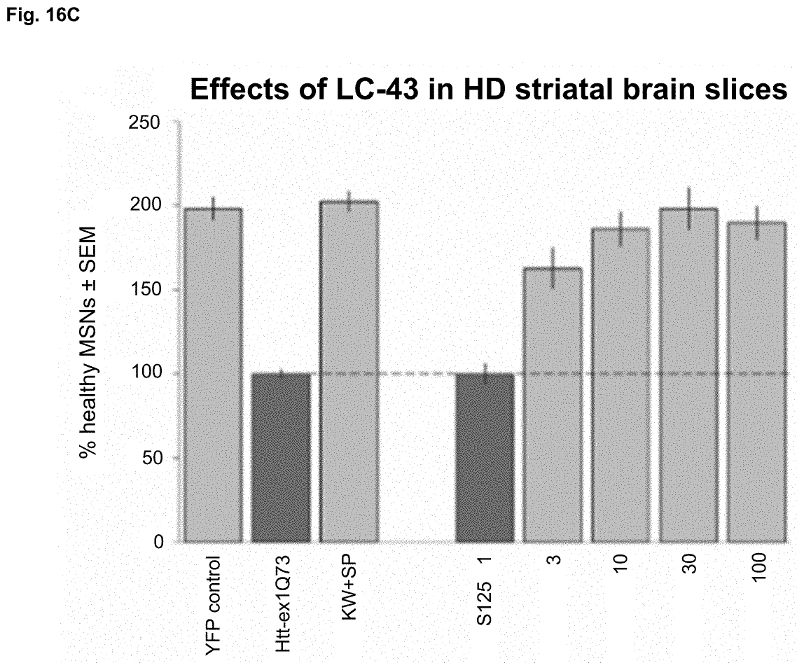

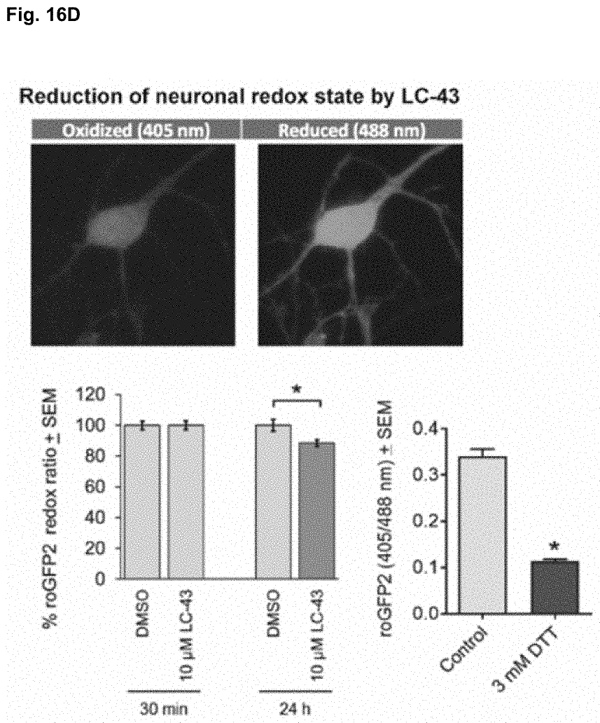

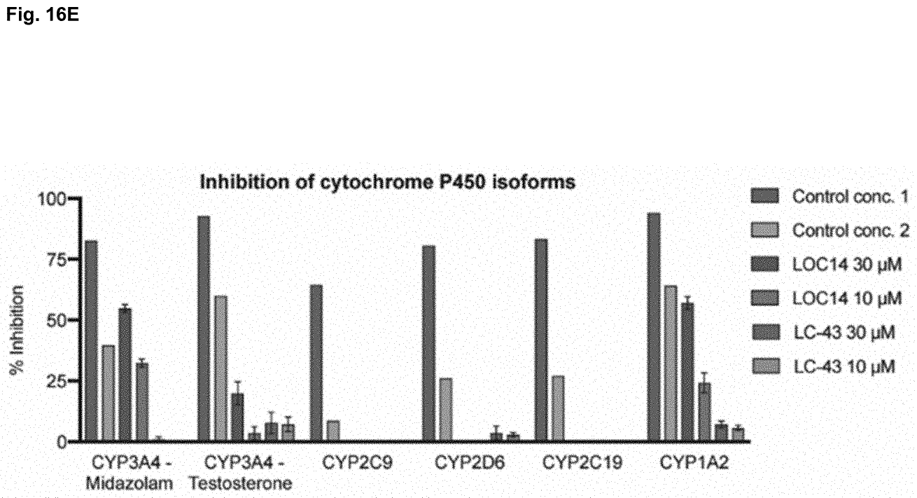

As mentioned, we synthesized several fluorine-containing analogs of LOC14 (analogs LC-43 and LC-40). Addition of fluorine was the only modification that was tolerated at the ortho position without a complete loss of activity in cells and in targeting PDI in vitro (Table 9). We also observed that fluorine-substituted analogs have decreased cellular toxicity, compared to LOC14 and that these compounds were able to inhibit the enzymatic activity of PDI but showed no binding to the protein by ITC. We hypothesized that these compounds might be binding to PDI covalently, and therefore would not show heat evolution by calorimetry. To test this hypothesis, we conducted MALDI-TOF mass-spectrometry experiments with wild-type PDIa and LC-43. However, we observed that there was no change in mass when we compared PDIa alone to PDIa treated with LC-43. We then performed ¹H-¹⁵N-HSQC studies with ¹⁵N-labeled PDIa and LC-43, and saw the same chemical shift changes in the active site as induced by LOC14 (FIG. 16A). This confirmed that LC-43 did indeed bind to PDIa, and that this binding was not covalent. The lack of binding by ITC might be again caused because LC-43 interaction with PDI is entropy-driven with small enthalpic changes. Furthermore, LC-43 showed dose-dependent rescue in AD and HD brain slices, at low micromolar concentrations (FIGS. 16B-16C). In fact, the neuroprotective efficacy appeared to be significantly more potent in the AD brain slice assay for cortical neurons compared to the HD brain slice model, which assays striatal MSNs. Additionally, we evaluated LC-43 using a genetically encoded redox reporter transfected into neurons. FIG. 16D shows fluorescent images of the reporter used for these studies, roGFP2, which is a ratiometric indicator of redox state based on the GFP core structure. As can be seen in FIG. 16D, 24 h treatment with 10 μM LC-43 (a concentration that provided peak rescue in both HD and AD brain slice assays) produced a measurable and significant reduction in neuronal redox state. There was no effect of 30 min treatment with LC-43, suggesting that the reduction in neuronal redox potential was not due to a direct compound effect on the reporter itself. In contrast, treatment with the non-selective reducing agent dithiothreitol (DTT) produces an immediate and dramatic reduction in the roGFP2 fluorescence ratio that leads rapidly to cell degeneration and death (FIG. 16D). Furthermore, we evaluated the effects of LOC14 and LC-43 in their ability to inhibit CYP450 enzymes. At high concentration of 30 μM, above the concentration that is neuroprotective, LOC14 inhibits CYP3A4 and CYP1A2 enzymes (FIG. 16E). LC-43 does not show any inhibition against any of the five isoforms of CYP450 (FIG. 16E). This single fluorine atom substitution in LOC14 structure seems to be an improvement in the design of compounds with more favorable drug properties.

### Dimers of Active Analogs

Seeing as not many modifications improved activity of analogs over LOC14, we hypothesized that if we synthesized compounds containing dimers of the active moieties, we could design a compound with tighter binding and more potent cellular response than LOC14. Along these lines, we synthesized analogs LC-9, LC-68, LC-69, LC-70, LC-71, and LC-72. Unfortunately, the solubility of dimer compounds dropped significantly in DMSO (Table 13). Also, with the exception of LC-9, no improvement of binding to PDI or cell potency was observed. Compound LC-9 had an eight-fold tighter K_{D} than LOC14 (K_{D} of 13 nM) and a two-fold improvement in cell viability potency (EC₅₀ of 173 nM) (Table 13).

TABLE 13
Activity of analogs containing piperazine linked dimers.

| | | Solubility | Kd | EC50 | Max |
|---|---|---|---|---|---|
| Compound | | (mM) | (nM) | (nM) | Viability (%) |
| LC-9 | 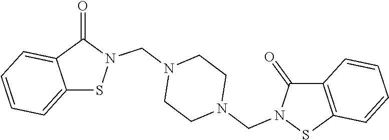 [SMILES: O=c1c2ccccc2sn1CN1CCN(Cn2sc3ccccc3c2=O)CC1] | 5 | 13 | 173 | 66 |
| LC-68 | 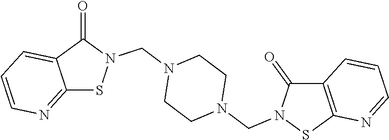 [SMILES: O=c1c2cccnc2sn1CN1CCN(Cn2sc3ncccc3c2=O)CC1] | 5 (NMP)¹ | NA | 2061 | 36 |
| LC-69 | 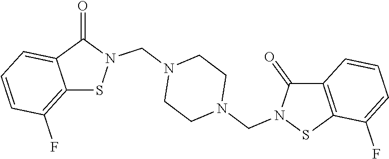 [SMILES: O=c1c2cccc(F)c2sn1CN1CCN(Cn2sc3c(F)cccc3c2=O)CC1] | 5 (NMP)¹ | 700 | 1974 | 46 |
| LC-70 | 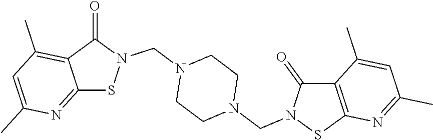 | 5 (NMP)¹ | 3800 | 3088 | 59 |
| LC-71 | 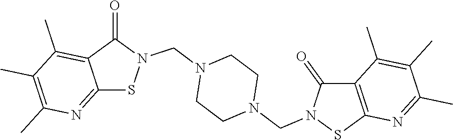 | 2.5 (NMP)¹ | NA² | 1019 | 41 |
| LC-72 | 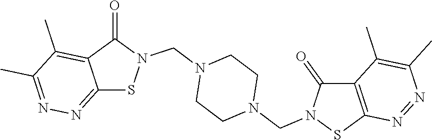 | 1 (NMP)¹ | NA² | NA | 10 |

¹Analogs were not soluble in DMSO, only in N-Methyl-2-Pyrrolidine (NMP)
²Analogs could not be tested in ITC binding assay due to poor solubility

### CONCLUSIONS

In this disclosure, we elucidated the mechanism of neuroprotection underlying the small molecule LOC14 that modulates the activity of PDI. We found that LOC14 is a selective agent and does not bind to other promiscuous protein targets or to cellular glutathione. We provide evidence that the mechanism of interaction of LOC14 is by binding to the active site cysteine 53 of PDI in a reversible manner. Furthermore, we found that LOC14 binding to PDI reduces the production of ROS, most likely by oxidizing PDI and eliminating the need for activation of ERO1 protein in the ER. Unlike other antioxidant agents, LOC14 reduces the formation of ROS in the ER, since PDI and ERO1 are predominantly localized in the ER. Reduction of ER ROS leads to neuroprotection in cell culture models of HD, with more potent rescue effects seen in striatal cell culture model. In addition, testing LOC14 in the brain slice model of AD with expanded Tau-4R, showed nice dose-dependent protection of pyramidal neurons. These results together support the notion that protein misfolding neuronal diseases, such as HD and AD, share common pathways and that PDI modulation with LOC14 is a promising strategy to combat these diseases.

Building upon the protective properties of LOC14, we explored several synthetic avenues to optimize LOC14. Thorough SAR analysis provided further insight into the mechanism of LOC14. We found that the cyclopropylcarbonylpiperazine moiety can tolerate many modifications without a significant effect on activity, and is necessary for tissue penetration and protection from expanded Tau4R-toxicity in a brain slice model of AD. Replacing the benzoisothiazolone ring with a pyridine or a pyridazine ring led to tighter binding with PDI, but loss of potency in cell culture assays. Shortening the methylene region, or adding a carbonyl group led to loss of cell activity and possibly selectivity for PDI, while addition of the methyl group or longer linker was tolerated, but not with pyridine analogs or benzyl groups. Generating a dimer of the active BIT moiety connected by a piperazine linker (analog LC-9) was the most successful attempt at improving the compound's engagement with PDI and cellular potency at rescuing from mHTT-induced toxicity.

Together with its analogs, LOC14 represents a useful tool for studying ER ROS in the context of neurodegenerative diseases and opens up a new therapeutic avenue for treatment of these diseases.

### DOCUMENTS CITED

Aiken C T, Tobin A J, & Schweitzer E S (2004) A cell-based screen for drugs to treat Huntington's disease. Neurobiol Dis 16(3):546-555.
Atkin J D, et al. (2008) Endoplasmic reticulum stress and induction of the unfolded protein response in human sporadic amyotrophic lateral sclerosis. Neurobiol Dis 30(3):400-407.
Barbouche R, Miquelis R, Jones I M, & Fenouillet E (2003) Protein-disulfide isomerase-mediated reduction of two disulfide bonds of HIV envelope glycoprotein 120 occurs post-CXCR4 binding and is required for fusion. J Biol Chem 278(5):3131-3136.
Behl, C., Alzheimer's disease and oxidative stress: implications for novel therapeutic approaches. Prog Neurobiol 1999, 57 (3), 301-23.
Bowes, J.; Brown, A. J.; Hamon, J.; Jarolimek, W.; Sridhar, A.; Waldron, G.; Whitebread, S., Reducing safety-related drug attrition: the use of in vitro pharmacological profiling. Nat Rev Drug Discov 2012, 11 (12), 909-22.
Branca C, Shaw D M, Belfiore R, Gokhale V, Shaw A Y, Foley C, Smith B, Hulme C, Dunckley T, Meechoovet B, Caccamo A, Oddo S. Dyrk1 inhibition improves Alzheimer's disease-like pathology. Aging Cell. 2017 Oct.; 16(5):1146-1154. doi: 10.1111/acel.12648. Epub 2017 Aug. 4
Calsolaro V. and Edison P. Novel GLP-1 (Glucagon-Like Peptide-1) Analogues and Insulin in the Treatment for Alzheimer's Disease and Other Neurodegenerative Diseases. CNS Drugs. 2015 Dec.; 29(12):1023-39. doi: 10.1007/s40263-015-0301-8
Cantley A M, et al. (2014) Small Molecule that Reverses Dexamethasone Resistance in T-cell Acute Lymphoblastic Leukemia (T-ALL). ACS Med Chem Lett 5(7):754-759.
Cardinale C J, et al. (2008) Termination factor Rho and its cofactors NusA and NusG silence foreign DNA in E. coli. Science 320(5878):935-938.
Cattaneo, E.; Conti, L., Generation and characterization of embryonic striatal conditionally immortalized ST14A cells. J Neurosci Res 1998, 53 (2), 223-34.
Cho J, Furie B C, Coughlin S R, & Furie B (2008) A critical role for extracellular protein disulfide isomerase during thrombus formation in mice. J Clin Invest 118(3):1123-1131.
Colla E, et al. (2012) Endoplasmic reticulum stress is important for the manifestations of alpha-synucleinopathy in vivo. J Neurosci 32(10):3306-3320.
Cui G, Shan L, Guo L, Chu I K, Li G, Quan Q, Zhao Y, Chong C M, Zhang Z, Yu P, Hoi M P, Sun Y, Wang Y, Lee S M. Novel anti-thrombotic agent for modulation of protein disulfide isomerase family member ERp57 for prophylactic therapy. Sci Rep. 2015 Jun. 3; 5:10353. doi: 10.1038/srep10353.
Darby N J & Creighton T E (1995) Functional properties of the individual thioredoxin-like domains of protein disulfide isomerase. Biochemistry 34(37):11725-11735.
Dixon S J, et al. (2012) Ferroptosis: an iron-dependent form of nonapoptotic cell death. Cell 149(5):1060-1072.
Ehrlich, M. E.; Conti, L.; Toselli, M.; Taglietti, L.; Fiorillo, E.; Taglietti, V.; Ivkovic, S.; Guinea, B.; Tranberg, A.; Sipione, S.; Rigamonti, D.; Cattaneo, E., ST14A cells have properties of a medium-size spiny neuron. Exp Neurol 2001, 167 (2), 215-26.
Evaluating Liraglutide in Alzheimer's Disease (ELAD). ClinicalTrials.gov Apr. 30, 2013
Forman, H. J.; Zhang, H.; Rinna, A., Glutathione: overview of its protective roles, measurement, and biosynthesis. Mol Aspects Med 2009, 30 (1-2), 1-12.
Fuchslueger U, Socher G, Grether H J, & Grasserbauer M (1999) Capillary supercritical fluid chromatography/mass spectrometry of phenolic mannich bases with dimethyl ether modified ethane as the mobile phase. Anal Chem 71(13):2324-2333.
Garbi N, Tanaka S, Momburg F, & Hammerling G J (2006) Impaired assembly of the major histocompatibility complex class I peptide-loading complex in mice deficient in the oxidoreductase ERp57. Nat Immunol 7(1):93-102.
Ge J, et al. (2013) Small molecule probe suitable for in situ profiling and inhibition of protein disulfide isomerase. ACS Chem Biol 8(11):2577-2585.
Geoghegan, K. F.; Dixon, H. B.; Rosner, P. J.; Hoth, L. R.; Lanzetti, A. J.; Borzilleri, K. A.;
Marr, E. S.; Pezzullo, L. H.; Martin, L. B.; LeMotte, P. K.; McColl, A. S.; Kamath, A. V.; Stroh, J. G., Spontaneous alpha-N-6-phosphogluconoylation of a “His tag” in Escherichia coli: the cause of extra mass of 258 or 178 Da in fusion proteins. Anal Biochem 1999, 267 (1), 169-84.
Glimcher L H and Hetz C. Modulation of neurogenerative disease by modulating xbp-1 activity. U.S. Published Patent Application No. 20110142799, filed Mar. 2, 2011
Hashida T, Kotake Y, & Ohta S (2011) Protein disulfide isomerase knockdown-induced cell death is cell-line-dependent and involves apoptosis in MCF-7 cells. J Toxicol Sci 36(1):1-7.
Hoffstrom B G, Kaplan A, Letso R, Schmid R S, Turmel G J, Lo D C, Stockwell B R. Inhibitors of protein disulfide isomerase suppress apoptosis induced by misfolded proteins. Nat Chem Biol. 2010 Dec.; 6(12): pp. 900-906.
Holla B S, Shivananda M K, Shenoy M S, & Antony G (1998) Studies on arylfuran derivatives. Part VII. Synthesis and characterization of some Mannich bases carrying halophenylfuryl moieties as promising antibacterial agents. Farmaco 53(8-9):531-535.
Huang S G, Oksenberg D and Urfer R. High-throughput turbidometric assay for screening inhibitors of protein disulfide isomerase activity. U.S. Pat. No. 6,977,142, filed Sep. 23, 2003.
Idhayadhulla A, Surendra Kumar R, Abdul Nasser A J, & Manilal A (2011) Synthesis and antimicrobial activity of some new Mannich base derivatives. J. Chem. Pharm. Res. 3(4):904-911.
Jasuja R, Passam F H, Kennedy D R, Kim S H, van Hessem L, Lin L, Bowley S R, Joshi S S, Dilks J R, Furie B, Furie B C, Flaumenhaft R. Protein disulfide isomerase inhibitors constitute a new class of antithrombotic agents. J Clin Invest. 2012 June; 122(6):2104-13. doi: 10.1172/JC161228. Epub 2012 May 8
Kaplan A. and Stockwell B R. Structural elucidation of a small molecule inhibitor of protein disulfide isomerase. ACS Med Chem Lett. 2015 Jul. 29; 6(9):966-971. eCollection 2015 Sep. 10.
Kaplan A., Gaschler M M., Dunn D E., Colligan R., Brown L M., Palmer A G III, Lo D C., Stockwell B R. Small molecule-induced oxidation of protein disulfide isomerase is neuroprotective. Proc Natl Acad Sci USA. 2015 Apr. 28; 112(17):E2245-52. doi: 10.1073/pnas.1500439112. Epub 2015 Apr. 6.
Karala A R & Ruddock L W (2010) Bacitracin is not a specific inhibitor of protein disulfide isomerase. FEBS J 277(11):2454-2462.
Kemmink J, Darby N J, Dijkstra K, Scheek R M, & Creighton T E (1995) Nuclear magnetic resonance characterization of the N-terminal thioredoxin-like domain of protein disulfide isomerase. Protein Science 4(12):2587-2593.
Khan M M, Simizu S, Lai N S, Kawatani M, Shimizu T, Osada H. Discovery of a small molecule PDI inhibitor that inhibits reduction of HIV-1 envelope glycoprotein gp120. ACS Chem Biol. 2011 Mar. 18; 6(3):245-51. doi: 10.1021/cb100387r. Epub 2011 Jan. 7
Koivu J, et al. (1987) A single polypeptide acts both as the beta subunit of prolyl 4-hydroxylase and as a protein disulfide-isomerase. J Biol Chem 262(14):6447-6449.
Kortemme, T.; Darby, N. J.; Creighton, T. E., Electrostatic interactions in the active site of the N-terminal thioredoxin-like domain of protein disulfide isomerase. Biochemistry 1996, 35 (46), 14503-11.
Kosuri P, Alegre-Cebollada J, Feng J, aplan A, Ingles-Prieto A, Badilla C L, Stockwell B R, Sanchez-Ruiz J M, Holmgren A, Fernandez J M. Protein folding drives disulfide formation. Cell. 2012 Nov. 9; 151(4): 794-806. doi: 10.1016/j.cell.2012.09.036.
Kozlov G, Määttänen P, Thomas D Y, Gehring K. A structural overview of the PDI family of proteins. FEBS J. 2010 October; 277(19):3924-36. doi: 10.1111/j.1742-4658.2010.07793.x. Epub 2010 Aug. 26.
Lipinski C A, Lombardo F, Dominy B W, & Feeney P J (2001) Experimental and computational approaches to estimate solubility and permeability in drug discovery and development settings. Adv Drug Deliv Rev 46(1-3):3-26.
Liu, D.; Tian, Z.; Yan, Z.; Wu, L.; Ma, Y.; Wang, Q.; Liu, W.; Zhou, H.; Yang, C., Design, synthesis and evaluation of 1,2-benzisothiazol-3-one derivatives as potent caspase-3 inhibitors. Bioorg Med Chem 2013, 21 (11), 2960-7.
Lovat P E, et al. (2008) Increasing melanoma cell death using inhibitors of protein disulfide isomerases to abrogate survival responses to endoplasmic reticulum stress. Cancer Res 68(13):5363-5369.
Lu J., Holmgren A. The thioredoxin superfamily in oxidative protein folding. Antioxid Redox Signal. 2014 Jul. 20; 21(3):457-70. doi: 10.1089/ars.2014.5849. Epub 2014 Mar. 6.
Reinhart P H, et al. (2011) Identification of anti-inflammatory targets for Huntington's disease using a brain slice-based screening assay. Neurobiol Dis 43(1):248-256.
Rigamonti, D.; Bauer, J. H.; De-Fraja, C.; Conti, L.; Sipione, S.; Sciorati, C.; Clementi, E.; Hackam, A.; Hayden, M. R.; Li, Y.; Cooper, J. K.; Ross, C. A.; Govoni, S.; Vincenz, C.; Cattaneo, E., Wild-type huntingtin protects from apoptosis upstream of caspase-3. J Neurosci 2000, 20 (10), 3705-13.
Rogelj S and Sklar L A. Inhibition of cell surface protein disulfide isomerase. International Patent Application No. W O/1998/051297, filed May 14, 1998
Ruddock, L. W.; Hirst, T. R.; Freedman, R. B., pH-dependence of the dithiol-oxidizing activity of DsbA (a periplasmic protein thiol:disulphide oxidoreductase) and protein disulphide-isomerase: studies with a novel simple peptide substrate. Biochem J 1996, 315 (Pt 3), 1001-5.
Smith A M, et al. (2004) A high-throughput turbidometric assay for screening inhibitors of protein disulfide isomerase activity. J Biomol Screen 9(7):614-620.
Sonam Parakh and Atkin J D. Novel roles for protein disulphide isomerase in disease states: a doubled edged sword? Front Cell Dev Biol. 2015; 3: p. 30.
Stockwell B R and Hooffstrom B G. Agents and assays for modulating neurodegeneration. International Patent Application No. W O/2008/143876, filed May 14, 2008
Stockwell B R and Kaplan A. Small molecule oxidizers of PDI and their use. U.S. Published Patent Application No. 20180092908, filed Jul. 20, 2017
Suhr S T, Gil E B, Senut M C, & Gage F H (1998) High level transactivation by a modified Bombyx ecdysone receptor in mammalian cells without exogenous retinoid X receptor. Proc Natl Acad Sci USA 95(14):7999-8004.
Vilaboa N, Boellmann F, & Voellmy R (2011) Gene Switches for Deliberate Regulation of Transgene Expression: Recent Advances in System Development and Uses. Journal of genetic syndrome & gene therapy 2(3):107.
Wetterau J R, Combs K A, Spinner S N, & Joiner B J (1990) Protein disulfide isomerase is a component of the microsomal triglyceride transfer protein complex. J Biol Chem 265(17):9800-9807.
Williamson M P (2013) Using chemical shift perturbation to characterise ligand binding. Prog Nucl Magn Reson Spectrosc 73:1-16.
Xu S, et al. (2012) Discovery of an orally active small-molecule irreversible inhibitor of protein disulfide isomerase for ovarian cancer treatment. Proc Natl Acad Sci USA 109(40):16348-16353.
Yang W S, et al. (2014) Regulation of ferroptotic cancer cell death by GPX4. Cell 156(1-2):317-331.
Yoo B C, et al. (2002) Overexpressed protein disulfide isomerase in brains of patients with sporadic Creutzfeldt-Jakob disease. Neurosci Lett 334(3):196-200. Zhang J H, Chung T D, & Oldenburg K R (1999) A Simple Statistical Parameter for Use in Evaluation and Validation of High Throughput Screening Assays. J Biomol Screen 4(2):67-73.
Zhou X, Li G, Kaplan A, Gaschler M M, Zhang X, Hou Z, Jiang M, Zott R, Crmers S, Stockwell B R, Duan W. Small molecule modulator of protein disulfide isomerase attenuates mutant huntingtin toxicity and inhibits endoplasmic reticulum stress in a mouse model of Huntington's disease. Hum Mol Genet. 2018 May 1; 27(9):1545-1555. doi: 10.1093/hmg/ddy061

All patents, patent applications, and publications cited above are incorporated herein by reference in their entirety as if recited in full herein.

The disclosure being thus described, it will be obvious that the same may be varied in many ways. Such variations are not to be regarded as a departure from the spirit and scope of the disclosure and all such modifications are intended to be included within the scope of the following claims.

What is claimed is:

1. A compound having the formula (I): 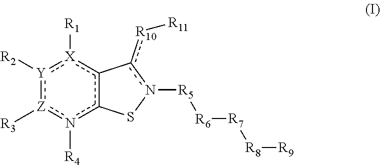
wherein a dashed line indicates the presence of an optional double bond,
wherein X, Y and Z are independently selected from the group consisting of C, N, S and O,
wherein R₁, R₂, R₃, and R₄ are independently selected from the group consisting of no atom, H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R₅, R₆, R₇, R₈ and R₁₀ are independently selected from the group consisting of no atom, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R₉ and R₁₁ are independently selected from the group consisting of H, NR, N(R)C(O), C(O)NR, O, C(O), C(O)O, OC(O); N(R)SO2, SO2N(R), S, SO, SO2, -(optionally substituted C₁₋₆ alkyl), -(optionally substituted mono- or polycyclic group containing 3 to 20 carbon atoms and optionally 1 to 4 heteroatoms selected from O, N and S), —C₁₋₄ alkyl-(optionally substituted mono- or polycyclic group containing 3 to 20 carbon atoms and optionally 1 to 4 heteroatoms selected from O, N and S),
wherein R is selected from the group consisting of H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
with the proviso that the compound is not 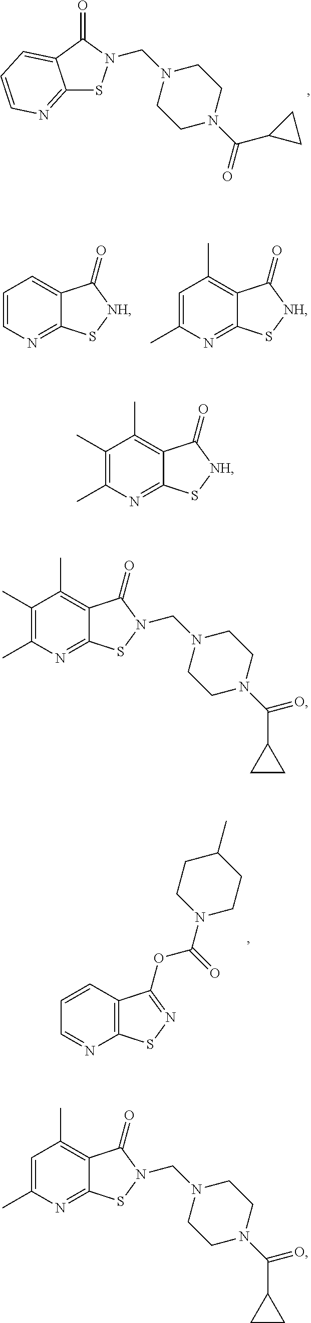 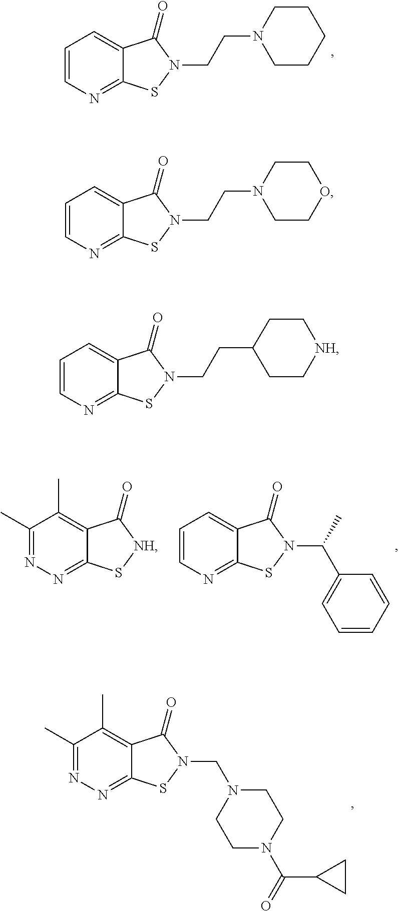
or an N-oxide, crystalline form, hydrate thereof, or a pharmaceutically acceptable salt thereof.

2. The compound of claim 1, which is selected from the group consisting of: 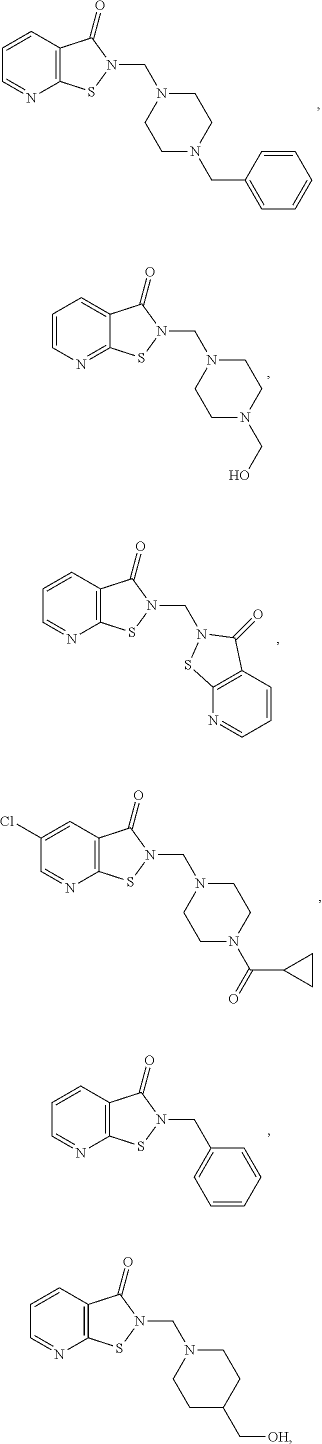 [SMILES: O=c1c2cccnc2sn1CN1CCN(Cc2ccccc2)CC1 ; O=c1c2cccnc2sn1CN1CCN(CO)CC1 ; O=c1c2cccnc2sn1Cn1sc2ncccc2c1=O ; O=C(C1CC1)N1CCN(Cn2sc3ncc(Cl)cc3c2=O)CC1 ; O=c1c2cccnc2sn1Cc1ccccc1 ; O=c1c2cccnc2sn1CN1CCC(CO)CC1] 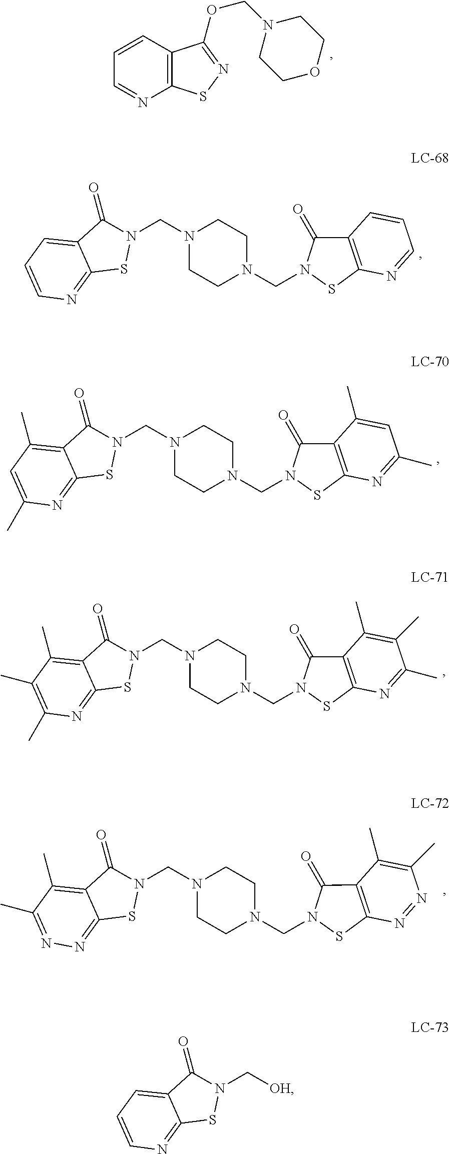
and combinations thereof,
or an N-oxide, crystalline form, hydrate thereof, or a pharmaceutically acceptable salt thereof.

3. A composition comprising a compound according to claim 1 and a pharmaceutically acceptable carrier, adjuvant or vehicle.

4. A pharmaceutically acceptable salt of a compound according to claim 1.

5. A composition comprising a pharmaceutically acceptable salt of a compound according to claim 1 and a pharmaceutically acceptable carrier, adjuvant or vehicle.

6. A method for treating or ameliorating the effects of a neurodegenerative disease in a subject in need thereof comprising administering to the subject an effective amount of a compound according to claim 1.

7. The method of claim 6, wherein the compound is selected from the group consisting of: 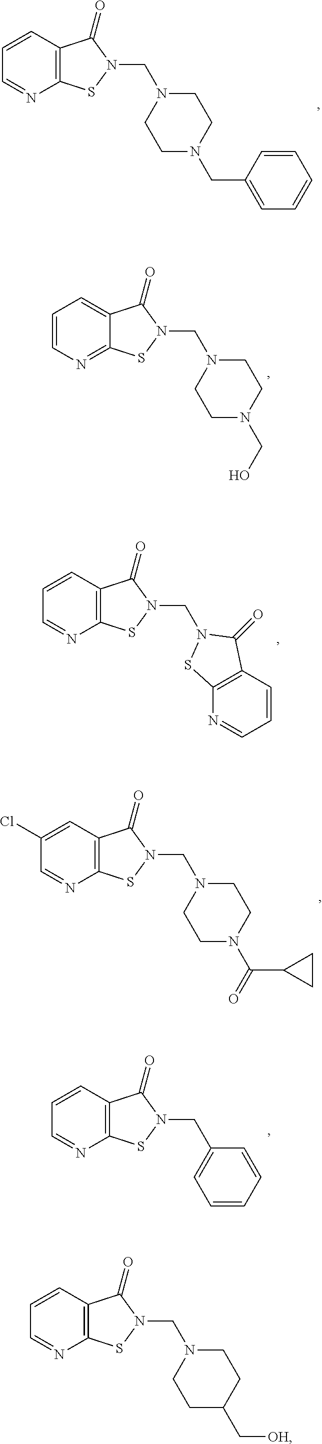 [SMILES: O=c1c2cccnc2sn1CN1CCN(Cc2ccccc2)CC1 ; O=c1c2cccnc2sn1CN1CCN(CO)CC1 ; O=c1c2cccnc2sn1Cn1sc2ncccc2c1=O ; O=C(C1CC1)N1CCN(Cn2sc3ncc(Cl)cc3c2=O)CC1 ; O=c1c2cccnc2sn1Cc1ccccc1 ; O=c1c2cccnc2sn1CN1CCC(CO)CC1] 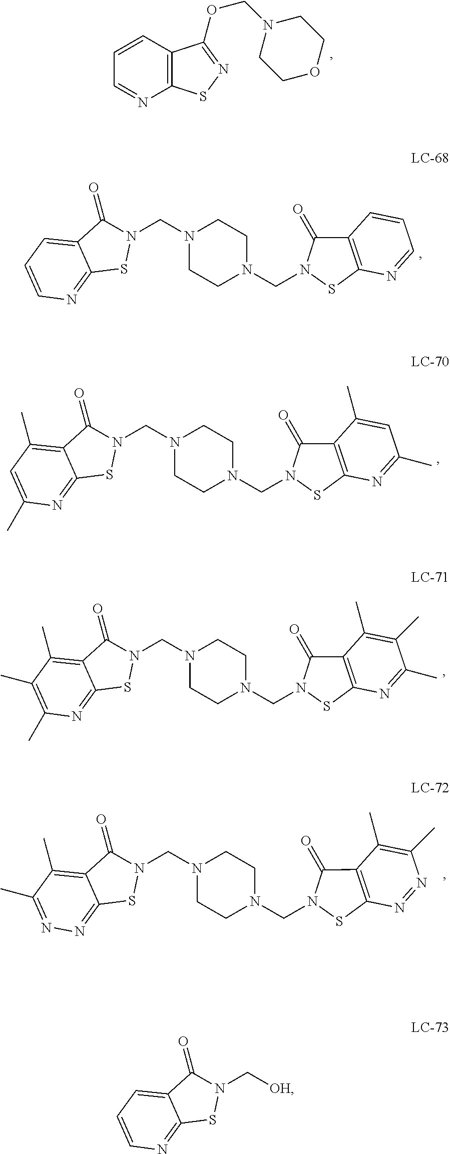
and combinations thereof,
or an N-oxide, crystalline form, hydrate thereof, or a pharmaceutically acceptable salt thereof.

8. The method of claim 6, wherein the neurodegenerative disease is selected from the group consisting of Alzheimer's disease, Parkinson's disease, amyotrophic lateral sclerosis (ALS), Friedreich's ataxia, multiple sclerosis, Huntington's disease, transmissible spongiform encephalopathy, Charcot-Marie-Tooth disease, dementia with Lewy bodies, corticobasal degeneration, progressive supranuclear palsy, and hereditary spastic paraparesis.

9. The method of claim 6, further comprising co-administering to the subject an effective amount of one or more additional therapeutic agents.

10. The method of claim 9, wherein the one or more additional therapeutic agents are selected from the group consisting of 5-hydroxytryptophan, Activase, AFQ056 (Novartis), Aggrastat, Albendazole, alpha-lipoic acid/L-acetyl carnitine, Alteplase, Amantadine (Symmetrel), amlodipine, Ancrod, Apomorphine (Apokyn), Arimoclomol, Arixtra, Armodafinil, Ascorbic acid, Ascriptin, Aspirin, atenolol, Avonex, baclofen (Lioresal), Banzel, Benztropine (Cogentin), Betaseron, BGG492 (Novartis Corp.), Botulinum toxin, Bufferin, Carbatrol®, Carbidopa/levodopa immediate-release (Sinemet), Carbidopa/levodopa oral disintegrating (Parcopa), Carbidopa/levodopa/Entacapone (Stalevo), CERE-110: Adeno-Associated Virus Delivery of NGF (Ceregene), cerebrolysin, CinnoVex, citalopram, citicoline, Clobazam, Clonazepam, Clopidogrel, clozapine (Clozaril), Coenzyme Q, Creatine, dabigatran, dalteparin, Dapsone, Davunetide, Deferiprone, Depakene®, Depakote ER®, Depakote®, Desmoteplase, Diastat, Diazepam, Digoxin, Dilantin®, Dimebon, dipyridamole, divalproex (Depakote), Donepezil (Aricept), EGb 761, Eldepryl, ELND002 (Elan Pharmaceuticals), Enalapril, enoxaparin, Entacapone (Comtan), epoetin alfa, Eptifibatide, Erythropoietin, Escitalopram, Eslicarbazepine acetate, Esmolol, Ethosuximide, Ethyl-EPA (Miraxion™), Exenatide, Extavia, Ezogabine, Felbamate, Felbatol®, Fingolimod (Gilenya), fluoxetine (Prozac), fondaparinux, Fragmin, Frisium, Gabapentin, Gabitril®, Galantamine, Glatiramer (Copaxone), haloperidol (Haldol), Heparin, human chorionic gonadotropin (hCG), Idebenone, Inovelon®, insulin, Interferon beta 1a, Interferon beta 1b, ioflupane 123I (DATSCAN®), IPX066 (Impax Laboratories Inc.), JNJ-26489112 (Johnson and Johnson), Keppra®, Klonopin, Lacosamide, L-Alpha glycerylphosphorylcholine, Lamictal®, Lamotrigine, Levetiracetam, liraglutide, Lisinopril, Lithium carbonate, Lopressor, Lorazepam, losartan, Lovenox, Lu AA24493, Luminal, LY450139 (Eli Lilly), Lyrica, Masitinib, Mecobalamin, Memantine, methylprednisolone, metoprolol tartrate, Minitran, Minocycline, mirtazapine, Mitoxantrone (Novantrone), Mysoline®, Natalizumab (Tysabri), Neurontin®, Niacinamide, Nitro-Bid, Nitro-Dur, nitroglycerin, Nitrolingual, Nitromist, Nitrostat, Nitro-Time, Norepinephrine (NOR), Carbamazepine, octreotide, Onfi®, Oxcarbazepine, Oxybutinin chloride, PF-04360365 (Pfizer), Phenobarbital, Phenytek®, Phenytoin, piclozotan, Pioglitazone, Plavix, Potiga, Pramipexole (Mirapex), pramlintide, Prednisone, Primidone, Prinivil, probenecid, Propranolol, PRX-00023 (EPIX Pharmaceuticals Inc.), PXT3003, Quinacrine, Ramelteon, Rasagiline (Azilect), Rebif, ReciGen, remacemide, Resveratrol, Retavase, reteplase, riluzole (Rilutek), Rivastigmine (Exelon), Ropinirole (Requip), Rotigotine (Neupro), Rufinamide, Sabril, safinamide (EMD Serono), Salagen, Sarafem, Selegiline (I-deprenyl, Eldepryl), SEN0014196 (Siena Biotech), sertraline (Zoloft), Simvastatin, Sodium Nitroprussiate (NPS), sodium phenylbutyrate, Stanback Headache Powder, Tacrine (Cognex), Tamoxifen, tauroursodeoxycholic acid (TUDCA), Tegretol®, Tenecteplase, Tenormin, Tetrabenazine (Xenazine), THR-18 (Thrombotech Ltd.), Tiagabine, Tideglusib, tirofiban, tissue plasminogen activator (tPA), tizanidine (Zanaflex), TNKase, Tolcapone (Tasmar), Tolterodine, Topamax®, Topiramate, Trihexyphenidyl (formerly Artane), Trileptal®, ursodiol, Valproic Acid, valsartan, Varenicline (Pfizer), Vimpat, Vitamin E, Warfarin, Zarontin®, Zestril, Zonegran®, Zonisamide, Zydis selegiline HCL Oral disintegrating (Zelapar), and combinations thereof.

11. A method for treating or ameliorating the effects of a condition associated with increased protein disulfide isomerase (PDI) activity in a subject in need thereof comprising administering to the subject an effective amount of a compound according to claim 1.

12. The method of claim 11, wherein the compound is selected from the group consisting of: 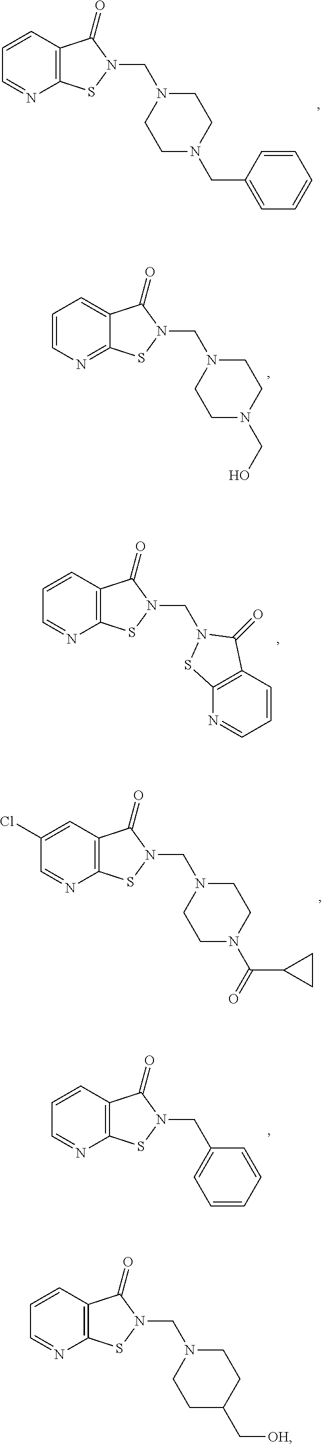 [SMILES: O=c1c2cccnc2sn1CN1CCN(Cc2ccccc2)CC1 ; O=c1c2cccnc2sn1CN1CCN(CO)CC1 ; O=c1c2cccnc2sn1Cn1sc2ncccc2c1=O ; O=C(C1CC1)N1CCN(Cn2sc3ncc(Cl)cc3c2=O)CC1 ; O=c1c2cccnc2sn1Cc1ccccc1 ; O=c1c2cccnc2sn1CN1CCC(CO)CC1] 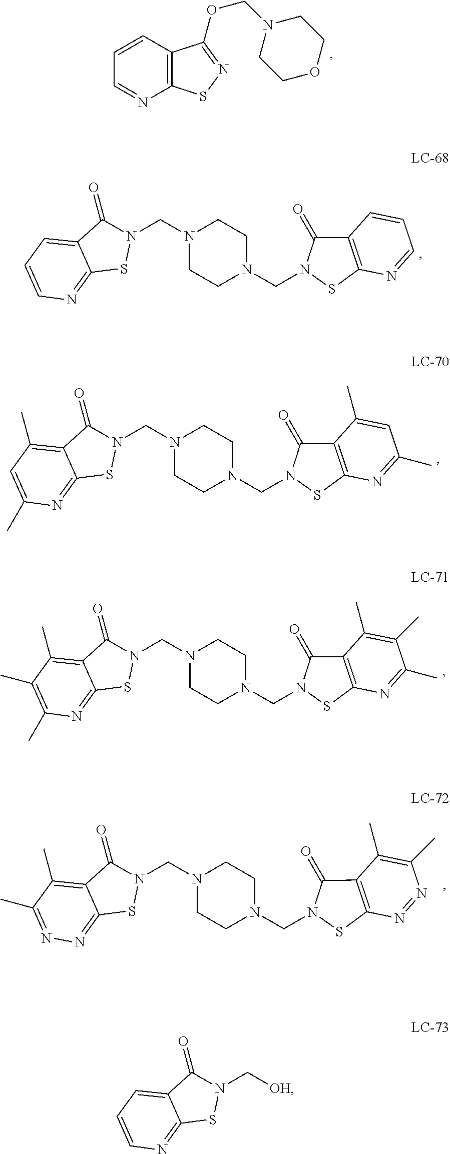
and combinations thereof, or an N-oxide, crystalline form, hydrate thereof, or a pharmaceutically acceptable salt thereof.

13. A method for treating or ameliorating the effects of a condition associated with increased protein disulfide isomerase (PDI) activity in a subject in need thereof comprising administering to the subject an effective amount of a composition of claim 3.

14. The method of claim 11, wherein the condition is selected from the group consisting of a protein folding disorder, cancer, HIV, infection and a blood clot.

15. The method of claim 14, wherein the infection is influenza infection.

16. A method of modulating PDI activity in a cell comprising administering to the subject an effective amount of a compound according to claim 1.

17. The method of claim 16, wherein the compound is selected from the group consisting of: 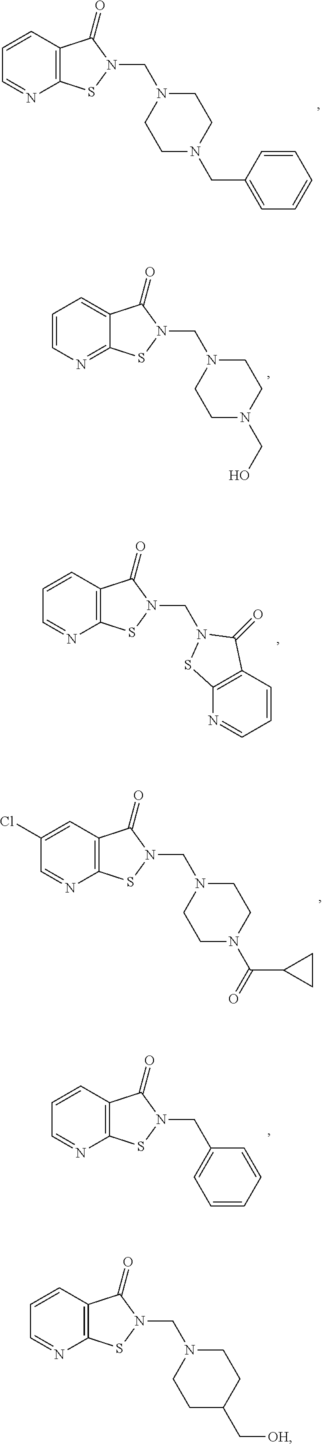 [SMILES: O=c1c2cccnc2sn1CN1CCN(Cc2ccccc2)CC1 ; O=c1c2cccnc2sn1CN1CCN(CO)CC1 ; O=c1c2cccnc2sn1Cn1sc2ncccc2c1=O ; O=C(C1CC1)N1CCN(Cn2sc3ncc(Cl)cc3c2=O)CC1 ; O=c1c2cccnc2sn1Cc1ccccc1 ; O=c1c2cccnc2sn1CN1CCC(CO)CC1] 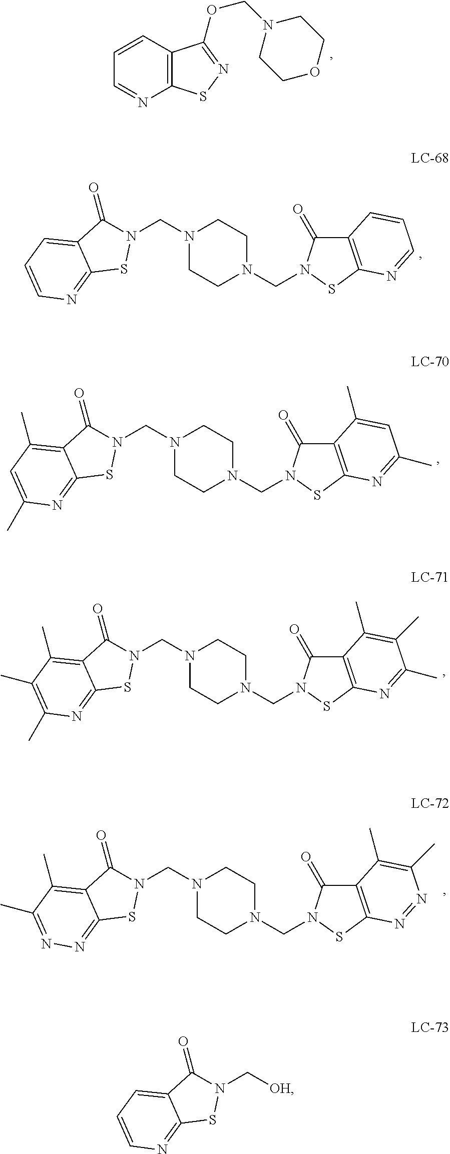
and combinations thereof, or an N-oxide, crystalline form, hydrate thereof, or a pharmaceutically acceptable salt thereof.

18. A method of modulating PDI activity in a cell comprising administering to the subject an effective amount of a composition of claim 3.


SEQUENCE LISTING (1 sequence)

SEQ ID NO: 1 - protein, 491 aa, Homo sapiens
DAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDEL